SEQUENCE LISTING (274 sequences)

SEQ ID NO: 1 - DNA, 73 bp, Artificial Sequence
  Description of Artificial Sequence Synthetic oligonucleotide
cgtgtaaagttaacctcgagggaggaaattacaccgacgcggggactgcgatatcaagcttatcgataatcaa

SEQ ID NO: 2 - DNA, 60 bp, Unknown
  Description of Unknown pre-injected SREBF1.g1 sequence
cagagcttcccagccccagccccaccgcagttcagctccacccctgtgttaggctacccc

SEQ ID NO: 3 - DNA, 40 bp, Unknown
  Description of Unknown pre-injected SREBF1.g1 sequence
cagccccagccccaccgcagttcagctccacccctgtgtt

SEQ ID NO: 4 - DNA, 40 bp, Artificial Sequence
  Description of Artificial Sequence Synthetic oligonucleotide
cagccccagccccaccgcagtttcagctccacccctgtgt

SEQ ID NO: 5 - DNA, 35 bp, Artificial Sequence
  Description of Artificial Sequence Synthetic oligonucleotide
cagccccagccccaccgcagctccacccctgtgtt

SEQ ID NO: 6 - DNA, 24 bp, Artificial Sequence
  Description of Artificial Sequence Synthetic oligonucleotide
cagccccagctccacccctgtgtt

SEQ ID NO: 7 - DNA, 24 bp, Artificial Sequence
  Description of Artificial Sequence Synthetic oligonucleotide
cagccccagccccaccgcagtgtt

SEQ ID NO: 8 - DNA, 39 bp, Artificial Sequence
  Description of Artificial Sequence Synthetic oligonucleotide
cagccccagccccaccgcagtcagctccacccctgtgtt

SEQ ID NO: 9 - DNA, 24 bp, Artificial Sequence
  Description of Artificial Sequence Synthetic oligonucleotide
cagccccagccccacccctgtgtt

SEQ ID NO: 10 - DNA, 38 bp, Artificial Sequence
  Description of Artificial Sequence Synthetic oligonucleotide
cagccccagccccaccgcagcagctccacccctgtgtt

SEQ ID NO: 11 - DNA, 24 bp, Artificial Sequence
  Description of Artificial Sequence Synthetic oligonucleotide
cagccccagccccaccgctgtgtt

SEQ ID NO: 12 - DNA, 40 bp, Artificial Sequence
  Description of Artificial Sequence Synthetic oligonucleotide
cagccccagccccaccgcagttttcagctccacccctgtg

SEQ ID NO: 13 - DNA, 36 bp, Artificial Sequence
  Description of Artificial Sequence Synthetic oligonucleotide
cagccccagccccaccgcaggctccacccctgtgtt

SEQ ID NO: 14 - DNA, 18 bp, Artificial Sequence
  Description of Artificial Sequence Synthetic oligonucleotide
cagctccacccctgtgtt

SEQ ID NO: 15 - DNA, 34 bp, Artificial Sequence
  Description of Artificial Sequence Synthetic oligonucleotide
cagccccagccccattcagctccacccctgtgtt

SEQ ID NO: 16 - DNA, 22 bp, Artificial Sequence
  Description of Artificial Sequence Synthetic oligonucleotide
cagccccagccccaccgcagtg

SEQ ID NO: 17 - DNA, 37 bp, Artificial Sequence
  Description of Artificial Sequence Synthetic oligonucleotide
cagccccagccccaccgcagagctccacccctgtgtt

SEQ ID NO: 18 - DNA, 24 bp, Artificial Sequence
  Description of Artificial Sequence Synthetic oligonucleotide
cagcctcagctccacccctgtgtt

SEQ ID NO: 19 - DNA, 33 bp, Artificial Sequence
  Description of Artificial Sequence Synthetic oligonucleotide
cagccccagccccttcagctccacccctgtgtt

SEQ ID NO: 20 - DNA, 40 bp, Artificial Sequence
  Description of Artificial Sequence Synthetic oligonucleotide
cagccccagccccaccgcagtattcagctccacccctgtg

SEQ ID NO: 21 - DNA, 38 bp, Artificial Sequence
  Description of Artificial Sequence Synthetic oligonucleotide
cagccccagccccaccgcagtagctccacccctgtgtt

SEQ ID NO: 22 - DNA, 37 bp, Artificial Sequence
  Description of Artificial Sequence Synthetic oligonucleotide
cagccccagccccaccgttcagctccacccctgtgtt

SEQ ID NO: 23 - DNA, 36 bp, Artificial Sequence
  Description of Artificial Sequence Synthetic oligonucleotide
cagccccagccccaccttcagctccacccctgtgtt

SEQ ID NO: 24 - DNA, 35 bp, Artificial Sequence
  Description of Artificial Sequence Synthetic oligonucleotide
cagccccagccccaccgcagcttcacccctgtgtt

SEQ ID NO: 25 - DNA, 40 bp, Artificial Sequence
  Description of Artificial Sequence Synthetic oligonucleotide
cagccccagccccaccgcagcttcagctccacccctgtgt

SEQ ID NO: 26 - DNA, 22 bp, Artificial Sequence
  Description of Artificial Sequence Synthetic oligonucleotide
cagccccagccccaccgcagtt

SEQ ID NO: 27 - DNA, 30 bp, Artificial Sequence
  Description of Artificial Sequence Synthetic oligonucleotide
cagccccagccccagctccacccctgtgtt

SEQ ID NO: 28 - DNA, 34 bp, Artificial Sequence
  Description of Artificial Sequence Synthetic oligonucleotide
cagccccagccccaccgcagtccacccctgtgtt

SEQ ID NO: 29 - DNA, 27 bp, Artificial Sequence
  Description of Artificial Sequence Synthetic oligonucleotide
cagccccagccccaccgctcctgtgtt

SEQ ID NO: 30 - DNA, 11 bp, Artificial Sequence
  Description of Artificial Sequence Synthetic oligonucleotide
acccctgtgtt

SEQ ID NO: 31 - DNA, 15 bp, Artificial Sequence
  Description of Artificial Sequence Synthetic oligonucleotide
ctccacccctgtgtt

SEQ ID NO: 32 - DNA, 36 bp, Artificial Sequence
  Description of Artificial Sequence Synthetic oligonucleotide
cagccccagccccaccgcagtctccacccctgtgtt

SEQ ID NO: 33 - DNA, 38 bp, Artificial Sequence
  Description of Artificial Sequence Synthetic oligonucleotide
cagccccagccccaccgcttcagctccacccctgtgtt

SEQ ID NO: 34 - DNA, 12 bp, Artificial Sequence
  Description of Artificial Sequence Synthetic oligonucleotide
cagccccagccc

SEQ ID NO: 35 - DNA, 32 bp, Artificial Sequence
  Description of Artificial Sequence Synthetic oligonucleotide
cagccccagccccaccgctccacccctgtgtt

SEQ ID NO: 36 - DNA, 18 bp, Artificial Sequence
  Description of Artificial Sequence Synthetic oligonucleotide
cagctccacccctgtgtt

SEQ ID NO: 37 - DNA, 34 bp, Artificial Sequence
  Description of Artificial Sequence Synthetic oligonucleotide
cagccccagcgcagtttcagctccacccctgtgt

SEQ ID NO: 38 - DNA, 31 bp, Artificial Sequence
  Description of Artificial Sequence Synthetic oligonucleotide
cagccccagccttcagctccacccctgtgtt

SEQ ID NO: 39 - DNA, 38 bp, Artificial Sequence
  Description of Artificial Sequence Synthetic oligonucleotide
cagccccagccccaccgcatcagctccacccctgtgtt

SEQ ID NO: 40 - DNA, 25 bp, Artificial Sequence
  Description of Artificial Sequence Synthetic oligonucleotide
cagccccagccccaccgcaggtgtt

SEQ ID NO: 41 - DNA, 38 bp, Artificial Sequence
  Description of Artificial Sequence Synthetic oligonucleotide
cagccccagccccaccgcagcagcaccacccctgtgtt

SEQ ID NO: 42 - DNA, 37 bp, Artificial Sequence
  Description of Artificial Sequence Synthetic oligonucleotide
cagccccagccccaccgcagtgctccacccctgtgtt

SEQ ID NO: 43 - DNA, 40 bp, Artificial Sequence
  Description of Artificial Sequence Synthetic oligonucleotide
cagccccagccccaccgcagtgttaggctaccccagctcc

SEQ ID NO: 44 - DNA, 14 bp, Artificial Sequence
  Description of Artificial Sequence Synthetic oligonucleotide
cagccccagcccca

SEQ ID NO: 45 - DNA, 40 bp, Artificial Sequence
  Description of Artificial Sequence Synthetic oligonucleotide
cagccccagccccaccgcagattcagctccacccctgtgt

SEQ ID NO: 46 - DNA, 40 bp, Artificial Sequence; identical to SEQ ID NO: 4
  Description of Artificial Sequence Synthetic oligonucleotide

SEQ ID NO: 47 - DNA, 39 bp, Artificial Sequence
  Description of Artificial Sequence Synthetic oligonucleotide
cagccccagccctccgggaggcttcttccctccacccct

SEQ ID NO: 48 - DNA, 18 bp, Artificial Sequence
  Description of Artificial Sequence Synthetic oligonucleotide
cagccccagccctgtgtt

SEQ ID NO: 49 - DNA, 13 bp, Artificial Sequence
  Description of Artificial Sequence Synthetic oligonucleotide
ccacccctgtgtt

SEQ ID NO: 50 - DNA, 39 bp, Artificial Sequence
  Description of Artificial Sequence Synthetic oligonucleotide
cagccccagccccaccgcattcagctccacccctgtgtt

SEQ ID NO: 51 - DNA, 20 bp, Artificial Sequence
  Description of Artificial Sequence Synthetic oligonucleotide
cagccccagccccatgtgtt

SEQ ID NO: 52 - DNA, 60 bp, Unknown; identical to SEQ ID NO: 2
  Description of Unknown brain-derived SREBF1.g1 sequence

SEQ ID NO: 53 - DNA, 40 bp, Unknown; identical to SEQ ID NO: 3
  Description of Unknown brain-derived SREBF1.g1 sequence

SEQ ID NO: 54 - DNA, 24 bp, Artificial Sequence
  Description of Artificial Sequence Synthetic oligonucleotide
cagccccagccccaccggggctcc

SEQ ID NO: 55 - DNA, 40 bp, Artificial Sequence
  Description of Artificial Sequence Synthetic oligonucleotide
cagccccagccccaccgcagtgttcagctccacccctgtg

SEQ ID NO: 56 - DNA, 24 bp, Artificial Sequence
  Description of Artificial Sequence Synthetic oligonucleotide
cagccccagccccacccctgtgtt

SEQ ID NO: 57 - DNA, 40 bp, Artificial Sequence; identical to SEQ ID NO: 4
  Description of Artificial Sequence Synthetic oligonucleotide

SEQ ID NO: 58 - DNA, 35 bp, Artificial Sequence; identical to SEQ ID NO: 5
  Description of Artificial Sequence Synthetic oligonucleotide

SEQ ID NO: 59 - DNA, 34 bp, Artificial Sequence; identical to SEQ ID NO: 15
  Description of Artificial Sequence Synthetic oligonucleotide

SEQ ID NO: 60 - DNA, 24 bp, Artificial Sequence
  Description of Artificial Sequence Synthetic oligonucleotide
cagccccagccccaccgcagtgtt

SEQ ID NO: 61 - DNA, 24 bp, Artificial Sequence
  Description of Artificial Sequence Synthetic oligonucleotide
cagccccagctccacccctgtgtt

SEQ ID NO: 62 - DNA, 39 bp, Artificial Sequence; identical to SEQ ID NO: 50
  Description of Artificial Sequence Synthetic oligonucleotide

SEQ ID NO: 63 - DNA, 40 bp, Artificial Sequence; identical to SEQ ID NO: 55
  Description of Artificial Sequence Synthetic oligonucleotide

SEQ ID NO: 64 - DNA, 24 bp, Artificial Sequence
  Description of Artificial Sequence Synthetic oligonucleotide
cagccccagccccacccctgtgtt

SEQ ID NO: 65 - DNA, 24 bp, Artificial Sequence
  Description of Artificial Sequence Synthetic oligonucleotide
cagccccagccccaccggggctcc

SEQ ID NO: 66 - DNA, 40 bp, Artificial Sequence
  Description of Artificial Sequence Synthetic oligonucleotide
cagccccagccccaccgcagtgctcagctccacccctgtg

SEQ ID NO: 67 - DNA, 60 bp, Unknown
  Description of Unknown pre-injected SREBF1.g2 sequence
actgtgaaggcggcaggtctcagtcccctggtctctggcaccactgtgcagacagggcct

SEQ ID NO: 68 - DNA, 40 bp, Unknown
  Description of Unknown pre-injected SREBF1.g2 sequence
cggcaggtctcagtcccctggtctctggcaccactgtgca

SEQ ID NO: 69 - DNA, 33 bp, Artificial Sequence
  Description of Artificial Sequence Synthetic oligonucleotide
cggcaggtctcagtcccctggcaccactgtgca

SEQ ID NO: 70 - DNA, 39 bp, Artificial Sequence
  Description of Artificial Sequence Synthetic oligonucleotide
cggcaggtctcagtcccctgtctctggcaccactgtgca

SEQ ID NO: 71 - DNA, 32 bp, Artificial Sequence
  Description of Artificial Sequence Synthetic oligonucleotide
cggcaggtctcagtctctggcaccactgtgca

SEQ ID NO: 72 - DNA, 37 bp, Artificial Sequence
  Description of Artificial Sequence Synthetic oligonucleotide
cggcaggtctcagtcccctggctggcaccactgtgca

SEQ ID NO: 73 - DNA, 24 bp, Artificial Sequence
  Description of Artificial Sequence Synthetic oligonucleotide
cggcaggtctcagtcccctgtgca

SEQ ID NO: 74 - DNA, 26 bp, Artificial Sequence
  Description of Artificial Sequence Synthetic oligonucleotide
cggcaggtctctggcaccactgtgca

SEQ ID NO: 75 - DNA, 24 bp, Artificial Sequence
  Description of Artificial Sequence Synthetic oligonucleotide
cggcaggtctcagtccactgtgca

SEQ ID NO: 76 - DNA, 37 bp, Artificial Sequence
  Description of Artificial Sequence Synthetic oligonucleotide
cggcaggtctcagtcccctgtctggcaccactgtgca

SEQ ID NO: 77 - DNA, 36 bp, Artificial Sequence
  Description of Artificial Sequence Synthetic oligonucleotide
cggcaggtctcagtcccctgctggcaccactgtgca

SEQ ID NO: 78 - DNA, 25 bp, Artificial Sequence
  Description of Artificial Sequence Synthetic oligonucleotide
cggcaggtctcagtcccactgtgca

SEQ ID NO: 79 - DNA, 40 bp, Artificial Sequence
  Description of Artificial Sequence Synthetic oligonucleotide
cggcaggtctcagtcccctggggtctctggcaccactgtg

SEQ ID NO: 80 - DNA, 25 bp, Artificial Sequence
  Description of Artificial Sequence Synthetic oligonucleotide
cggcaggtctcagtcccctggtgca

SEQ ID NO: 81 - DNA, 39 bp, Artificial Sequence
  Description of Artificial Sequence Synthetic oligonucleotide
cggcaggtctcagtcccctggctctggcaccactgtgca

SEQ ID NO: 82 - DNA, 34 bp, Artificial Sequence
  Description of Artificial Sequence Synthetic oligonucleotide
cggcaggtctcagtcccctgtgcaccactgtgca

SEQ ID NO: 83 - DNA, 35 bp, Artificial Sequence
  Description of Artificial Sequence Synthetic oligonucleotide
cggcaggtctcagtcccctgtggcaccactgtgca

SEQ ID NO: 84 - DNA, 26 bp, Artificial Sequence
  Description of Artificial Sequence Synthetic oligonucleotide
cggcaggtctcagtccccactgtgca

SEQ ID NO: 85 - DNA, 32 bp, Artificial Sequence
  Description of Artificial Sequence Synthetic oligonucleotide
cggcaggtctcagtcccctgcaccactgtgca

SEQ ID NO: 86 - DNA, 15 bp, Artificial Sequence
  Description of Artificial Sequence Synthetic oligonucleotide
cggcaccactgtgca

SEQ ID NO: 87 - DNA, 33 bp, Artificial Sequence
  Description of Artificial Sequence Synthetic oligonucleotide
cggcaggtctcagtcctctggcaccactgtgca

SEQ ID NO: 88 - DNA, 35 bp, Artificial Sequence
  Description of Artificial Sequence Synthetic oligonucleotide
cggcaggtctcagtcccctggggcaccactgtgca

SEQ ID NO: 89 - DNA, 31 bp, Artificial Sequence
  Description of Artificial Sequence Synthetic oligonucleotide
cggcaggtctcgtctctggcaccactgtgca

SEQ ID NO: 90 - DNA, 40 bp, Artificial Sequence
  Description of Artificial Sequence Synthetic oligonucleotide
cggcaggtctcagtcccctgtgagaccaggtctctggcac

SEQ ID NO: 91 - DNA, 38 bp, Artificial Sequence
  Description of Artificial Sequence Synthetic oligonucleotide
cggcaggtctcagtcccctggtctggcaccactgtgca

SEQ ID NO: 92 - DNA, 40 bp, Artificial Sequence
  Description of Artificial Sequence Synthetic oligonucleotide
cggcaggtctcagtcccctcagtctctggcaccactgtgc

SEQ ID NO: 93 - DNA, 36 bp, Artificial Sequence
  Description of Artificial Sequence Synthetic oligonucleotide
cggcaggtctcagtcccctggtggcaccactgtgca

SEQ ID NO: 94 - DNA, 21 bp, Artificial Sequence
  Description of Artificial Sequence Synthetic oligonucleotide
cggcaggtctcagtcccctgg

SEQ ID NO: 95 - DNA, 35 bp, Artificial Sequence
  Description of Artificial Sequence Synthetic oligonucleotide
cggcaggtctcagtcccctctggcaccactgtgca

SEQ ID NO: 96 - DNA, 12 bp, Artificial Sequence
  Description of Artificial Sequence Synthetic oligonucleotide
caccactgtgca

SEQ ID NO: 97 - DNA, 23 bp, Artificial Sequence
  Description of Artificial Sequence Synthetic oligonucleotide
cggcaggtctcagtcccctggca

SEQ ID NO: 98 - DNA, 40 bp, Artificial Sequence
  Description of Artificial Sequence Synthetic oligonucleotide
cggcaggtctcagtcccctgtctcagtcctcagtctctgg

SEQ ID NO: 99 - DNA, 38 bp, Artificial Sequence
  Description of Artificial Sequence Synthetic oligonucleotide
cggcaggtctcagtcccctgggctggcaccactgtgca

SEQ ID NO: 100 - DNA, 21 bp, Artificial Sequence
  Description of Artificial Sequence Synthetic oligonucleotide
cggcaggtctcagtcccctga

SEQ ID NO: 101 - DNA, 20 bp, Artificial Sequence
  Description of Artificial Sequence Synthetic oligonucleotide
cggcaggtctcagtcccctg

SEQ ID NO: 102 - DNA, 25 bp, Artificial Sequence
  Description of Artificial Sequence Synthetic oligonucleotide
cggcaggtctctggaccactgtgca

SEQ ID NO: 103 - DNA, 16 bp, Artificial Sequence
  Description of Artificial Sequence Synthetic oligonucleotide
cggcaggtctcacaaa

SEQ ID NO: 104 - DNA, 31 bp, Artificial Sequence
  Description of Artificial Sequence Synthetic oligonucleotide
cggcaggtctcagtcccctggccactgtgca

SEQ ID NO: 105 - DNA, 11 bp, Artificial Sequence
  Description of Artificial Sequence Synthetic oligonucleotide
cggcaggtgca

SEQ ID NO: 106 - DNA, 40 bp, Artificial Sequence
  Description of Artificial Sequence Synthetic oligonucleotide
cggcaggtctcagtcccctgagacttggtgtctctggcac

SEQ ID NO: 107 - DNA, 20 bp, Artificial Sequence
  Description of Artificial Sequence Synthetic oligonucleotide
cggcaggtctcagtcccctg

SEQ ID NO: 108 - DNA, 19 bp, Artificial Sequence
  Description of Artificial Sequence Synthetic oligonucleotide
cggcaggtctcagtcccct

SEQ ID NO: 109 - DNA, 23 bp, Artificial Sequence
  Description of Artificial Sequence Synthetic oligonucleotide
cggcaggtctcagtccctgtgca

SEQ ID NO: 110 - DNA, 40 bp, Artificial Sequence
  Description of Artificial Sequence Synthetic oligonucleotide
cggcaggtctcagtcccctgggtctctggcaccactgtgc

SEQ ID NO: 111 - DNA, 34 bp, Artificial Sequence
  Description of Artificial Sequence Synthetic oligonucleotide
cggcaggtctcagtcccctgggcaccactgtgca

SEQ ID NO: 112 - DNA, 23 bp, Artificial Sequence
  Description of Artificial Sequence Synthetic oligonucleotide
cggcaggtctcagtcccctgggg

SEQ ID NO: 113 - DNA, 24 bp, Artificial Sequence
  Description of Artificial Sequence Synthetic oligonucleotide
cggcaggtctggcaccactgtgca

SEQ ID NO: 114 - DNA, 37 bp, Artificial Sequence
  Description of Artificial Sequence Synthetic oligonucleotide
cggcaggtctcagtcccctctctggcaccactgtgca

SEQ ID NO: 115 - DNA, 37 bp, Artificial Sequence
  Description of Artificial Sequence Synthetic oligonucleotide
cggcaggtctcagtcccgtctctggcaccactgtgca

SEQ ID NO: 116 - DNA, 39 bp, Artificial Sequence
  Description of Artificial Sequence Synthetic oligonucleotide
cggcaggtctcagtccccactcaccagggtctgcagggc

SEQ ID NO: 117 - DNA, 60 bp, Unknown; identical to SEQ ID NO: 67
  Description of Unknown brain-derived SREBF1.g2 sequence

SEQ ID NO: 118 - DNA, 40 bp, Unknown; identical to SEQ ID NO: 68
  Description of Unknown brain-derived SREBF1.g2 sequence

SEQ ID NO: 119 - DNA, 39 bp, Artificial Sequence; identical to SEQ ID NO: 70
  Description of Artificial Sequence Synthetic oligonucleotide

SEQ ID NO: 120 - DNA, 30 bp, Artificial Sequence
  Description of Artificial Sequence Synthetic oligonucleotide
cggcaggtctcagtcccctggcactgtgca

SEQ ID NO: 121 - DNA, 26 bp, Artificial Sequence
  Description of Artificial Sequence Synthetic oligonucleotide
cggcaggtctctggcaccactgtgca

SEQ ID NO: 122 - DNA, 37 bp, Artificial Sequence; identical to SEQ ID NO: 72
  Description of Artificial Sequence Synthetic oligonucleotide

SEQ ID NO: 123 - DNA, 33 bp, Artificial Sequence; identical to SEQ ID NO: 69
  Description of Artificial Sequence Synthetic oligonucleotide

SEQ ID NO: 124 - DNA, 30 bp, Artificial Sequence
  Description of Artificial Sequence Synthetic oligonucleotide
cggcaggtctcagtctggcaccactgtgca

SEQ ID NO: 125 - DNA, 38 bp, Artificial Sequence
  Description of Artificial Sequence Synthetic oligonucleotide
cggcaggtctcagtcccctgctctggcaccactgtgca

SEQ ID NO: 126 - DNA, 30 bp, Artificial Sequence
  Description of Artificial Sequence Synthetic oligonucleotide
cggcaggtctcagtcccctgagcatgtgca

SEQ ID NO: 127 - DNA, 37 bp, Artificial Sequence
  Description of Artificial Sequence Synthetic oligonucleotide
cggcaggtctcagtcctgtgctgcaccacagtctgca

SEQ ID NO: 128 - DNA, 38 bp, Artificial Sequence; identical to SEQ ID NO: 91
  Description of Artificial Sequence Synthetic oligonucleotide

SEQ ID NO: 129 - DNA, 25 bp, Artificial Sequence
  Description of Artificial Sequence Synthetic oligonucleotide
cggcaggtctcagtcccactgtgca

SEQ ID NO: 130 - DNA, 38 bp, Artificial Sequence; identical to SEQ ID NO: 99
  Description of Artificial Sequence Synthetic oligonucleotide

SEQ ID NO: 131 - DNA, 32 bp, Artificial Sequence; identical to SEQ ID NO: 71
  Description of Artificial Sequence Synthetic oligonucleotide

SEQ ID NO: 132 - DNA, 39 bp, Artificial Sequence; identical to SEQ ID NO: 70
  Description of Artificial Sequence Synthetic oligonucleotide

SEQ ID NO: 133 - DNA, 15 bp, Artificial Sequence
  Description of Artificial Sequence Synthetic oligonucleotide
cggcaccactgtgca

SEQ ID NO: 134 - DNA, 60 bp, Unknown; identical to SEQ ID NO: 2
  Description of Unknown pre-injected SREBF1.g1 sequence

SEQ ID NO: 135 - DNA, 40 bp, Unknown; identical to SEQ ID NO: 3
  Description of Unknown pre-injected SREBF1.g1 sequence

SEQ ID NO: 136 - DNA, 40 bp, Artificial Sequence; identical to SEQ ID NO: 4
  Description of Artificial Sequence Synthetic oligonucleotide

SEQ ID NO: 137 - DNA, 35 bp, Artificial Sequence; identical to SEQ ID NO: 5
  Description of Artificial Sequence Synthetic oligonucleotide

SEQ ID NO: 138 - DNA, 24 bp, Artificial Sequence
  Description of Artificial Sequence Synthetic oligonucleotide
cagccccagctccacccctgtgtt

SEQ ID NO: 139 - DNA, 24 bp, Artificial Sequence
  Description of Artificial Sequence Synthetic oligonucleotide
cagccccagccccacccctgtgtt

SEQ ID NO: 140 - DNA, 24 bp, Artificial Sequence
  Description of Artificial Sequence Synthetic oligonucleotide
cagccccagccccaccgcagtgtt

SEQ ID NO: 141 - DNA, 40 bp, Artificial Sequence; identical to SEQ ID NO: 45
  Description of Artificial Sequence Synthetic oligonucleotide

SEQ ID NO: 142 - DNA, 24 bp, Artificial Sequence
  Description of Artificial Sequence Synthetic oligonucleotide
cagccccagccccaccgctgtgtt

SEQ ID NO: 143 - DNA, 40 bp, Artificial Sequence
  Description of Artificial Sequence Synthetic oligonucleotide
cagccccagccccaccgcagtggggctccacccctgtgtt

SEQ ID NO: 144 - DNA, 39 bp, Artificial Sequence; identical to SEQ ID NO: 8
  Description of Artificial Sequence Synthetic oligonucleotide

SEQ ID NO: 145 - DNA, 40 bp, Artificial Sequence; identical to SEQ ID NO: 12
  Description of Artificial Sequence Synthetic oligonucleotide

SEQ ID NO: 146 - DNA, 34 bp, Artificial Sequence; identical to SEQ ID NO: 15
  Description of Artificial Sequence Synthetic oligonucleotide

SEQ ID NO: 147 - DNA, 18 bp, Artificial Sequence
  Description of Artificial Sequence Synthetic oligonucleotide
cagctccacccctgtgtt

SEQ ID NO: 148 - DNA, 19 bp, Artificial Sequence
  Description of Artificial Sequence Synthetic oligonucleotide
cagccccagcctgtgtgtt

SEQ ID NO: 149 - DNA, 38 bp, Artificial Sequence; identical to SEQ ID NO: 10
  Description of Artificial Sequence Synthetic oligonucleotide

SEQ ID NO: 150 - DNA, 33 bp, Artificial Sequence; identical to SEQ ID NO: 19
  Description of Artificial Sequence Synthetic oligonucleotide

SEQ ID NO: 151 - DNA, 22 bp, Artificial Sequence
  Description of Artificial Sequence Synthetic oligonucleotide
cagccccagccccaccgcagtt

SEQ ID NO: 152 - DNA, 40 bp, Artificial Sequence; identical to SEQ ID NO: 20
  Description of Artificial Sequence Synthetic oligonucleotide

SEQ ID NO: 153 - DNA, 35 bp, Artificial Sequence
  Description of Artificial Sequence Synthetic oligonucleotide
cagccccagccccaccgctgctccacccctgtgtt

SEQ ID NO: 154 - DNA, 28 bp, Artificial Sequence
  Description of Artificial Sequence Synthetic oligonucleotide
cagccccagccagctccacccctgtgtt

SEQ ID NO: 155 - DNA, 17 bp, Artificial Sequence
  Description of Artificial Sequence Synthetic oligonucleotide
cagccccagccccaccg

SEQ ID NO: 156 - DNA, 40 bp, Artificial Sequence
  Description of Artificial Sequence Synthetic oligonucleotide
cagccccagccccaccgcagtgttaggctccacccctgtg

SEQ ID NO: 157 - DNA, 30 bp, Artificial Sequence
  Description of Artificial Sequence Synthetic oligonucleotide
cagccccagccccaccgcagtgttatgtgt

SEQ ID NO: 158 - DNA, 34 bp, Artificial Sequence
  Description of Artificial Sequence Synthetic oligonucleotide
cagccccagccccaccgcagctccaccctgtgtt

SEQ ID NO: 159 - DNA, 30 bp, Artificial Sequence; identical to SEQ ID NO: 27
  Description of Artificial Sequence Synthetic oligonucleotide

SEQ ID NO: 160 - DNA, 39 bp, Artificial Sequence
  Description of Artificial Sequence Synthetic oligonucleotide
cagccccagccccacccagcccccagccccagctccacc

SEQ ID NO: 161 - DNA, 31 bp, Artificial Sequence
  Description of Artificial Sequence Synthetic oligonucleotide
cagccccagccccaccgcacacccctgtgtt

SEQ ID NO: 162 - DNA, 40 bp, Artificial Sequence; identical to SEQ ID NO: 4
  Description of Artificial Sequence Synthetic oligonucleotide

SEQ ID NO: 163 - DNA, 40 bp, Artificial Sequence; identical to SEQ ID NO: 25
  Description of Artificial Sequence Synthetic oligonucleotide

SEQ ID NO: 164 - DNA, 37 bp, Artificial Sequence
  Description of Artificial Sequence Synthetic oligonucleotide
cagccccagccccaccgcagtgtaacacccctgtgtt

SEQ ID NO: 165 - DNA, 19 bp, Artificial Sequence
  Description of Artificial Sequence Synthetic oligonucleotide
cagccccagcccctgtgtt

SEQ ID NO: 166 - DNA, 37 bp, Artificial Sequence; identical to SEQ ID NO: 17
  Description of Artificial Sequence Synthetic oligonucleotide

SEQ ID NO: 167 - DNA, 35 bp, Artificial Sequence; identical to SEQ ID NO: 5
  Description of Artificial Sequence Synthetic oligonucleotide

SEQ ID NO: 168 - DNA, 18 bp, Artificial Sequence
  Description of Artificial Sequence Synthetic oligonucleotide
cagctccacccctgtgtt

SEQ ID NO: 169 - DNA, 23 bp, Artificial Sequence
  Description of Artificial Sequence Synthetic oligonucleotide
cagccccagcccacccctgtgtt

SEQ ID NO: 170 - DNA, 34 bp, Artificial Sequence; identical to SEQ ID NO: 28
  Description of Artificial Sequence Synthetic oligonucleotide

SEQ ID NO: 171 - DNA, 40 bp, Artificial Sequence; identical to SEQ ID NO: 3
  Description of Artificial Sequence Synthetic oligonucleotide

SEQ ID NO: 172 - DNA, 11 bp, Artificial Sequence
  Description of Artificial Sequence Synthetic oligonucleotide
acccctgtgtt

SEQ ID NO: 173 - DNA, 38 bp, Artificial Sequence
  Description of Artificial Sequence Synthetic oligonucleotide
cagccccagccccaccgtttcagctccacccctgtgtt

SEQ ID NO: 174 - DNA, 20 bp, Artificial Sequence
  Description of Artificial Sequence Synthetic oligonucleotide
cagccccagccccaccgcag

SEQ ID NO: 175 - DNA, 38 bp, Artificial Sequence
  Description of Artificial Sequence Synthetic oligonucleotide
cagccccagccccgcctccccagctccacccctgtgtt

SEQ ID NO: 176 - DNA, 15 bp, Artificial Sequence
  Description of Artificial Sequence Synthetic oligonucleotide
cagccccagcccgtt

SEQ ID NO: 177 - DNA, 60 bp, Unknown; identical to SEQ ID NO: 2
  Description of Unknown brain-derived SREBF1.g1 sequence

SEQ ID NO: 178 - DNA, 40 bp, Unknown; identical to SEQ ID NO: 3
  Description of Unknown brain-derived SREBF1.g1 sequence

SEQ ID NO: 179 - DNA, 24 bp, Artificial Sequence
  Description of Artificial Sequence Synthetic oligonucleotide
cagccccagccccacccctgtgtt

SEQ ID NO: 180 - DNA, 24 bp, Artificial Sequence
  Description of Artificial Sequence Synthetic oligonucleotide
cagccccagccccaccgcagtgtt

SEQ ID NO: 181 - DNA, 20 bp, Artificial Sequence
  Description of Artificial Sequence Synthetic oligonucleotide
ttcagctccacccctgtgtt

SEQ ID NO: 182 - DNA, 35 bp, Artificial Sequence; identical to SEQ ID NO: 5
  Description of Artificial Sequence Synthetic oligonucleotide

SEQ ID NO: 183 - DNA, 40 bp, Artificial Sequence; identical to SEQ ID NO: 4
  Description of Artificial Sequence Synthetic oligonucleotide

SEQ ID NO: 184 - DNA, 24 bp, Artificial Sequence
  Description of Artificial Sequence Synthetic oligonucleotide
cagccccagctccacccctgtgtt

SEQ ID NO: 185 - DNA, 38 bp, Artificial Sequence; identical to SEQ ID NO: 10
  Description of Artificial Sequence Synthetic oligonucleotide

SEQ ID NO: 186 - DNA, 24 bp, Artificial Sequence
  Description of Artificial Sequence Synthetic oligonucleotide
cagccccagccccacccctgtgtt

SEQ ID NO: 187 - DNA, 40 bp, Artificial Sequence; identical to SEQ ID NO: 12
  Description of Artificial Sequence Synthetic oligonucleotide

SEQ ID NO: 188 - DNA, 73 bp, Artificial Sequence
  Description of Artificial Sequence Synthetic oligonucleotide
cgtgtaaagttaacctcgagggaaccaaaacgctgcagctggcctacgcgatatcaagcttatcgataatcaa

SEQ ID NO: 189 - DNA, 73 bp, Artificial Sequence; identical to SEQ ID NO: 1
  Description of Artificial Sequence Synthetic oligonucleotide

SEQ ID NO: 190 - DNA, 73 bp, Artificial Sequence
  Description of Artificial Sequence Synthetic oligonucleotide
cgtgtaaagttaacctcgagggagtcgctgcgggttcaatcctggatgcgatatcaagcttatcgataatcaa

SEQ ID NO: 191 - DNA, 73 bp, Artificial Sequence
  Description of Artificial Sequence Synthetic oligonucleotide
cgtgtaaagttaacctcgagggcatgcagccagacgccctaaggtcatcgatatcaagcttatcgataatcaa

SEQ ID NO: 192 - DNA, 73 bp, Artificial Sequence
  Description of Artificial Sequence Synthetic oligonucleotide
cgtgtaaagttaacctcgagggccgacgcgagggttgttctgtgatgacgatatcaagcttatcgataatcaa

SEQ ID NO: 193 - DNA, 73 bp, Artificial Sequence
  Description of Artificial Sequence Synthetic oligonucleotide
cgtgtaaagttaacctcgaggggtccgaagacgtctcgcctgcatcaacgatatcaagcttatcgataatcaa

SEQ ID NO: 194 - DNA, 73 bp, Artificial Sequence
  Description of Artificial Sequence Synthetic oligonucleotide
cgtgtaaagttaacctcgaggggtgtgacagaaattcctgcaggcggccgatatcaagcttatcgataatcaa

SEQ ID NO: 195 - DNA, 73 bp, Artificial Sequence
  Description of Artificial Sequence Synthetic oligonucleotide
cgtgtaaagttaacctcgagggttcggccgctcgaaccacgtaagtcacgatatcaagcttatcgataatcaa

SEQ ID NO: 196 - DNA, 73 bp, Artificial Sequence; identical to SEQ ID NO: 190
  Description of Artificial Sequence Synthetic oligonucleotide

SEQ ID NO: 197 - DNA, 73 bp, Artificial Sequence; identical to SEQ ID NO: 193
  Description of Artificial Sequence Synthetic oligonucleotide

SEQ ID NO: 198 - DNA, 73 bp, Artificial Sequence; identical to SEQ ID NO: 195
  Description of Artificial Sequence Synthetic oligonucleotide

SEQ ID NO: 199 - DNA, 73 bp, Artificial Sequence
  Description of Artificial Sequence Synthetic oligonucleotide
cgtgtaaagttaacctcgagggacagctttcgacgggtccaagcagcccgatatcaagcttatcgataatcaa

SEQ ID NO: 200 - DNA, 73 bp, Artificial Sequence
  Description of Artificial Sequence Synthetic oligonucleotide
cgtgtaaagttaacctcgagggacgtccggcccctcacaagcacattccgatatcaagcttatcgataatcaa

SEQ ID NO: 201 - DNA, 73 bp, Artificial Sequence
  Description of Artificial Sequence Synthetic oligonucleotide
cgtgtaaagttaacctcgagggataagcggcgctcggtagactgcggtcgatatcaagcttatcgataatcaa

SEQ ID NO: 202 - DNA, 73 bp, Artificial Sequence
  Description of Artificial Sequence Synthetic oligonucleotide
cgtgtaaagttaacctcgagggcctgggcattcgtgtgtccaccccttcgatatcaagcttatcgataatcaa

SEQ ID NO: 203 - DNA, 73 bp, Artificial Sequence
  Description of Artificial Sequence Synthetic oligonucleotide
cgtgtaaagttaacctcgagggcgccgaggttgaagcacggttggaaccgatatcaagcttatcgataatcaa

SEQ ID NO: 204 - DNA, 73 bp, Artificial Sequence
  Description of Artificial Sequence Synthetic oligonucleotide
cgtgtaaagttaacctcgagggctggcgggcaatacctgcgagtaacgcgatatcaagcttatcgataatcaa

SEQ ID NO: 205 - DNA, 73 bp, Artificial Sequence; identical to SEQ ID NO: 191
  Description of Artificial Sequence Synthetic oligonucleotide

SEQ ID NO: 206 - DNA, 73 bp, Artificial Sequence; identical to SEQ ID NO: 192
  Description of Artificial Sequence Synthetic oligonucleotide

SEQ ID NO: 207 - DNA, 73 bp, Artificial Sequence; identical to SEQ ID NO: 194
  Description of Artificial Sequence Synthetic oligonucleotide

SEQ ID NO: 208 - DNA, 73 bp, Artificial Sequence
  Description of Artificial Sequence Synthetic oligonucleotide
cgtgtaaagttaacctcgagggagacttgtccagccgcggcgtagatccgatatcaagcttatcgataatcaa

SEQ ID NO: 209 - DNA, 73 bp, Artificial Sequence
  Description of Artificial Sequence Synthetic oligonucleotide
cgtgtaaagttaacctcgagggcaatcaggtagacgggacgcgtgacgcgatatcaagcttatcgataatcaa

SEQ ID NO: 210 - DNA, 73 bp, Artificial Sequence
  Description of Artificial Sequence Synthetic oligonucleotide
cgtgtaaagttaacctcgagggcaggcacctcgtagcagtgctttgcccgatatcaagcttatcgataatcaa

SEQ ID NO: 211 - DNA, 73 bp, Artificial Sequence
  Description of Artificial Sequence Synthetic oligonucleotide
cgtgtaaagttaacctcgagggccccactgtgcccgttcaccagtactcgatatcaagcttatcgataatcaa

SEQ ID NO: 212 - DNA, 73 bp, Artificial Sequence
  Description of Artificial Sequence Synthetic oligonucleotide
cgtgtaaagttaacctcgagggccgcctgccagagctaaggtcggttacgatatcaagcttatcgataatcaa

SEQ ID NO: 213 - DNA, 73 bp, Artificial Sequence
  Description of Artificial Sequence Synthetic oligonucleotide
cgtgtaaagttaacctcgagggcggccctcggtatcctcagatgtccacgatatcaagcttatcgataatcaa

SEQ ID NO: 214 - DNA, 73 bp, Artificial Sequence
  Description of Artificial Sequence Synthetic oligonucleotide
cgtgtaaagttaacctcgagggcgtagcagcaagcgcctagccagtctcgatatcaagcttatcgataatcaa

SEQ ID NO: 215 - DNA, 20 bp, Artificial Sequence
  Description of Artificial Sequence Synthetic oligonucleotide
agtctgaagcactaagaagg

SEQ ID NO: 216 - DNA, 20 bp, Artificial Sequence
  Description of Artificial Sequence Synthetic oligonucleotide
gttatgacttgctgtcaaga

SEQ ID NO: 217 - DNA, 20 bp, Artificial Sequence
  Description of Artificial Sequence Synthetic oligonucleotide
agagcaattcgagattacca

SEQ ID NO: 218 - DNA, 20 bp, Artificial Sequence
  Description of Artificial Sequence Synthetic oligonucleotide
gccagcgggagcacatcggt

SEQ ID NO: 219 - DNA, 20 bp, Artificial Sequence
  Description of Artificial Sequence Synthetic oligonucleotide
tatctaaagtatcacatcca

SEQ ID NO: 220 - DNA, 20 bp, Artificial Sequence
  Description of Artificial Sequence Synthetic oligonucleotide
gtggtgaagagtaaccaatg

SEQ ID NO: 221 - DNA, 20 bp, Artificial Sequence
  Description of Artificial Sequence Synthetic oligonucleotide
agcatccatagtacgccacg

SEQ ID NO: 222 - DNA, 20 bp, Artificial Sequence
  Description of Artificial Sequence Synthetic oligonucleotide
ttccaaatgagcataaccaa

SEQ ID NO: 223 - DNA, 20 bp, Artificial Sequence
  Description of Artificial Sequence Synthetic oligonucleotide
ggcgcccaccaggagcacca

SEQ ID NO: 224 - DNA, 20 bp, Artificial Sequence
  Description of Artificial Sequence Synthetic oligonucleotide
aagccgtcagaaatcagcca

SEQ ID NO: 225 - DNA, 20 bp, Artificial Sequence
  Description of Artificial Sequence Synthetic oligonucleotide
ttcactatcagttggaacag

SEQ ID NO: 226 - DNA, 20 bp, Artificial Sequence
  Description of Artificial Sequence Synthetic oligonucleotide
aggataaaacagaccaactg

SEQ ID NO: 227 - DNA, 20 bp, Artificial Sequence
  Description of Artificial Sequence Synthetic oligonucleotide
ggttaccacccaatgtcacg

SEQ ID NO: 228 - DNA, 20 bp, Artificial Sequence
  Description of Artificial Sequence Synthetic oligonucleotide
gctgaccaagaagttctgtg

SEQ ID NO: 229 - DNA, 20 bp, Artificial Sequence
  Description of Artificial Sequence Synthetic oligonucleotide
gttctcgcataggggtcacg

SEQ ID NO: 230 - DNA, 20 bp, Artificial Sequence
  Description of Artificial Sequence Synthetic oligonucleotide
ttgctgaagagagtttggtg

SEQ ID NO: 231 - DNA, 20 bp, Artificial Sequence
  Description of Artificial Sequence Synthetic oligonucleotide
cccacaagggaacagcacat

SEQ ID NO: 232 - DNA, 20 bp, Artificial Sequence
  Description of Artificial Sequence Synthetic oligonucleotide
agccaggtttcacaatctgg

SEQ ID NO: 233 - DNA, 20 bp, Artificial Sequence
  Description of Artificial Sequence Synthetic oligonucleotide
ccacgacatctcgcacaacg

SEQ ID NO: 234 - DNA, 20 bp, Artificial Sequence
  Description of Artificial Sequence Synthetic oligonucleotide
ctccttcaagaagtaccacg

SEQ ID NO: 235 - DNA, 20 bp, Artificial Sequence
  Description of Artificial Sequence Synthetic oligonucleotide
ctggctcaataggtccaagg

SEQ ID NO: 236 - DNA, 20 bp, Artificial Sequence
  Description of Artificial Sequence Synthetic oligonucleotide
ccaggcccatgataccatgg

SEQ ID NO: 237 - DNA, 20 bp, Artificial Sequence
  Description of Artificial Sequence Synthetic oligonucleotide
gatgtattcaaatgactcag

SEQ ID NO: 238 - DNA, 20 bp, Artificial Sequence
  Description of Artificial Sequence Synthetic oligonucleotide
gagcatgctgaacgacatcg

SEQ ID NO: 239 - DNA, 20 bp, Artificial Sequence
  Description of Artificial Sequence Synthetic oligonucleotide
ctgctggacataagccaccg

SEQ ID NO: 240 - DNA, 20 bp, Artificial Sequence
  Description of Artificial Sequence Synthetic oligonucleotide
tgttcctgggaagtacagcg

SEQ ID NO: 241 - DNA, 20 bp, Artificial Sequence
  Description of Artificial Sequence Synthetic oligonucleotide
caggtgtagaacttgcaggt

SEQ ID NO: 242 - DNA, 20 bp, Artificial Sequence
  Description of Artificial Sequence Synthetic oligonucleotide
atgatcagaaagagccgtag

SEQ ID NO: 243 - DNA, 20 bp, Artificial Sequence
  Description of Artificial Sequence Synthetic oligonucleotide
gatcctcataattcccgacg

SEQ ID NO: 244 - DNA, 20 bp, Artificial Sequence
  Description of Artificial Sequence Synthetic oligonucleotide
ccgtcggacttatgccagtg

SEQ ID NO: 245 - DNA, 20 bp, Artificial Sequence
  Description of Artificial Sequence Synthetic oligonucleotide
gtggcagaagtgaacctttg

SEQ ID NO: 246 - DNA, 20 bp, Artificial Sequence
  Description of Artificial Sequence Synthetic oligonucleotide
gatacttggccaaaggacgt

SEQ ID NO: 247 - DNA, 20 bp, Artificial Sequence
  Description of Artificial Sequence Synthetic oligonucleotide
gacattgccgtctatcccag

SEQ ID NO: 248 - DNA, 20 bp, Artificial Sequence
  Description of Artificial Sequence Synthetic oligonucleotide
tgcccttcaagacttcagtg

SEQ ID NO: 249 - DNA, 20 bp, Artificial Sequence
  Description of Artificial Sequence Synthetic oligonucleotide
agtcagccaatatttctgtg

SEQ ID NO: 250 - DNA, 20 bp, Artificial Sequence
  Description of Artificial Sequence Synthetic oligonucleotide
cttgcccccgaccgtctacg

SEQ ID NO: 251 - DNA, 20 bp, Artificial Sequence
  Description of Artificial Sequence Synthetic oligonucleotide
cttgaaggtgaggataacgc

SEQ ID NO: 252 - DNA, 20 bp, Artificial Sequence
  Description of Artificial Sequence Synthetic oligonucleotide
gaaaaaggtcccgagttctg

SEQ ID NO: 253 - DNA, 20 bp, Artificial Sequence
  Description of Artificial Sequence Synthetic oligonucleotide
cctccagaaccatctgacgg

SEQ ID NO: 254 - DNA, 20 bp, Artificial Sequence
  Description of Artificial Sequence Synthetic oligonucleotide
aagtagagagagaatgacca

SEQ ID NO: 255 - DNA, 20 bp, Artificial Sequence
  Description of Artificial Sequence Synthetic oligonucleotide
tcatcaggatgattccacac

SEQ ID NO: 256 - DNA, 20 bp, Artificial Sequence
  Description of Artificial Sequence Synthetic oligonucleotide
cagttccagatcgcgcactg

SEQ ID NO: 257 - DNA, 20 bp, Artificial Sequence
  Description of Artificial Sequence Synthetic oligonucleotide
ttaaacatagatcgctccca

SEQ ID NO: 258 - DNA, 20 bp, Artificial Sequence
  Description of Artificial Sequence Synthetic oligonucleotide
ggacgagagcacgttggaca

SEQ ID NO: 259 - DNA, 20 bp, Artificial Sequence
  Description of Artificial Sequence Synthetic oligonucleotide
ccgtcccaagaacgccacca

SEQ ID NO: 260 - DNA, 20 bp, Artificial Sequence
  Description of Artificial Sequence Synthetic oligonucleotide
gaagtaggtgtagagatcag

SEQ ID NO: 261 - DNA, 20 bp, Artificial Sequence
  Description of Artificial Sequence Synthetic oligonucleotide
catgctcaaggccatctgtg

SEQ ID NO: 262 - DNA, 20 bp, Artificial Sequence
  Description of Artificial Sequence Synthetic oligonucleotide
ctttgacatgctgtaccctg

SEQ ID NO: 263 - DNA, 20 bp, Artificial Sequence
  Description of Artificial Sequence Synthetic oligonucleotide
tgtggacagagcaccaatac

SEQ ID NO: 264 - DNA, 20 bp, Artificial Sequence
  Description of Artificial Sequence Synthetic oligonucleotide
caagtgctccagtttcagag

SEQ ID NO: 265 - DNA, 20 bp, Artificial Sequence
  Description of Artificial Sequence Synthetic oligonucleotide
aggaattggattcaagtacg

SEQ ID NO: 266 - DNA, 20 bp, Artificial Sequence
  Description of Artificial Sequence Synthetic oligonucleotide
tcttggaaattgactccgtg

SEQ ID NO: 267 - DNA, 20 bp, Artificial Sequence
  Description of Artificial Sequence Synthetic oligonucleotide
aaacaaggccttctgcaccg

SEQ ID NO: 268 - DNA, 20 bp, Artificial Sequence
  Description of Artificial Sequence Synthetic oligonucleotide
acaggggtggagctgaactg

SEQ ID NO: 269 - DNA, 20 bp, Artificial Sequence
  Description of Artificial Sequence Synthetic oligonucleotide
acagtggtgccagagaccag

SEQ ID NO: 270 - DNA, 20 bp, Artificial Sequence
  Description of Artificial Sequence Synthetic oligonucleotide
tactgctgttcagcggcaag

SEQ ID NO: 271 - DNA, 20 bp, Artificial Sequence
  Description of Artificial Sequence Synthetic oligonucleotide
cgggaaggacttcgtgaccg

SEQ ID NO: 272 - DNA, 20 bp, Artificial Sequence
  Description of Artificial Sequence Synthetic oligonucleotide
tgtcacccgcttcctcaacg

SEQ ID NO: 273 - DNA, 20 bp, Artificial Sequence
  Description of Artificial Sequence Synthetic oligonucleotide
acaggggtggagctgaactg

SEQ ID NO: 274 - DNA, 20 bp, Artificial Sequence
  Description of Artificial Sequence Synthetic oligonucleotide
ctccgttatgtggcatgaga

(19) United States
(12) Patent Application Publication
(10) Pub. No.: US 2022/0218847 A1
(43) Pub. Date: Jul. 14, 2022

(54) COMPOSITIONS AND METHODS CHARACTERIZING METASTASIS

(71) Applicants: The Broad Institute, Inc., Cambridge, MA (US); Dana-Farber Cancer Institute, Inc., Boston, MA (US)

(72) Inventors: Xin JIN, Cambridge, MA (US); Todd R. GOLUB, Boston, MA (US)

(73) Assignees: The Broad Institute, Inc., Cambridge, MA (US); Dana-Farber Cancer Institute, Inc., Boston, MA (US)

(21) Appl. No.: 17/605,207

(22) PCT Filed: Apr. 23, 2020
(86) PCT No.: PCT/US2020/029584

§ 371 Date: Oct. 20, 2021

Related U.S. Application Data
(60) Provisional application No. 62/837,525, filed on Apr. 23, 2019.

(51) Int. Cl. A61K 49/00; G01N 33/50

(52) U.S. Cl. CPC A61K 49/0047; G01N 33/5088; A61K 49/0008

(57) ABSTRACT

The invention features compositions and methods for determining the metastatic potential of cancer cell lines and tumors. Also provided is MetMap, a comprehensive database of the metastatic potential of cancer cell lines.

### CROSS-REFERENCE TO RELATED APPLICATION

This application claims the benefit of the following U.S. Provisional Application No. 62/837,525, filed Apr. 23, 2019, the entire contents of which are incorporated herein by reference.

### BACKGROUND OF THE INVENTION

Human cancer cell lines have been a driving force in cancer research and a useful tool for discovering oncogenic mechanisms and new therapeutic targets. However, large-scale characterization of cell lines has been limited to rudimentary metrics, such as viability in cell culture, because more complex phenotypes, e.g., in vivo behaviors, have not been tractable at scale. Most studies of metastasis rely on only a small number of experimental models, which make it difficult to extrapolate findings to genetically diverse human tumors. While there are hundreds of human cancer cell lines, the prospect of in vivo testing of each cell line, one-by-one, is unattractive not only because of its labor intensity, but also because of the difficulty in sufficiently controlling for variability between animal experiments. Thus, there is an urgent and demonstrated need for improved methods for characterizing the metastatic potential of cancer cell lines in vivo.

### SUMMARY OF THE INVENTION

As described below, the present invention features methods and compositions for characterizing the metastatic potential of cancer cell lines, as well as an interactive metastasis map featuring information that defines such cancer cell lines (e.g., their propensity to metastasize, organs where metastasis is typically observed, sequence data, genomic data, transcriptomic data, proteomic data, metabolomic data, drug sensitivity data, CRISPR knockout viability data, shRNA knockdown data, and annotated data relating to the cell of origin).

In one aspect, the present invention provides a method of characterizing the metastatic potential of a mixture of cancer cells in vivo, the method including systemically delivering to a non-human subject the plurality of cancer cells, where each cell contains a vector encoding as a single transcript a barcode, a detectable marker suitable for in vivo imaging, and a detectable marker suitable for cell selection and/or sorting. This method also includes imaging the cells and their descendants subsequent to delivery to locate where in the body the cell and/or its descendants are present, thereby characterizing the metastatic potential.

In another aspect, the invention provides a method of characterizing the metastatic potential of a mixture cancer cells in vivo, the method including systemically delivering to a non-human subject the plurality of cancer cells, each cell comprising a vector encoding a barcode; and subsequent to delivery detecting the bar code in a cell, tissue, or organ to determine where in the body the cell and/or its descendants are present, thereby characterizing the metastatic potential.

In another aspect, the invention provides a method of generating a metastasis map, the method including systemically delivering to a non-human subject a plurality of cells, each cell containing a vector encoding as a single transcript, a barcode, a detectable marker suitable for in vivo imaging, and a detectable marker suitable for cell selection and/or sorting, detecting the cells and their descendants subsequent to delivery to identify where in the body the cell and/or its descendants are present, compiling the detection data in a database, and associating the data with the cell's identity, thereby generating a metastasis map.

In yet another aspect, the invention provides a method for generating a metastasis map, the method including systemically delivering to a non-human subject a plurality of cells, each cell comprising a vector encoding as a barcode and detecting and quantitating expression of the barcode, compiling the expression data in a database and associating the expression data with the cell's identity, thereby generating a metastasis map.

In some embodiments of these inventions, the methods also include allowing the plurality of cells to proliferate in the subject for a period of time (e.g., days, weeks, and months). In some embodiments, the methods also include isolating the cells from the subject and characterizing the identity of the cells and their abundance. In some embodiments, the method also includes sorting the isolated cells. In embodiments of the above aspects or any other aspect of the invention, the identity and quantity of the cells or the sorted cells is assessed by next-generation sequencing or quantitative PCR. In some embodiments, the methods include carrying out single cell RNA sequencing on each cell, thereby generating a transcriptome for each cell. In some embodiments, the cells are isolated from brain, lung, liver, bone, and/or another organ or tissue. In one embodiment of the methods presented above, the plurality of cells is derived from two or more distinct cell lines. In some embodiments, the plurality of cells is derived from at least about 50, 100, 200, 300, 400, 500 or more cell lines. In some embodiments of the methods wherein the cell has a vector encoding marker suitable for imaging, the marker is a bioluminescent marker. In some embodiments, the imaging is used to monitor metastatic growth of the cells in vivo. In some embodiments, the expression levels of the barcode, the detectable marker suitable for in vivo imaging, and the detectable marker suitable for cell selection and/or sorting are correlated. In some embodiments, the abundance of the barcodes reflects the metastatic potentials of different cells. In some embodiments, barcode-enriched cells are characterized as highly metastatic, barcode-present cells are characterized as weakly metastatic, and barcode-depleted cells are characterized as non-metastatic. In some embodiments, the methods also include harvesting tissue of the non-human subject. In some embodiments, the methods also include preparing a lysate from the tissue, and in some embodiments, the methods also include isolating the cells from the lysate and characterizing the identity and quantity of the cells. In some embodiments of the above aspects, the cells are isolated from the subject, characterized as to their identity and abundance, and the data included in the metastasis map. In some embodiments, a genomic, transcriptomic or proteomic profile of the cell is included in the metastasis map. In some embodiments, the identity of the cells or the sorted cells and their quantity is assessed by next-generation sequencing or quantitative PCR, and the data included in the metastasis map. In some embodiments, the data is used to generate a metastasis map that includes a visual representation of the anatomical position of the cells and their proliferation over time. In some embodiments, drug sensitivity data, CRISPR knockout viability data, shRNA knockdown data, annotated cell line data, a metabolite profile, a genomic profile, a transcriptomic profile, or a proteomic profile of the cell is included as an interactive feature within the visual representation.

In another aspect, the invention provides a vector containing a single transcription cassette containing a detectable marker suitable for cell selection and/or sorting, a marker suitable for imaging a cell in vivo, and a barcode. In some embodiments, the vector is a viral vector, and in some instances the viral vector is a lentiviral vector. In some embodiments, the expression levels of the markers and the barcode are correlated. In some embodiments, the marker suitable for cell selection and/or sorting is GFP or mCherry. In some embodiments, the marker suitable for imaging is luciferase.

In yet another aspect, the invention provides a method for identifying the molecular features characteristic of a metastatic cell, wherein the method includes using the metastasis map generated using any of the methods disclosed herein to identify organ-specific patterns of metastasis. In some embodiments, the method also includes utilizing the organ specific patterns of metastasis to identify molecular features that distinguish brain-metastatic from non-metastatic cell lines. In some embodiments, the method also includes using genomic data from each cell to identify a mutation associated with brain metastasis.

In yet another aspect, the invention provides a computer implemented method of generating a metastasis map quantifying metastatic potential, the method involving receiving, by a processor, a listing of vectors encoded as barcodes, the vectors being associated with a plurality of cells systemically delivered to a non-human subject; receiving, from an imaging device, images of the plurality of cells and their descendants within the non-human subject; storing, by the processor, the images of the plurality of cells and their descendants in a database and identifying, by the processor, locations of the plurality of cells and their descendants from the images using the barcodes; and generating, by the processor, the metastasis map based on the locations of the plurality of cells and their descendants. In some embodiments, the method also includes comparing the location of the plurality of cells and their descendants from an image at a first point in time to the location of the plurality of cells and their descendants from an image at a second point in time. In some embodiments, the method also includes isolating cells at a particular location for presentation within the metastasis map. In some embodiments, the method also includes identifying cell types from for the plurality of cells and their descendants from the images, and in some embodiments, the method also includes isolating cell types for presentation within the metastasis map.

In other embodiments of the above aspects or any other aspect of the invention, the methods involve generating a visual representation of an anatomical position of the plurality of cells and their proliferation over time within the metastasis map. In some embodiments, the method also involves generating a genomic, transcriptomic or proteomic profile for the plurality of cells as an interactive feature within in the metastasis map. In some embodiments, the method further includes analyzing the plurality of cells and their descendants to characterize at least one of their identity, quantity, and abundance for visualization within the metastasis map. In some embodiments, comparing the location of the plurality of cells and their descendants at the first point in time and the second point in time is used to monitor metastatic growth of the cells over time in vivo. In some embodiments, the metastasis map is generated as a heat map for particular locations within the non-human subject. In some embodiments, the metastasis map is generated as at least one of a heat map, a pie chart, a bar graph, a PCA plot, and a radar plot. In yet another embodiment, the metastasis map can be generated showing quantities of each cell type from the plurality of cells at a particular location.

In another aspect, the invention provides a system for generating a metastasis map quantifying metastatic potential, the system containing a CPU, a computer readable memory and a computer readable storage medium, program instructions to receive a listing of vectors encoded as barcodes, the vectors being associated with a plurality of cells systemically delivered to a non-human subject; program instructions to receive images of the plurality of cells and their descendants within the non-human subject from an imaging device; program instructions to store the images of the plurality of cells and their descendants in a database and program instructions to identify locations of the plurality of cells and their descendants from the images using the barcodes; program instructions to generate the metastasis map based on the locations of the plurality of cells and their descendants.

The invention provides methods and compositions for determining the metastatic potential of cancer cell lines in an efficient and large-scale manner. Compositions and articles defined by the invention were isolated or otherwise manufactured in connection with the examples provided below. Other features and advantages of the invention will be apparent from the detailed description, and from the claims.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale &

Marham, The Harper Collins Dictionary of Biology (1991). As used herein, the following terms have the meanings ascribed to them below, unless specified otherwise.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Examples of diseases include cancer (e.g., metastatic cancer). Examples of cancers include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma).

The invention provides a number of targets that are useful for the development of highly specific drugs to treat or a disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “genomic profile” is meant a collection of information relating to single nucleotide alterations and copy number alterations. A genomic profile may include all or a portion of the genomic sequence of one or more cells. A genomic profile may include deviations from a reference genomic sequence. For example, a genomic profile of a cancer cell may include single nucleotide variants or other mutations that are not present in a normal, non-cancerous cell.

By “harvesting” is meant collecting a biological sample from a subject. In some instances, harvesting includes excision of an organ. In other instances, harvesting includes a biopsy.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any analyte (e.g., protein or polynucleotide) having an alteration in expression level or activity that is associated with a disease or disorder.

By “Metastasis Map” or “MetMap” is meant a collection of data related to the cancer cell lines. In one embodiment, a MetMap delineates the metastatic potential of each cell line in the collection.

“Metastatic potential” refers to the propensity of a cancer to develop secondary malignant growths at a distance from a primary site of cancer.

By “metastatic tumor” is meant a malignant growth that originates from a single cell that has survived in circulation, undergone extravasation, initiated tumor formation, and/or induced blood vessel remodeling.

As used herein, “obtaining” as in “obtaining an agent” includes synthesizing, purchasing, or otherwise acquiring the agent.

By “proteomic profile” is meant information about the expression of proteins. A proteomic profile may include all or a portion of the proteins present in a cell (e.g., cancer cell). A proteomic profile may include information about alterations in protein expression relative in a cancer cell relative to the protein expression of a reference cell. In some embodiments, the alteration is the presence or absence of a protein relative to a reference cell. The proteomic profile may include alterations in the amount of one or more proteins present in a cell compared to a reference cell. In some embodiments, a reference cell is a normal, non-cancerous cell derived from the same tissue the cancerous cell is derived from.

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

By “transcriptomic profile” is meant information about the expression levels of RNAs. In some embodiments, a transcriptomic profile includes expression profiling or splice variant analysis. In other embodiments, the transcriptomic profile includes information relating to mRNAs, tRNAs, of sRNAs. A transcriptomic profile may include all or a portion of the genes expressed in a cell. A transcriptomic profile may include alterations in gene expression relative to a reference cell, wherein the alteration can be the presence of a transcript not observed in the reference cell or the absence of a transcript that is present in the reference cell. The transcriptomic profile may include alterations in the amount of one or more transcripts present in a cell compared to a reference cell. A reference cell is a normal, non-cancerous cell derived from the same tissue the cancerous cell is derived from.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A to 1I illustrate the scalable in vivo metastatic potential mapping with pools of barcoded cell lines and co-capturing of cancer compositions and transcriptomes by RNA-Seq of polyclonal metastases. “FP” represents fluorescent protein “Luc” represents luciferase; “BC” represents barcode; “G” represents green fluorescent protein (GFP); and “R” represents mCheRry.

FIG. 1A is a schematic showing the workflow of determining the in vivo metastatic potential profiling using barcoded cell line pools. Three key elements of the labeling vector including fluorescent protein (FP), luciferase (Luc) and barcode (BC) are presented. G, GFP; R, mCheRry.

FIG. 1B is an example of a gating strategy to isolate GFP⁺ barcoded cancer cells. Infected cell lines expressed GFP at different levels as shown in the histogram, and a fixed gate was utilized to enrich cells with close GFP expression levels. Numbers correspond to cell percentage.

FIG. 1C is a schematic showing the workflow of metastatic cancer cell isolation from different organs and RNA-Seq to readout cancer cell barcode and in vivo transcriptomes. FIG. 1D is an example of a barcode mapping result visualized by Integrative Genomics Viewer (IGV).

FIG. 1E is a graph of the distribution of the barcode read count abundance versus all gene transcript counts. Barcodes are among the top 10% highly expressed genes, allowing robust quantification.

FIG. 1F is an example of a barcode abundance measurement in the pre-injected population and metastasis samples. MDAMB231: BC1 and BCS; HCC1954: BC2 and BC6; BT549: BC3 and BC7. CAL851, BC4 and BC8. “G” represents the GFP portion; “R” represents the mCheRry portion; “cpk” represents counts per kilo; and “BC” represents barcode.

FIG. 1G is a set of images of real-time bioluminescence imaging (BLI) and a graph summarizing the results observed in the images.

FIG. 1H is a graph illustrating total cancer cell numbers isolated by fluorescence assisted cell sorting (FACS) from different organs.

FIG. 1I is a graph of cancer cell composition of metastases from different organs as determined by barcode abundance from the pooled cells. “Preinj” represents pre-injection. Cells expressing GFP and mCheRry are lighter and darker colored bars, respectively, in the brain, lung, liver, kidney, and bone. The identifiers (e.g., S67) refer to the sample number. FIGS. 2A and 2B illustrate quantification of barcode abundance using a Taqman RT-qPCR assay.

FIG. 2A is a matrix showing the results of a Taqman assay on in vitro cultured barcoded cells. The signal is very specific to each barcode and there is no detectable crosstalk. “BC” represents barcode.

FIG. 2B is a graph illustrating the quantification of barcode abundance and cancer cell composition using the Taqman RT-qPCR assay in the pre-injected population and in the metastasis samples from different organs.

FIGS. 3A to 3D illustrate single cell RNA-Seq of metastases from different organs.

FIG. 3A provides a work flow showing that single cancer cells (SCs) isolated from each organ were sorted into 96-well plates, with 90 cells per plate (the remaining 6 wells were used for positive and negative controls) and subjected to Smart-Seq2. 360 cells were profiled. 176 cells passed quality control and were subjected to Principal Component Analysis (PCA). PC1 maximally separated the cancer cells into two populations, with one population enriched in cells isolated from brain, and the other population enriched in cells isolated from lung, liver and bone.

FIG. 3B is a heatmap showing gene expressions associated with PC1 and clustering of cells.

FIG. 3C is a series of PCA plots. The differential expression of these marker genes suggest that the left group is HCC1954 (ERBB2+, CDH1+), the right group is MDAMB231 (CDKN2A loss, VIM+).

FIG. 3D is a graph illustrating cancer cell composition based on single cell RNA-Seq data. The results agree with barcode quantification from bulk RNA-Seq (see FIG. 1I).

FIGS. 4A to 4H demonstrate mapping metastatic behaviors of basal-like breast cancer cell lines.

FIG. 4A is a PCA plot of transcriptomic expression of the breast cancer collection from Cancer Cell Line Encyclopedia (CCLE) and the pooling schemes focusing on basal-like breast cancer.

FIG. 4B is a series of bioluminescence imaging and graphs summarizing the data in the images for Group 1 cell line pools.

FIG. 4C is a series of bioluminescence imaging images and graphs summarizing the data in the images for Group 2 cell line pools.

FIG. 4D is a graph depicting isolated total cancer cell number in Group 1 cell line pools.

FIG. 4E comprises graphs illustrating cancer cell composition in Group 1 cell line pools as quantitated by barcodes from preinjected pools and from in vivo metastasis in mice and five organs. Error bars indicate SEM. Each group contained 8 mice. Different shades represent different barcodes.

FIG. 4F is a graph depicting isolated total cancer cell number in Group 2 cell line pools.

FIG. 4G comprises graphs illustrating cancer cell composition of Group 2 cell line pools as quantitated by barcodes from preinjected cell lines and from in vivo metastasis in mice and m five organs. Error bars indicate SEM. Each group contains 8 mice. The data shown in FIGS. 4C, 4D, 4F, and 4G were used to quantify the metastatic potential of breast cancer cell lines, as shown in FIG. 4H.

FIG. 4H is a set of diagrams illustrating the metastatic patterns of 21 basal-like breast cancer cell lines. Metastatic potentials quantify inferred cell numbers detected from the target organs. Data are presented on log10 scale as the legend in FIG. 1A

FIGS. 5A and 5B illustrate the metastatic potential measured from pooled cell line experiments agree with individual cell line measurements.

FIG. 5A is a series of real-time bioluminescence imaging that monitored metastasis progression of the 8 cell lines that were individually tested. Each plot highlights one of the eight lines. Error bars indicate SEM. Each group contains four mice.

FIG. 5B is a scatter plot showing the correlation of overall metastatic potential (5 organs combined) from pooled cell line experiments with whole body bioluminescence imaging of metastases measured individually line by line.

FIGS. 6A to 6E illustrate the MetMap of 125 cancer cell lines.

FIG. 6A is a schematic of experimental workflow of metastatic potential mapping using PRISM. A PRISM pool of 25 cell lines was used for testing the need of GFP labeling and cancer cell purification. The barcode abundance substantially altered compared to the unlabeled population after GFP labeling as shown by the pie chart.

FIG. 6B is a line-by-line comparison of barcode abundance before and after GFP labeling. The unlabeled cell pool had a more even distribution. Post labeling, several cell lines showed strong dropout, but all lines were still detectable. “BC” denotes barcode throughout the figures.

FIG. 6C is a scatter plot comparing the barcode enrichment after normalizing to the pre-injected input from the two experiments. Strong positive correlation was observed with the exception of one cell line, U205.

FIG. 6D is a schematic of a simplified workflow using pan-cancer PRISM cell line pools for high-throughput metastatic potential profiling.

FIG. 6E is a chart showing the cancer lineage distribution of the profiled 500 cancer cell lines. Each dot represents a cell line. If the cell line was derived from primary tumor or metastasis is indicated.

FIGS. 7A-7T illustrate the MetMap125 and MetMap500.

FIG. 7A is a schematic comparing experimental conditions between MetMap500 and MetMap125.

FIG. 7B comprises a chart and a graph of the initial barcode abundance in the pre-injected population of MetMap125. “BC” denotes barcode throughout the figures.

FIG. 7C comprises a chart and a graph of the initial barcode abundance in the pre-injected population of MetMap500.

FIG. 7D comprises scatter plots comparing raw barcode abundance from in vivo organs versus the data normalized to the pre-injected input (FIG. 7B). A strong linear relationship was observed, indicating that subtle differences in the initial abundance mattered little, and that barcode abundance from in vivo was likely biology-driven.

FIG. 7E comprises scatter plots comparing raw barcode abundance from in vivo organs versus the data normalized to the pre-injected input (FIG. 7C). A strong linear relationship was observed, indicating that subtle differences in the initial abundance mattered little, and that barcode abundance from in vivo was likely biology-driven.

FIG. 7F is a scatter plots showing overall metastatic potential as determined in MetMap500 and MetMap125. Highly strong correlation is observed between the two experiments. Each dot represents a cell line. Cancer lineage is tracked by shading.

FIG. 7G comprises scatter plots showing organ-specific metastatic potential as determined in MetMap500 and MetMap125. Highly strong correlation is observed between the two experiments. Each dot represents a cell line. Cancer lineage is tracked by shading.

FIGS. 7H-7K illustrate observed results from subcutaneous injection of PRISM cell line pool.

FIG. 7H comprises a schematic showing that the same PRISM pool of 498 cell lines used for MetMap500 profiling was tested with subcutaneous (subQ) injection on a cohort of 6 mice. A graph of survival curves compared animal survival in subQ and intracardiac (IC) injections is also provided.

FIG. 7I comprises pie charts and graphs showing the total numbers of cell lines detected in animals from the subQ and IC injections.

FIG. 7J is a scatter plot showing barcode-quantitated tumorigenic potential and metastatic potential from subQ and IC experiments.

FIG. 7K comprises a schematic of Group 1 of basal breast cancer pool subjected to mammary fat pad injection, barcode quantitation through RNA-Seq, and cell number inference. A graph is also provided that shows the inferred cell number per cell line.

FIG. 7L comprises box plots showing single variate correlation of cancer lineage with overall metastatic potential from MetMap500 data.

FIG. 7M comprises box plots showing single variate correlation of the cell lines was derived from primary tumor or metastasis. “Primary with met” denotes that the cell line was derived from primary tumor and patient demonstrated metastasis at diagnosis or later.

FIG. 7N comprises box plots showing single variate correlation of the age of the patient with overall metastatic potential from MetMap500 data.

FIG. 7O comprises box plots showing single variate correlation of the gender of the patient with overall metastatic potential from MetMap500 data.

FIG. 7P comprises box plots showing single variate correlation of the ethnicity of the patient with overall metastatic potential from MetMap500 data.

FIG. 7Q is a scatter plot showing single variate correlation of cell doubling with overall metastatic potential from MetMap500 data.

FIG. 7R comprises scatter plots showing the correlation of metastatic potential with patient age, stratified by cancer lineage. An inverse correlation was observed in several cancer types.

FIG. 7S is an example view of MetMap portal showing the top metastatic lines from diverse lineages.

FIG. 7T comprises radar plots that show the MetMap of melanoma, pancreatic, prostate and brain cancer.

FIG. 8A is a scatter plot showing single variate correlation of mutation burden with overall metastatic potential from MetMap500 data. Mutation burden was quantified by total somatic mutation counts from exon-seq data.

FIG. 8B is a scatter plot showing single variate correlation of aneuploidy status with overall metastatic potential from MetMap500 data. Aneuploidy was quantified by chromosome arm-level events from exon-seq data.

FIG. 8C comprises bar plots showing the significance of single variate and multi variate association analysis with metastatic potential. Dotted lines indicate 0.05.

FIGS. 9A to 9D illustrate the correlation of overall metastatic potential with origin site, derivation length, mutation burden, and doubling speed in the 21 basal-like breast cancer cohort.

FIG. 9A is a graph illustrating the association of metastatic potential with the site of origin of cancer cell lines.

FIG. 9B is a scatter plot showing the correlation between metastatic potential with time in culture to derive the cell lines.

FIG. 9C is a scatter plot showing the correlation between metastatic potential with mutation rate of lines.

FIG. 9D is a scatter plot showing the correlation between metastatic potential with in vitro doubling time (in hours).

FIGS. 10A to 10F illustrate genomic alterations that associate with brain metastatic potential in basal-like breast cancer cohort.

FIG. 10A is a graph depicting single nucleotide mutations that associate with brain metastatic potential. The top gene PIK3CA reaches statistical significance (FDR<0.05). Known oncogenes or tumor suppressors in basal-like breast cancer are presented for comparison. Each dot represents a gene, positive association depicted in darker color, negative association depicted in lighter color.

FIG. 10B provides a graph showing copy number alterations that are associated with brain metastatic potential. The top line correlates all clusters in chr 8p12-8p21.2 (FDR=0.0017, highlighted in bold). JIMT1 has deletions in ADAM28 and LEPROTL1.

FIG. 10C is a chart illustrating the amplification status of genes surrounding HER2 and their association with brain metastatic potential.

FIG. 10D comprises a graph and box plots that show copy number alterations that associate with brain metastatic potential. Genes residing in chromosome 8p score on top and reaches statistical significance (FDR<0.05). Each dot represents a gene, positive association depicted in darker color, negative association depicted in lighter color.

FIG. 10E is a map of chromosome 8p (chr8p) deletions and amplifications for 21 cell lines. The deleted chr8p region (ADAM28˜WRN) best associates with brain metastatic potential. Gene-by-gene status of the 21 cell lines are presented. BNIP3L, EPHX2, and LEPROTL1 repression induces altered lipid metabolism as reported by Cai et al. DEL, deletion, cutoff<=−1. AMP, amplification, cutoff>=1.

FIGS. 10F-10L illustrate that Chr 8p gene low status associates with brain metastasis in clinical breast cancer specimens.

FIG. 10F comprises heatmaps showing that coordinated expression of chr 8p genes mirrored their copy number status in the two large breast cancer datasets, METABRIC and TCGA. The 8p^{low} cluster was defined by CNA data. CNA, Copy Number Alteration. Exp, RNASeq Expression.

FIG. 10G comprises tables and charts showing the distribution of 8p^{low} cluster in different breast cancer subtypes and its association with disease specific survival in the METABRIC and TCGA datasets.

FIG. 10H is a heatmap showing the hierarchical clustering of primary breast tumors by 8p gene expression in the EMC-MSK dataset. The 8p^{low} cluster is enriched in tumors that developed brain metastasis, but not lung or bone metastasis.

FIG. 10I comprises a table and graphs showing that metastasis free survival curves stratified by 8p^{low} status in EMC-MSK. The 8p^{low} cluster displayed poorer brain metastasis compared to the 8p^{WT} cluster.

FIG. 10J comprises graphs showing brain metastasis free survival curves stratified by 8p^{low} status in subtypes of EMC-MSK.

FIG. 10K comprises a table and heatmap showing the hierarchical clustering of breast cancer metastases by 8p gene expression, with the 8p^{low} cluster being enriched in brain metastases.

FIG. 10L comprises graphs showing Chr 8p CNA status determined by Targeted Seq in the MSK metastatic breast cancer dataset. Brain metastases are enriched in chr 8p deletion compared to primary tumor, local recurrence, and metastases at other sites. The 8p^{low} cluster predicts poor brain metastasis free survival.

FIGS. 10M-10R illustrate that the PI3K-response signatures associate with brain metastasis in clinical breast cancer specimens.

FIG. 10M comprises heatmaps showing co-regulated patterns of two independent PI3K-response signatures in METABRIC and TCGA breast cancer datasets. PI3Ksig.1 was generated by overexpression of PIK3CA^{mut} in breast epithelial cells. PI3Ksig.2 was generated by PI3K inhibitor treatment in the CMap database.

FIG. 10N comprises tables and graphs showing the distribution of PI3Ksig^{high} cluster in different breast cancer subtypes and its association with disease specific survival in the METABRIC and TCGA datasets.

FIG. 10O is a heatmap that shows the hierarchical clustering of primary breast tumors by PI3K signatures in the EMC-MSK dataset. The PI3Ksig^{high} cluster is enriched in tumors that developed brain metastasis.

FIG. 10P comprises a table and graphs showing metastasis free survival curves stratified by PI3K signatures in EMC-MSK. The PI3Ksig^{high} cluster displayed poorer brain metastasis.

FIG. 10Q comprises graphs showing brain metastasis free survival curves stratified by PI3K signatures in subtypes of EMC-MSK.

FIG. 10R comprises a table and heatmaps showing hierarchical clustering of breast cancer metastases by PI3K signature, with the PI3Ksig^{high} cluster being enriched in brain metastases.

FIGS. 10S-10V illustrate 8p^{low} and PI3Ksig^{high} co-occurrence in clinical breast cancer specimens.

FIG. 10S comprises heatmaps showing significant yet non-complete overlap between 8p^{low} and PI3Ksig^{high} clusters in the EMC-MSK dataset.

FIG. 10T comprises a table and graphs showing 8p^{low} and PI3Ksig^{high} clusters co-capture a subset of patients with the worst brain metastasis prognosis.

FIG. 10U is graph showing the Cox proportional-hazards model of brain metastasis free survival using multi variates—8p, PI3Ksig, and breast cancer subtype. The 8p^{low}-PI3Ksig^{high} cluster is the most associated with brain metastasis.

FIG. 10V comprises heatmaps showing that 8p^{low} and PI3Ksig^{high} clusters co-capture the majority of brain metastasis samples.

FIG. 11 comprises graphs showing the top gene expression signatures that associate with brain metastatic potential. Bars indicate p values. Expression signature (MSigDB) scores were projected for each cell line using their in vitro RNASeq data.

FIGS. 12A to 12H illustrate in vivo transcriptome data of breast cancer metastases.

FIG. 12A is a schematic showing the differential analysis approach for in vivo transcriptomes with mixed cancer cell line compositions. An in silico transcriptome model was based on single cell line in vitro transcriptomes and cell line composition of the metastasis sample. The in silico profile was then compared with the actual in vivo data in a paired-wise manner.

FIG. 12B is a series of scatter plots comparing in silico modeled in vitro expression to the actual pre-injected (direct mixture of in vitro cell lines) or in vivo metastasis samples.

FIG. 12C is a series of scatter plots depicting the log2 fold changes (FC) of all genes. “Pilot” refers to the pilot group; “g1” represents group 1; and “g2” represents group 2 (see FIG. 8A).

FIG. 12D is a series of boxplots showing log2 fold changes of SCGB2A2 and MUCL1 expression in the studies of three pools. Each point represents a sample.

FIG. 12E is a heatmap showing log2 fold change of lung metastasis genes (Minn et al., Nature 436: 518-24 (2005)) in lung, liver, kidney, and bone metastasis samples from the pilot study, where MDAMB231 dominated the population.

FIG. 12F comprises a scatter plot and a heat map that show lower expression of TGFβ signature score and representative genes, respectively, in brain metastases than other metastasis sites.

FIG. 12G comprises a scatter plot and a heat map that show lower expression of EMT signature score and representative genes, respectively, in brain metastases compared to other organs.

FIG. 12H depicts the results of GSEA analysis with all RNA-Seq samples combined by metastasis organ sites irrespective of sample or cell line composition. Gene sets related to lipid metabolism are selectively enriched on top in the brain but not in other organs or in vitro.

FIGS. 13A and 13B indicate a role lipid synthesis in metastasis.

FIG. 13A comprises a chart and graph showing lipid metabolite species that associate with brain metastatic potential. Bars indicate p values. Lipid metabolites were grouped by species, and enrichment analysis of the species was performed using fgsea. CE, cholesterol ester; PC, phosphatidylcholine; SM, sphingomyelin; LPC, lysophosphatidylcholine; LPE, lysophosphatidylethanolamine; DAG, diacylglycerol; TAG, triacylglycerol; PPP, pentose phosphate pathway metabolites pathway genes in bran metastases, including the rate-limiting enzyme G6PD.

FIG. 13B is a graph depicting triacylglycerol (TAG) abundance in different mouse tissues. Brain is uniquely low in TAG, by orders of magnitude.

FIGS. 14A to 14I illustrate that SREBF1-mediated lipid metabolism is tied to breast cancer brain metastatic potential.

FIG. 14A comprises a graph showing CRISPR gene dependencies that associate with brain metastatic potential. The top gene SREBF1 (FDR=0.001) is a selective dependency in highly brain metastatic lines.

FIG. 14B is a scatter plot showing the relations between SREBF1 dependency and brain metastatic potential. CERES scores of gene dependency were used: ceres=-1 indicates essential as pan-essential genes, and ceres=0 indicates non-essential. Pearson correlation coefficient (cor) and test p value are presented.

FIG. 14C comprises two graphs that show the distribution of SREBF1 (top) and SREBF2 (bottom) dependencies across 435 human cancer cell lines. The positions of highly brain metastatic cells including HCC1806, HCC1954, JIMT1, and MDAMB231 are indicated with arrows, whereas weakly- or non-brain metastatic breast cancer cells are not indicated with arrows.

FIG. 14D is a series of scatter plots showing association of SREBF1 dependency with metastatic potential at different organ sites. Strong correlation was observed with brain but not with others. Each dot represents a cell line.

FIG. 14E comprises scatter plots showing correlation of SREBF1 gene dependency and brain metastatic potential in MetMap500 and MetMap125. Strong inverse correlation was observed for breast cancer. Each dot represents a cell line.

FIG. 14F comprises graphs showing consensus alterations in lipid species abundance upon SREBF1 knockout (KO) in JIMT1 and HCC1806, two brain metastatic cell lines. Bars indicate adjusted p values. Lipid metabolites were grouped by species, and enrichment analysis of the species was performed using fgsea.

FIG. 14G comprises heatmaps showing lipid metabolite profile changes upon SREBF1 KO. Heatmaps showing relative lipid abundance in cells cultured in medium supplemented with serum or delipidated serum. SREBF1-WT and SREBF1-KO of JIMT1 (PIK3CA^{mut}) and HCC1806 (8p^{low}) were used. Lipid species grouping and lipid desaturation level are also presented.

FIG. 14H is a volcano plot showing consensus gene expression changes upon SREBF1 KO in JIMT1, HCC1806, HCC1954, MDAMB231, four brain metastatic cell lines. The two top genes are SREBF1 and SCD (FDR<0.05, highlighted in bold).

FIG. 14I is a graph showing the co-dependencies of SREBF1 across 739 human cancer cell lines in a genome-wide CRISPR viability screen. The two top genes are SCD and

SCAP (FDR<1e-79, highlighted in bold)

FIGS. 15A-15J illustrate analyses of expression profiles.

FIG. 15A is a heatmap from a GSEA analysis of lipid metabolism gene sets in JIMT1 in vivo samples. Each tick represents a lipid metabolism gene set from MSigDB. ***, p=0.0001.

FIG. 15B is a heatmap from a GSEA analysis of lipid metabolism gene sets in GTEX normal tissue. Each tick represents a lipid metabolism gene set from MSigDB. ***, p=0.0001.

FIG. 15C is a bubble plot showing enrichment of Hallmark gene pathways (MSigDB) and comparing in vivo expression of metastases at different organ sites to their in vitro counterparts.

FIG. 15D comprises a bubble plot and a graph showing in vivo upregulation of SREBF1, SCD and SREBF1-response signature in brain metastases.

FIGS. 15E-15G illustrate TGFβ signaling, EMT status, SREBF1 target, and PPP gene expression in clinical breast cancer metastasis specimens.

FIG. 15E comprises a graph and a heatmap that show lower expression of TGFβ signature score and representative genes in brain metastases than other metastasis sites.

FIG. 15F comprises a graph and a heatmap that show lower expression of EMT signature score and representative genes in brain metastases compared to other organs. FIG. 15G is a heatmap that shows enriched expression of selective SREBF1 target genes in brain metastases, including FASN, SCD and SREBF1 itself.

FIG. 15H-15J illustrate gene expression comparison of paired primary breast tumor and brain metastasis clinical specimens.

FIG. 15H comprises heatmaps that illustrate a strategy to remove brain stroma contamination effect from brain metastasis expression profiles. A gene signature indicating brain stroma contamination was derived from comparison of brain with breast and breast cancer brain metastasis. Arrowheads indicate a few brain metastasis samples with noticeable brain stroma contamination. A brain contamination score was calculated and its effect was then regressed out in the paired RNASeq of primary tumor and brain metastasis dataset. The heatmap shows expression of brain stroma indicator before and after removal of the contamination effect.

FIG. 15I comprises graphs that show paired comparison of selective lipid metabolism and PPP genes after removal of brain stroma contamination. Lipid metabolism genes: SREBF1, SCAP, SCD, FADS2, FASN, PMVK, HMGCL. PPP genes: G6PD, PGD, TPI1, TALDO1. P, Primary breast tumor; M, brain Metastasis.

FIG. 15J comprises graphs that show paired comparison of selective pathway signatures after removal of brain stroma contamination. Adipogenesis and fatty acid metabolism signatures showed up-regulation, whereas TGFβ, EMT, inflammatory response, and TNFa signatures showed down-regulation. Signature scores were projected for each sample using the corrected RNA-Seq profiles.

FIGS. 16A-16P illustrate interrogation of lipid metabolism genes in breast cancer brain metastasis.

FIG. 16A is a schematic of in vivo CRISPR screen investigating relative gene fitness in brain metastasis outgrowth.

FIG. 16B comprises box plots that show the top hits from the in vivo CRISPR screen interrogating a mini-library targeting 29 lipid metabolism related genes. Thirteen genes scored at FDR<0.05. Each dot represents an animal. On average 2 guides per gene were used.

FIG. 16C comprises BLI radiance images and graphs that show one-by-one gene validation of selective hits by intracranial injection of JIMT1-edited cells. Cell outgrowth in brain metastasis was monitored by real-time BLI. Two independent guides per gene were tested, in a one guide one mouse fashion. WT, wild type; KO, knockout; g1, guide 1 and g2, guide 2 (see Table 3).

FIG. 16D comprises BLI imaging and graphs that quantify relative difference in brain metastasis load in mice receiving intracarotid injection of SREBF1-WT or -KO JIMT1 cells. Each group contains 7˜8 mice. Error bars indicate SEM.

FIG. 16E comprises BLI imaging and graphs of one-by-one assessment of lipid metabolism gene fitness in an independent brain metastatic cell line HCC1806. Cell outgrowth in brain metastasis was monitored by real-time BLI. Two independent guides per gene were tested, in a one guide one mouse fashion.

FIG. 16F comprises pie charts that summarize CRISPR-seq quantification of SREBF1 gene editing efficiencies of brain-derived and pre-injected HCC1806 and JIMT1.

FIG. 16G is an alignment showing CRISPR-seq analysis assessment of gene editing mutant alleles of SREBF1.g1 in pre-injected and brain-derived HCC1806 cells. Major mutant alleles and allele frequencies are presented. A strong reduction in allele diversity was observed in brain-derived cells, suggesting a subset of clones were selected in the brain.

FIG. 16H is an alignment showing CRISPR-seq analysis assessment of gene editing mutant alleles of SREBF1 in pre-injected and brain-derived HCC1806 cells. Major mutant alleles and allele frequencies are presented. A strong reduction in allele diversity was observed in brain-derived cells, suggesting a subset of clones were selected in the brain.

FIG. 16I is a graph showing the allele frequencies of preinjected SREBF1.g1 and SREBF1.g2 (left) and the allele frequencies of brain-derived SREBF1.g1 and SREBF1.g2 (right)

FIG. 16J is an alignment showing CRISPR-seq analysis assessment of gene editing mutant alleles of SREBF1 in pre-injected and brain-derived JIMT1cells. Major mutant alleles and allele frequencies are presented. A strong reduction in allele diversity was observed in brain-derived cells, suggesting a subset of clones were selected in the brain.

FIG. 16K is graph showing the gene editing mutant allele frequencies of SREBF1 in pre-injected and brain-derived JIMT1 cells. Major mutant alleles and allele frequencies are presented. A strong reduction in allele diversity was observed in brain-derived cells, suggesting a subset of clones were selected in the brain.

FIG. 16L comprises images of Western blots for quantifying SREBF1 protein level of brain-derived and pre-injected HCC1806 and JIMT1, at precursor and mature level.

FIG. 16M comprises graphs that show RT-qPCR quantification of relative expression of SREBF1, SCD, CD36, FABP6 in brain-derived and pre-injected HCC1806 and JIMT1. Pre-injected WT HCC1806 was used as reference.

FIG. 16N is a series of bioluminescence imaging (BLI) images and graphs that quantify the relative difference in metastasis load in the organs of mice receiving SREBF1-WT or -KOJIMT1 cells as detected in the BLI images. Each group contains five mice. Error bars indicate standard error of the mean (SEM).

FIG. 16O is a series of images of fluorescently labeled metastases in serial brain sections containing metastasis lesions by SREBF1-WT or -KO cells. Circles highlight macro-metastatic lesions and arrows indicate micro lesions.

FIG. 16P is a confocal tile scan of representative brain sections from mice receiving SREBF1-WT or -KO cells. GFP⁺ signal indicates cancer lesions.

FIG. 17 is a diagram showing correlation of gene expression changes in different metastasis sites. Pre-injected population had no expression change thus showed no correlation with in vivo samples. Brain metastases showed weaker correlations with extracranial metastases

FIG. 18 comprises a side-by-side comparison of 4 brain metastatic cell lines with intracranial injection of SREBF1-WT and -KO cells. Cell outgrowth in brain metastasis was monitored by real-time BLI. Two independent guides per gene were tested, in a one guide one mouse fashion. WT, wild type; KO, knockout.

FIG. 19 is a diagrammatic illustration of a high-level architecture for implementing processes in accordance with aspects of the invention.

### DETAILED DESCRIPTION OF THE INVENTION

The invention features compositions and methods that are useful for determining the metastatic potential of cancer cell lines, as well as an interactive metastasis map featuring information that defines such cancer cell lines (e.g., their propensity to metastasize, organs where metastasis is typically observed, sequence data, genomic data, transcriptomic data, proteomic data, metabolomic data, drug sensitivity data, CRISPR knockout viability data, shRNA knockdown data, and annotated data relating to the cell of origin).

The invention is based, at least in part, on the discovery that a cancer cell's metastatic potential can be ascertained by systemically delivering the cell, in a modified form to allow detection, to a non-human subject. Accordingly, the invention provides compositions and methods for determining the metastatic potential of a plurality of cancer cell lines in vivo. These methods and compositions have been used to generate a map of the metastatic properties of individual cell lines, and this Metastasis Map (or MetMap) represents a novel and important tool for the study of metastatic cancer.

Nucleic Acid Constructs Methods and compositions are provided herein for tracking cancer cells administered to a non-human subject in vivo. Compositions of the present invention can be used to modify cancer cells prior to administration to the subject so that the cells express identifying markers. Thus, one aspect of the present disclosure provides a nucleic acid construct comprising a barcode, a first detectable marker, and a second detectable marker. The first detectable marker allows in vivo imaging of the cells after administration to a non-human subject. In some embodiments, the first detectable marker is a bioluminescent marker, such as a luciferase. Luciferases, unlike fluorescent proteins, do not require an external light source to generate a signal, which makes this family of bioluminescent markers suitable for in vivo imaging.

The second detectable marker allows for cell selection, sorting, or both. Markers suitable for cell selection and/or sorting include, but are not limited to, fluorescent proteins. In some embodiments, the second marker is a green, red, blue, or yellow fluorescent protein (GFP, RFP, BFP, or YFP, respectively). In some embodiments, the second marker is mCherry. In some embodiments, the second detectable marker comprises an epitope to which an antibody specifically binds. In some embodiments, the antibody that specifically binds to the epitope is labeled.

In some embodiments of the present invention, the nucleic acid construct encodes a barcode but no detectable markers. In some embodiments, other selectable markers (e.g., antibiotic resistance genes) are encoded in the nucleic acid construct to enable efficient selection of transformed or transduced cells. In some embodiments, a surface protein on the cancer cell can be used to isolate or detect the cancer cell. In some embodiments, the surface protein comprises an epitope to which an antibody can specifically bind and mediate isolation of the cancer cell. In some embodiments, the antibody is labeled. In some embodiments, the label is a fluorescent or other visually detectable label.

The barcode between 10 and 30 nucleotides. For example, the barcode contemplated herein may comprise 10, 11, 12, 13, 14, 15, 16, 17, 18, 19, 20, 21, 22, 23, 24, 25, 26, 27, 28, 29, or 30 nucleotides. The barcodes are designed to reduce or eliminate nonspecific binding to the cancer cell's nucleic acid molecules (i.e., genomic DNA, RNA, etc.). In some embodiments, the barcode comprises a nucleic acid sequence that is not substantially complementary to any endogenous nucleic acid sequence present in the cancer cell. In some embodiments, the barcode is designed to diverge from perfect complementarity from an endogenous nucleic acid sequence present in the cancer cell by 2, 3, or 4 or more nucleotides. In some embodiments, the barcode is designed so that the most complementary sequences in an endogenous nucleic acid molecule present in the cancer cell have a conformation that disfavors barcode binding to the endogenous nucleic acid molecule.

In some embodiments, the nucleic acid construct encoding the barcode and markers is a single expression cassette. Thus, the expression of each encoded element is correlated with the expression of the other elements. In some embodiments, the nucleic acid construct is a vector (e.g., recombinant plasmids). The term “recombinant vector” includes a vector (e.g., plasmid, phage, phasmid, virus, cosmid, fosmid, or other purified nucleic acid vector) that has been altered, modified or engineered such that it contains greater, fewer or different nucleic acid sequences than those included in the native or natural nucleic acid molecule from which the recombinant vector was derived. For example, a recombinant vector may include a nucleotide sequence encoding a polypeptide (i.e., the markers) and/or a polynucleotide (i.e., the barcode), or fragment thereof, operatively linked to regulatory sequences such as promoter sequences, terminator sequences, long terminal repeats, untranslated regions, and the like, as defined herein. Recombinant expression vectors allow for expression of the genes or nucleic acids included in them.

In some embodiments of the present disclosure, one or more nucleic acid constructs having a nucleotide sequence encoding one or more of the polypeptides or polynucleotides described herein are operatively linked to one or more regulatory sequences that can integrate the nucleic acid construct into a cancer cell genome. In some embodiments, cancer cells are stably transfected or transduced by the introduced nucleic acid construct. Modified cells can be selected, for example, by detecting the first or second marker. In some embodiments, barcode, and at least one of the marker gene are encoded in different nucleic acid constructs, and will be introduced into the same cell by co-transfection or co-transduction. Any additional elements needed for optimal synthesis of polynucleotides or polypeptides described herein would be apparent to one of ordinary skill in the art.

In some embodiments, the nucleic acid construct comprises at least one adapter nucleic acid sequence that has a sequence complementary to that of a nucleic acid molecule used in a downstream sequencing reaction. For example, the adapters used in some embodiments are designed to be compatible with next-generation sequencing including, but not limited to, Ion Torrent and MiSeq platforms. In some embodiments, the length of the adapter is between 8 and 20 nucleotides. In some embodiments, the length of the adapter is 8, 9, 10, 11, 12, 13, 14, 15, 16, 17, 18, 19, or 20 nucleotides. The adapter's sequence is designed to reduce or eliminate nonspecific binding of the adapter to an endogenous nucleic acid molecule. In some embodiments, the adapter is designed to have a sequence that is not substantially complementary to any nucleic acid sequence present in an endogenous nucleic acid molecule. In some embodiments, the adapter is designed to diverge from perfect complementarity with the endogenous nucleic acid molecule by 2, 3, or 4 or more nucleotides.

### Methods for Characterizing Metastatic Potential

One aspect of the present disclosure provides a method for characterizing the metastatic potential of a mixture of cancer cell lines in vivo. In one embodiment, the method comprises modifying the cells to comprise a nucleic acid construct encoding a barcode, a first detectable marker, and a second detectable marker, such as the constructs described above. Each distinct cell line in the mixture of cell lines will be modified to express a unique barcode, and each barcode will only be used with a single cell line. The modified cells are systemically administered to a non-human subject and allowed to propagate in the non-human subject. After a period of time, the non-human subject is imaged to detect at least one of the markers encoded in the nucleic acid construct, which allows the location of the cells in the body of the non-human subject to be determined.

The non-human subject can be any non-human mammal. In some embodiments, the non-human mammal is a mouse, rat, rabbit, pig, goat, or other domesticated mammal. In some embodiments, the non-human animal is immunocompromised. In some embodiments, the non-human subject is an immunocompromised mouse, such as a NOD scid gamma (NSG) mouse.

Methods of introducing exogenous nucleic acid molecules into a cell are known in the art. For example, eukaryotic cells can take up nucleic acid molecules from the environment via transfection (e.g., calcium phosphate-mediated transfection). Transfection does not employ a virus or viral vector for introducing the exogenous nucleic acid into the recipient cell. Stable transfection of a eukaryotic cell comprises integration into the recipient cell's genome of the transfected nucleic acid, which can then be inherited by the recipient cell's progeny.

Eukaryotic cells (e.g., human cancer cells) can be modified via transduction, in which a virus or viral vector stably introduces an exogenous nucleic acid molecule to the recipient cell. Eukaryotic transduction delivery systems are known in the art. Transduction of most cell types can be accomplished with retroviral, lentiviral, adenoviral, adeno-associated, and avian virus systems, and such systems are well-known in the art. In some embodiments of the present disclosure, the viral vector system is a lentiviral system.

In some embodiments, the viral vectors are assembled or packaged in a packaging cell prior to contacting the intended recipient cell. In some embodiments, the vector system is a self-inactivating system, wherein the viral vector is assembled in a packaging cell, but after contacting the recipient cell, the viral vector is not able to be produced in the recipient cell. In some embodiments, the first detectable marker allows in vivo imaging of the cells after delivery to a non-human subject. In some embodiments, the first detectable marker is a bioluminescent marker, such as a luciferase. Luciferases, unlike fluorescent proteins, do not require an external light source to generate a signal, which makes this family of bioluminescent markers suitable for in vivo imaging. In some embodiments, luciferin or an analogous substrate is administered to the non-human subject, which is acted upon by the luciferase to generate bioluminescence. In some embodiments, in vivo imaging comprises bioluminescence imaging. Many imaging methodologies are known in the art that can be utilized in the methods presented herein. Examples of such methodologies include, but are not limited to, those disclosed in U.S. Publication Nos. 20180160099, 20170220733, 20170212986, 20170038574, 20160370295, 20160202185, 20140333750, 20140326922, 20140063194, and 20140038201, the contents of each are incorporated herein by reference in their entirety.

The second detectable marker is used to isolate and/or sort modified cancer cells from other cells. A technique for isolating or sorting cancer cells comprising a nucleic acid construct as described herein is flow cytometry. In fluorescence activated cell sorting

(FACS), a fluorescent marker is used to distinguish modified from unmodified cells. In some embodiments, the second marker is a fluorescent polypeptide suitable for cell sorting. In some embodiments, the second marker is a polypeptide having an epitope that is specifically bound by a fluorescently labelled antibody. A gating strategy appropriate for the cells expressing the marker (or otherwise labeled) is used to segregate the cells. For example, modified cancer cells expressing a fluorescent protein (e.g., GFP or mCherry) can be separated from other cells in a sample by using a corresponding gating strategy. In one embodiment, a GFP gating strategy is employed. In some embodiments, an mCherry gating strategy is used. Other methods of isolating cells are known in the art and may be used to segregate modified cancer cells from non-modified cells and from cells derived from a non-human subject.

To determine from which cell line a particular modified cancer cell is derived from, the barcode within the modified cell is sequenced. Sequencing of the barcodes within the modified cancer cells is accomplished using a next-generation sequencing platform such as IonTorrent or MiSeq, but other platforms are contemplated herein. Additionally, single cell analysis (e.g., single cell RNA sequencing (RNA-seq)) can be used to determine barcode sequences and identify the cell lines from which the modified cancer cells present at a location or in a sample derived. RNA-seq may also be used to generate transcriptome data for the modified cancer cells.

The abundance of modified cancer cells present in a metastatic lesion is indicative of the metastatic potential of the cell lines from which the cells are derived. In some embodiments, the abundance of modified cancer cells is determined during cell isolation and/or cell sorting. In some embodiments, the modified cells are quantitated during next-generation sequencing or RNA-seq. Other methods of quantitating cells in a sample or tissue are known in the art.

Generating Metastasis maps

Another aspect of the present disclosure provides methods for generating a metastasis map of cancer cell lines. These methods include systemically delivering a mixture of cells derived from cancer lines to a non-human animal, wherein the cells are modified to comprise a vector encoding a barcode or a vector encoding a barcode and at least one marker as described above. The method for generating the map further involves detecting and quantitating the expression of the barcode, and these steps are also described above. The data derived from quantitating the expression of the barcode is then compiled in a database and associated with the cell's identity (i.e., identifying the cell line from which the cell derived).

The metastasis map may also include a genomic, transcriptomic, or proteomic profiles of the cell line. In some embodiments, the metastasis map also includes drug sensitivity data, CRISPR knockout viability data, shRNA knockdown data, annotated cell line data, and/or a metabolite profile of the cell line. The data that constitutes the profiles may be generated de novo using methods known in the art.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### Assessing the Feasibility and Reliability of In Vivo Barcoding to Monitor Metastasis

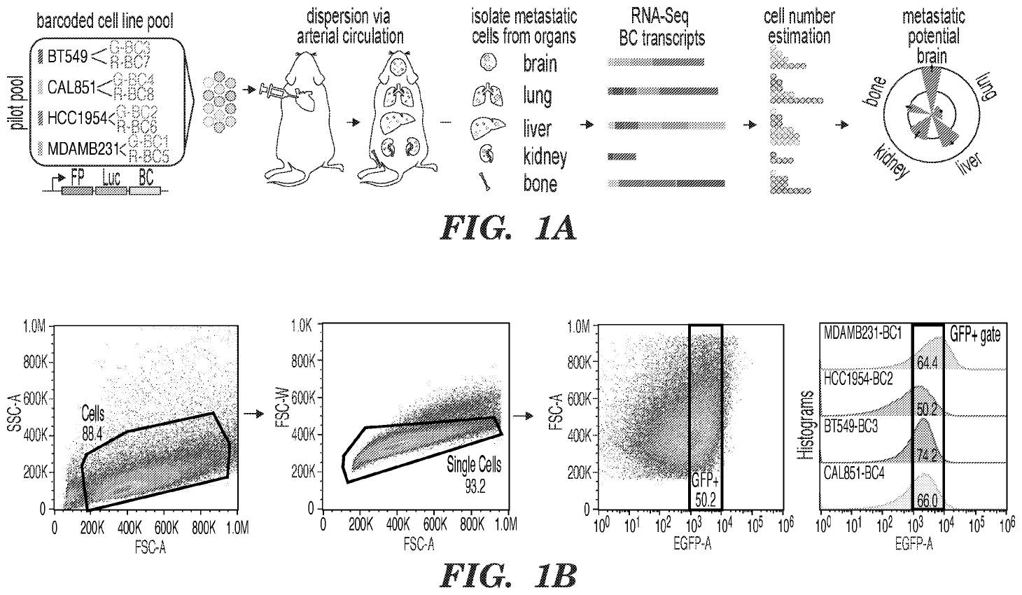

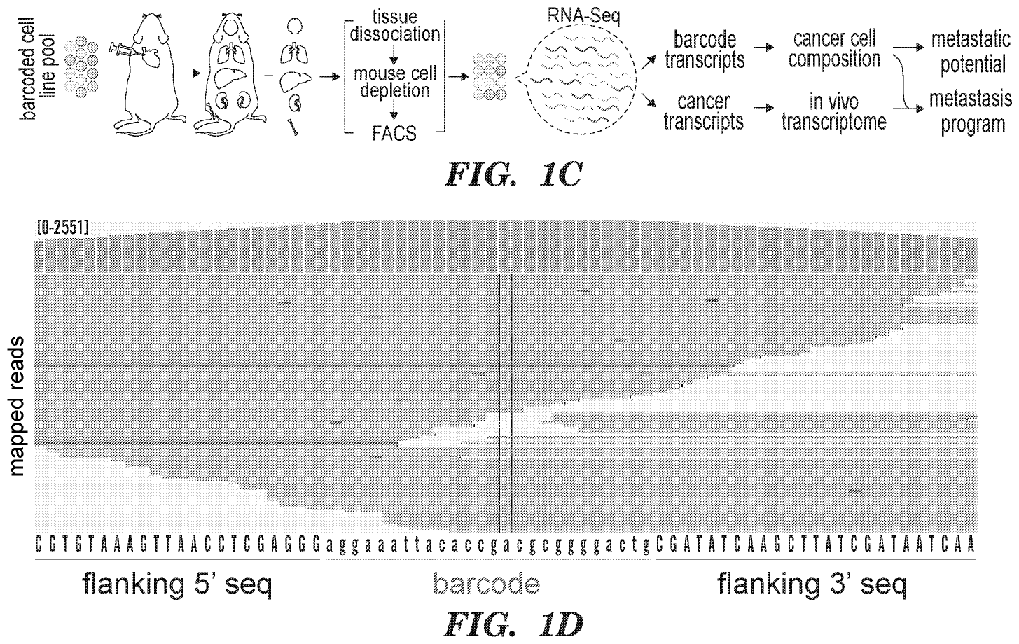

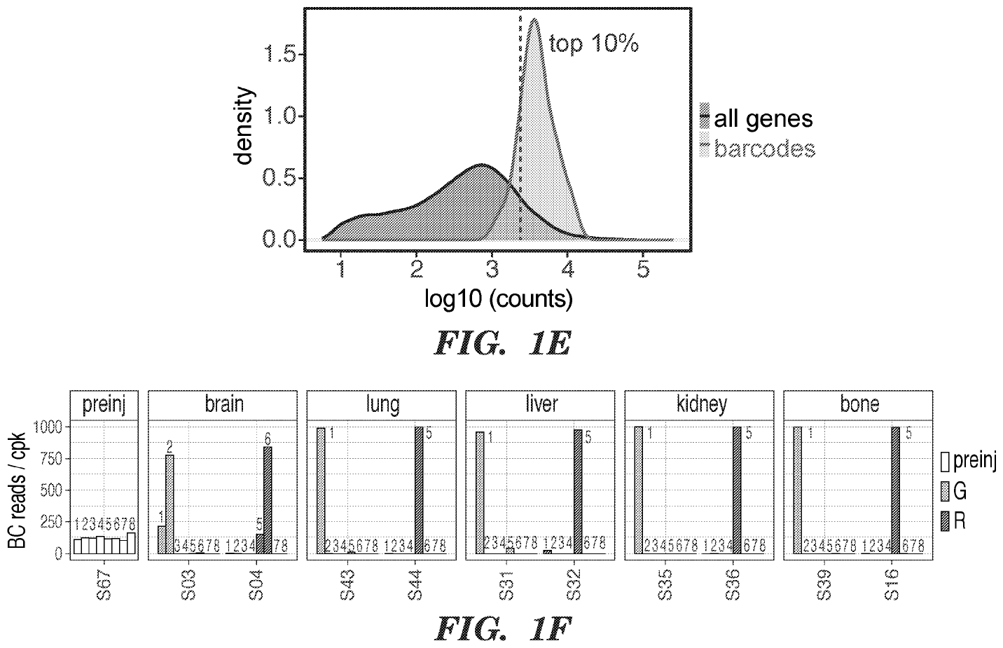

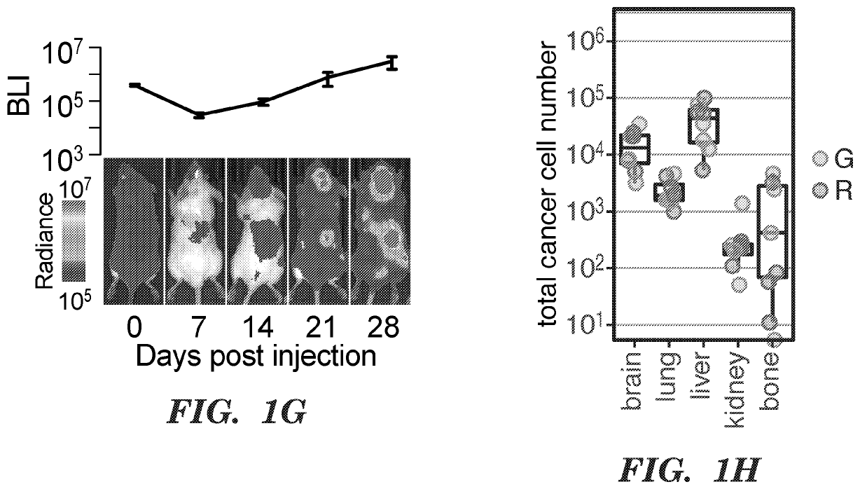

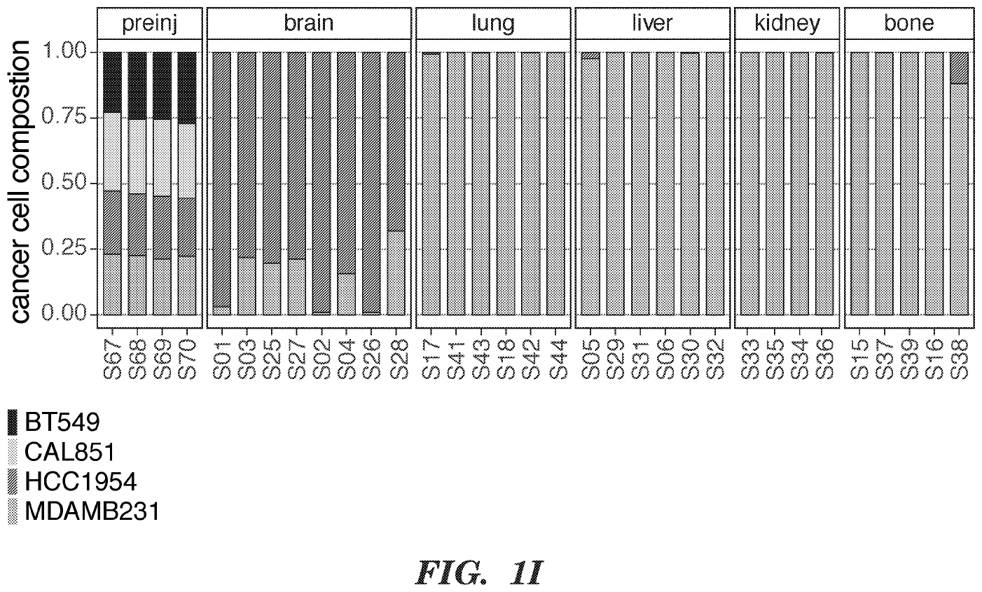

Methods of monitoring metastasis are needed to better understand similarities and differences between different types of cancer. To test the feasibility and reliability of in vivo barcoding to monitor metastasis, a pilot study of four breast cell lines was performed (FIGS. 1A to 1C). The cell lines were BT549, CAL851, HCC1954, and MDAMB231. Each cell line was engineered to express three elements—a unique 26 nucleotide-long barcode together with luciferase for in vivo imaging and either GFP or mCherry to facilitate cell sorting and for measuring reproducibility within a single mouse (FIG. 1A). The three elements constituted a single transcription cassette, which ensured that the labeled cell lines harbored similar expression levels (and thus similar copy numbers) of barcodes through gating the fluorescence expression by fluorescence assisted cell sorting (FACS) (FIG. 1B). The designed barcodes could be analyzed at either the DNA or RNA level by a TaqMan assay or by next-generation sequencing, both of which are suitable for both low-throughput and high-throughput applications.

The transcribing barcode design allowed co-capturing of cancer barcodes and cancer transcriptomes of metastases from bulk RNA-Seq analysis, and a workflow was developed that analyzed both (FIG. 1C). The resulting transcriptomic profiles represent an ensemble from multiple constituent cell lines and yielded consensus gene programs and generalizable molecular insights about organ-specific metastases. An example of barcode mapping from the pilot experiment is presented in FIG. 1D. The barcodes were expressed at high levels (i.e., among the top 10% highly expressed genes) allowing robust quantification (FIGS. 1E and 1F).

Eight barcoded cell lines (the four cell lines modified to express either GFP or mCheRry) were injected as a pool into the left ventricle of recipient mice. Bioluminescence imaging (BLI) revealed metastatic lesions throughout the body (FIG. 1G). Five weeks post injection, brain, lung, liver, kidney and bone were collected, human tumor cells isolated by FACS for GFP or mCherry (FIG. 1H), and barcodes enumerated by RNA-Seq (FIGS. 1A to 1F). While barcode abundances were similar pre-injection, certain barcodes were enriched in particular organs after injection (FIG. 1F). Patterns of metastatic spread were distinct from cell line to cell line, and highly similar patterns in the GFP or mCherry versions of the same cell line were seen across multiple mice, demonstrating reproducibility of the pooled approach (FIG. 1I). For example, HCC1954 was most strongly detected in brain, whereas extracranial metastases were dominated by MDAMB231 (FIG. 1I).

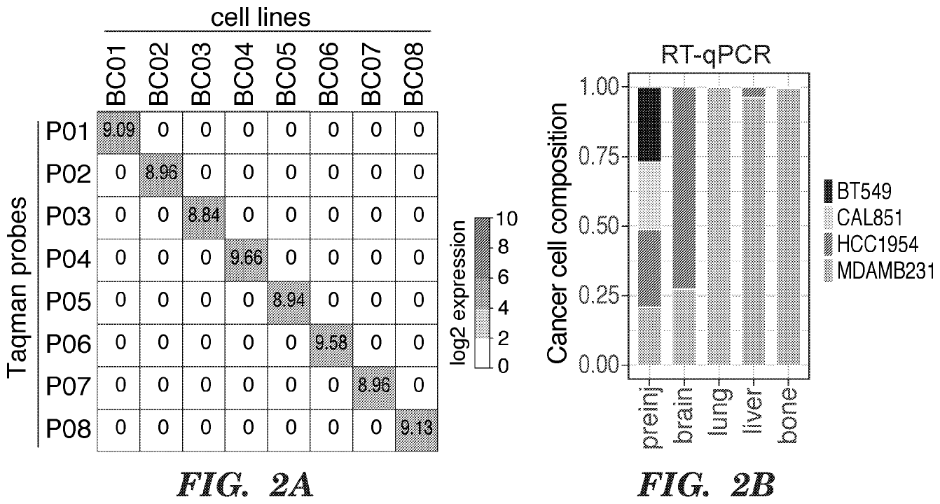

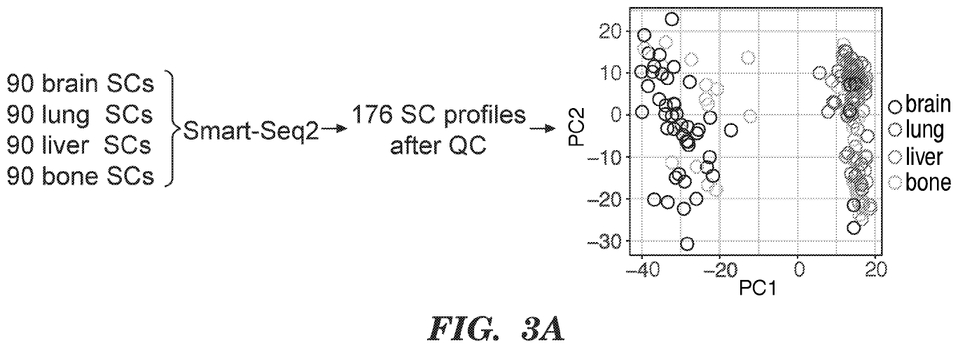

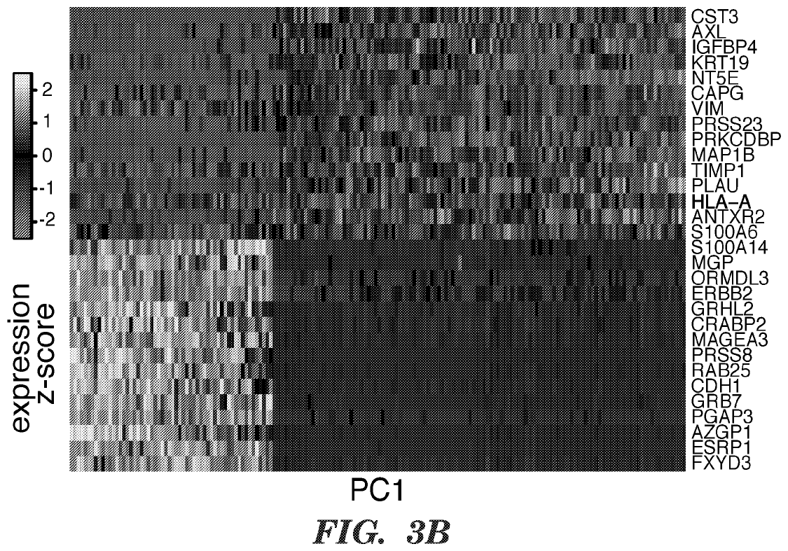

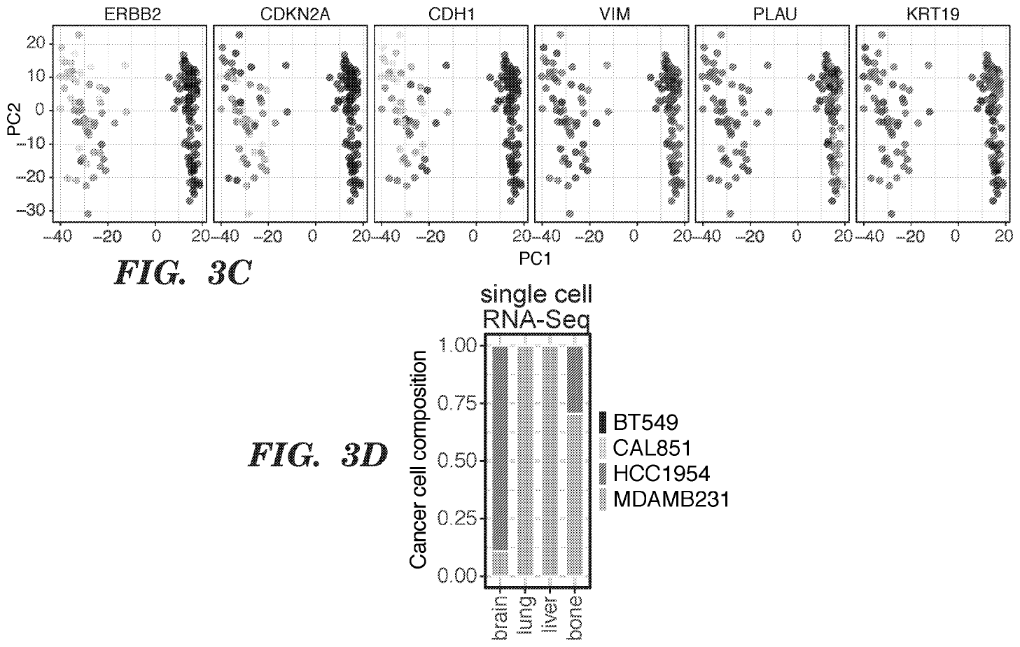

The results observed for barcodes quantitated by bulk RNA-Seq were validated by two methods: quantitative RT-PCR and single cell RNA sequencing (FIGS. 2A, 2B, and 3A to 3D). An examination of individual barcoded lines showed that the Taqman probes were highly specific to the engineered barcodes, and there was no crosstalk in detection (FIG. 2A). Consistent with RNA-Seq (FIG. 1I), RT-qPCR showed even distribution of the cell lines in the pre-injected pool, but selective enrichment of cell lines in different organs (FIG. 2B). To validate at single cell resolution, single cell RNA-Seq was performed on the cancer cells isolated from different organs (FIG. 3A). Principal component analysis (PCA) stratified cell lines into two clusters. One cluster was characterized by high expression of genes on the HER2 amplicon (ERBB2, ORMDL3, GRB7, PGAP3), consistent with the HCC1954 (HER2+) identity (FIGS. 3B, 3C). The other cluster was characterized by high expression of vimentin (VIM) and low expression of CDKN2A (P16), consistent with MDAMB231 harboring P16-loss and being VIM-high (FIG. 3C). Mapping cell line identities to their organ origins indicated that HCC1954 was abundant in the brain, whereas MDAMB231 dominated lung, liver, and bone (FIG. 3D). In both approaches, BT549 and CAL851 were not detected. Collectively, these results validated the results observed in the pilot study.

### Example 2

### Characterizing Metastatic Behavior of Basal-Like Breast Cancer Cell Lines

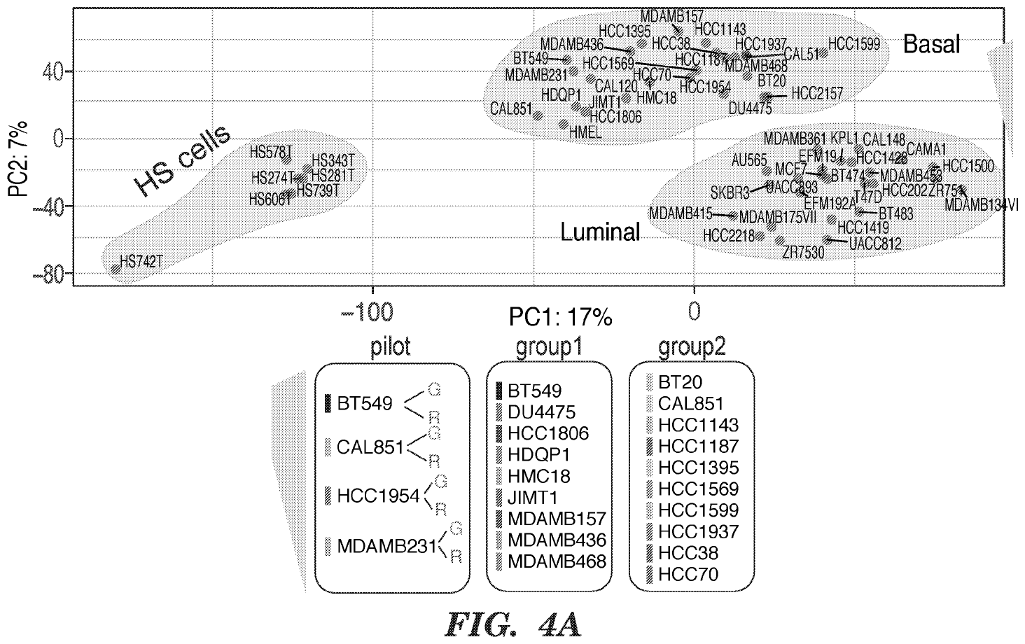

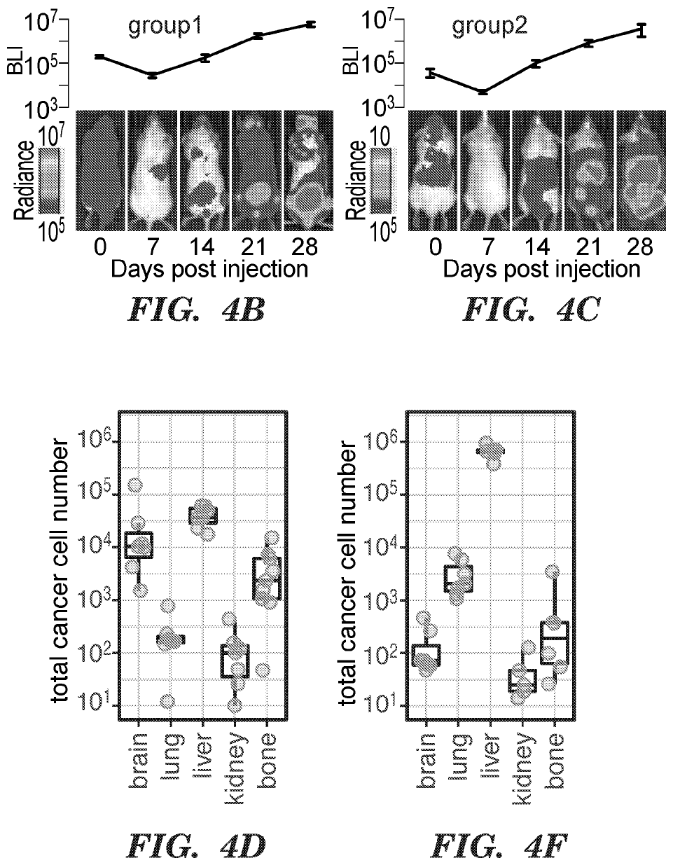

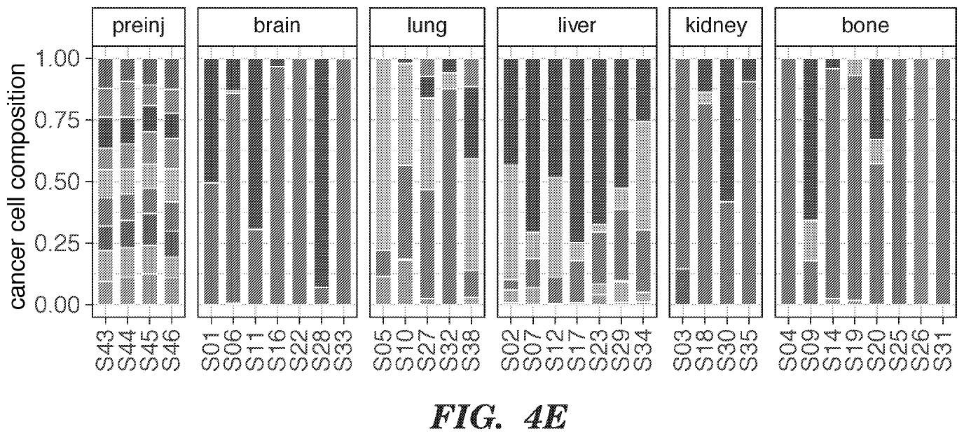

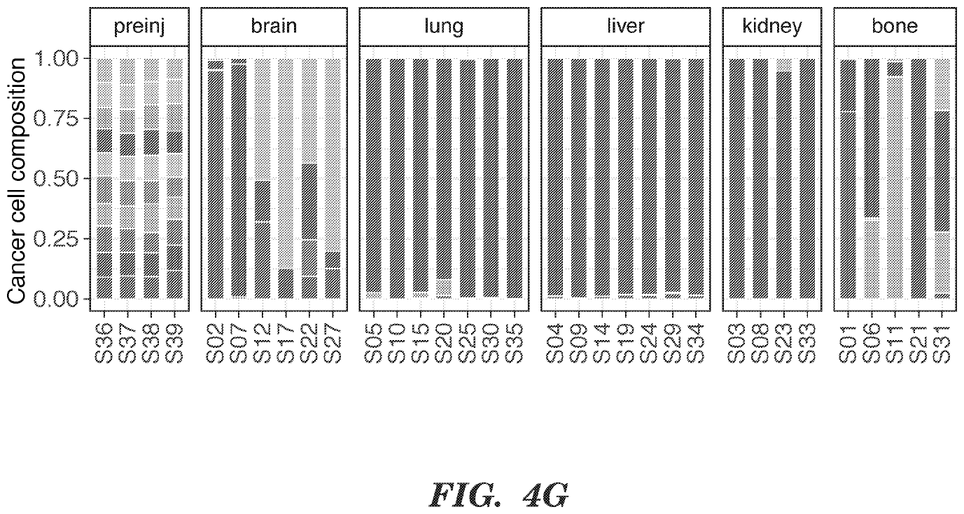

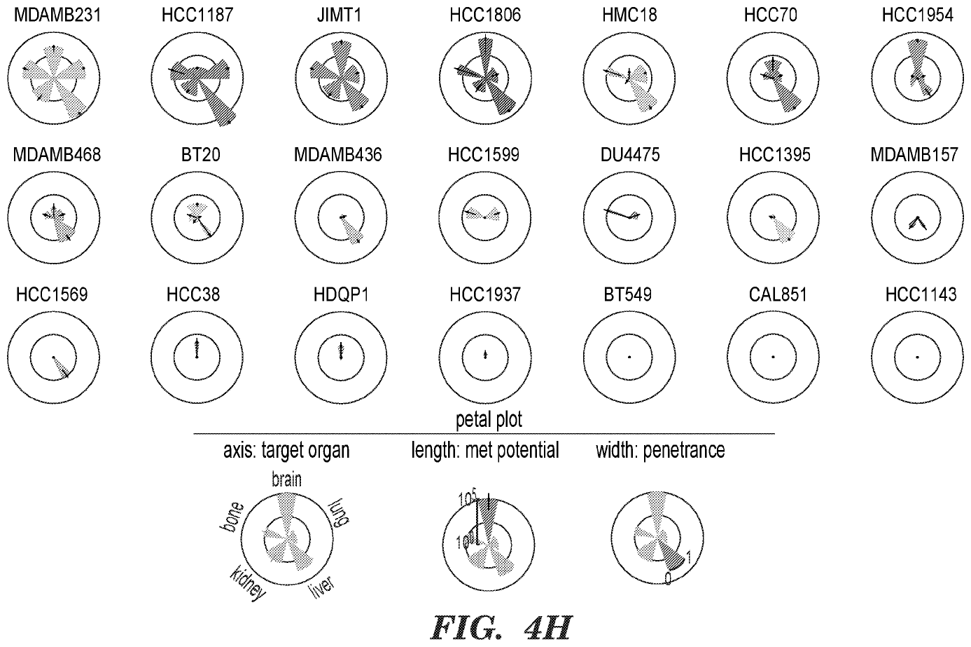

Having validated the method for in vivo barcoding to monitor metastasis, a larger subset of breast cancer cells was evaluated for metastatic behaviors. Principal component analysis (PCA) of expression profiles stratified the breast cancer cell lines in the Cancer Cell Line Encyclopedia (CCLE) collection into 3 categories: (1) expression initiated with HS (termed HS cells), displaying fibroblast morphology and characteristics, (2) enriched in luminal subtype, and (3) enriched in basal subtype (FIG. 4A). 21 basal-like breast cancer cell lines were chosen for evaluation and divided into two pools (group 1 and group 2). The two non-metastatic lines BT5649 and CAL851 from the pilot study were also included in the pools for reassessment (FIG. 4A). These basal-like cell lines are derived from breast cancer subtypes known to have diverse metastatic abilities in patients (Kennecke, H. et al., J. Clin. Oncol. 28: 3271-77 (2010), the contents of which are herein incorporated in their entirety).

Cell lines were individually barcoded, pooled at equal numbers, and injected into mice (FIG. 1C). Bioluminescence imaging indicated comparable tumor progression kinetics observed in the pilot study (FIGS. 4B, 4C). All mice were sacrificed five weeks post injection in a time-matched manner. The total cell numbers and barcode-quantitated cell line composition from each organ sample are presented in FIGS. 4D to 4G.

To quantify the cell line metastatic potentials on an absolute scale, the cell count for each cell line in different organs was inferred based on the total number of isolated cancer cells and their compositions as measured by barcode abundance. This metric was then used to compare cell lines across the three pools analyzed (pilot, group 1, and group 2) (FIG. 4H, Table 1). A diversity of metastatic patterns and differential aggressiveness were observed. Aggressiveness can be characterized by determining the rate at which cancer cells proliferate after colonizing an organ or by determining the number or percentage of cells from the initial pool that colonize an organ or organs.

TABLE 1
Cell Line Comparison

| | Cl.05 | Cl.95 | mean | penetrance |
|---|---|---|---|---|
| BT20_BREAST | 0.041739182 | 1.14120279 | 0.80999195 | 0.6 |
| BT549_BREAST | 0 | 0 | 0 | 0 |
| CAL851_BREAST1 | 0 | 0 | 0 | 0 |
| DU4475_BREAST | 0 | 2.94871853 | 2.47257363 | 0.125 |
| HCC1143_BREAST | 0 | 0 | 0 | 0 |
| HCC1187_BREAST | 1.514757066 | 3.32027789 | 2.88141499 | 1 |
| HCC1395_BREAST | 0 | 0.48729684 | 0.22798267 | 0.2 |
| HCC1569_BREAST | 0 | 0 | 0 | 0 |
| HCC1599_BREAST | 1.043079445 | 2.32452193 | 2.00075199 | 0.6 |
| HCC1806_BREAST | 0.977933067 | 3.57631234 | 3.1808527 | 0.5 |
| HCC1937_BREAST | 0 | 0 | 0 | 0 |
| HCC1954_BREAST | 0.398355305 | 0.74985535 | 0.60319272 | 1 |
| HCC38_BREAST | 0 | 0 | 0 | 0 |
| HCC70_BREAST | 0 | 1.42579855 | 1.00116042 | 0.4 |
| HDQP1_BREAST | 0 | 0 | 0 | 0 |
| HMC18_BREAST | 0.935278877 | 2.97359658 | 2.5970876 | 0.375 |
| JIMT1_BREAST | 3.14528796 | 3.59363725 | 3.41008012 | 0.875 |
| MDAMB157_BREAST | 0 | 0 | 0 | 0 |
| MDAMB231_BREAST | 2.919645172 | 3.53553331 | 3.33308226 | 1 |
| MDAMB436_BREAST | 0 | 0 | 0 | 0 |
| MDAMB468_BREAST | 0.377576368 | 1.25085467 | 0.97301991 | 0.375 |

*Data are presented on a log10 scale

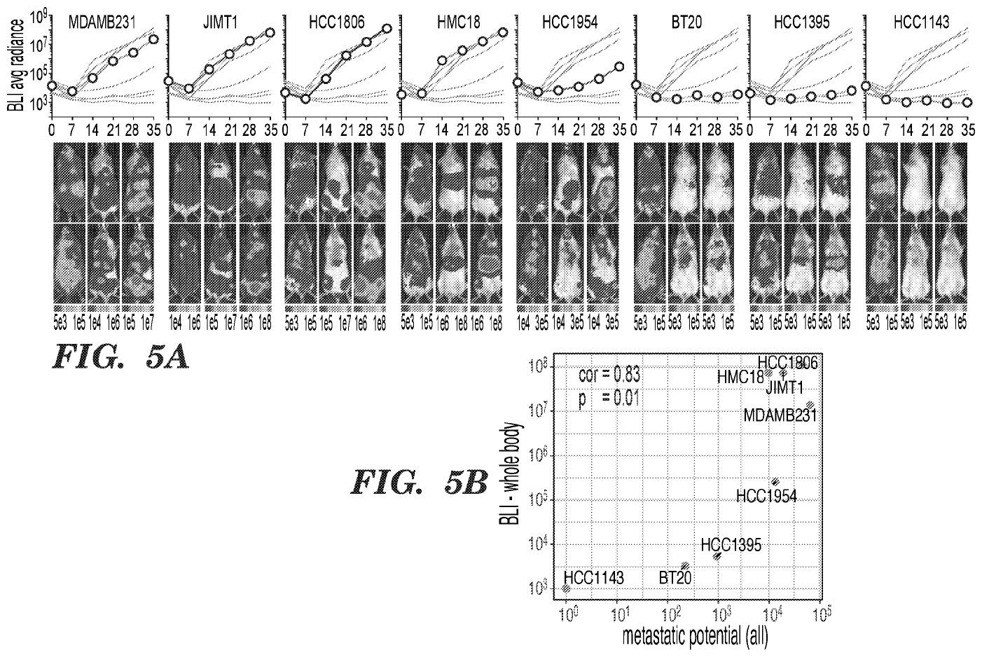

The analysis characterized some cell lines as pan-metastatic. For example, four cell lines, MDAMB231, HCC1187, JIMT1, and HCC1806 displayed pan-metastatic behaviors. Some showed a propensity for liver, lung, bone, or brain, and others were not metastatic (FIG. 4H). Other cell lines displayed more selective patterns. Among the 21 different cell lines in the three pools, DU4475 and HCC1599 were suspension cells, and both displayed selective colonization towards bone and lung. Interestingly, one cell line (BT20) was detected in multiple organs but all at very low abundance, reflecting its ability to colonize but not expand in different micro-environments. Whether the in vivo pattern was associated with cell culture status remained unclear. To validate the patterns of metastasis observed in the pooled in vivo system, eight cell lines were characterized individually. The pooled and individual results were highly correlated (FIGS. 5A, 5B).

### Example 3

### High-Throughput Characterization of Metastatic Potential

Having demonstrated feasibility, the metastatic potential was mapped for 500 cancer cell lines spanning 21 cancer types to generate a pan-cancer Metastasis Map (MetMap). To facilitate high throughput profiling, cell lines were used that had been barcoded for use in the PRISM method, which was previously developed for in vitro testing of drug sensitivities (Yu et al., Nat. Biotechnol. 34: 419-23 (2016) the contents of each are hereby incorporated by reference in their entirety).

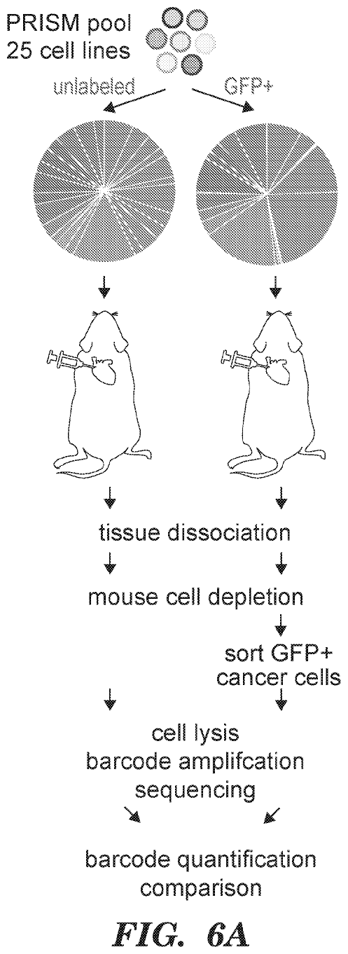

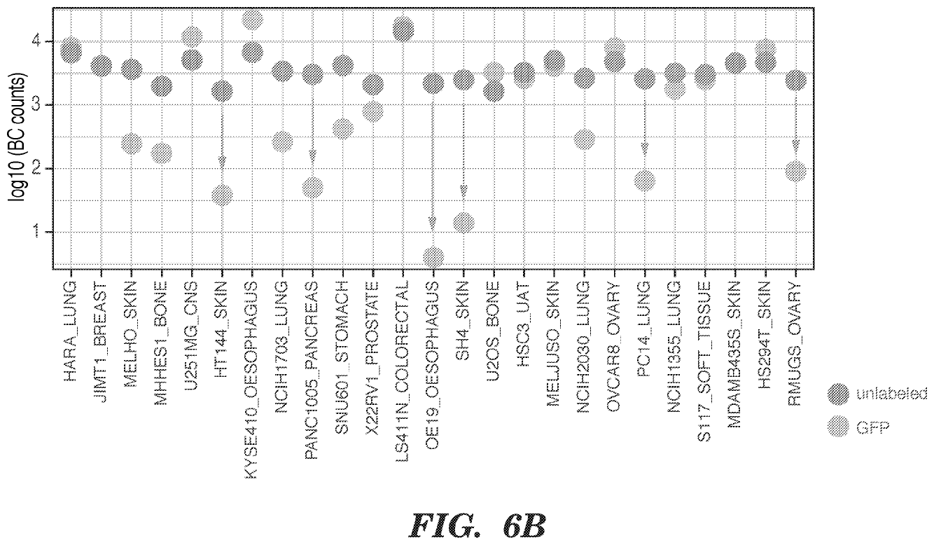

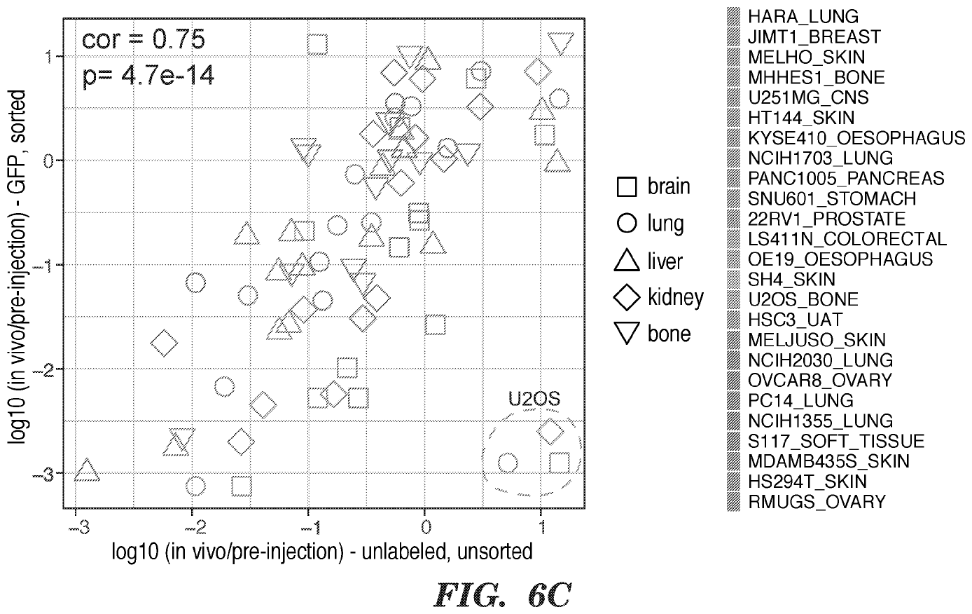

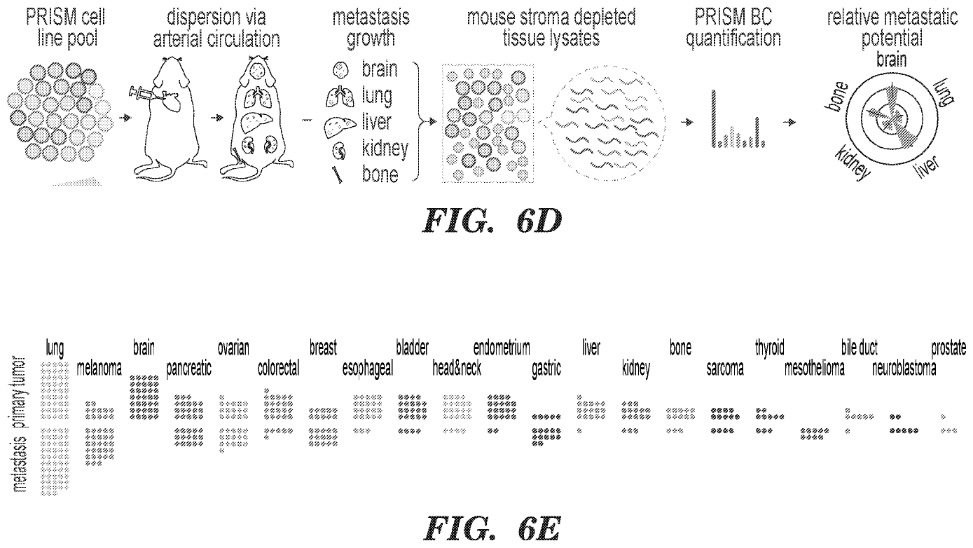

PRISM lines were pooled based on their in vitro doubling speed across mixed lineages, with 25 cell lines per pool. Because PRISM barcoded cells did not express GFP or luciferase, introducing labeling markers for cancer cell purification was analyzed to determine if it was critical for the method. One PRISM pool (of 25 cell lines) that contained the JIMT1 cell line was transformed with a GFP-luciferase vector, and cells were sorted by GFP expression (FIG. 6A). Consistent with different susceptibilities of cell lines to virus infection, 6 of the 25 cell lines showed strong dropout after GFP labeling, but all lines remained detectable (FIG. 6B). In contrast, cell lines prior to labeling displayed a more even barcode distribution, close to equal ratio pooling.

The GFP-labeled and unlabeled cell pools were subjected to the same animal workflow, tissue dissociation, and mouse cell depletion. The GFP-labeled group was further sorted to purify cancer cells. Isolated GFP-labeled cancer cells or tissue lysates from the unlabeled cell lines were subjected to barcode amplification and sequencing. A comparison of the two experiments showed highly concordant results. Although the initial barcode distribution of the pre-injected pools had altered (FIG. 6B), the enrichment (fold change) of barcode abundance showed strong positive correlation after normalizing to the pre-injected input (FIG. 6C), one exception was U205). Importantly, cell lines such as MELHO, MHHES1, and PC14 substantially dropped in their initial abundance after GFP labeling, yet they showed in vivo enrichment to the similar extent as in the non-labeled experiment. These results suggested that one could quantitatively detect barcodes from crude lysates without the need of cancer cell isolation. The simplified workflow shown in FIG. 6A was employed to generate the pan-cancer

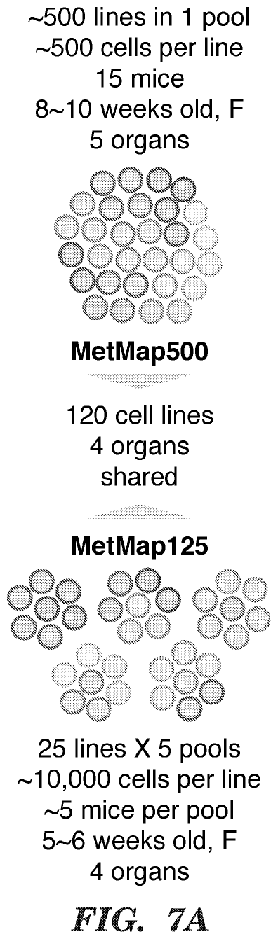

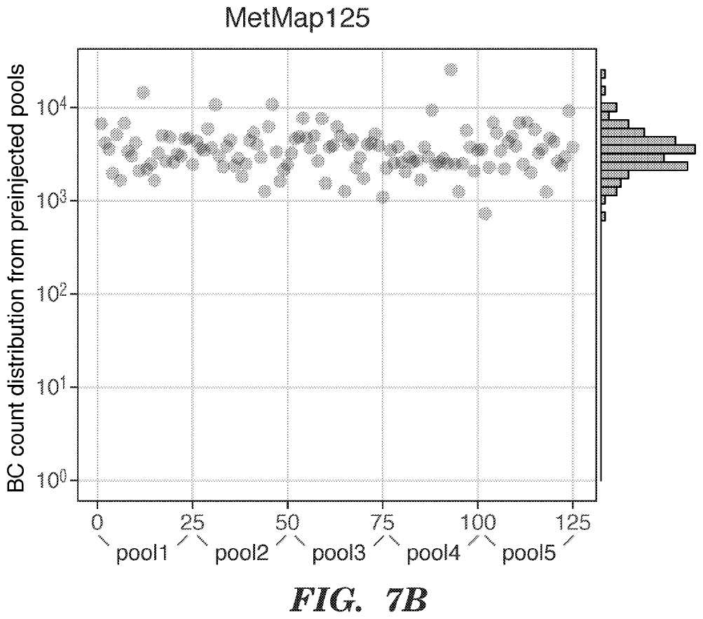

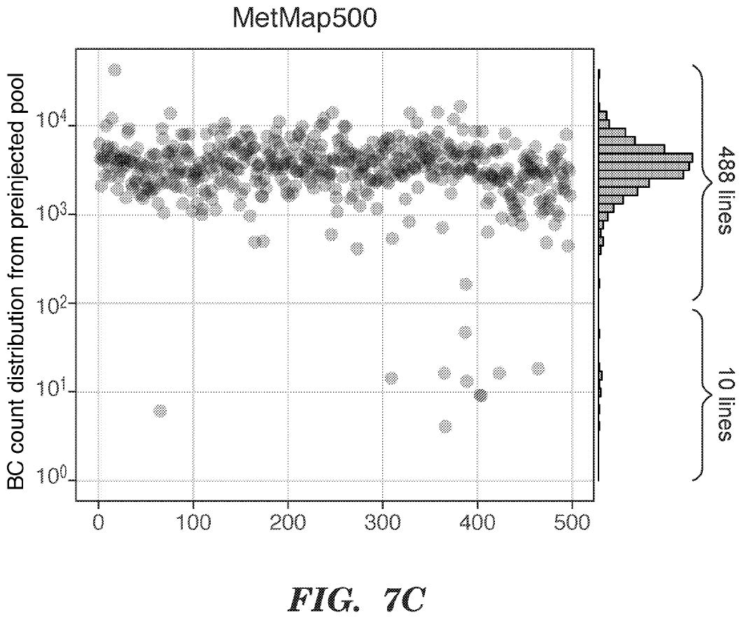

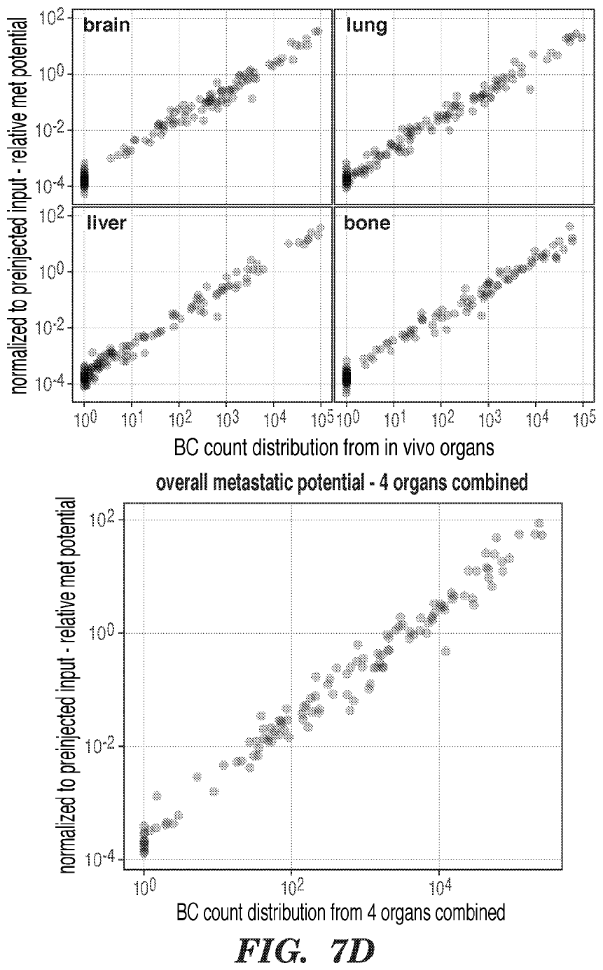

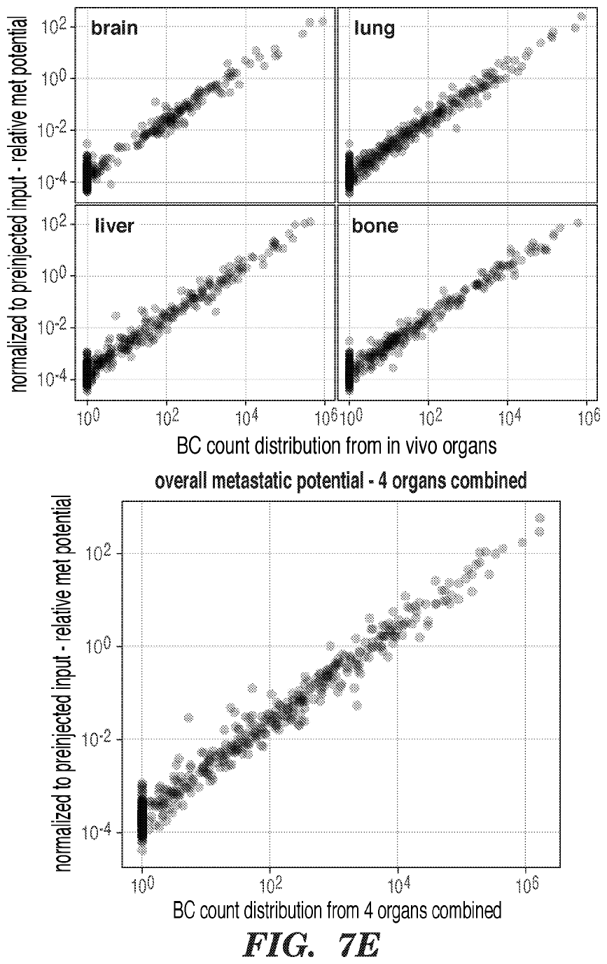

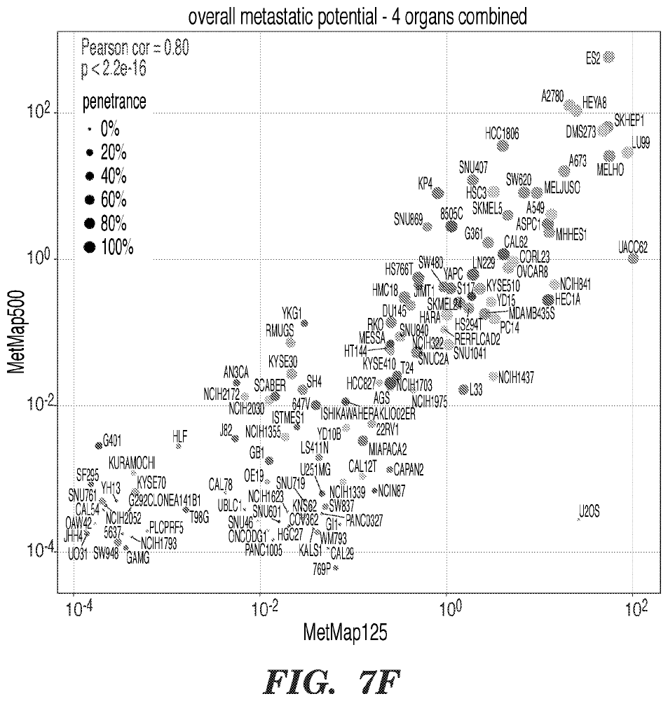

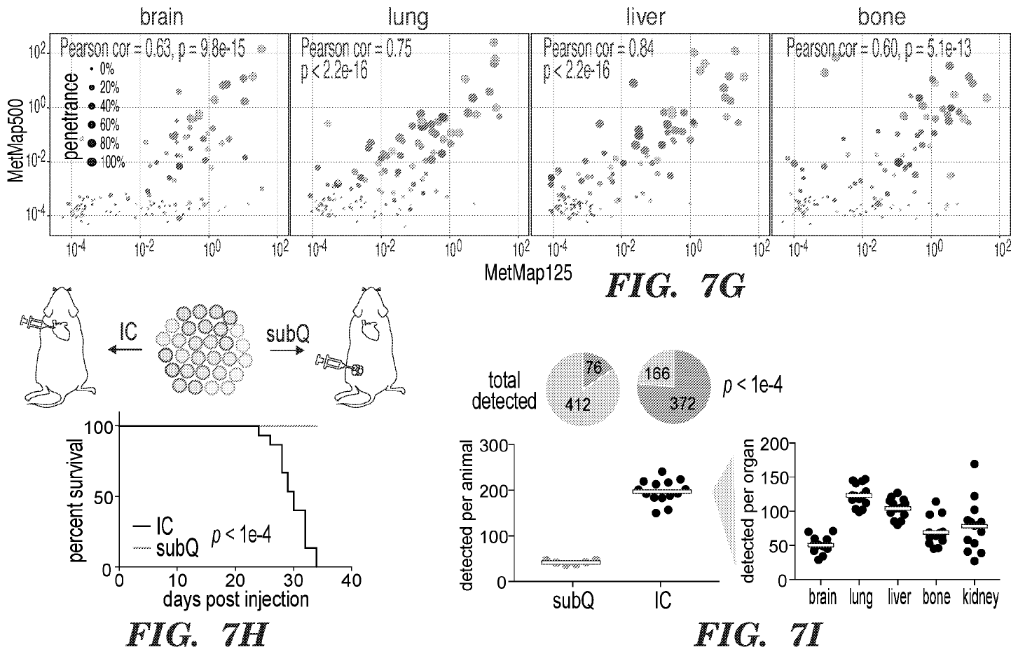

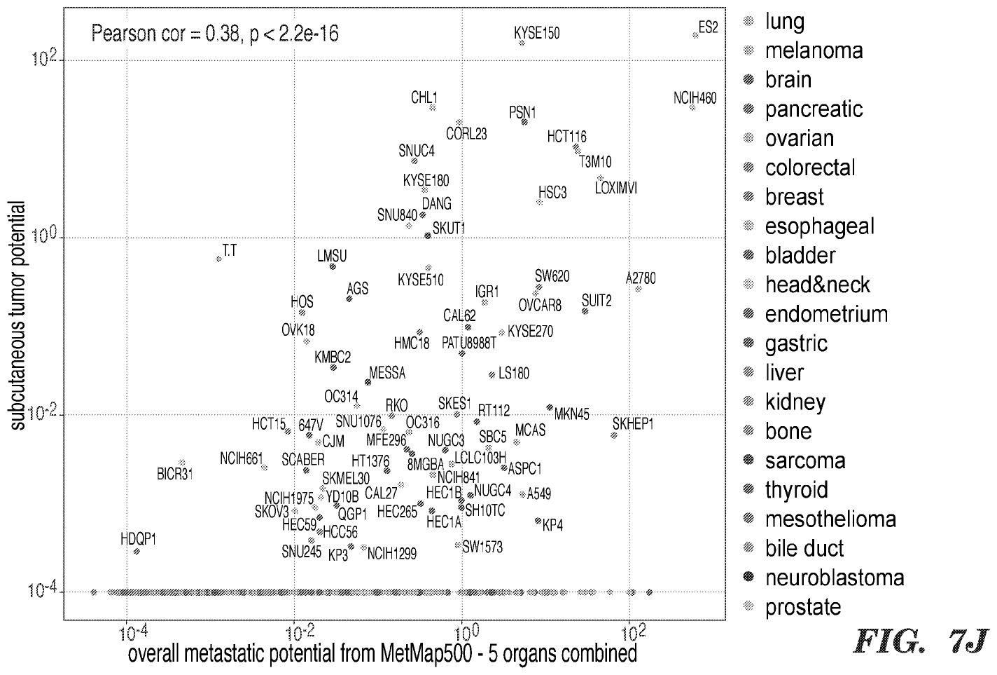

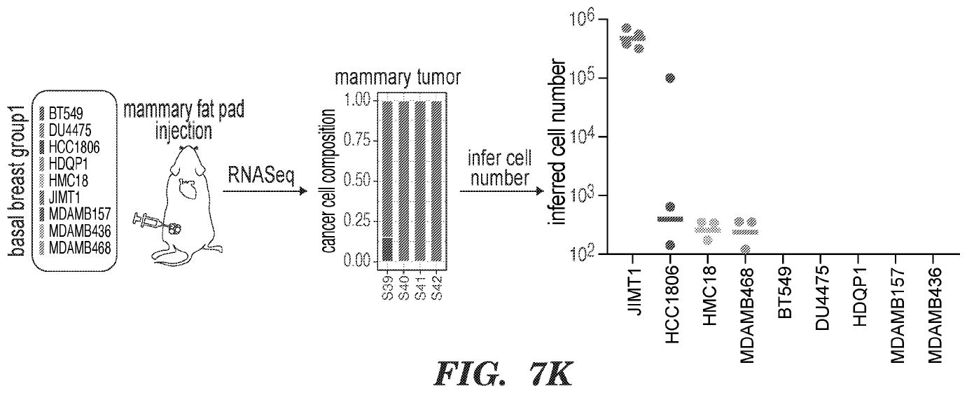

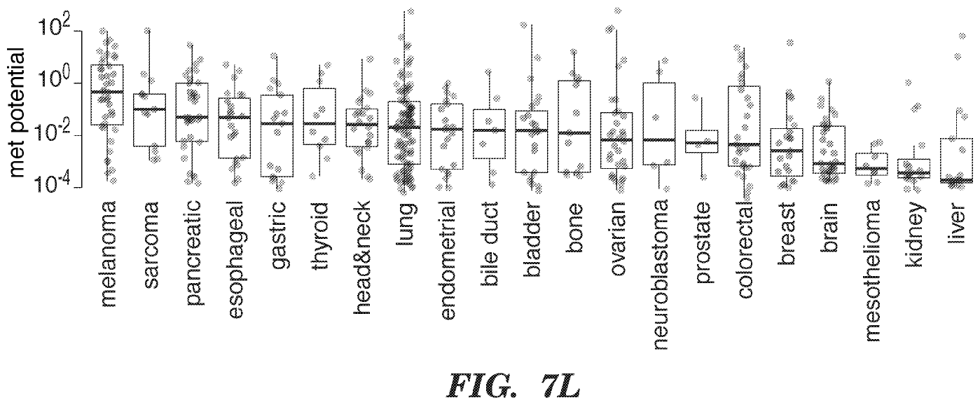

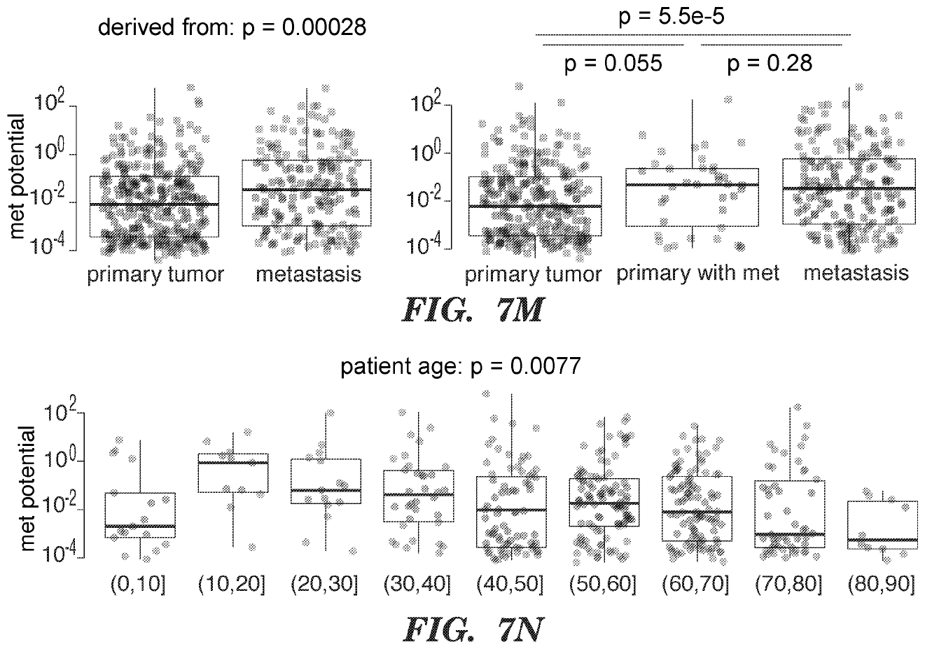

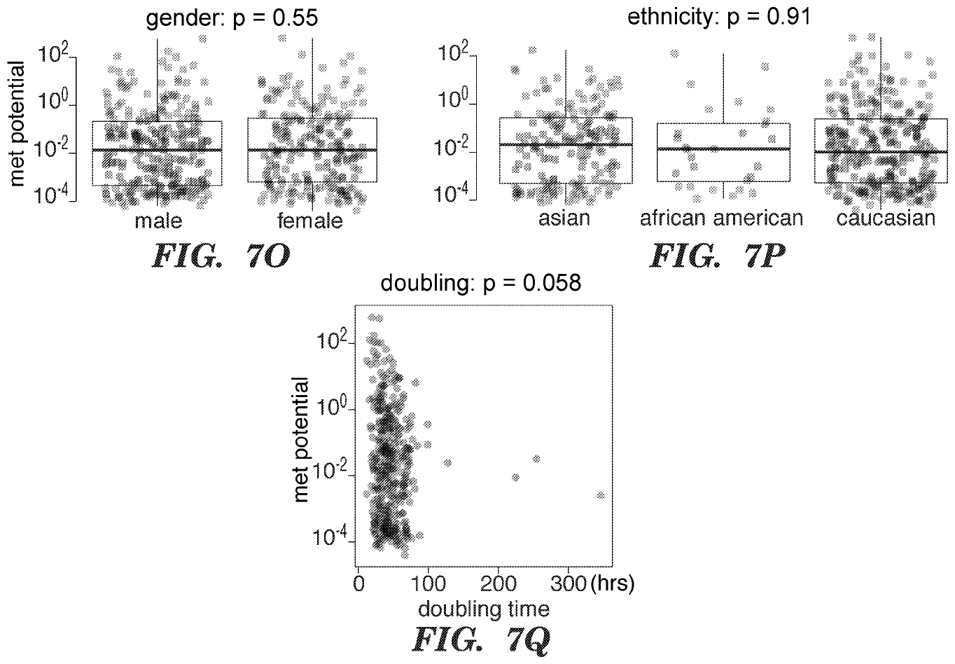

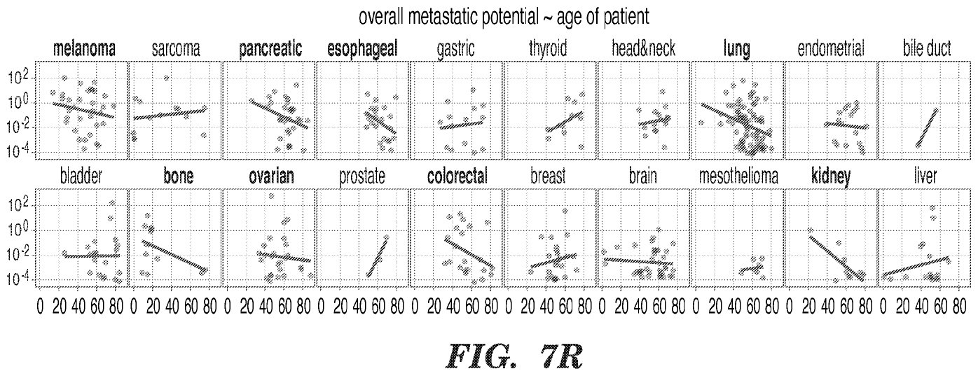

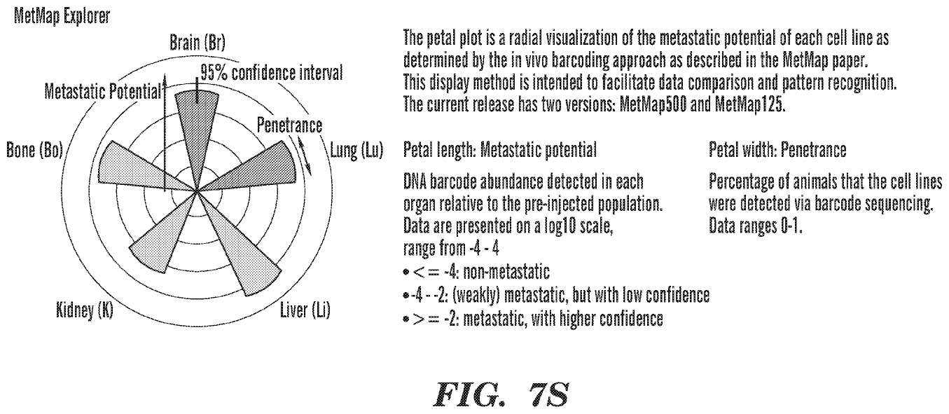

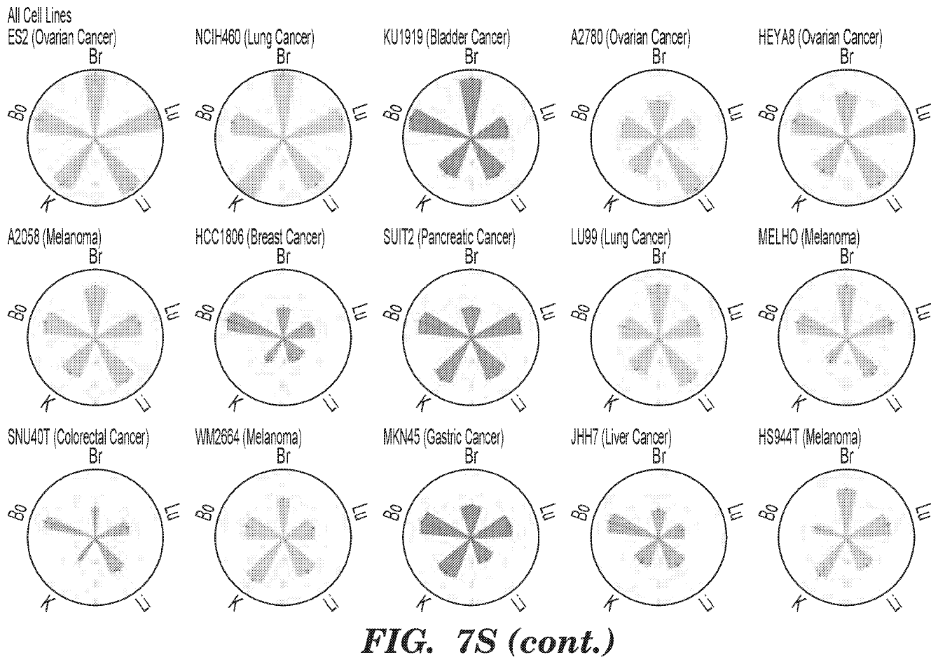

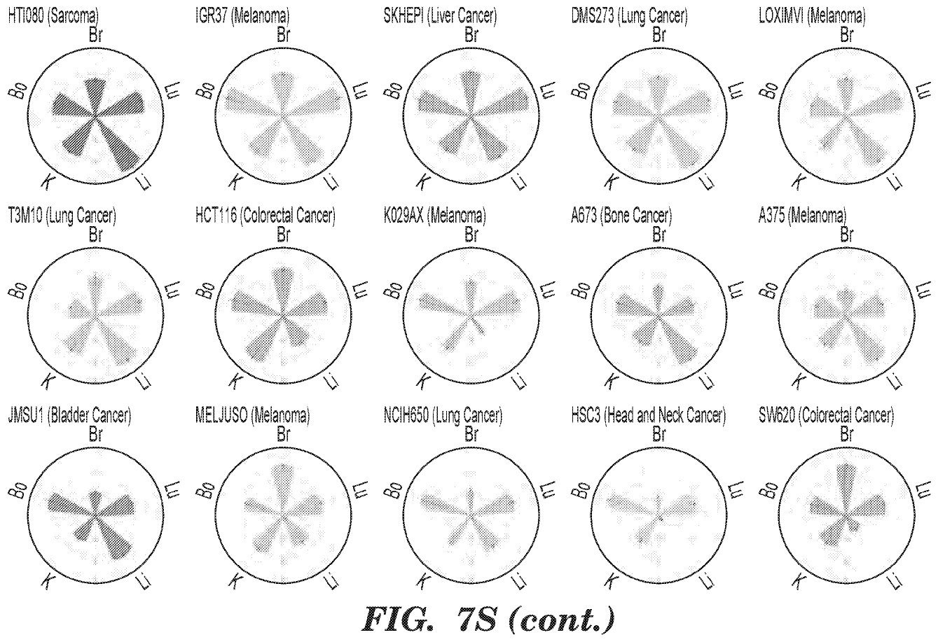

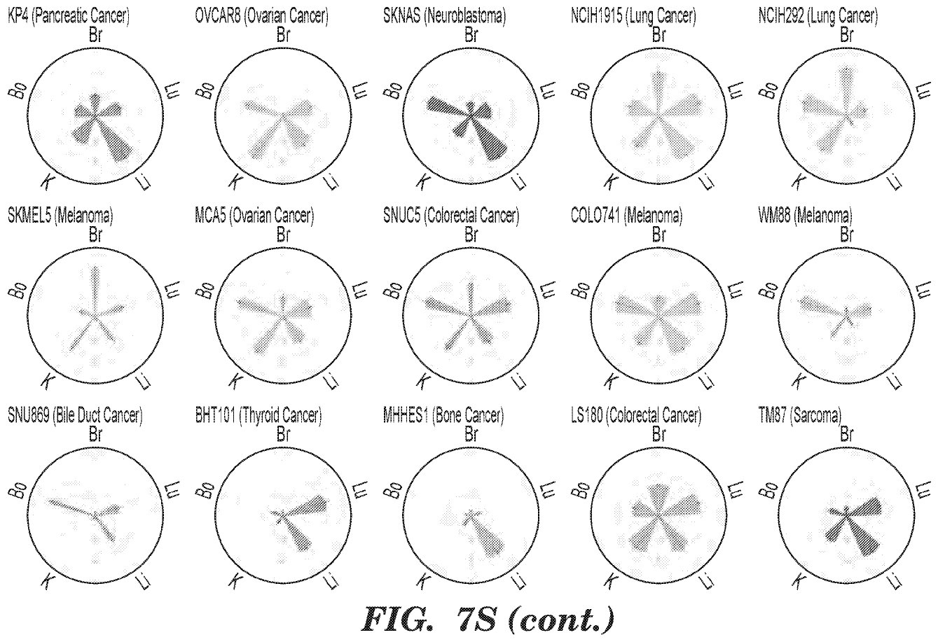

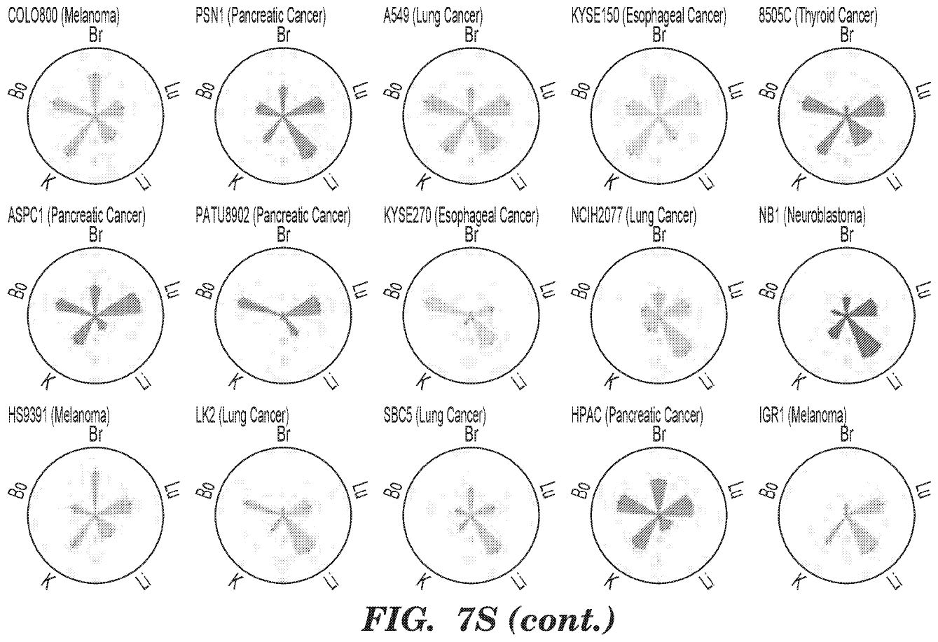

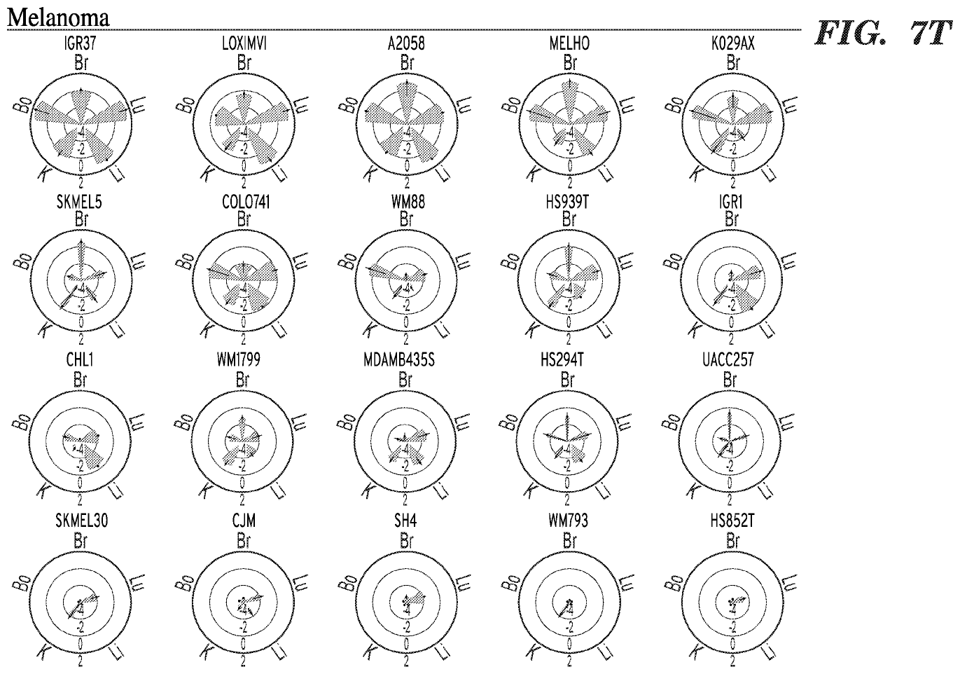

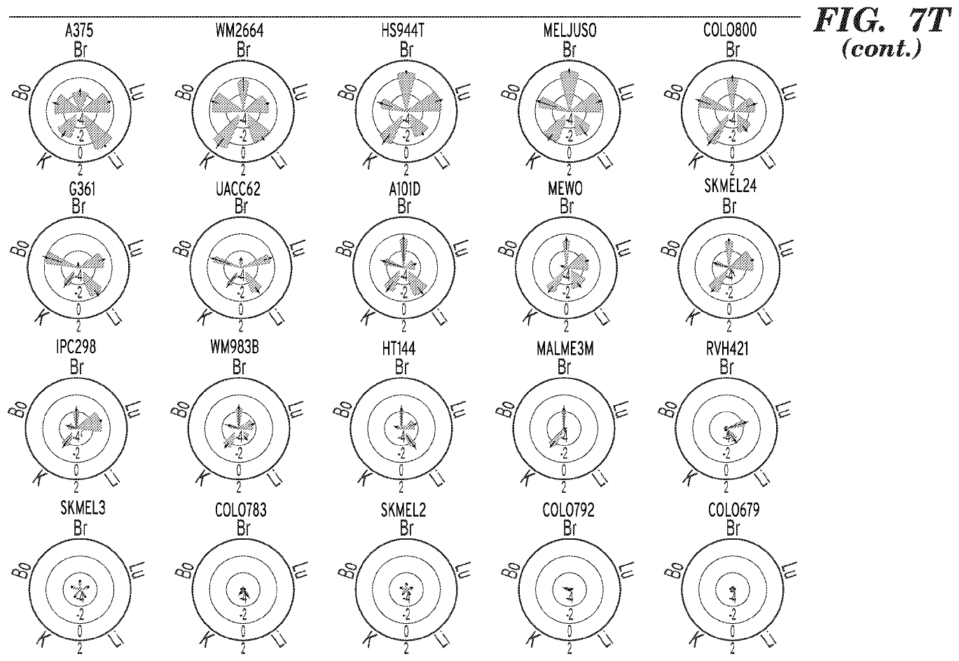

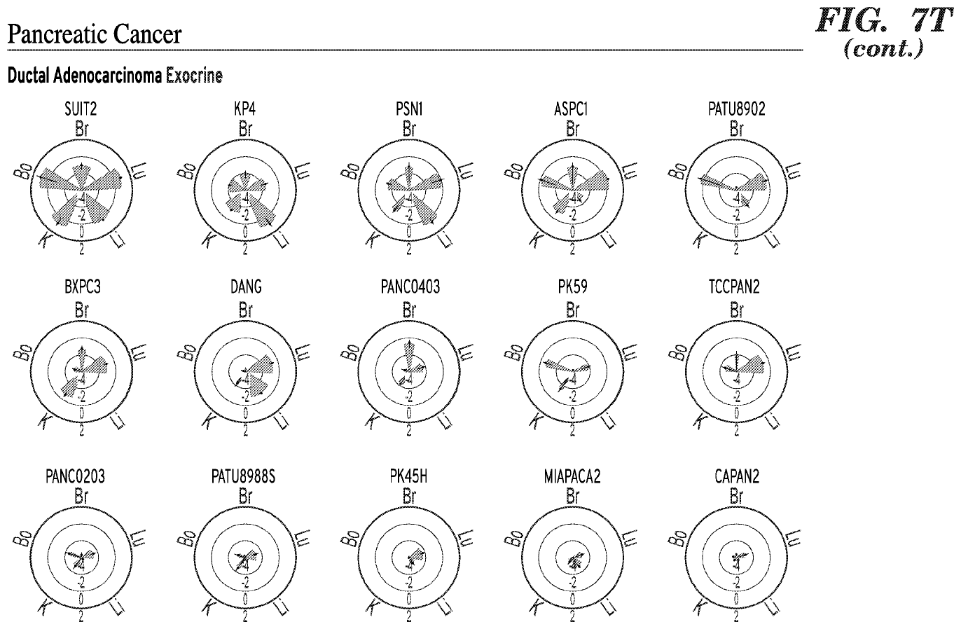

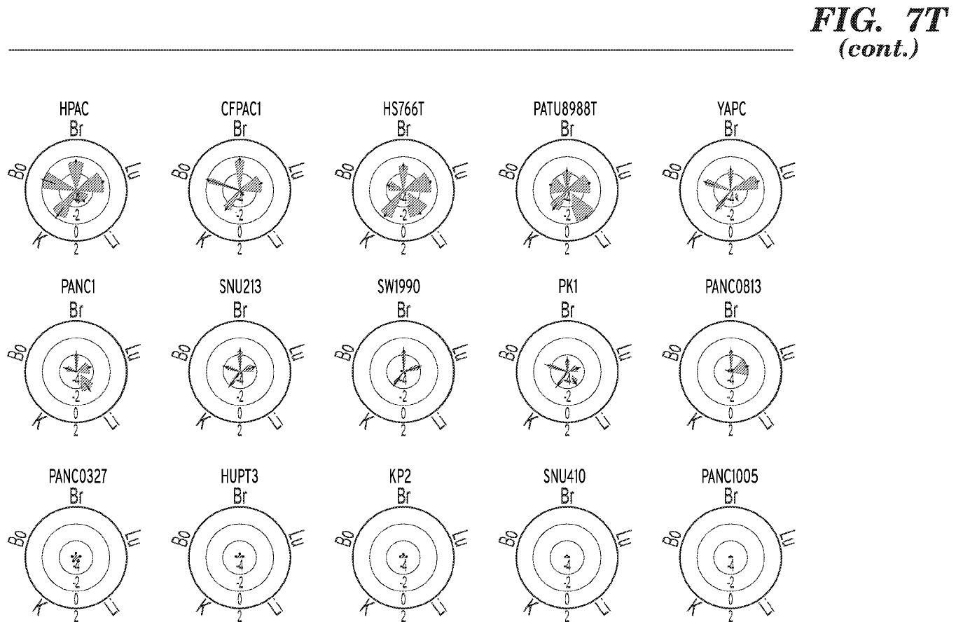

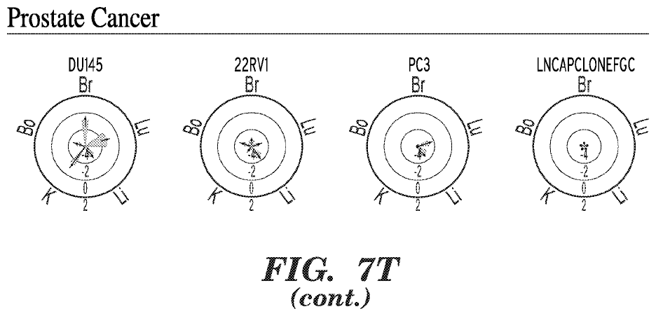

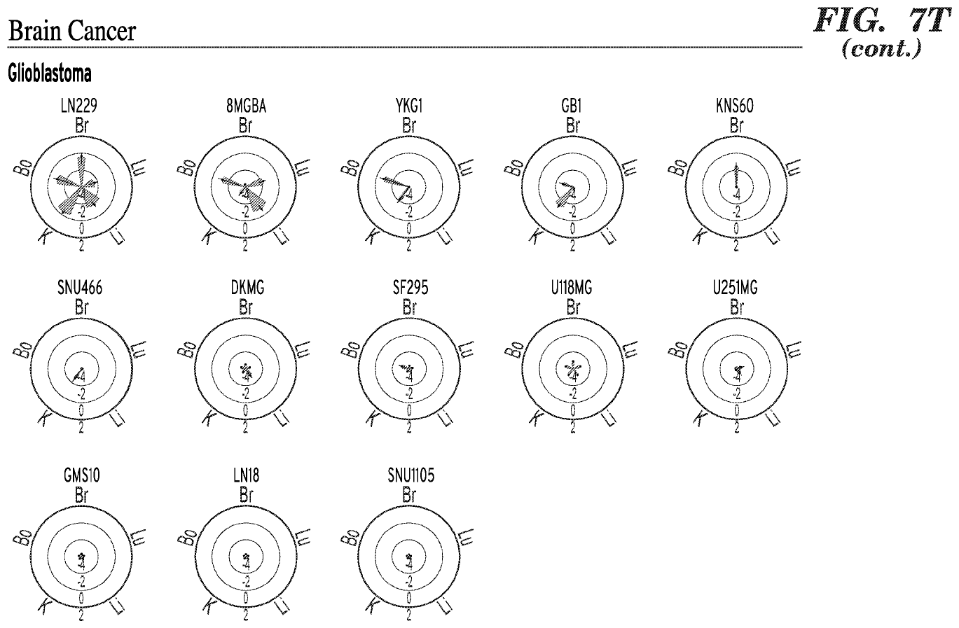

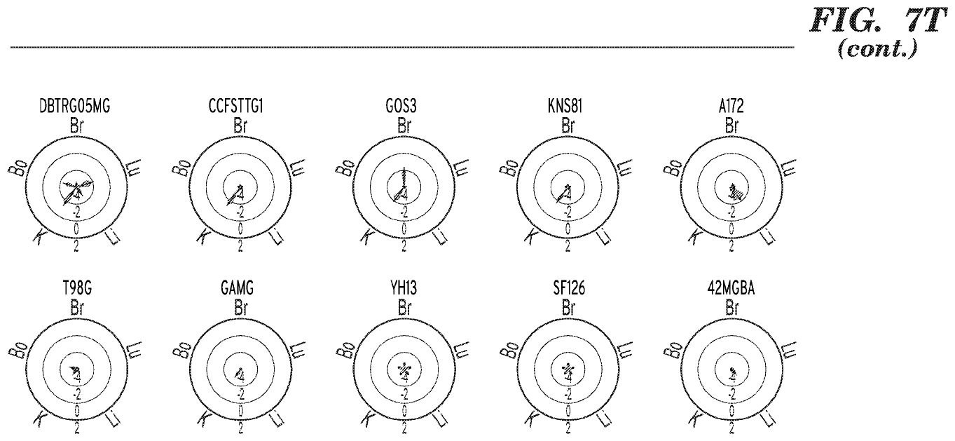

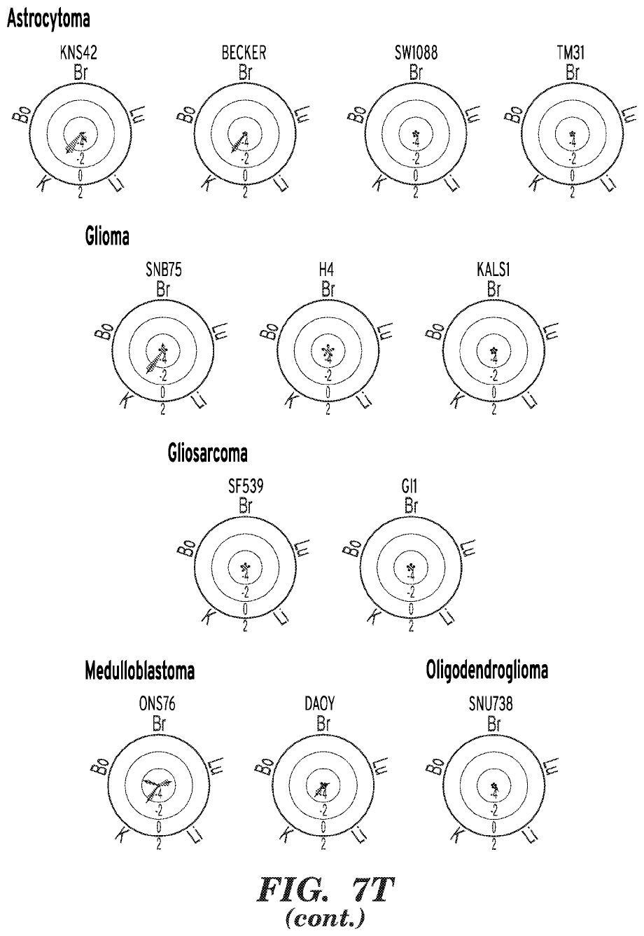

MetMap (FIG. 6E). This workflow allowed for the quantitative detection of barcodes from crude tissue lysates without the need of FACS-based tumor cell purification (FIG. 6D). The relative metastatic potential was quantified by enrichment of barcodes in in vivo metastases relative to the pre-injected input and was used as a metric to compare cell lines. Profiling was conducted in two pooling formats, with 500 cell lines profiled as a single pool in one, and in the other, with 125 profiled in 5 pools of 25 lines, each pool into different mice. The two experiments also differed in cell number, cohort size, and animal age (FIG. 7F). Strong correlation of the metastatic potential was observed despite differences in experimental conditions (Pearson correlation=0.8, p <2.2e-16, (FIGS. 7G, 7H), highlighting the strong robustness of the approach.

The resulting metastasis map (MetMap) is the largest ever generated (FIG. 7T). Data and interactive visualization are publicly accessible at pubs.www.broadinstitute.org/metmap.

It was also noted that the intracardiac injection approach allowed for the evaluation of far more cell lines in vivo compared to traditional subcutaneous (subQ) injection (FIGS. 7H-7J). Specifically, an average of 197 cell lines per mouse were recovered following intracardiac injection, whereas only an average of 42 cell lines were recovered following subQ injection (FIG. 7I). This difference may be explained by the local competition for nutrients and other microenvironmental factors in the subQ setting, whereas the spatial separation of tumor cells in the metastasis models minimizes such competition. This finding of local competition was also seen in the orthotopic setting, where injection of a pool of 9 breast cancer cell lines into the mammary fat pad resulted in a single cell line dominating the resulting tumor (FIG. 7K). These results highlight the advantage of using pooled cell lines for investigating the metastasis phenotype.

To assess if MetMap reflects the metastatic behavior of various cancers the metastatic potential was compared with clinical annotations of cell lines. Significant association with (1) cancer lineage, (2) where the cell line was derived from, (3) patient age, but not with gender or ethnicity were found (FIGS. 7L-7T). As expected, metastatic potential differed substantially as the cancer type varied. Melanoma and pancreatic cancer lines were widely metastatic (FIG. 7T), which is consistent with these cancers' propensities to develop metastases in patients (Quintana et al., Sci. Transl. Med. 4: 159ra149 (2012); Damsky et al., Oncogene 33: 2413-22 (2014); Ryan et al., N. Engl. J. Med. 371: 1039-49 (2014), the contents of each are hereby incorporated by reference in their entirety). In contrast, brain tumor-derived cell lines were generally non-metastatic, which is reflective of their tendency not to undergo hematogenous spread (Fonkem et al., J. Clin. Oncol. 29: 4594-95 (2011); Muller et al., Sci. Transl. Med. 6: 247ra101 (2014), the contents of each are hereby incorporated by reference in their entirety). Similarly, the DU145 prostate cancer cell line, derived from a brain metastasis lesion, demonstrated brain metastasis (FIG. 7T).

Cell lines derived from metastases showed higher metastatic potential than lines derived from primary tumors. Interestingly, multiple cell lines derived from primary tumors known to give rise to metastases in patients were metastatic as xenografts (FIGS. 7M), consistent with previously reported suggestions that metastatic potential is encoded in primary tumors (Ramaswamy et al., Nat. Genet. 33: 49-54 (2003); Zhang et al., Cell 154, 1060-73 (2013); Vanharanta, et al., Cancer Cell 24: 410-21 (2013); Puram et al., Cell 171: 1611-24 (2017), the contents of each are hereby incorporated by reference in their entirety).

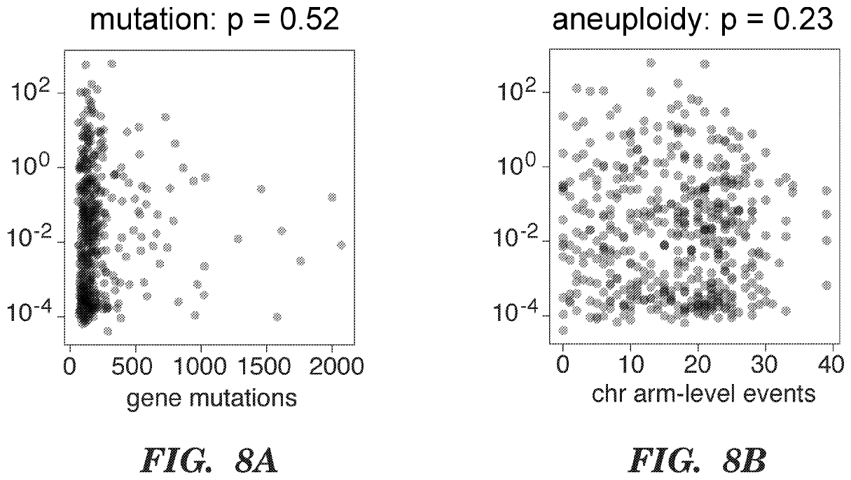

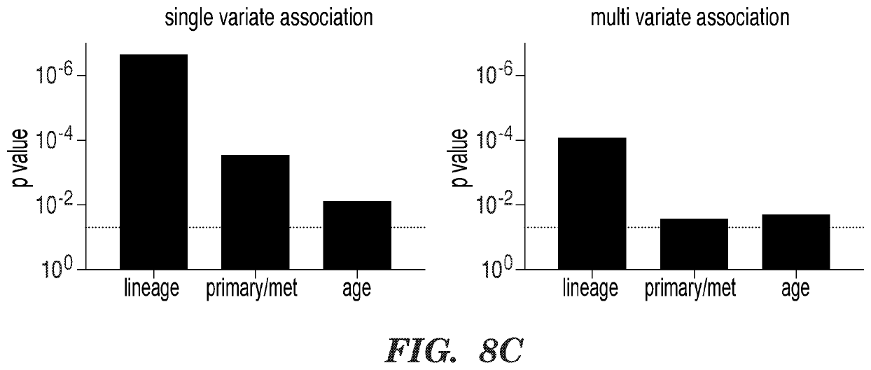

The association with aging of patients was unexpected, where a gradual decline of metastatic potential was observed as the age of cancer patient increased (FIG. 7N). Multivariate analysis indicated that it is a contributing factor independent of other associating factors (FIGS. 7R, 8C). Of note, such association was impossible to capture from a small cohort study with only a few cell lines, highlighting the power of MetMap.

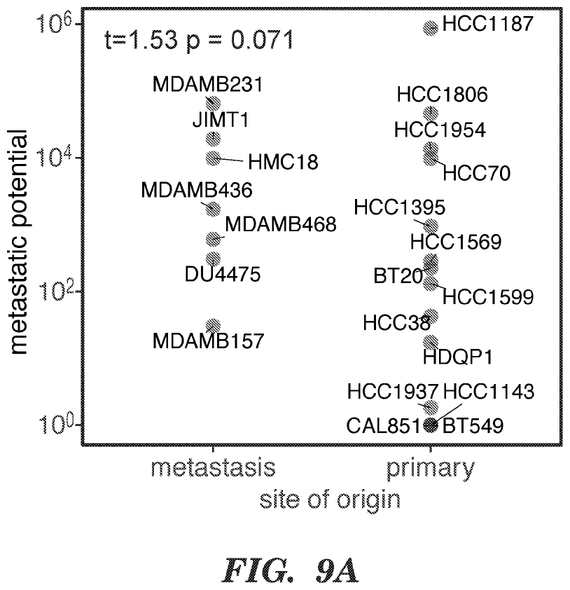

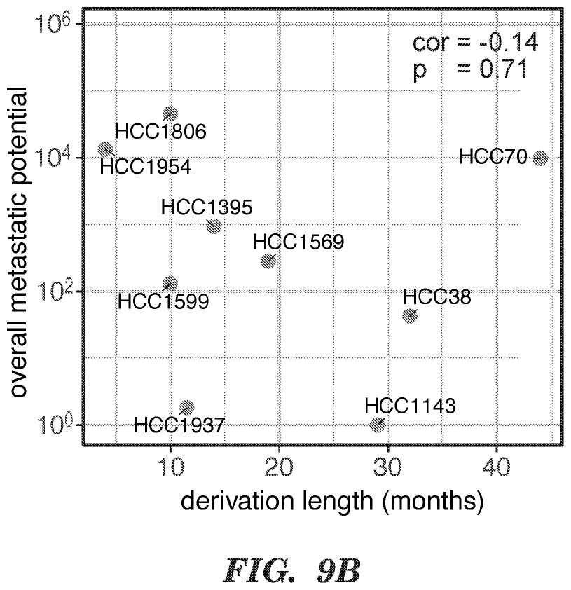

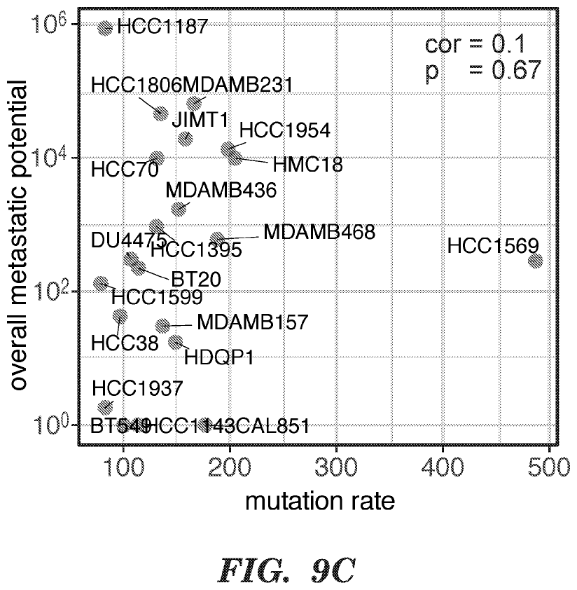

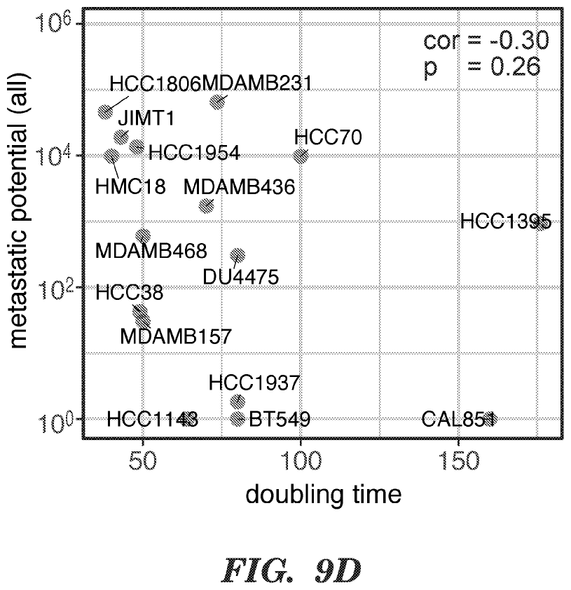

Perhaps most importantly, extensive variation in metastatic potential was observed within individual lineages, thereby making it possible to search for associations between metastasis propensity and genomic features of the tumors. Of note, metastatic potential was not simply explained by cell line proliferation rate or mutational burden (FIGS. 8A to 8C and 9A to 9D), suggesting that subtler molecular determinants of metastasis were at play.

### Example 4

### DNA Mutations Associated with Brain Metastasis

To investigate mechanisms involved in metastasis, efforts were focused on breast cancer and its potential for brain metastasis (see FIG. 4H), because brain metastasis is a feature of some, but not all, breast cancers, and because little is known that might inform therapeutic approaches (Valiente et al., Trends Cancer 4: 176-96 (2018); Witzel, et al., Breast Cancer Res. 18: 8 (2016), the contents of each are incorporated herein by reference in their entirety). A systematic and unbiased comparison was performed of the molecular features that distinguished brain-metastatic from non-metastatic breast cancer cell lines. As described below, these analyses consistently pointed to a connection between metastatic potential and lipid metabolism.

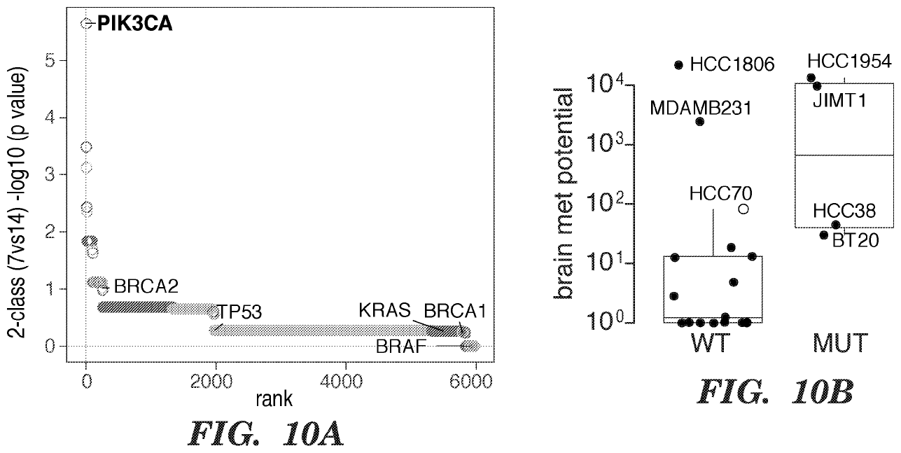

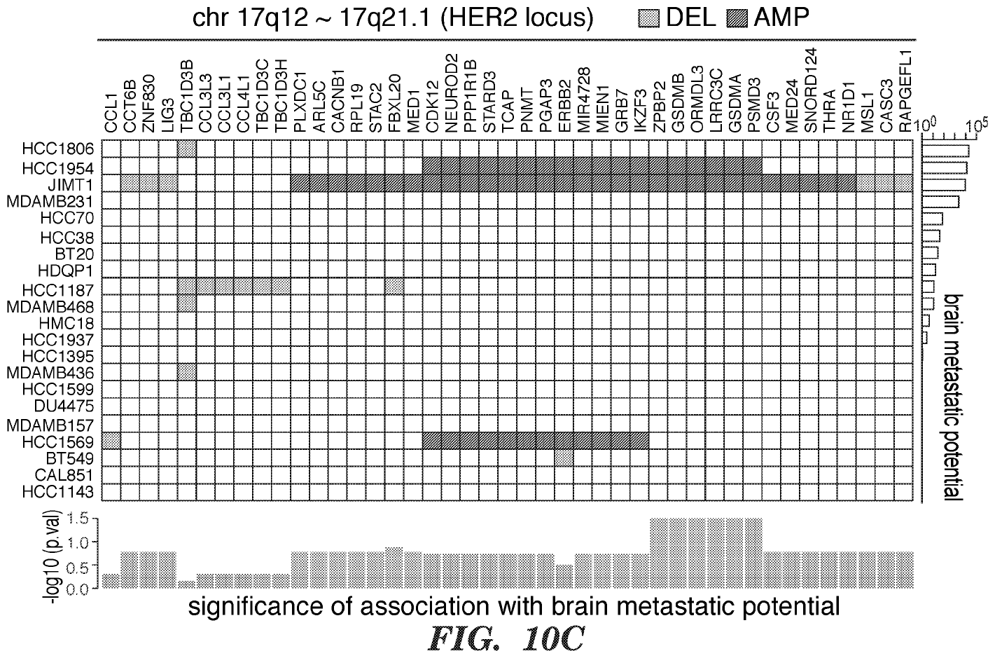

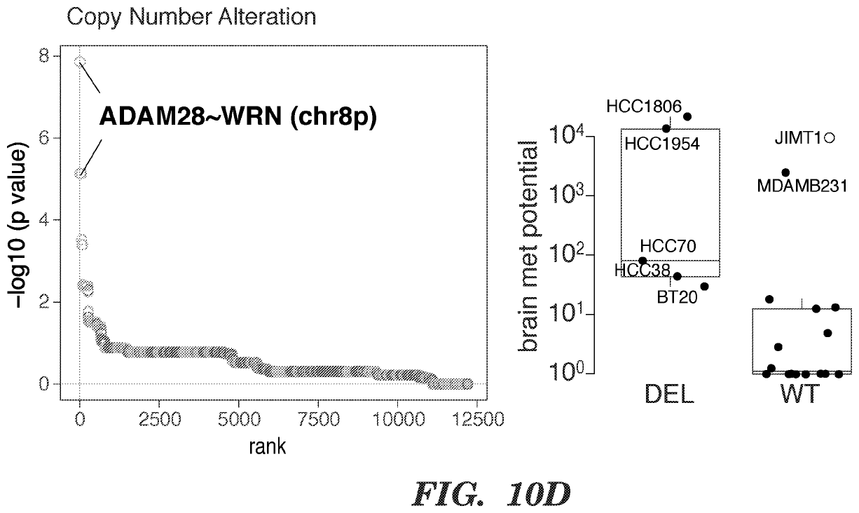

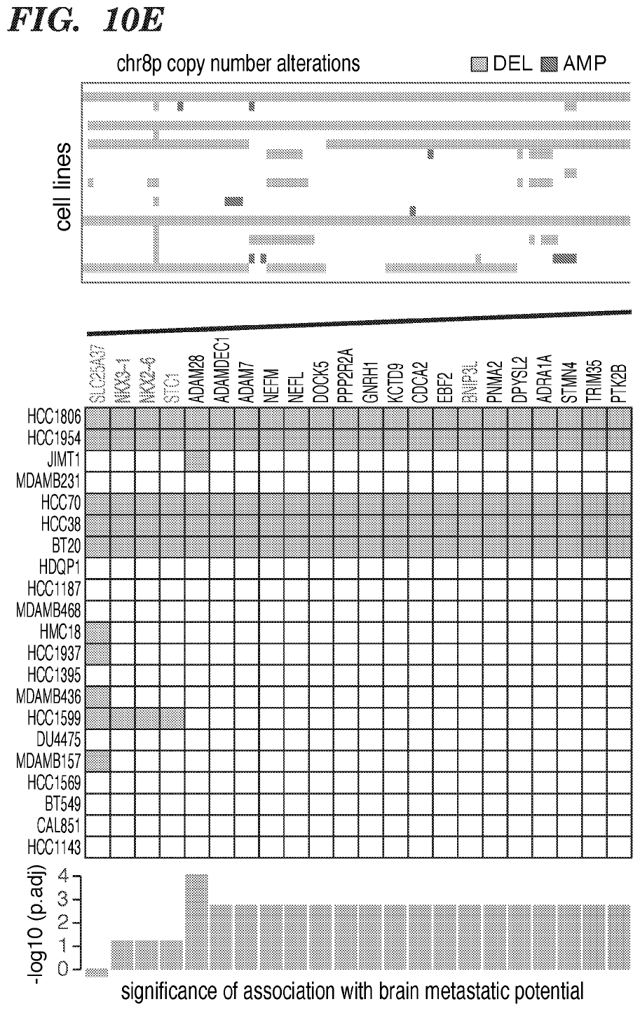

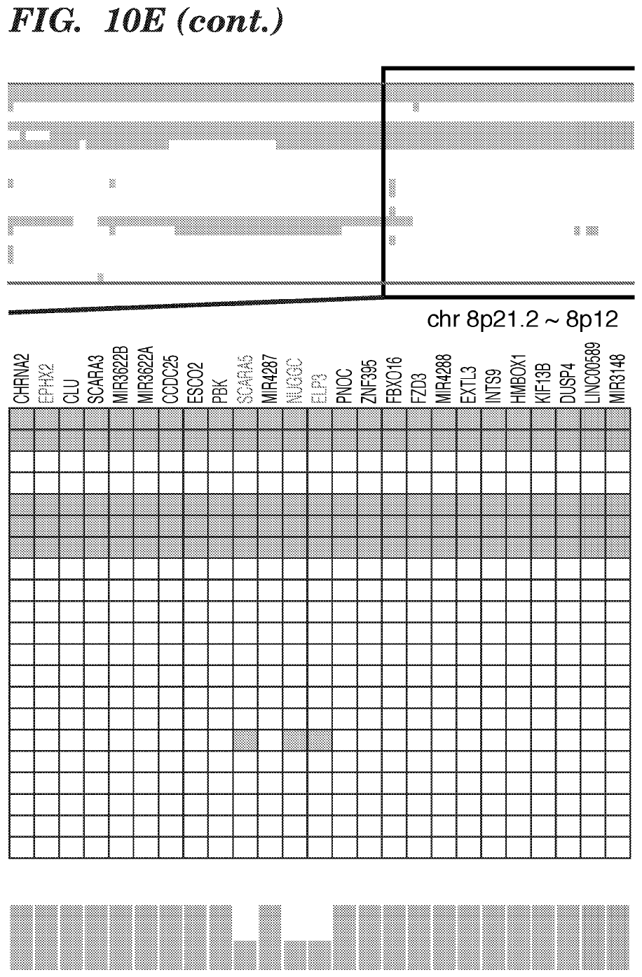

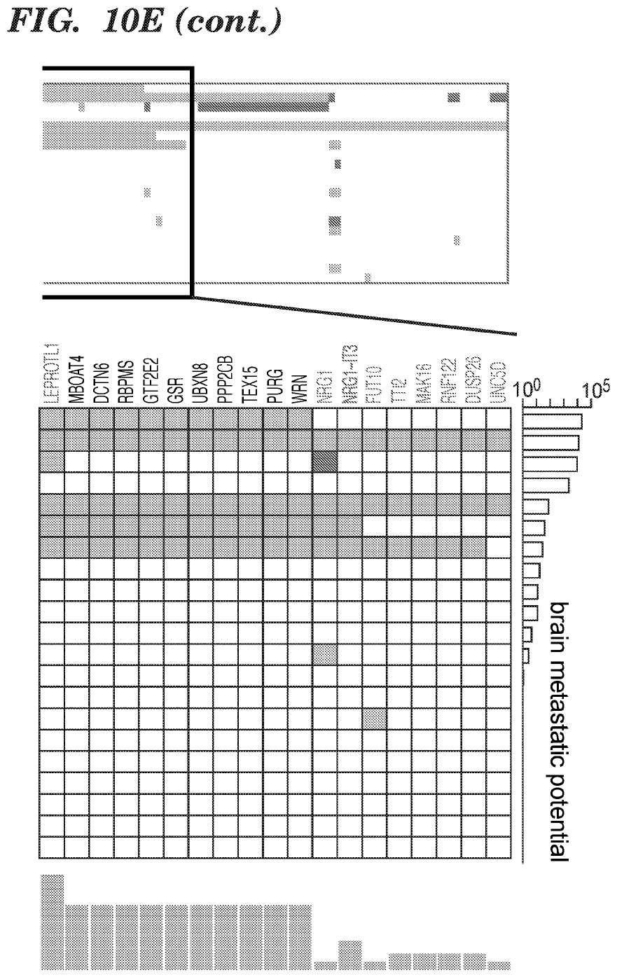

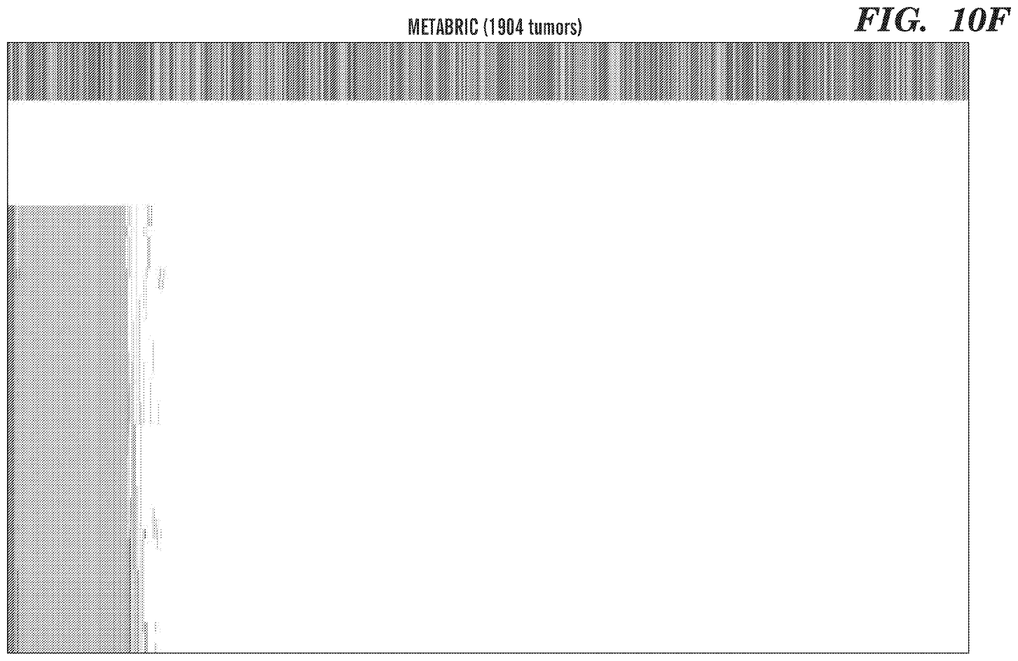

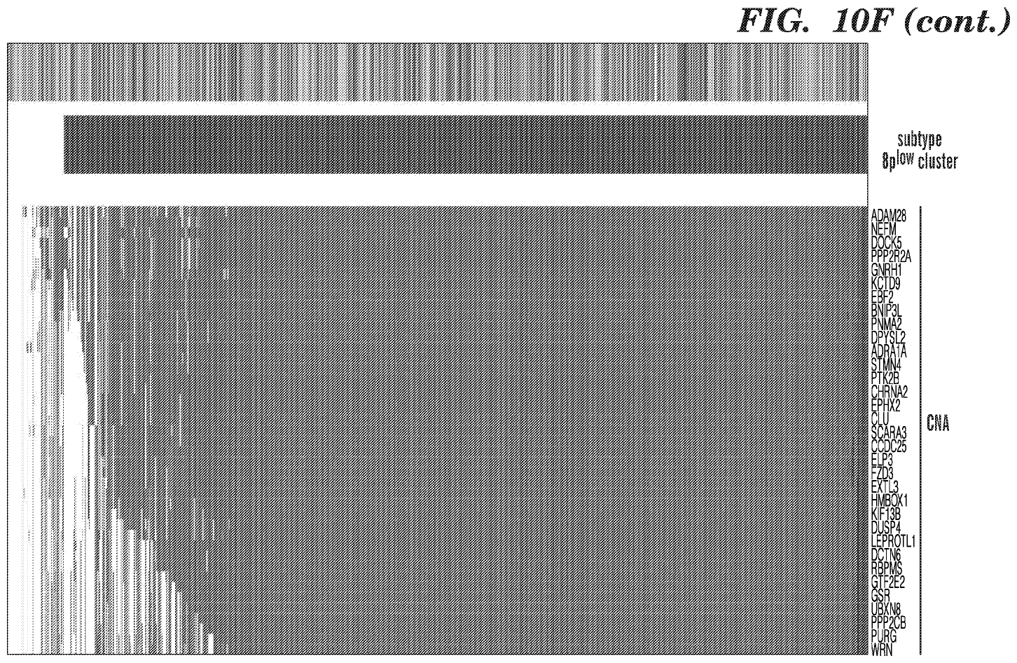

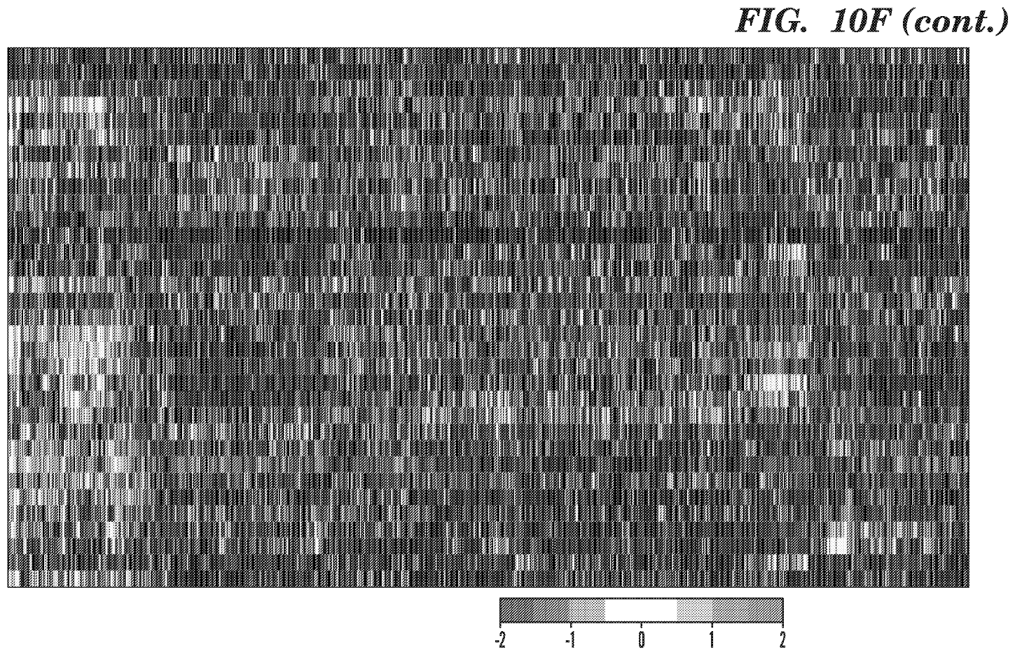

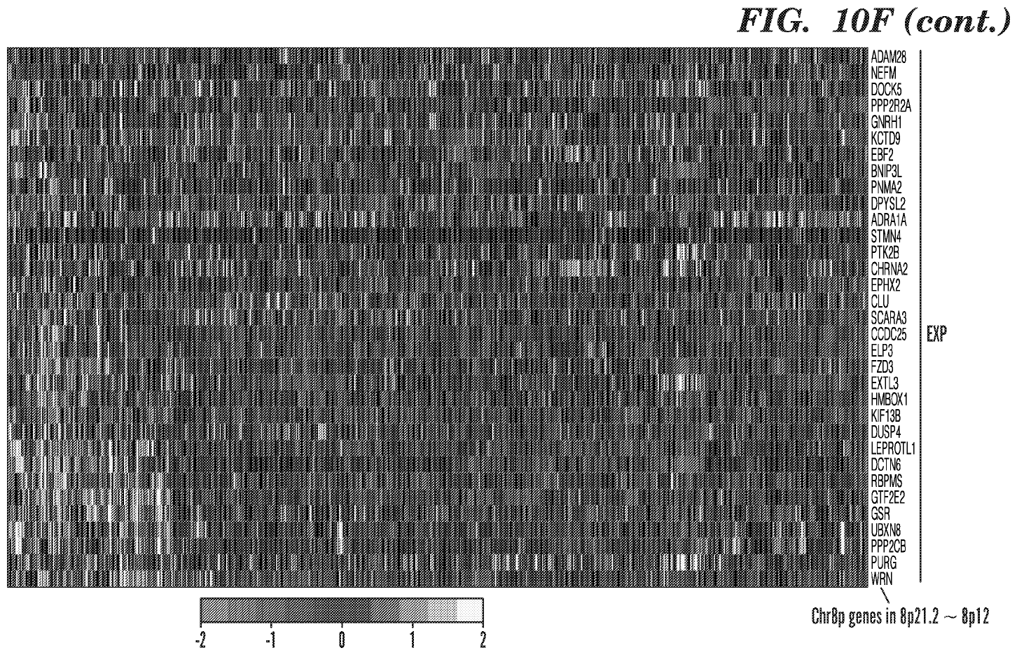

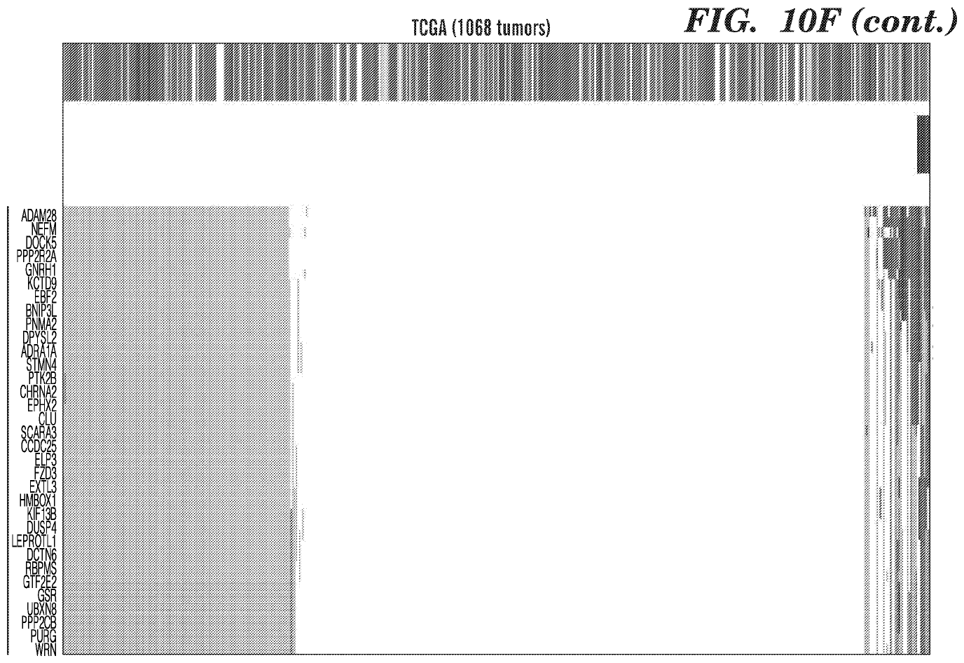

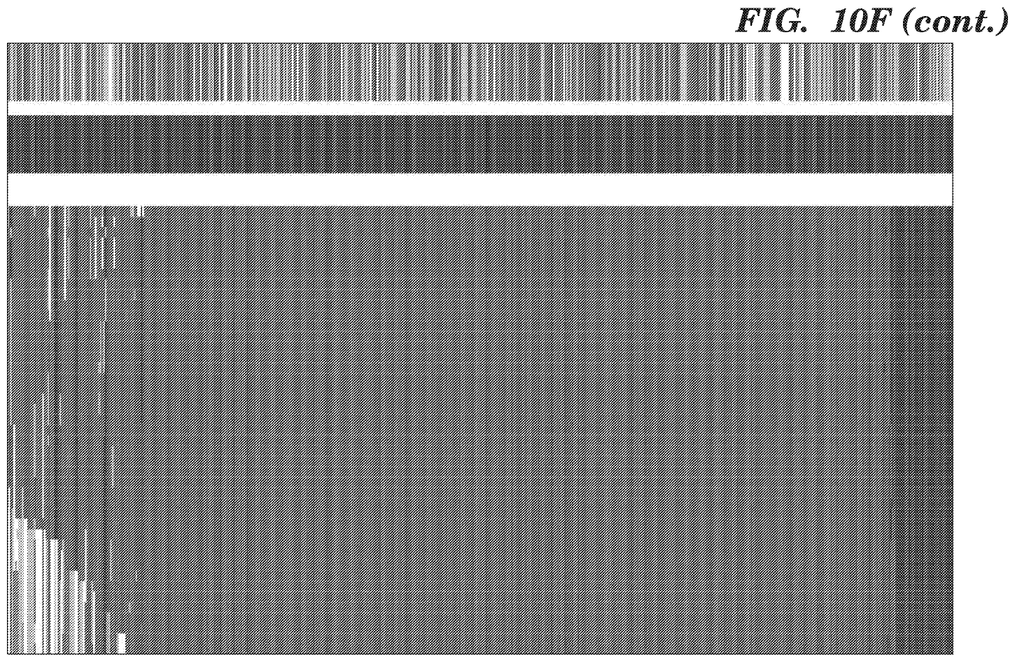

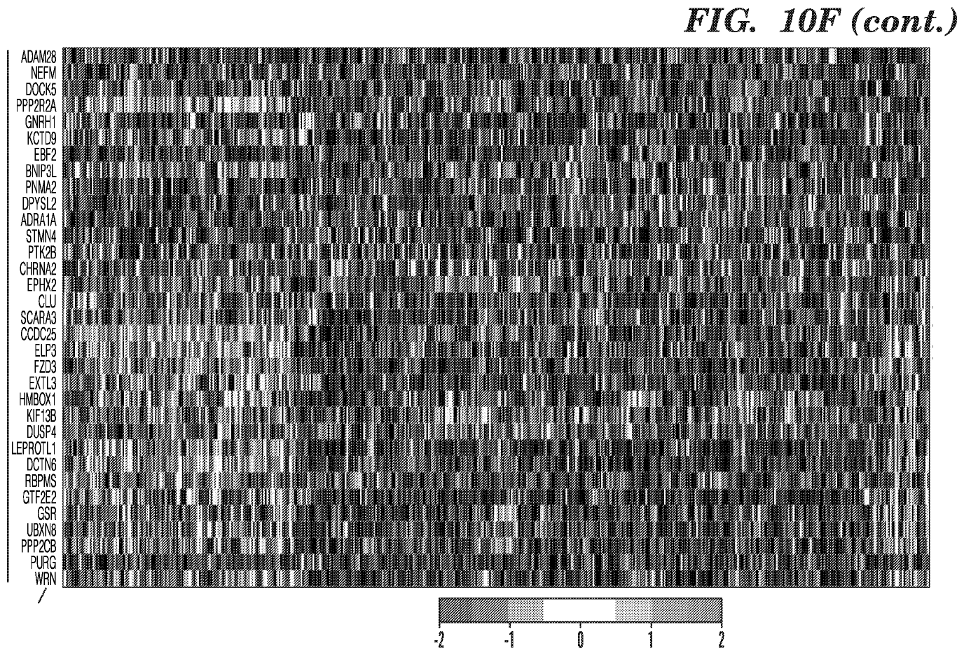

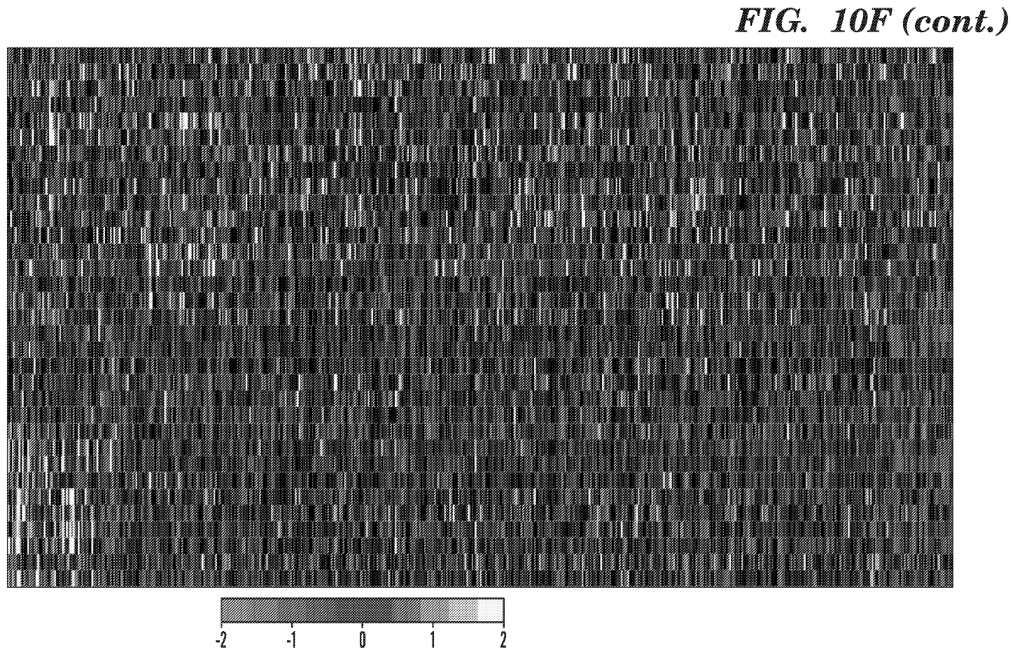

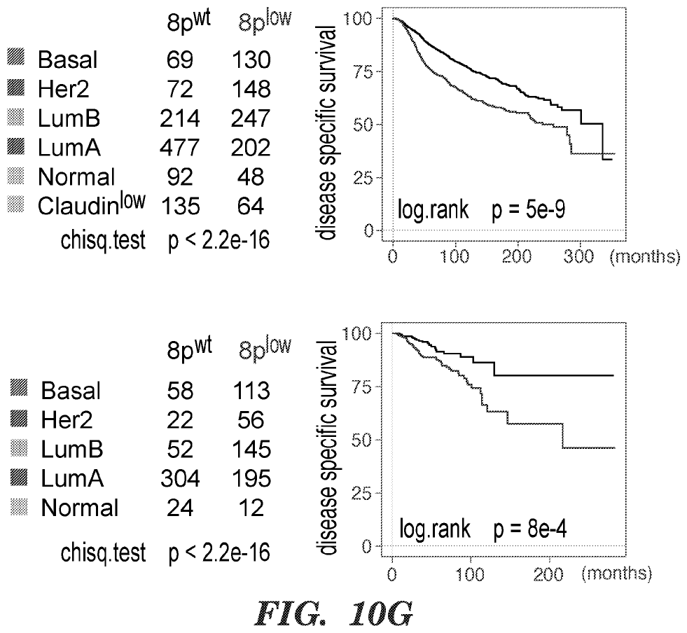

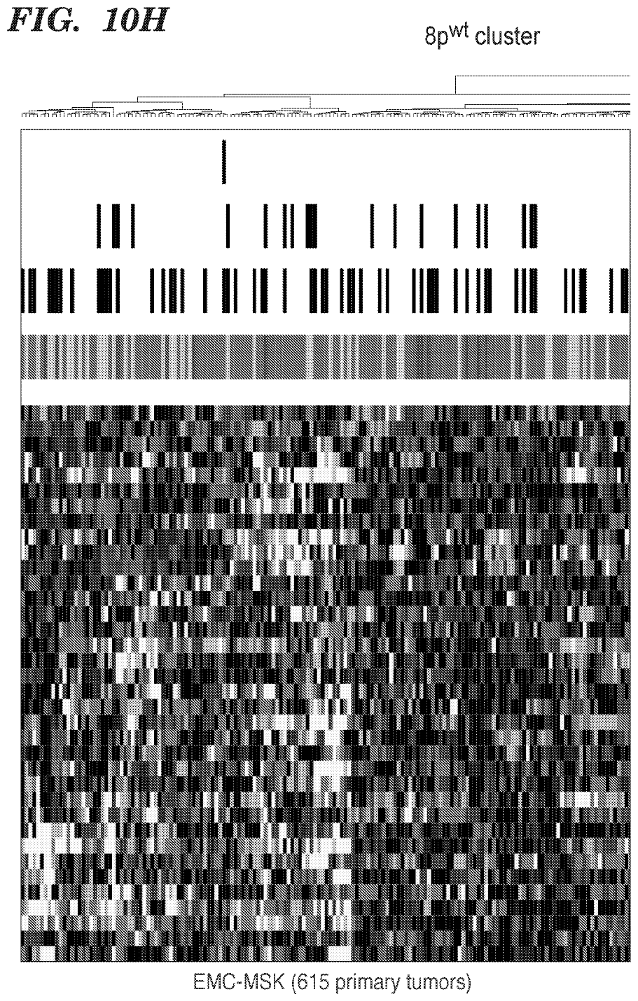

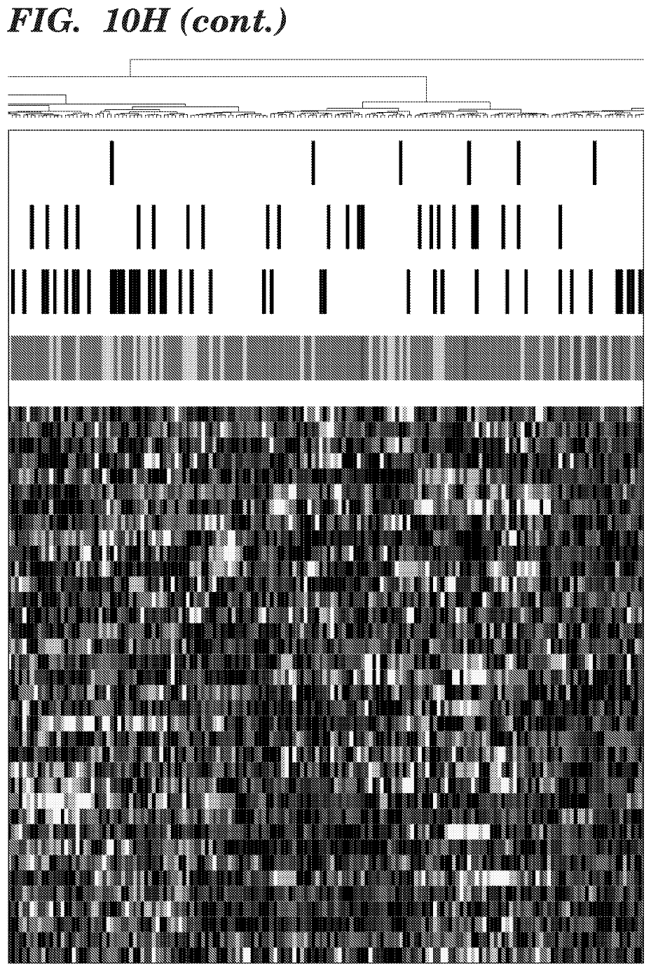

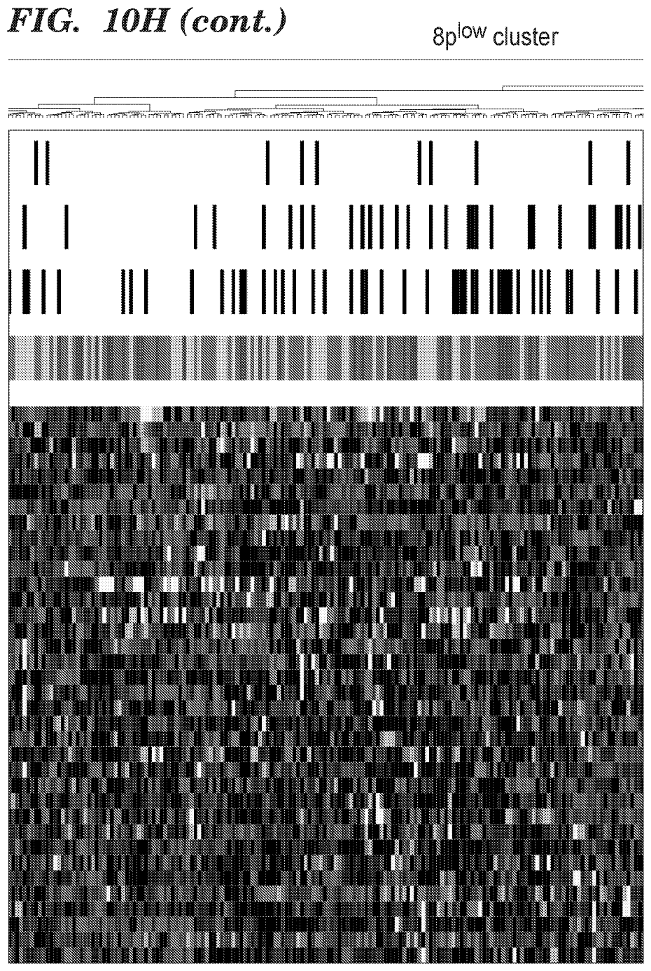

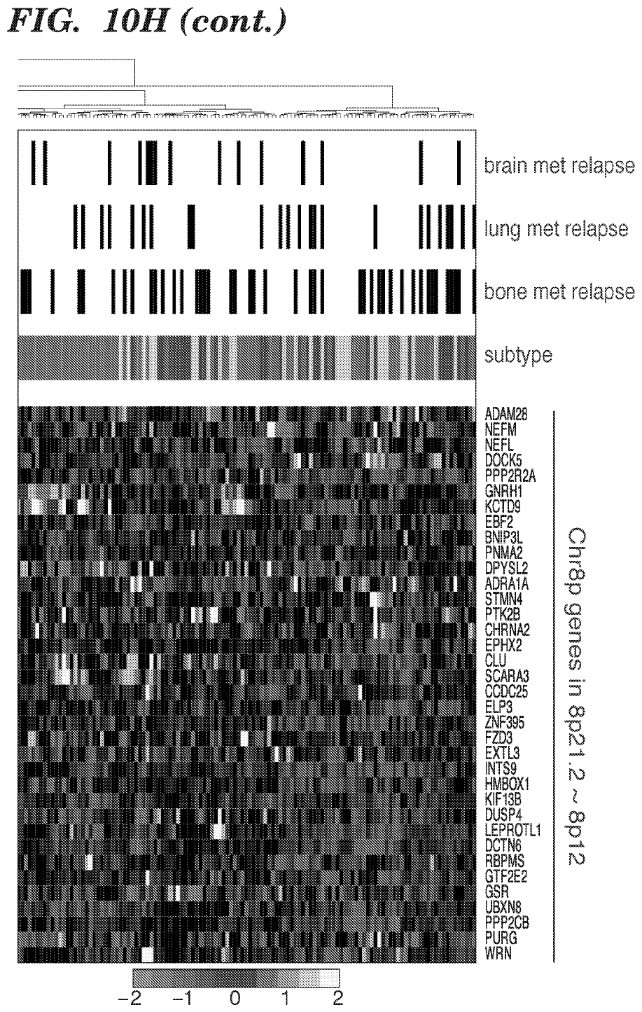

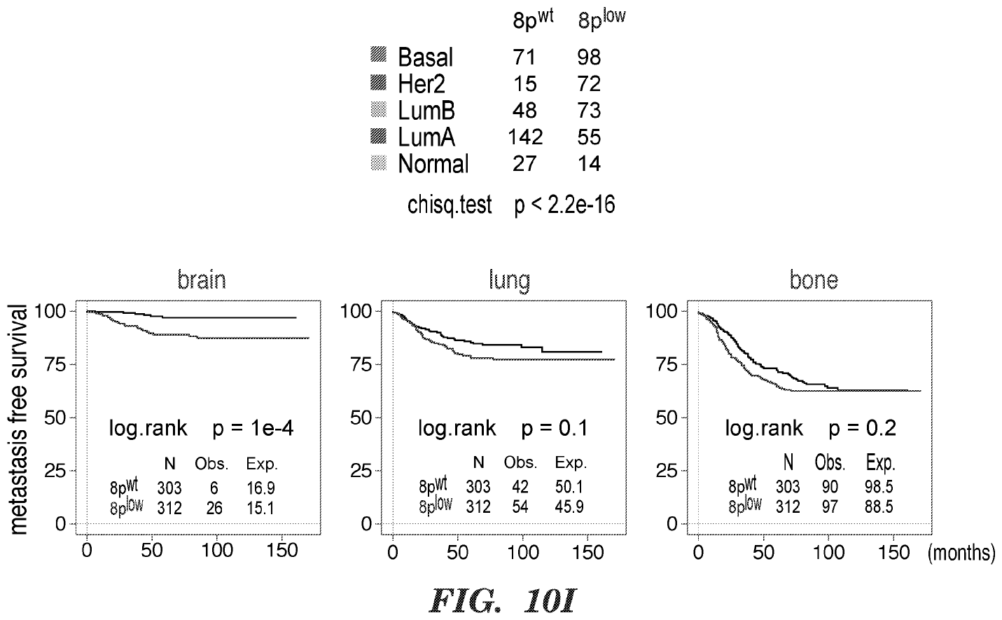

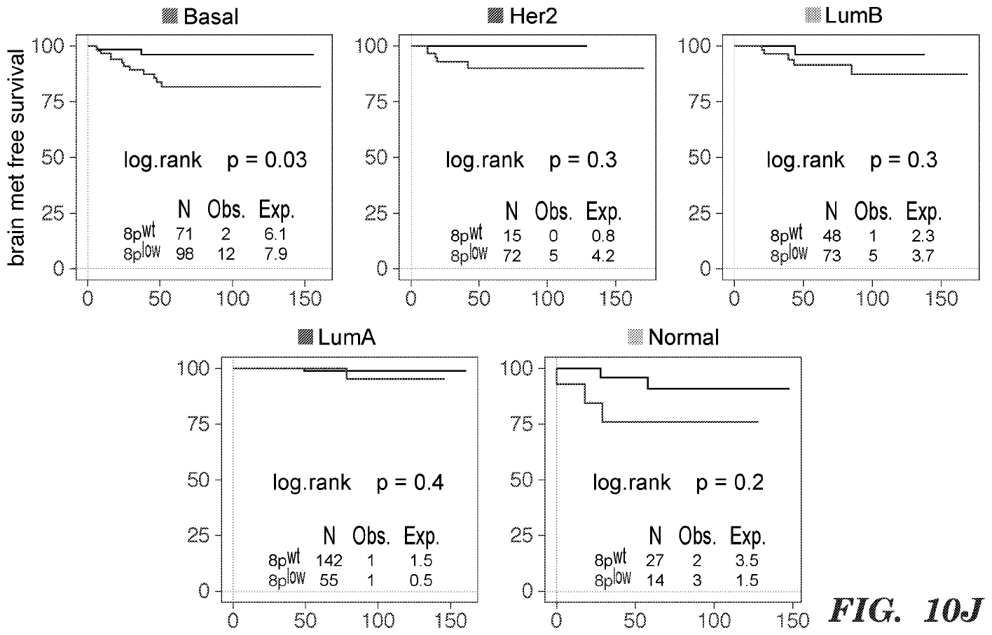

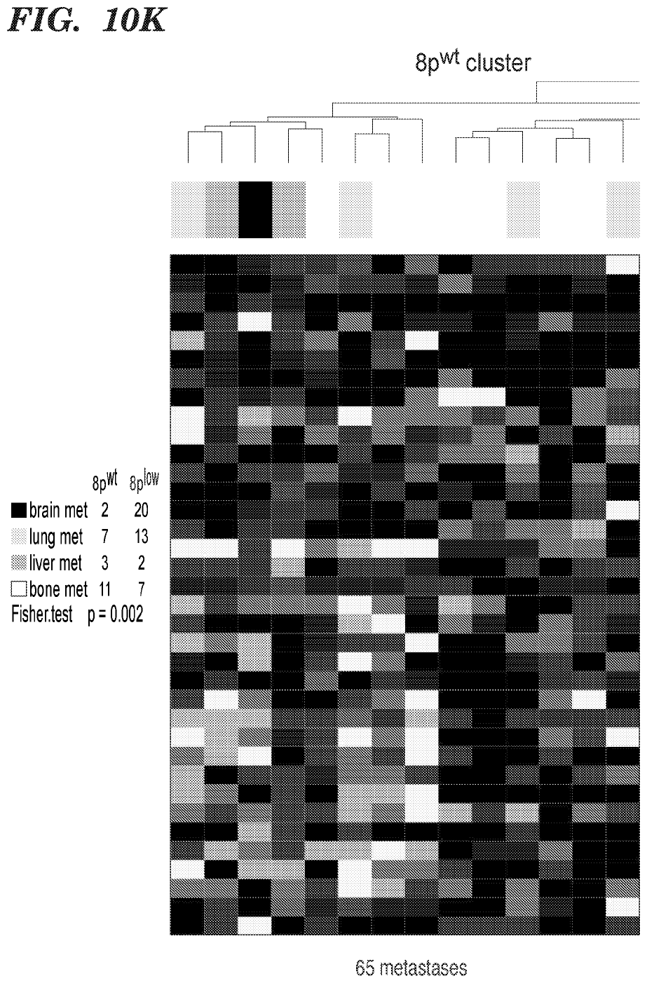

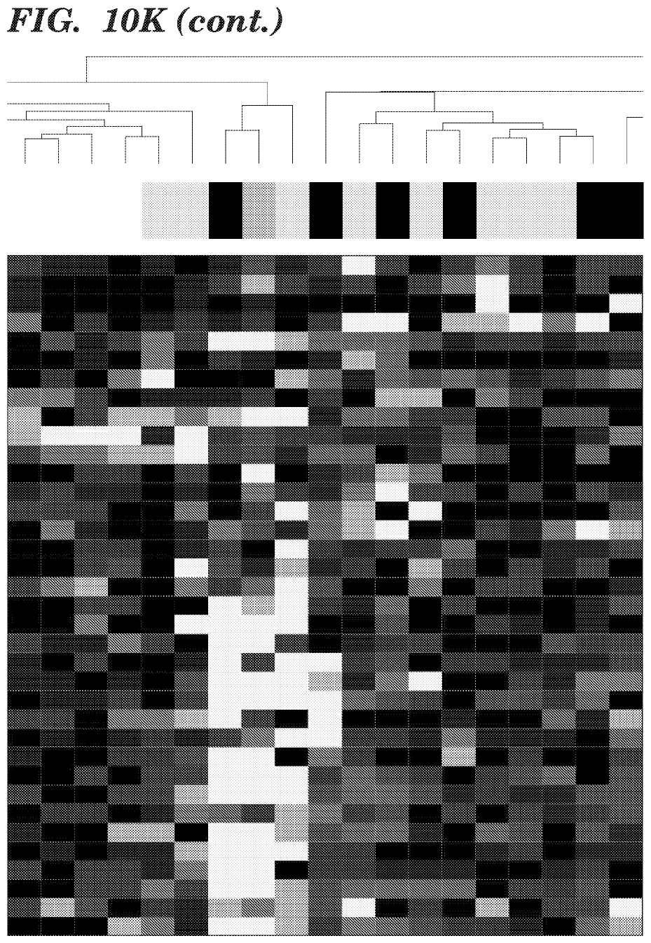

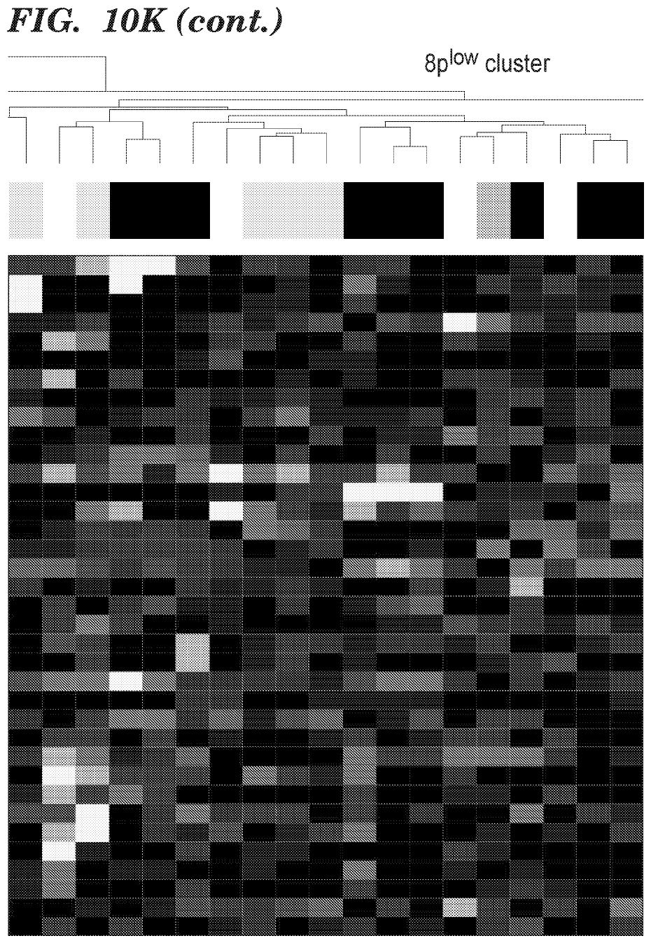

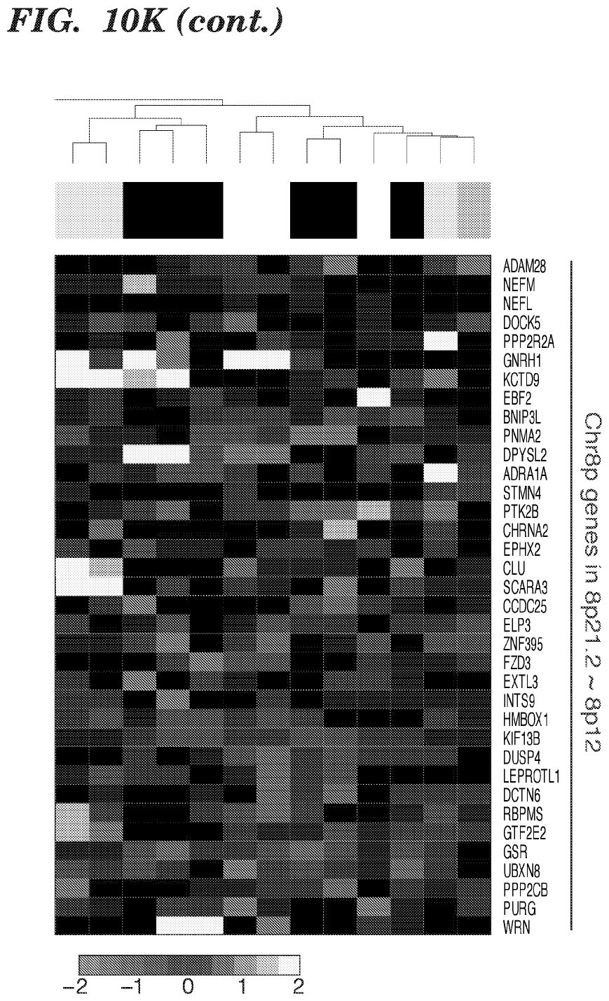

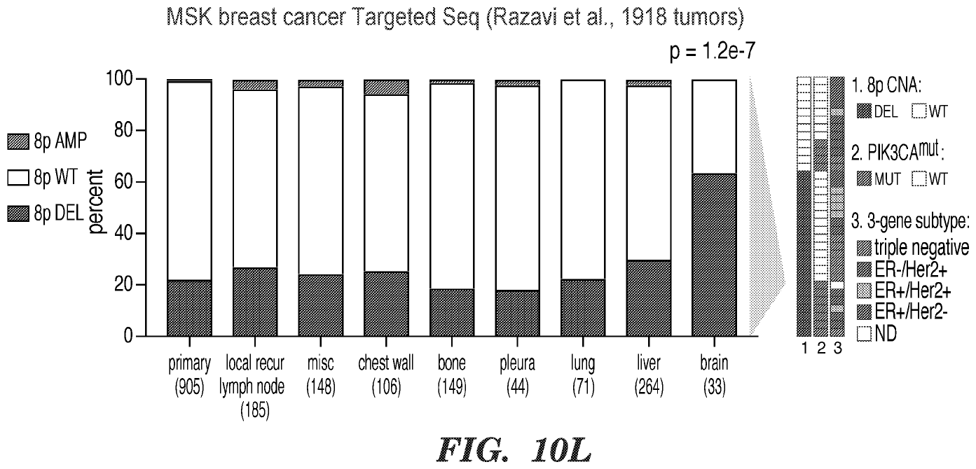

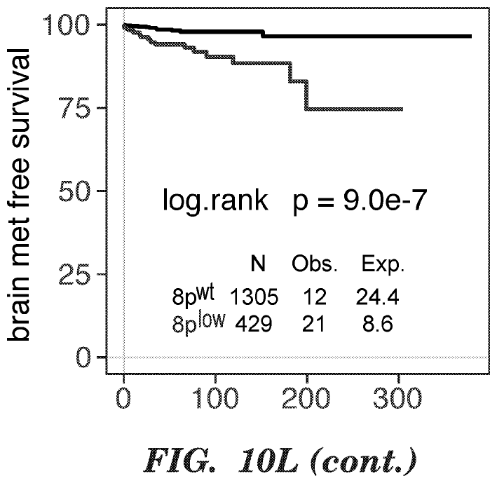

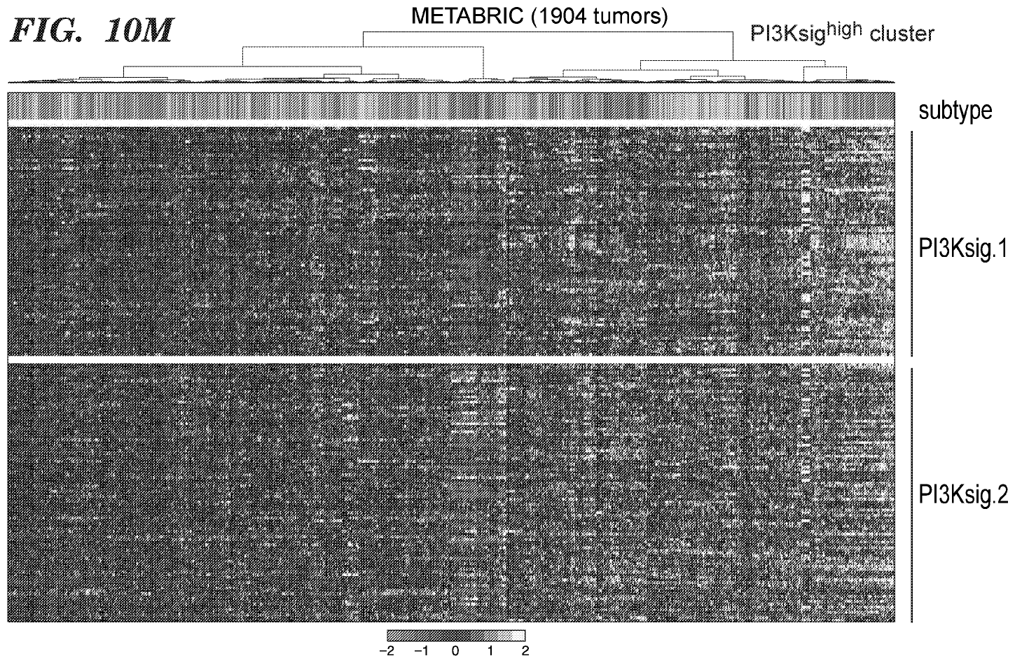

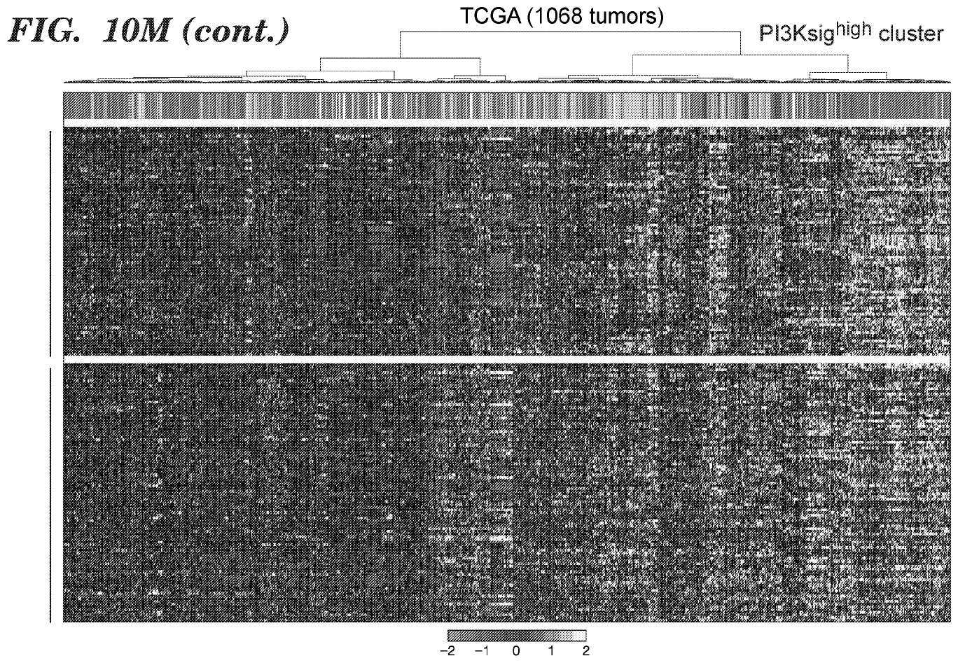

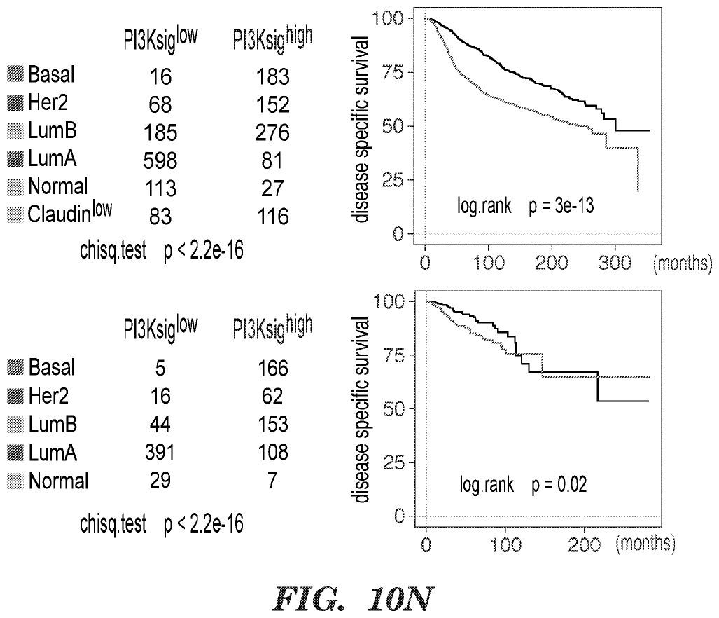

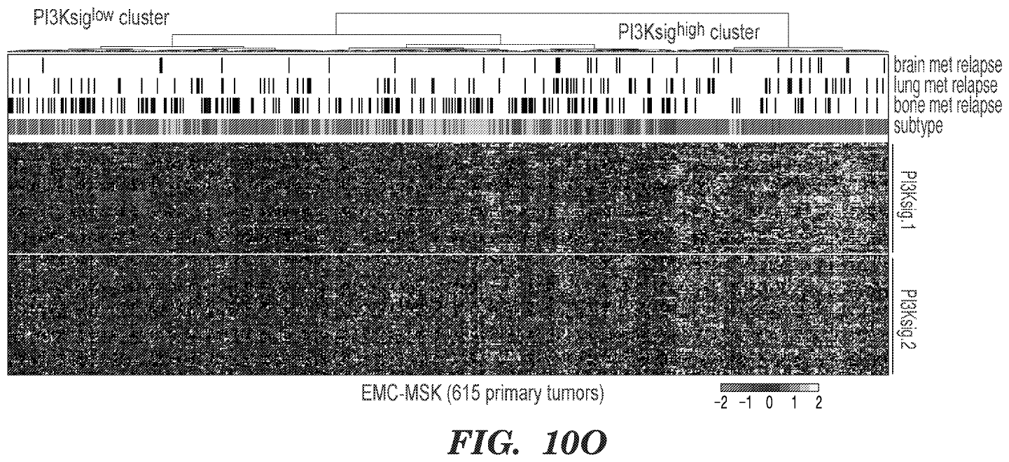

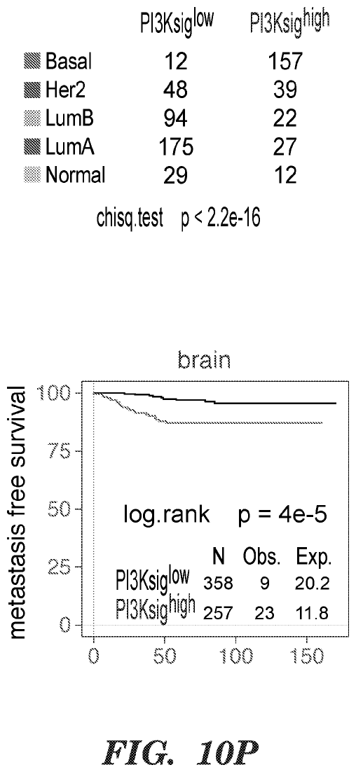

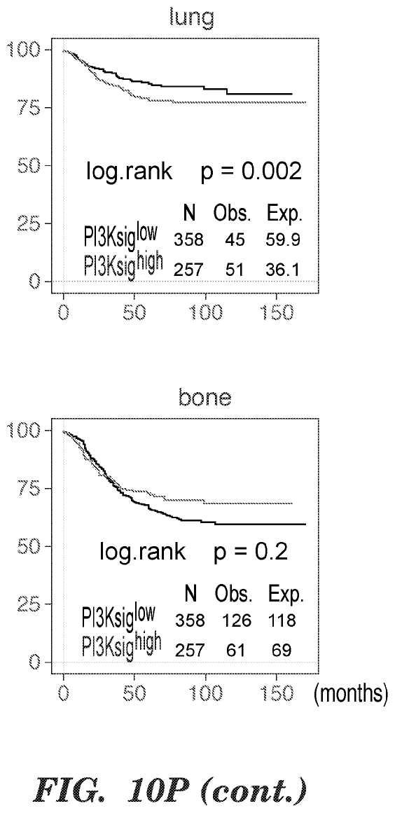

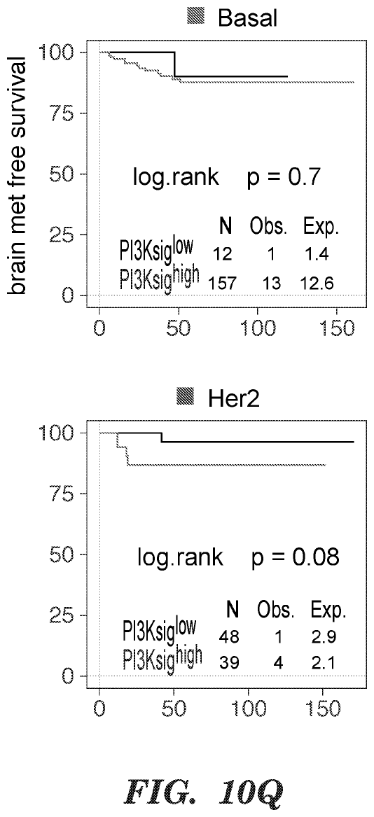

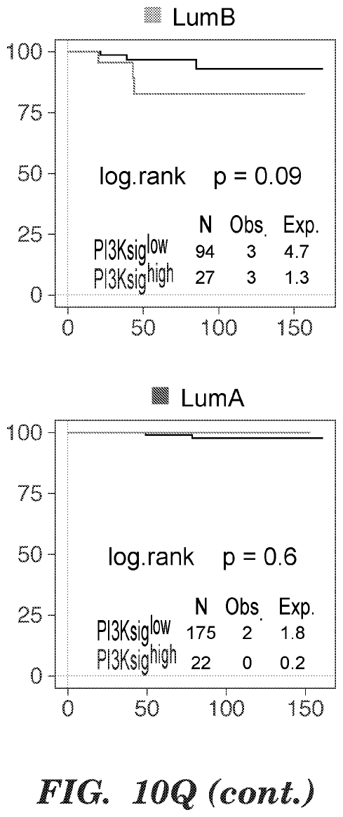

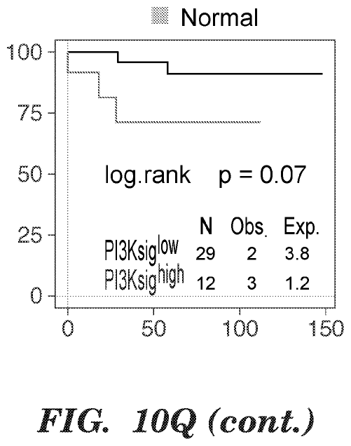

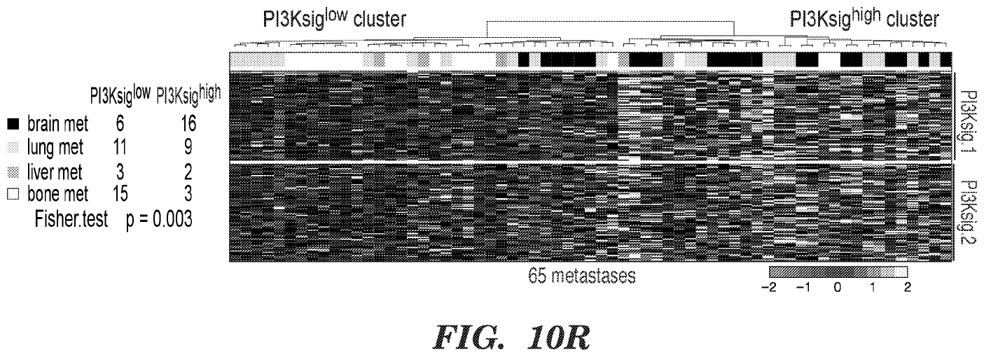

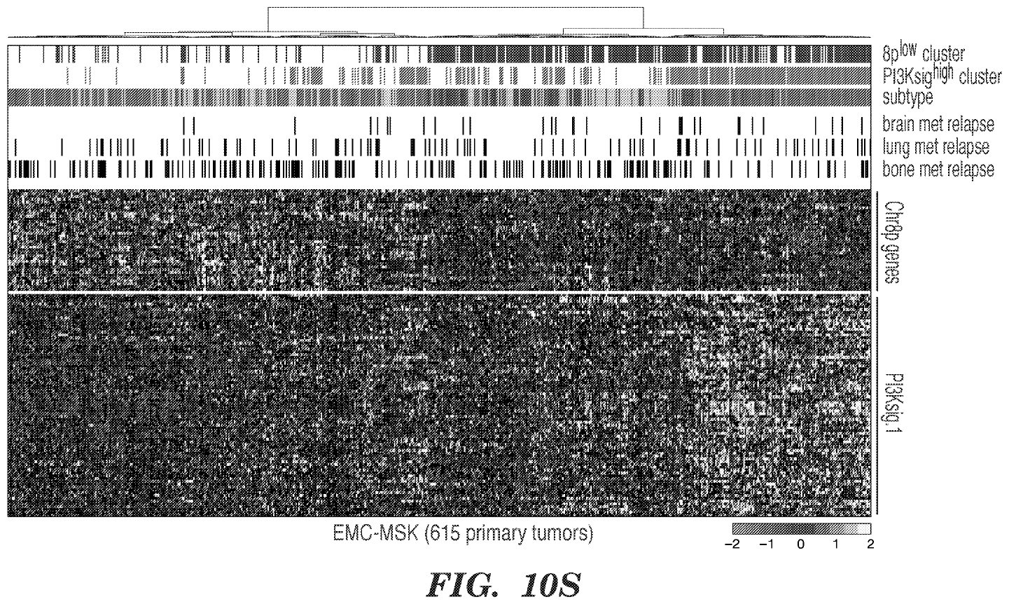

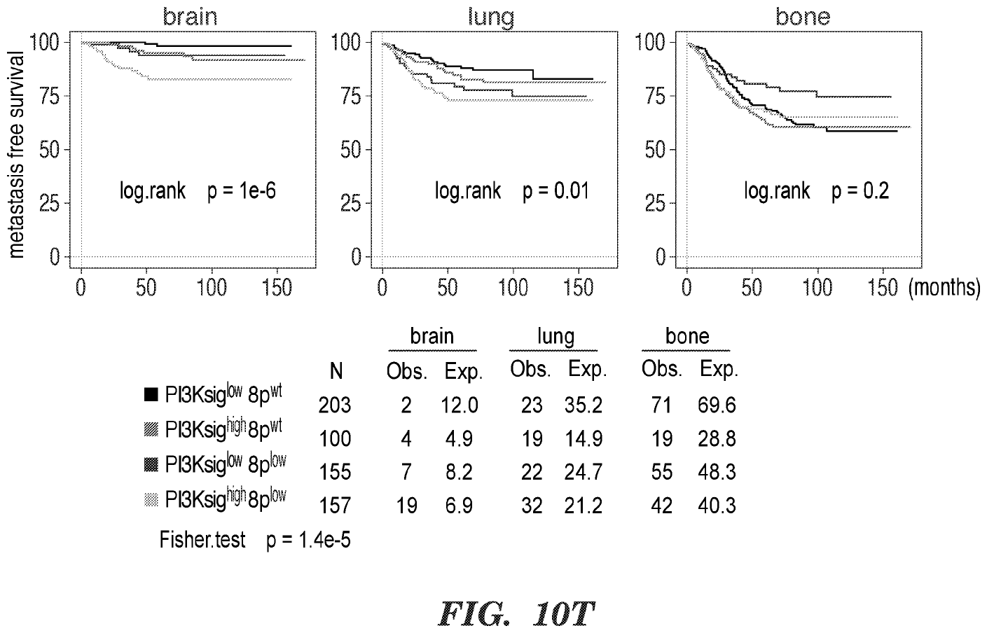

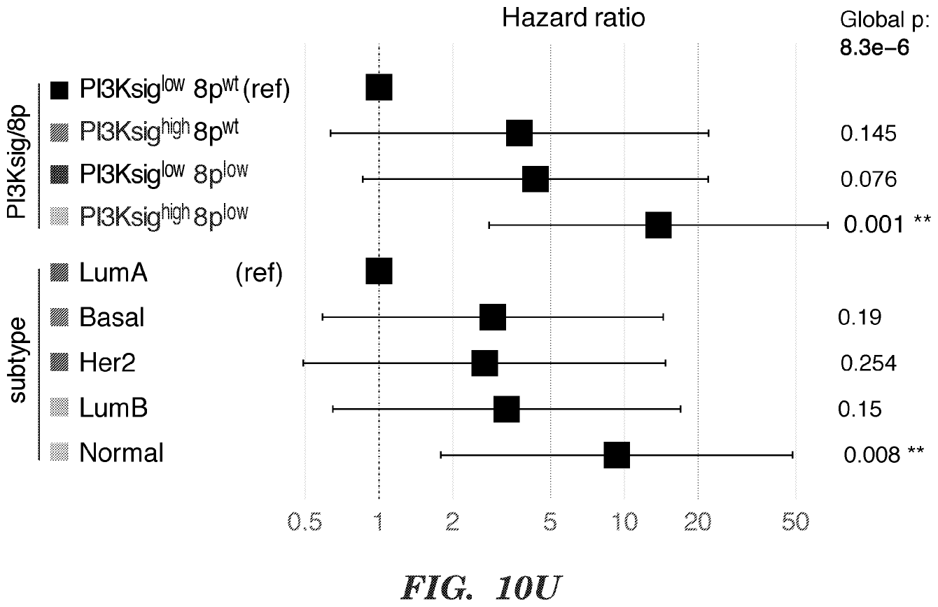

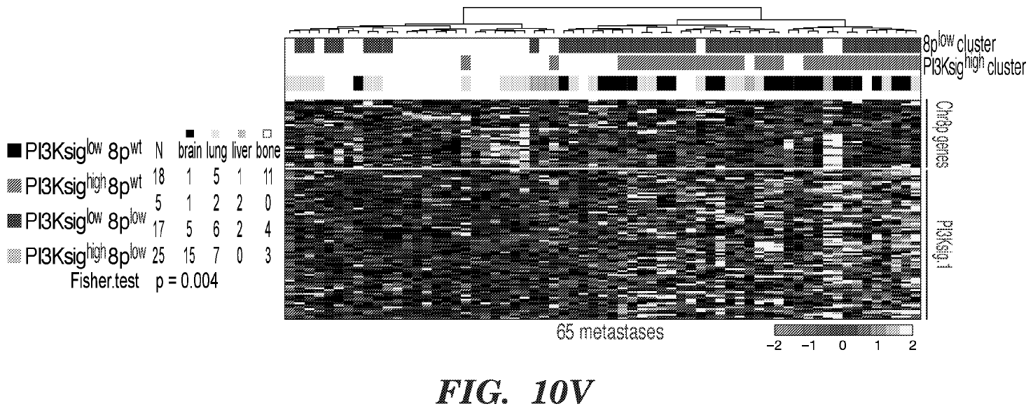

Genomic data available for each of the cell lines was used to search for evidence of DNA-level mutations associated with brain metastasis. At the level of single nucleotide variant (SNV) mutations, Phosphatidylinositol-4,5-Bisphosphate 3-Kinase (PIK3CA) mutations were significantly associated with metastasis. 4 of 7 metastatic lines harbored PIK3CA mutation, compared to 0 of 14 non- or weakly-metastatic lines (p=2.3e-06, FDR =0.01, FIGS. 9A, 9B). A fifth line (HCC70) is a PTEN mutant line. PI3K is a principle downstream mediator of (Erb-B2 Receptor Tyrosine Kinase 2) ERBB2 (HER2), which itself has been reported to be associated with brain metastasis in patients (Kennecke et al., Witzel et al.). Indeed, two of the brain-metastatic cell lines (JIMT1 and HCC1954) also harbor typical HER2 gene amplifications (FIGS. 10A-10C). Importantly, PIK3CA mutation and PI3K pathway dysregulation are enriched in tumors sampled from patients with brain metastases compared to primary tumors (Brastianos et al., Cancer Discov. 5: 1164-77 (2015), the contents of which are incorporated herein by reference in their entirety). Interestingly, PIK3CA-activating mutations have been reported to induce Sterol Regulatory Element Binding Transcription Factor 1 (SREBP)-dependent lipid synthesis in breast epithelial cells (Ricoult et al., Oncogene 35: 1250-60 (2016), the contents of which are incorporated herein by reference in their entirety).

Unbiased analysis of the DNA copy number landscape similarly pointed to an association with lipid metabolism. An association was observed between metastatic potential and deletions of chromosome 8p12-8p21.2 (p=7.3e-06, FDR=0.0017, FIGS. 10A, 10B, 10D). 5 of 7 brain-metastatic breast cancer cell lines harbored deletions in this region, compared to 0 of 14 non-metastatic lines (FIG. 10A, 10B, 10E). A sixth metastatic line (JIMT1) had small deletions within the commonly deleted region. Although the number was small, co-occurrence of PI3K activation and 8p-loss in the same brain metastatic lines was noticed. Importantly, it has been reported that experimental 8p deletion results in activation of cholesterol and fatty acid biosynthesis, which, like PI3K activation, occurs in an SREBP-dependent manner (Cai et al., Cancer Cell 29: 751-66 (2016)). The key genes on 8p that mediate this phenomenon are unclear, but 8p deletion is associated with poor patient prognosis.

### Example 5

### Clinical Relevance of MetMap Data

To ascertain the clinical relevance of these associations, clinical tumor datasets of breast cancer, among which EMC-MSK contains organ-specific metastasis relapse information for each patient were analyzed (FIG. 10F-10V). A strong correlation was observed between 8p gene expression and its copy number status in both METABRIC and TCGA datasets (FIG. 10F), thereby validating 8p expression as a surrogate for copy number in datasets where copy number data were not available. The 8p-loss is more common in the more aggressive Basal, HER2, and LumB subtypes, but less enriched in LumA or Normal subtypes (FIG. 10G). In EMC-MSK dataset, coordinated expression of 8p genes stratified clinical samples into two clusters, with the low-expressing cluster showing enrichment in brain metastasis and lower brain-metastasis-free survival. Concordant with these findings, the 8p-low signature was strongly enriched in brain metastasis lesions obtained from patients with breast cancer.

To assess PI3K activity in these clinical cohorts, we utilized two PI3K-response signatures, one generated with PIK3CA mutant overexpression, and the other with PI3K-inhibitor treatment. Although the gene identities overlapped little between the two signatures, strong co-regulated patterns were observed in patient tumors (FIG. 10M). Consistent with the previous report, PI3Ksig-high tumors were enriched in Basal, Her2, and LumB, in comparison to LumA and Normal subtypes (FIG. 10N). Significant association between PI3Ksig-high and brain metastasis was observed (FIGS. 10O-10R), similar to the 8p-low state. Since both genetic features were associated to brain metastasis, we further queried the relationship between the two. Strong co-occurrence was observed, and the overlapping events captured the majority of patients with poor brain metastasis relapse (FIGS. 10S, 10T). The two features were stronger brain metastasis predictors than subtypes per se (FIGS. 10U, 10V). These results together provided strong clinical validation of MetMap experimental findings.

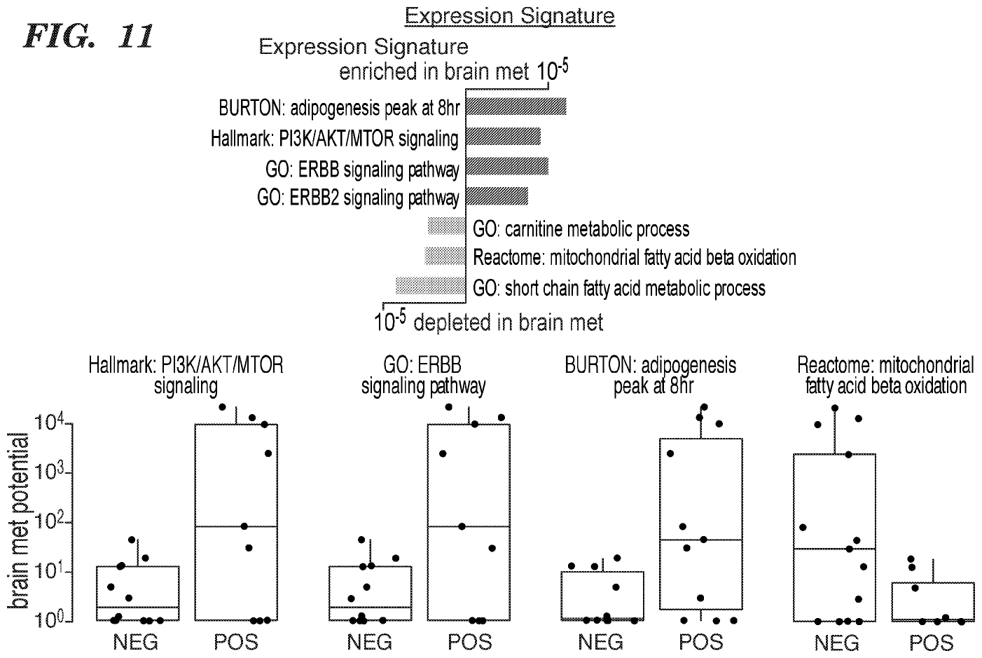

Consistent with associations at the genetic level, expression analysis similarly showed an enrichment of a PI3K activation signature in the brain metastatic cell lines (FIG. 11). Furthermore, a lipid synthesis signature was observed to be strongly associated with brain metastatic potential. Importantly, experimental PI3K activation and 8p-deletion both have been reported to result in induction of cholesterol and lipid synthesis in breast epithelial cells. These results suggested that an increased lipid synthesis phenotype might underlie the commonalities between PI3K-activation and 8p-loss.

### Example 6

### Lipid Biosynthesis Associated with Brain Metastasis in Transcriptome Analysis

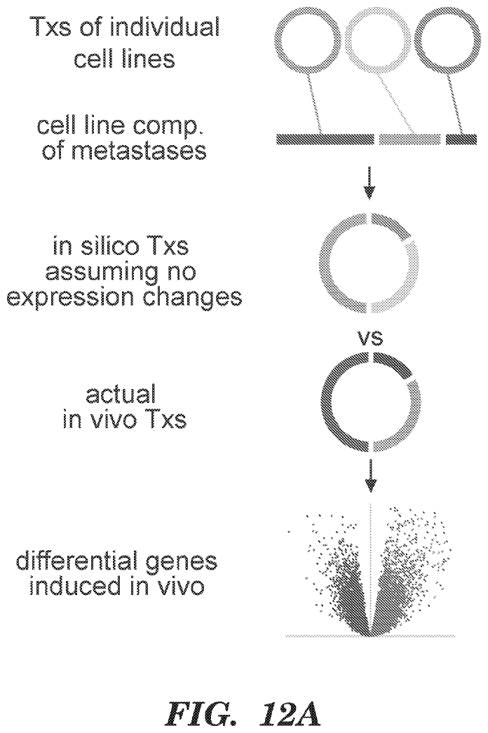

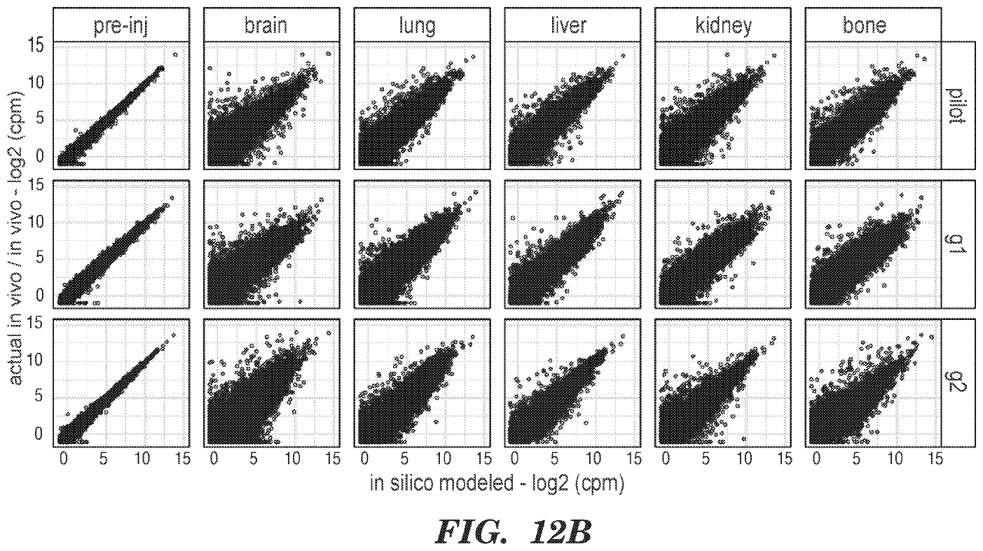

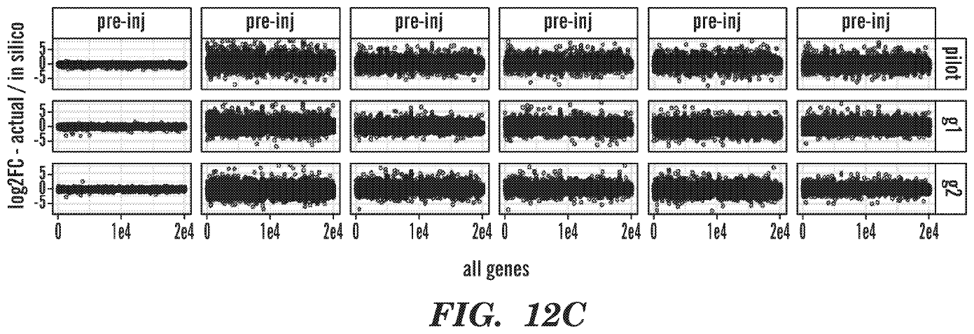

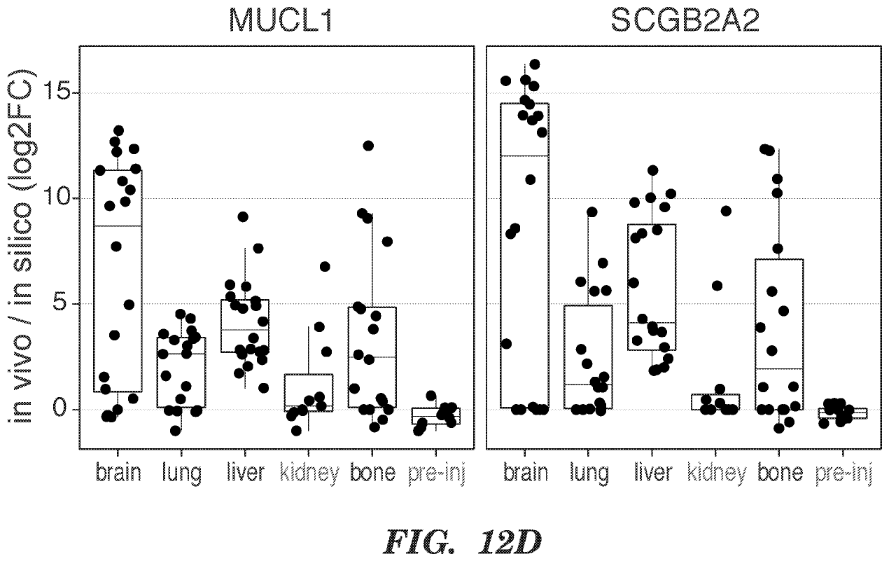

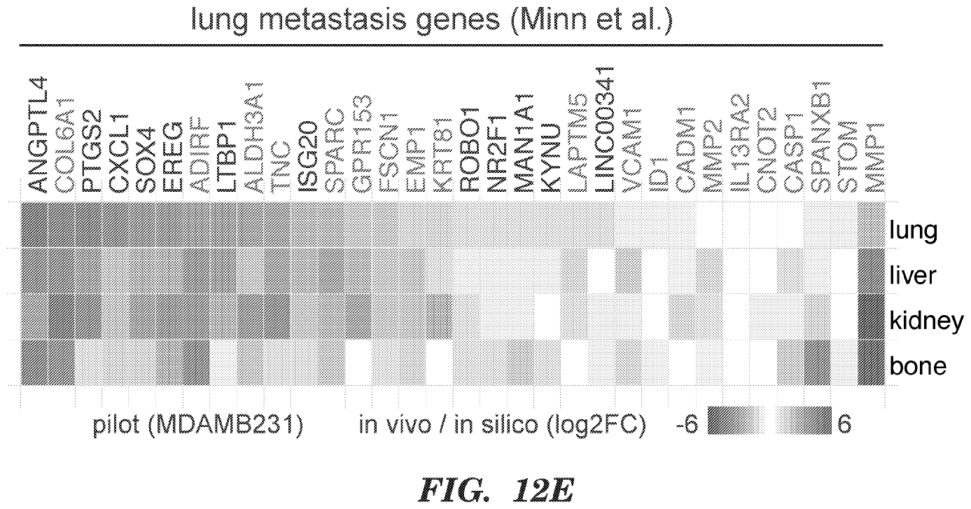

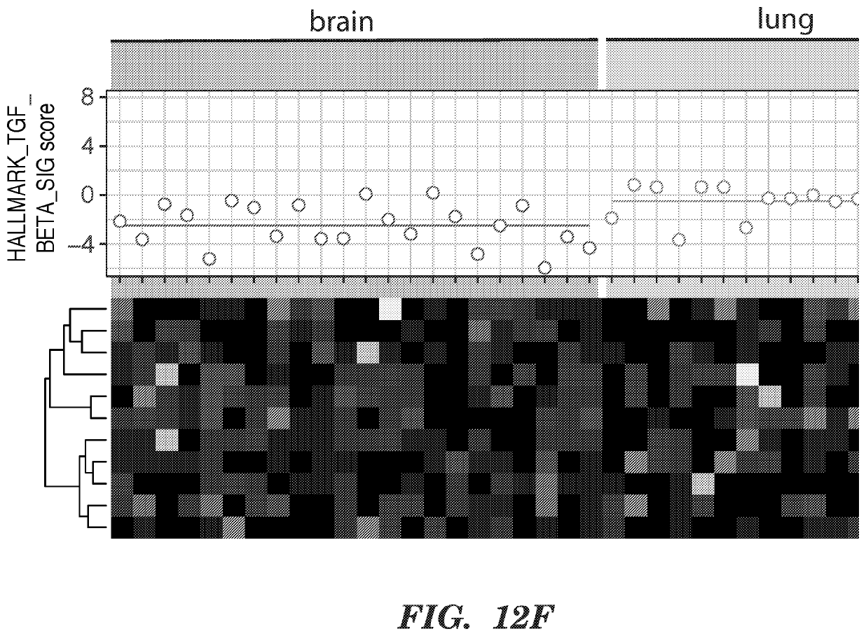

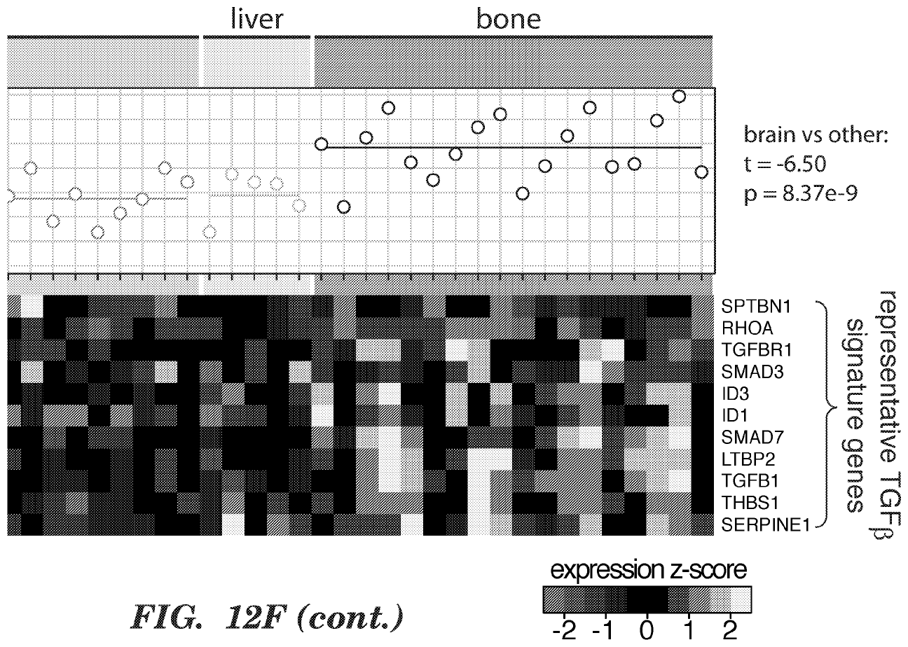

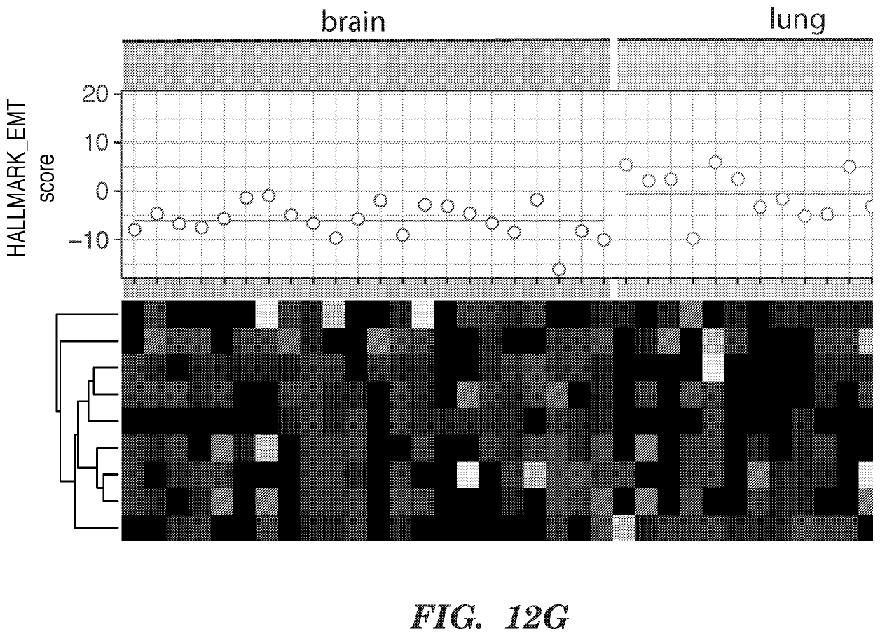

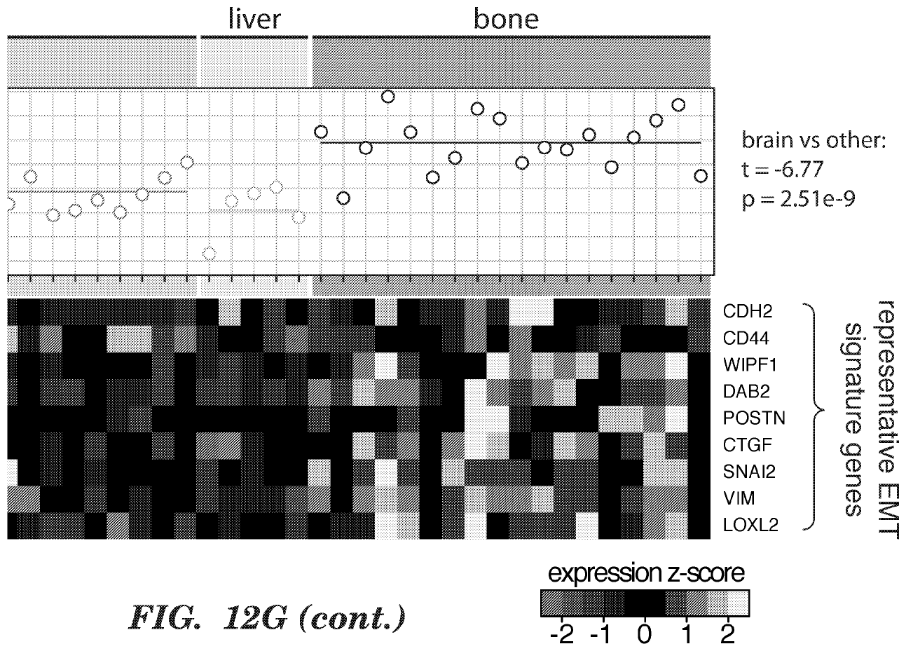

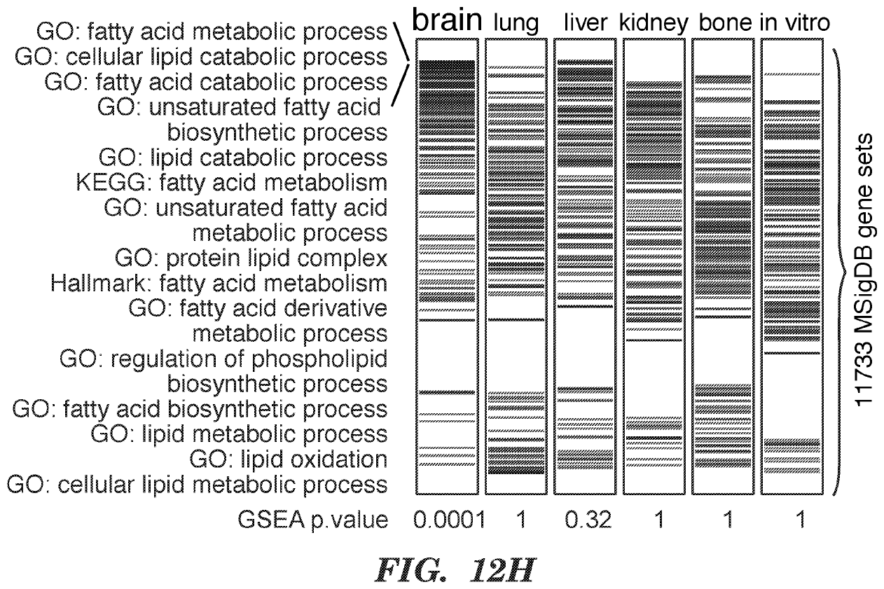

Transcriptomes of the breast cancer cell lines were analyzed to detect associations with brain metastasis. For this analysis, gene expression profiles of cell lines growing in vitro were compared to their profiles in in vivo metastatic lesions (see FIGS. 12A to 12E for detailed analyses).

RNA-Seq was used to characterize the transcriptomes, and this protocol captured cancer cell compositions and averaged in vivo transcriptomes of metastases from cell line pools in the breast cancer cohort study. To understand what metastases the transcriptomes encoded, differential expression analysis was performed on the in vivo transcriptomes to cells in vitro. To properly account for the different cell line compositions in each metastasis, a composite in vitro transcriptome was modeled using the barcode composition and single cell line in vitro transcriptomes and then compared to the in vivo results (FIG. 12A). Differentially expressed genes were uniquely attributed to the in vivo context, but not due to cell composition differences. These genes were either commonly induced (or selected for) in multiple cell lines or were uniquely enriched in the dominant line. The transcriptomes of the pre-injected population, which were a direct mixture of in vitro cell lines showed a very tight correlation with in silico profiles and few genes were differentially expressed (FIGS. 12B, 12C). In contrast, the transcriptomes from in vivo samples showed genes with large fold changes, and the correlation was weaker with the in silico profiles. These results validated the comparison method and showed that the in vivo environment induces substantial transcriptional changes. To assess whether such comparison identified genes relevant to metastasis, the top differentially expressed genes were inspected. Notably, Secretoglobin Family 2A Member 2 (SCGB2A2) (also known as Mammaglobin (MGB1)) and Mucin Like 1 (MUCL1) (also termed small breast epithelial mucin (SBEM)) were strongly induced in brain metastases, as well as in other sites (FIGS. 12D). These genes are breast lineage markers whose expression is known to be induced during breast tumorigenesis from clinical specimens. Their expression has been used as a marker to indicate hematogenous spread, micrometastasis (Barretina et al., Nature 483: 603-07 (2012)., Garnett et al., Nature 483: 570-75 (2012)), and to distinguish breast cancer metastasis in the brain from primary brain tumors (Iorio et al., Cell 166: 740-54 (2016)). The results suggested that although these genes were non-expressed or expressed at a low level in vitro, their expression could be induced in the in vivo metastasis context. These results highlight the biological relevance of the in vivo transcriptomic results.

In the pilot group experiments, MDAMB231 dominated lung, liver, kidney, and bone metastases in most samples (FIG. 1I). Thus, the majority of the gene expression changes were attributed to MDAMB231. In light of the MDAMB231's dominance in the pilot study and because MDAMB231 is the most investigated cell line in breast cancer metastasis, it was necessary to determine if genes previously identified and validated as metastasis mediators were induced in the in vivo transcriptomic profiles. 27 of 32 lung metastasis genes reported by Minn et al. were upregulated in the lung metastasis profiles and showed very strong agreement (p value=3.9e-16, FIG. 12E). These genes were also enriched in metastases at other sites but to a lesser extent. Although these genes were initially identified as lung metastasis mediators, many were shown to function in a pleiotropic fashion, mediating primary tumor growth or metastasis at other sites. For example, Vascular Cell Adhesion Molecule 1 (VCAM1) has been shown to mediate both lung and bone metastasis through juxtacrine interaction with myeloid lineage cells (Minn et al., Malladi et al., Cell 165: 45-60 (2016), the contents of which are incorporated herein by reference in their entirety). Tenascin C (TNC), which is a secreted molecule that boosts breast cancer stemness, promotes lung and bone metastasis (Chen et al., Cell 160: 1246-60 (2015), the contents of which are incorporated herein by reference in their entirety). Collectively, these results suggested that the in vivo “induced” genes not only included metastasis associated markers but also previously validated mediators.

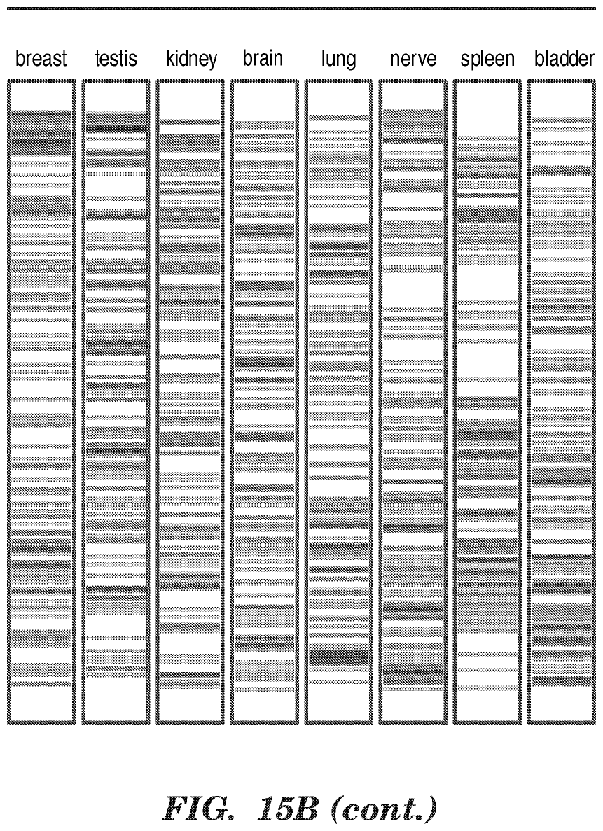

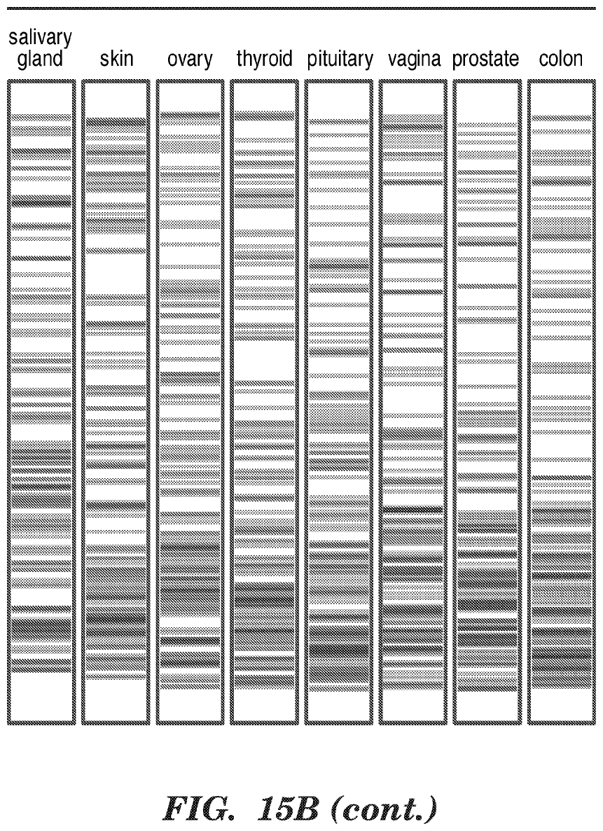

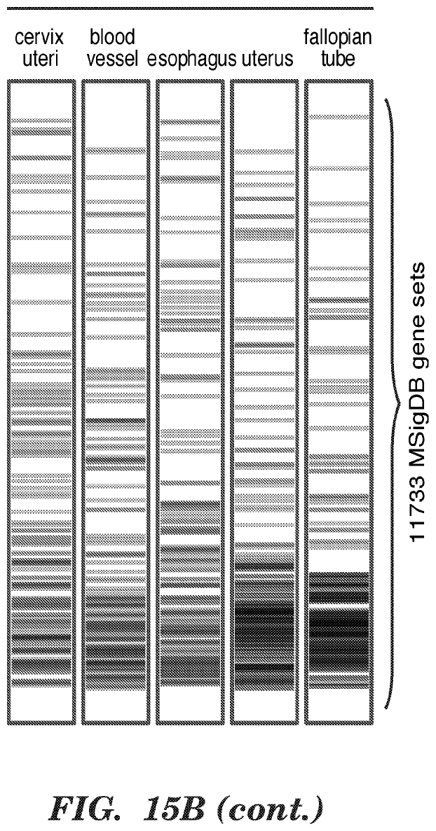

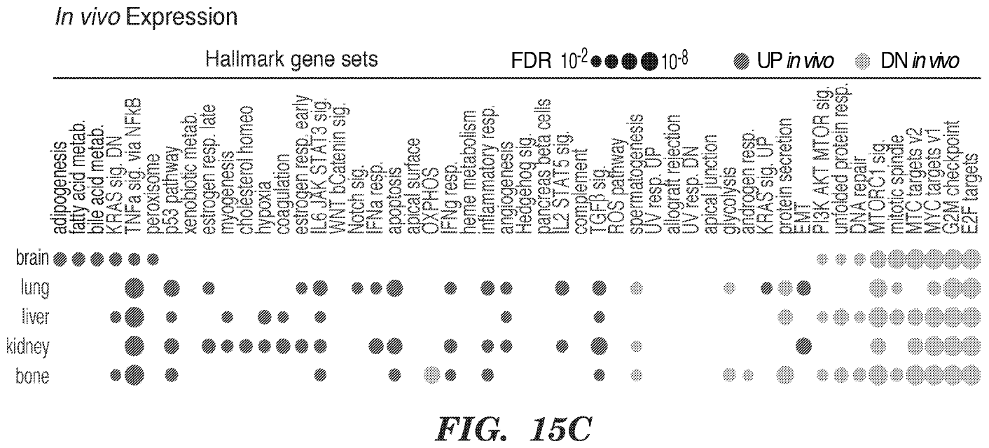

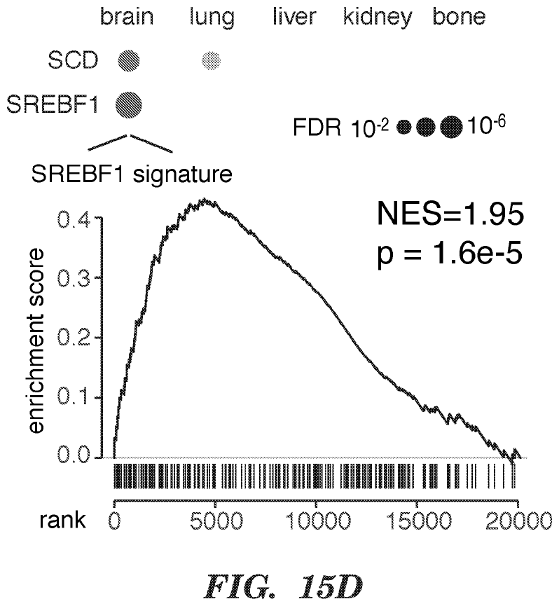

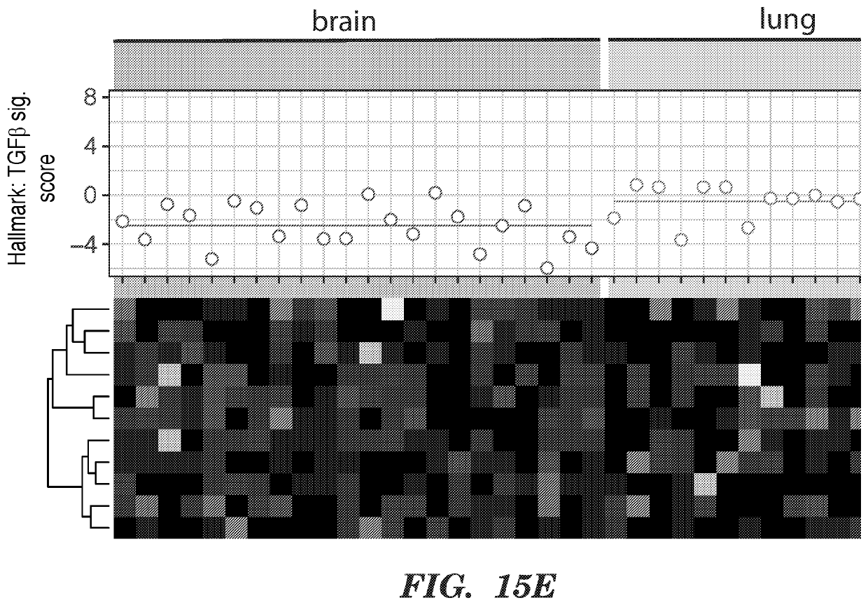

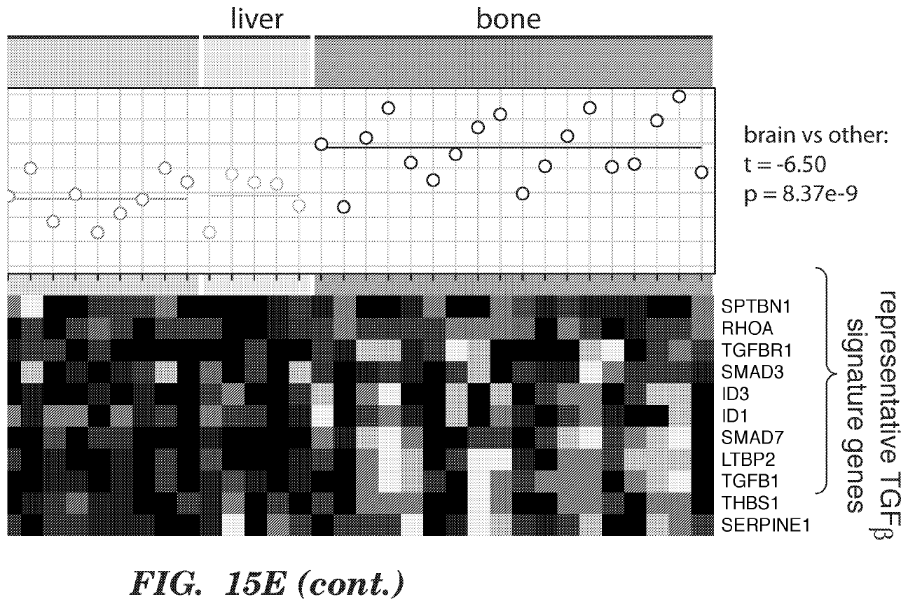

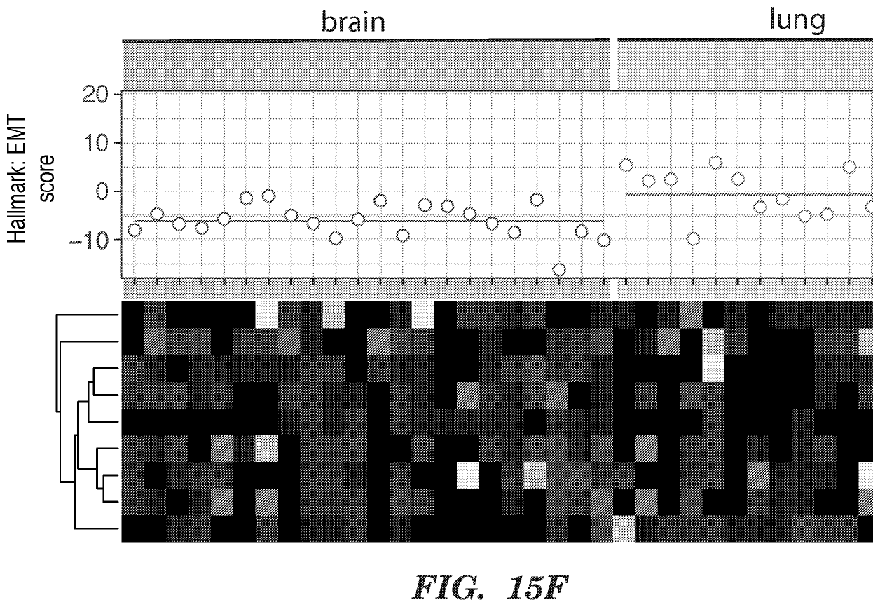

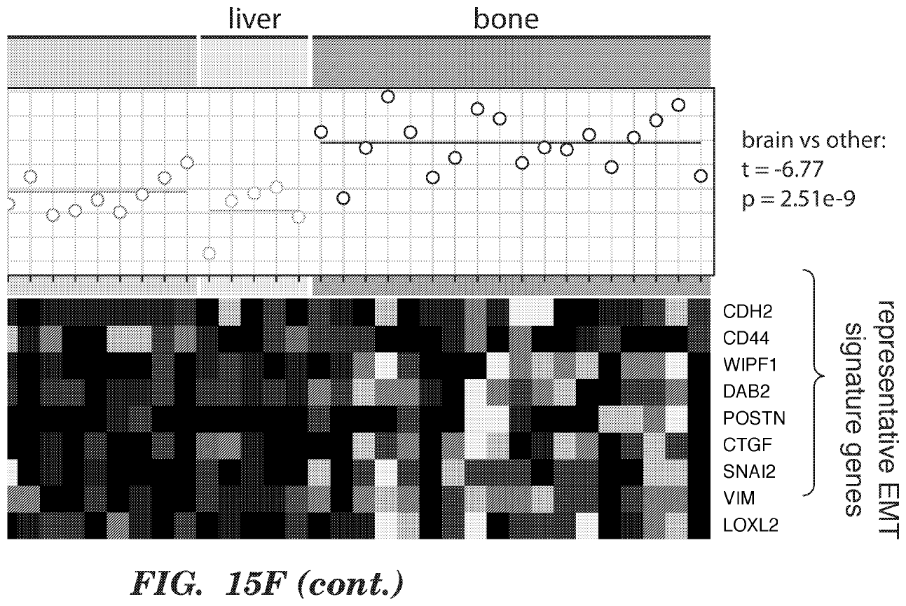

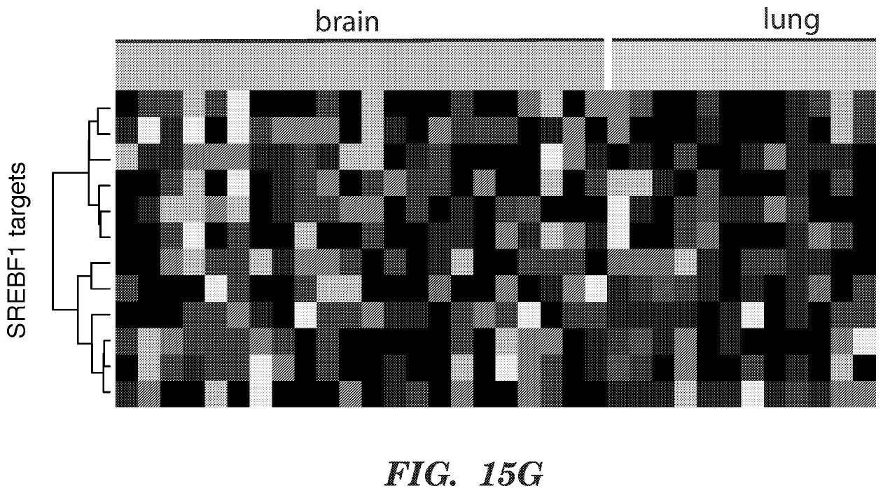

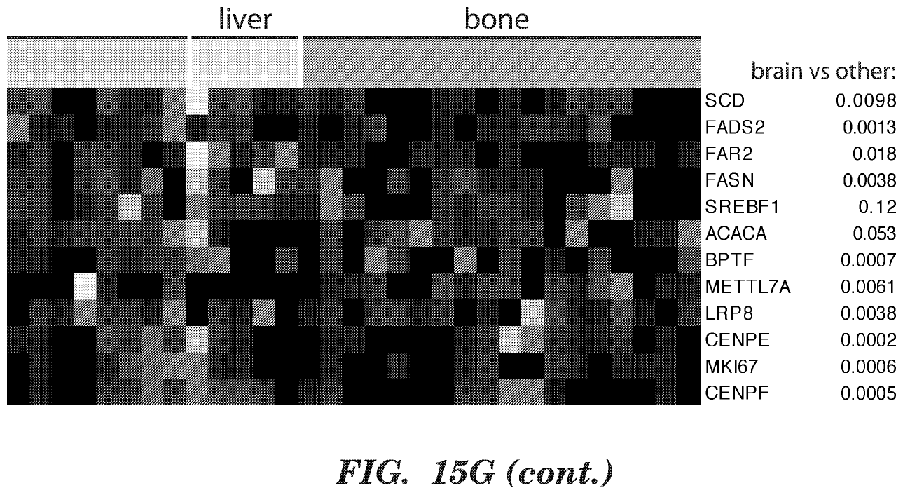

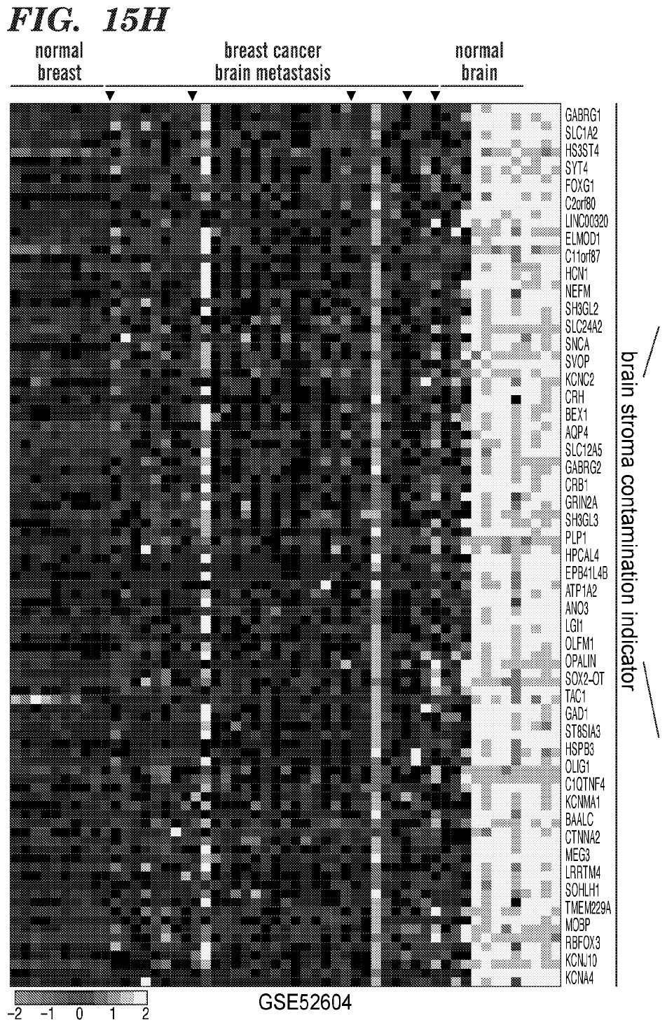

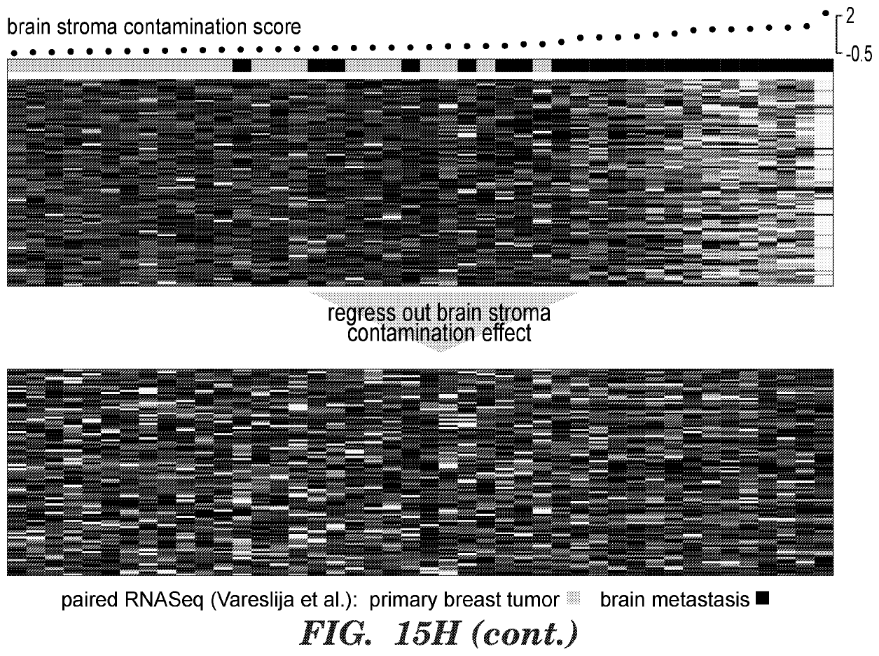

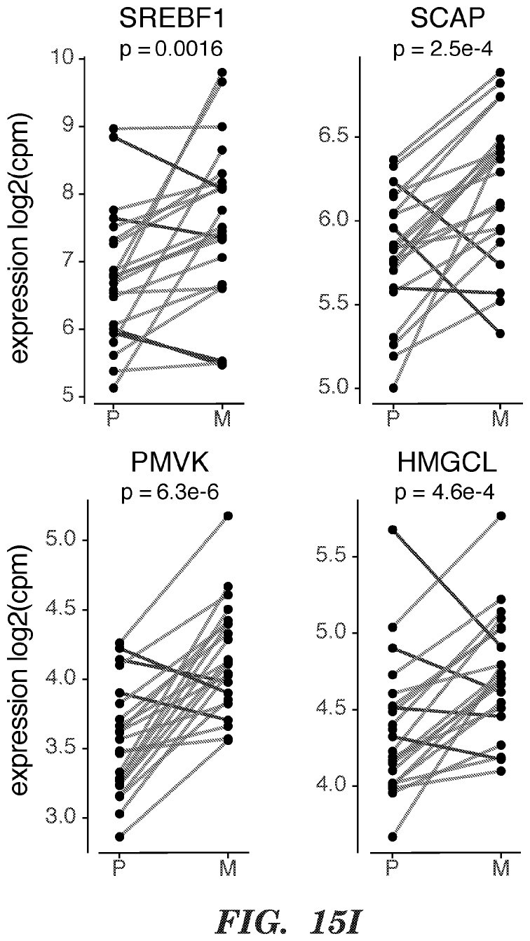

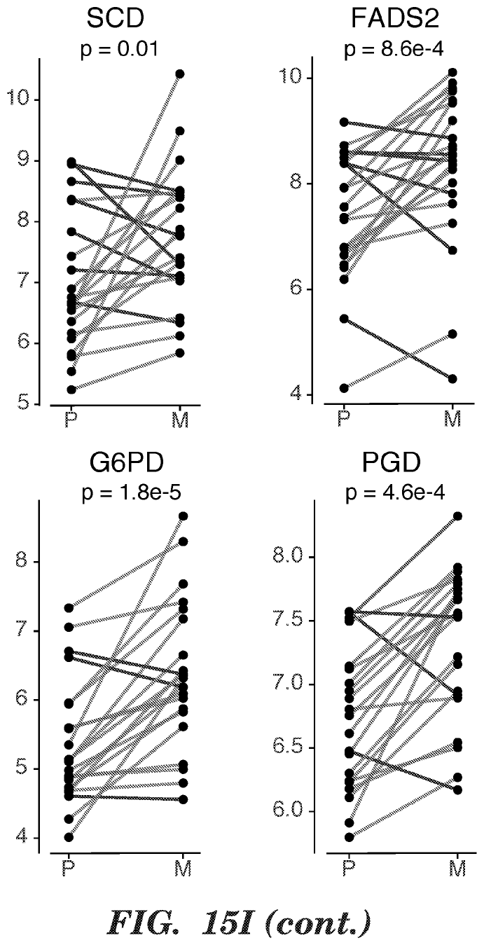

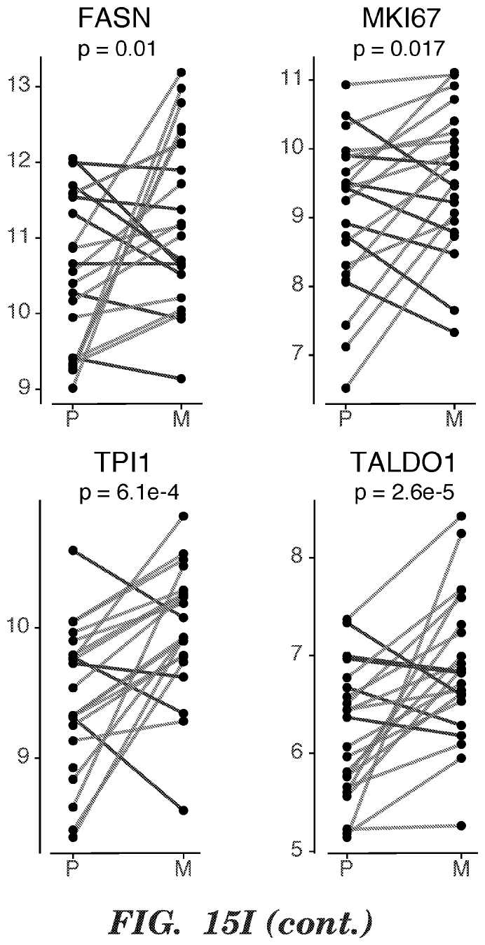

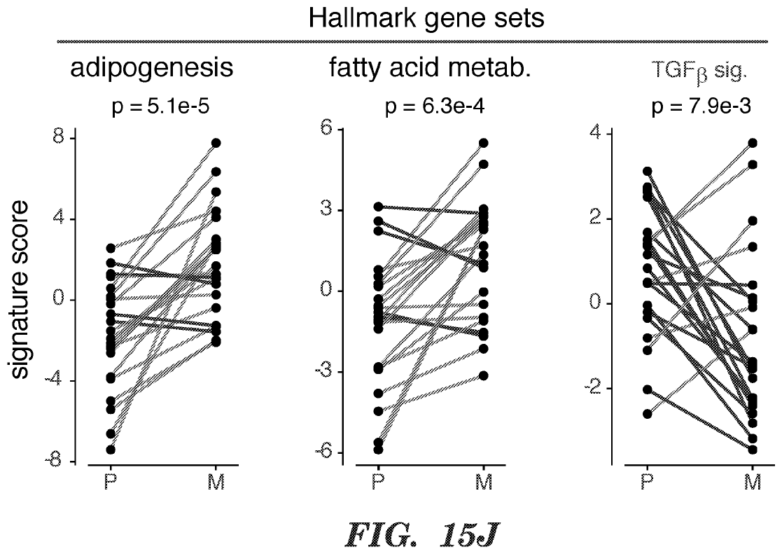

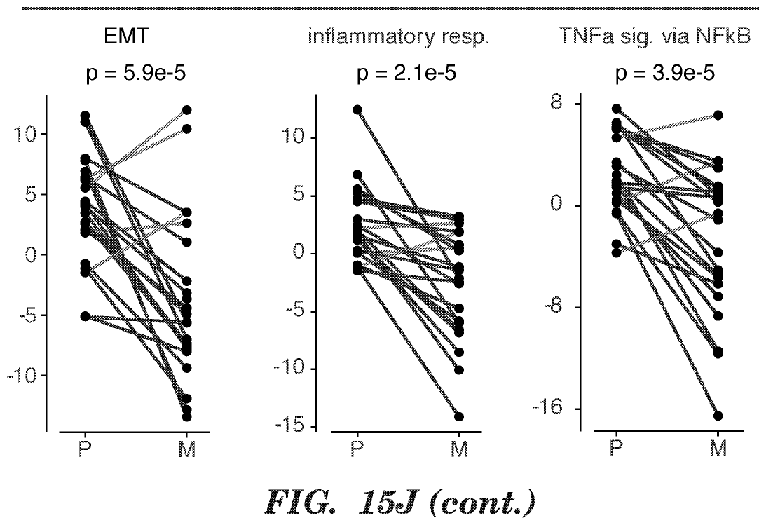

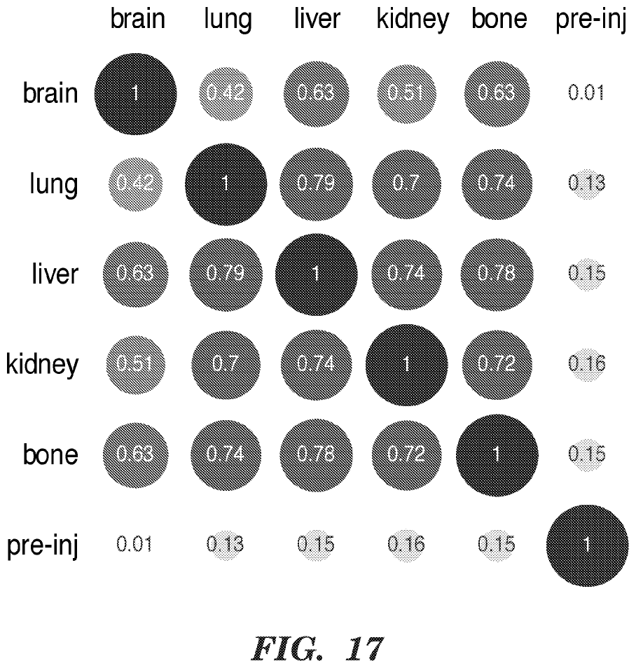

Having confirmed the validity of these profiles, pathway enrichment analysis was performed to query consensus programs that the differential genes encode at the 5 metastasis sites (FIG. 15C). The results revealed a diverse in vivo response to external stimuli, suggestive of much richer environmental factors in the animal. In contrast, proliferation and cycling pathways are much attenuated in vivo compared to in vitro cells (FIG. 15C). Consistent with this result, in vitro culture media is optimized for maximal cell proliferation by supplementing excess nutrients and supportive elements (Yao, T. & Asayama, Y. Animal-cell culture media: History, characteristics, and current issues. Reprod Med Biol 16, 99-117 (2017).). Comparing between organs, brain metastases shared less commonality and weaker correlation with metastases in extracranial organs (FIG. 17), suggesting a more unique microenvironment in the brain. More specifically, inflammatory responses including TNF, interleukin and interferon signaling were more prominent in lung, liver, kidney, bone than in brain, consistent with less immune response in the brain compared to extracranial organs (Louveau, A., Harris, T. H. & Kipnis, J. Revisiting the Mechanisms of CNS Immune Privilege. Trends Immunol 36, 569-577 (2015).). Similarly, evidence was observed of TGFβ activation and epithelial-mesenchymal transition (EMT) in extracranial metastatic lesions, but not in brain (FIG. 15C). Confirming such experimental observations, brain metastasis samples from patients showed less TGFβ response and EMT, in comparison to extracranial metastases or matched primary breast tumors (FIGS. 15E, 15F, 15H, 15J). Instead, breast cancer cells growing in brain acquired gene expression signatures of adipogenesis, fatty acid metabolism, and xenobiotic metabolism (FIG. 15C), a phenomenon also observed in patient samples (FIGS. 15H, 15J). Notably, this lipid metabolism signature was unique to cancer cells growing in the brain (FIG. 12H, 15A), as normal brain does not show such a signature (FIG. 15B). Together, these results revealed a distinct cell transcriptional state in brain metastasis.

### Example 7

### Metabolite Profiles Indicate a Role Lipid Synthesis in Metastasis

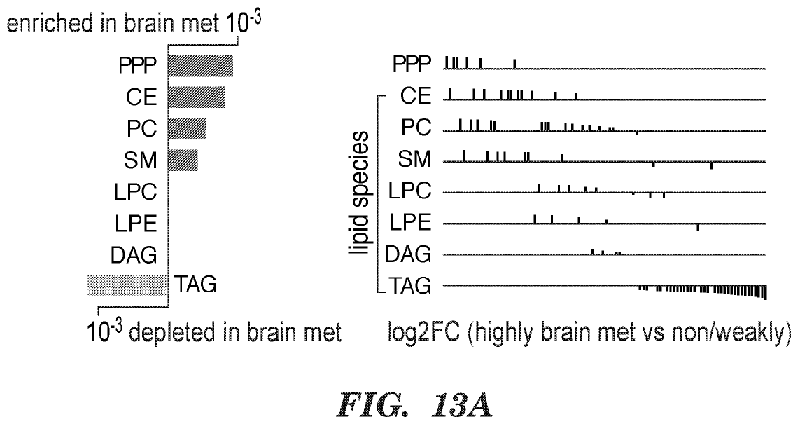

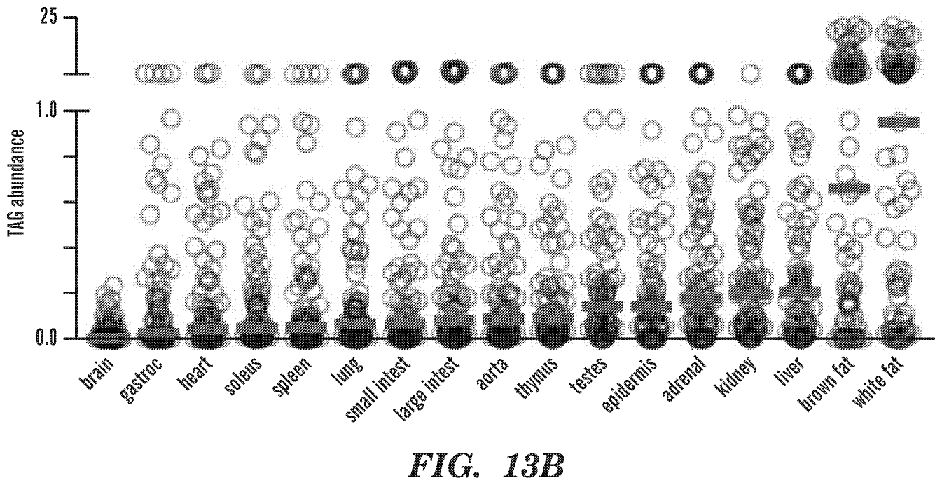

To determine if a metabolite profile paralleled the gene expression profiles associated with brain metastatic potential, the abundance of 226 metabolites was analyzed across the breast cancer cell lines (Barretina et al.). As predicted from mRNA profiling, upregulation of cholesterol species in highly brain metastatic cells was observed (FIG. 13A). In addition to cholesterols, membrane lipids including phosphatidylcholine (PC), lysophosphatidylcholine (LPC), and sphingomyelin (SM) were similarly upregulated (FIG. 13A), as were metabolites of the pentose phosphate pathway (PPP), which is required for cholesterol and fatty acid synthesis (Patra et al., Trends Biochem. Sci. 39: 347-54 (2014), the contents of which are incorporated herein by reference in their entirety).

In contrast, global downregulation was observed for triglycerides (triacylglycerols, TAGs) in brain metastatic cells (FIG. 13A). These results suggest that brain metastatic cells are in a low TAG state in culture and that the lipid pool is primarily funneled to cholesterol and membrane lipid synthesis. Non-brain metastatic cells however adopt a TAG-high state and as a result harbor a higher fatty acid oxidation signature (FIG. 11D), consistent with that TAG is an input material of fatty acid oxidation. Intriguingly, metabolite profiling of normal mouse tissues shows that brain has dramatically lower TAG abundance compared to other tissues (FIG. 13B) (Jain et al., Am. J. Physiol. Endocrinol. Metab. 306: E854-68 (2014), the contents of which are incorporated herein by reference in their entirety). Instead, most lipid species in brain cells are in the form of membrane and cholesterol, which are integral to brain structure and function. Breast cancer cells that are already in a low TAG state may be uniquely able to survive in the low TAG environment of the brain, which is consistent with the seed-and-soil hypothesis (Paget et al., Cancer Metastasis Rev., 8: 98-101 (1989), the contents of which are incorporated herein by reference in their entirety).

### Example 8

### SREBF1-Mediated Lipid Metabolism is Associated with Brain Metastasis

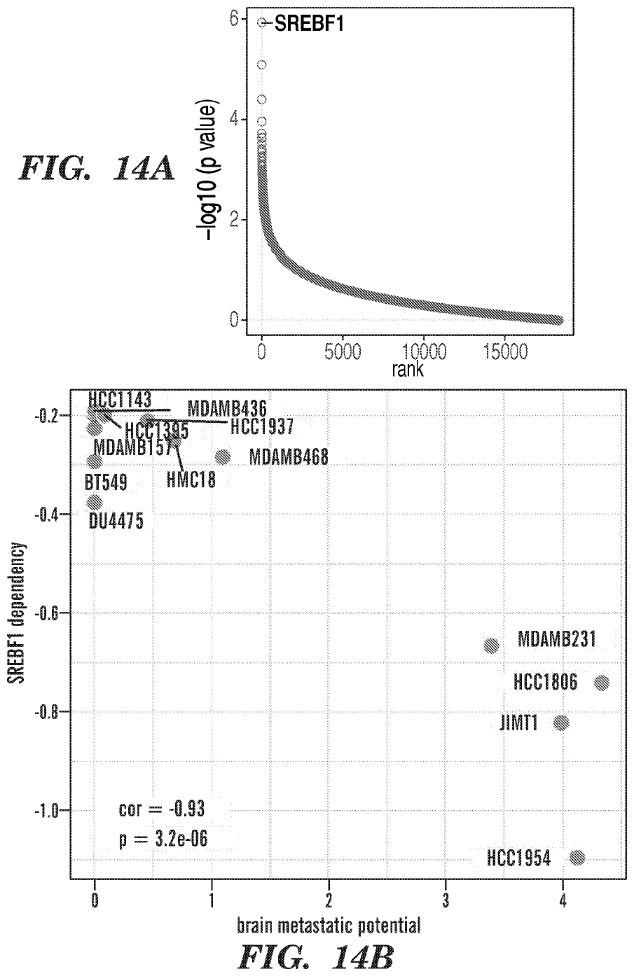

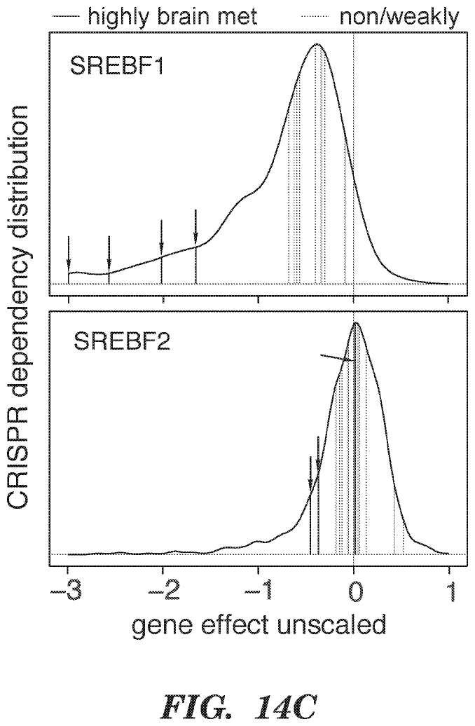

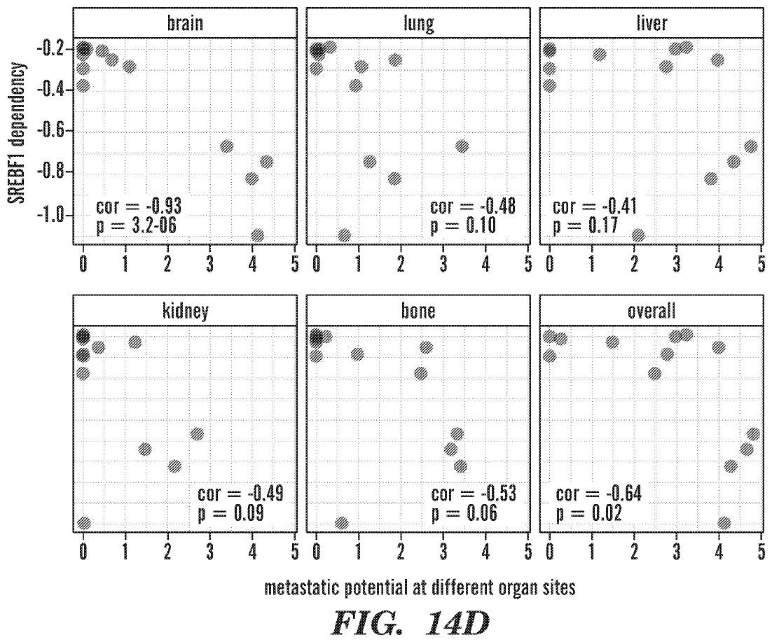

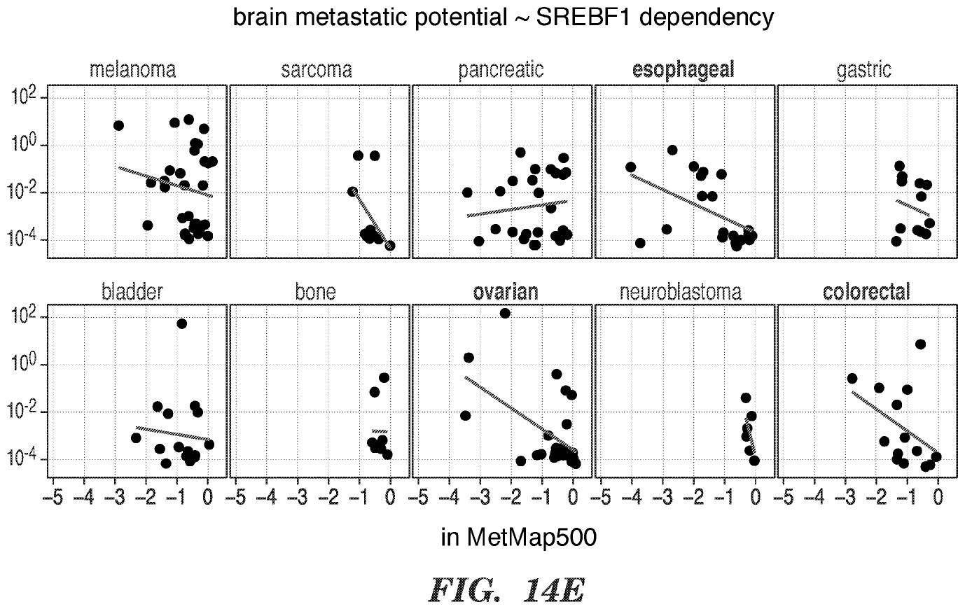

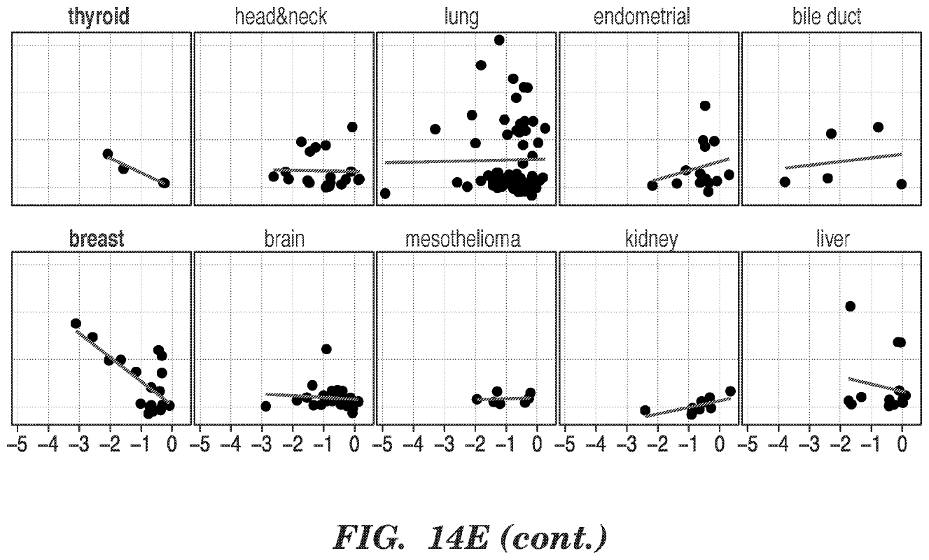

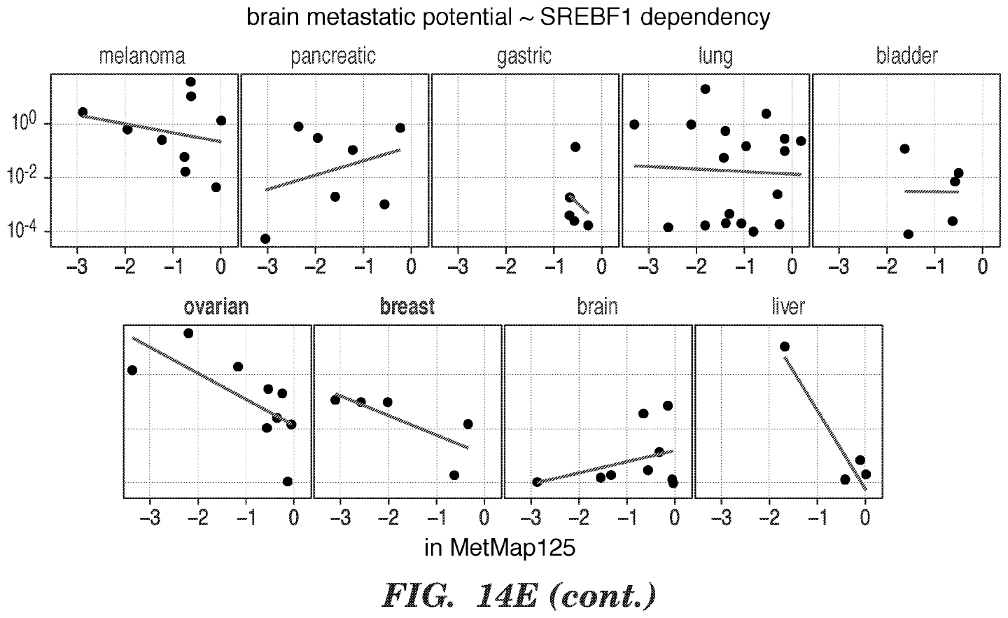

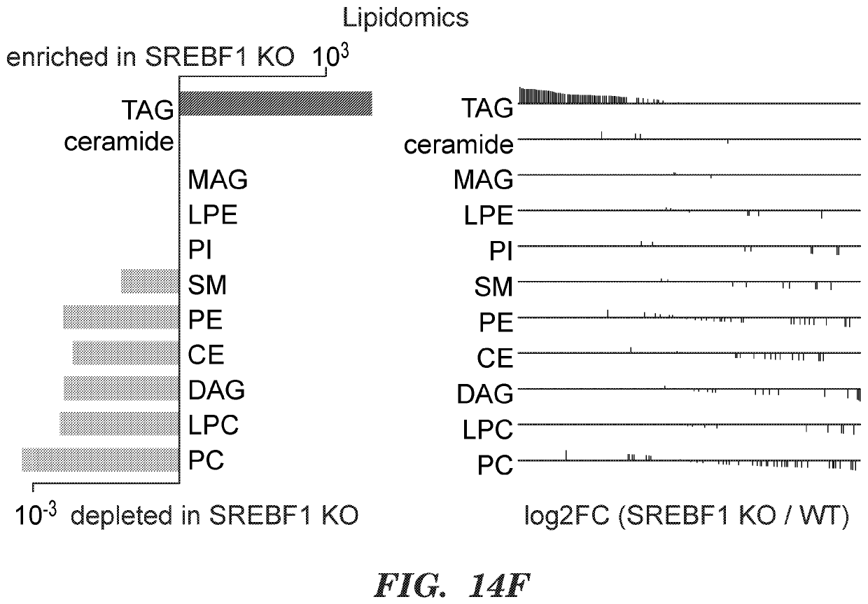

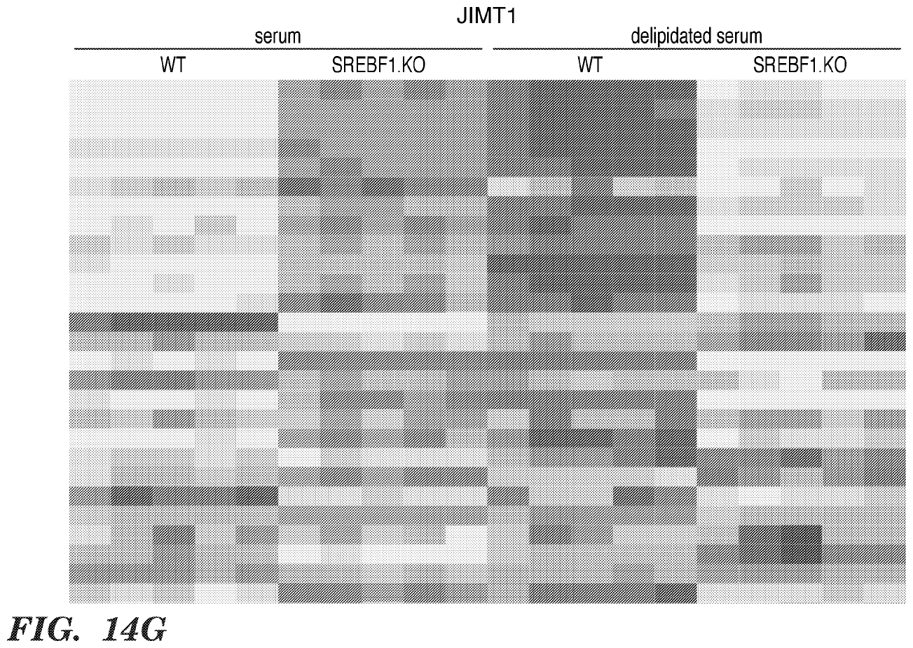

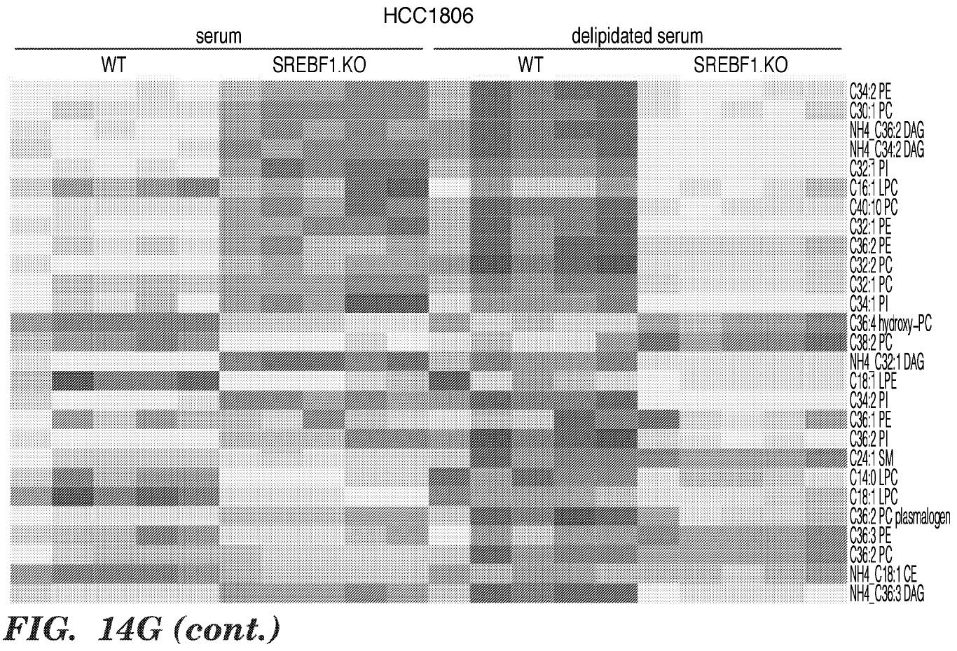

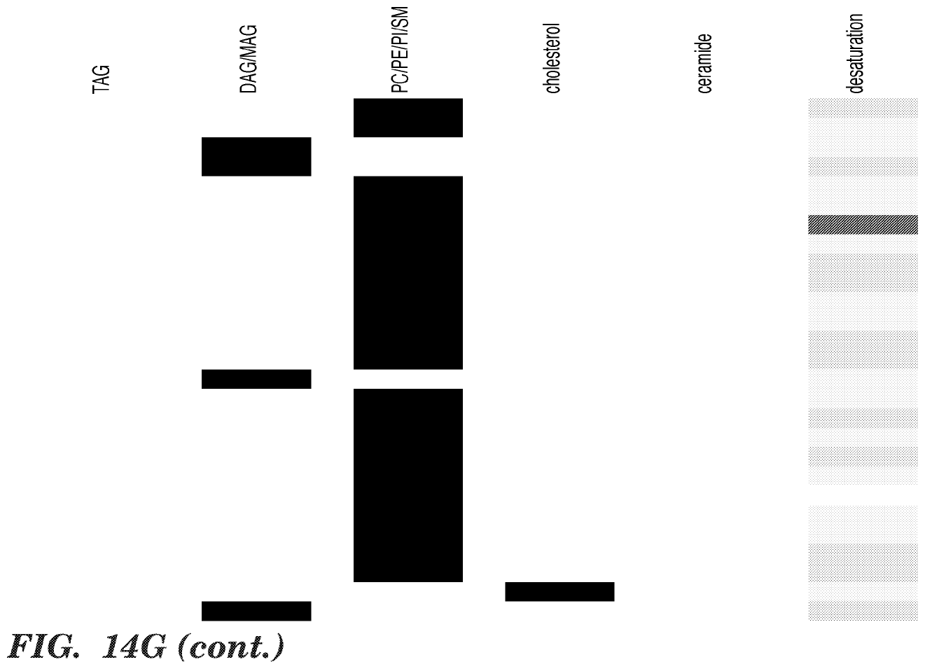

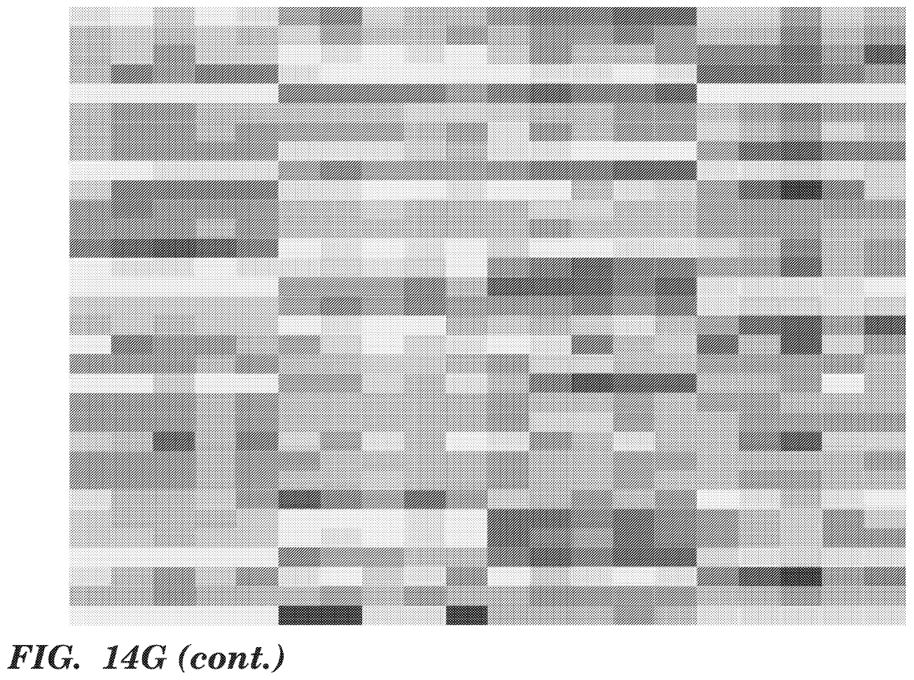

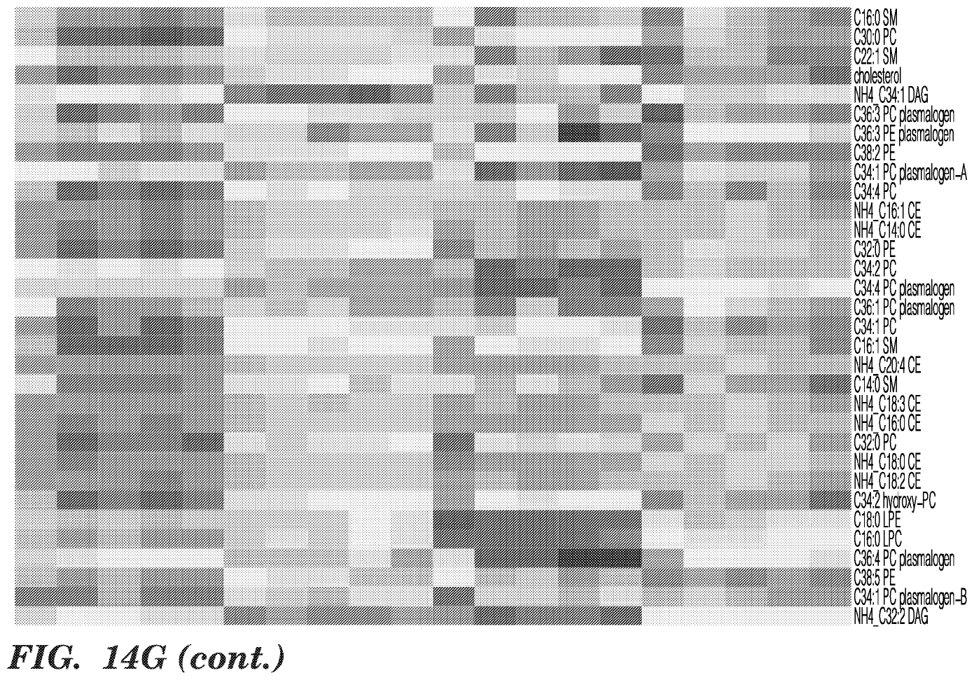

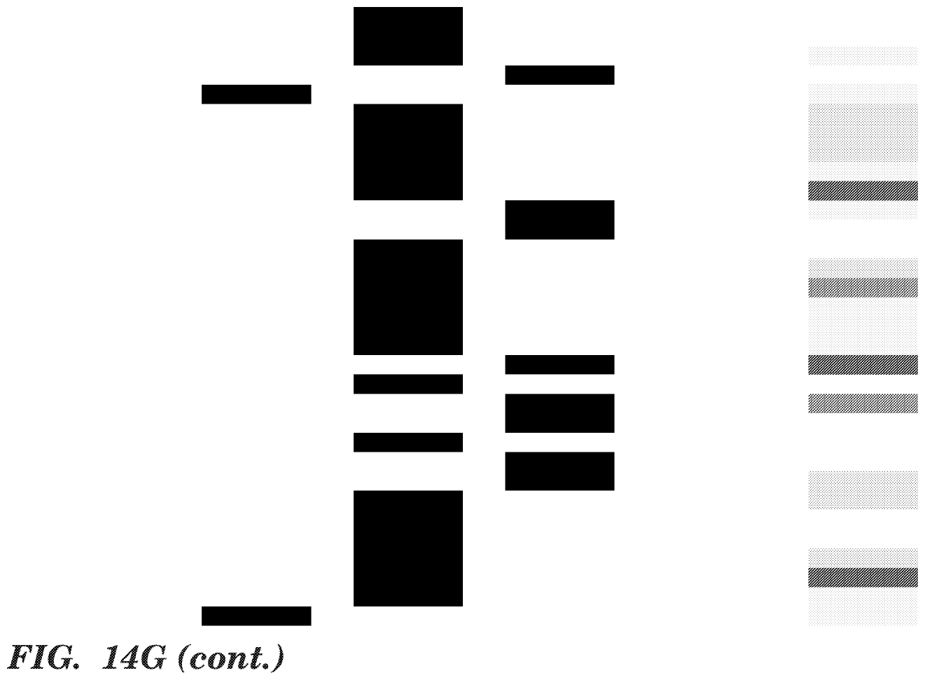

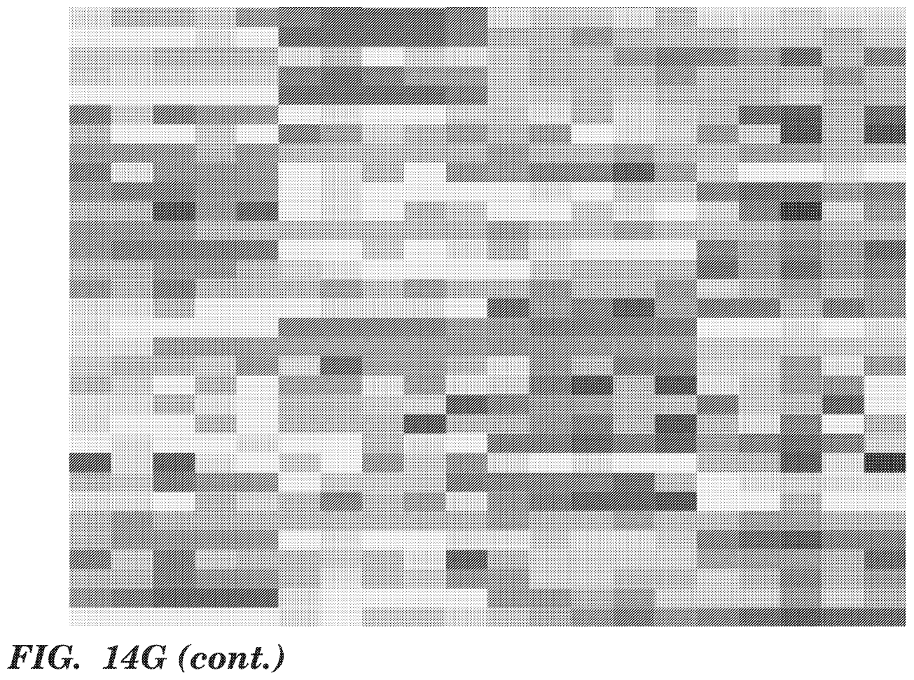

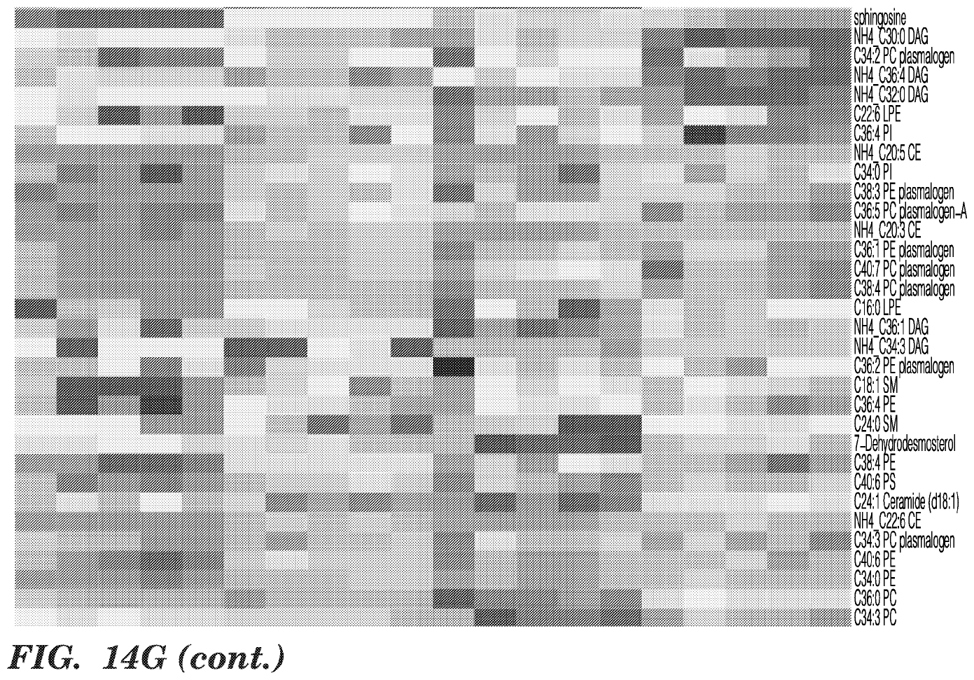

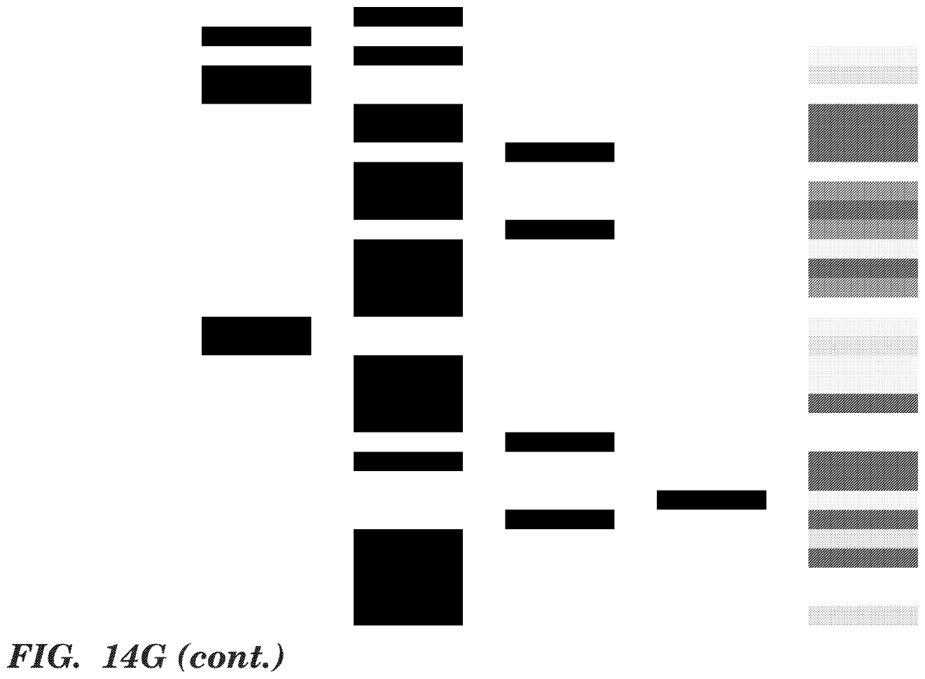

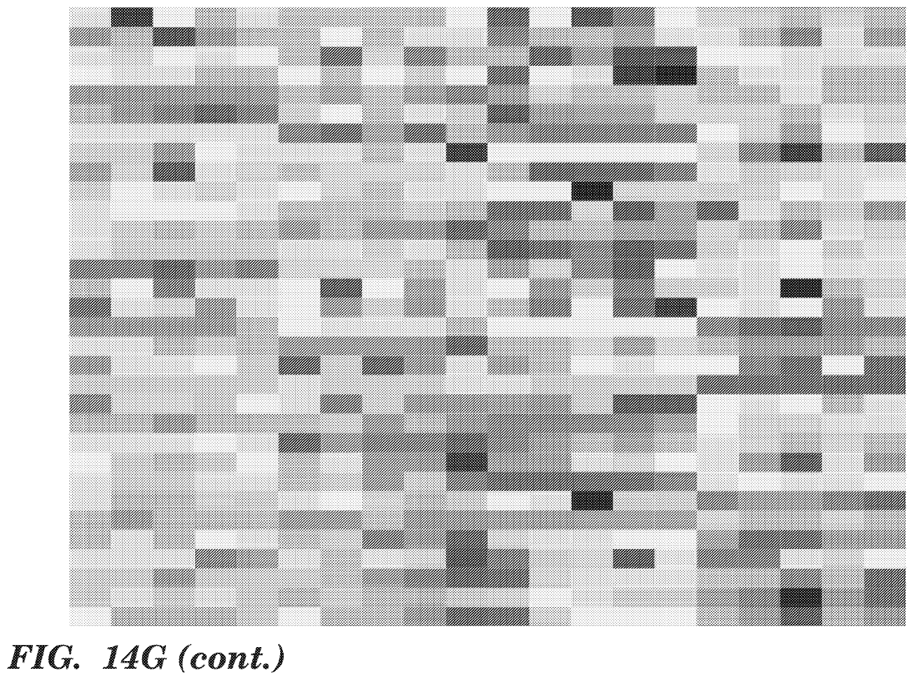

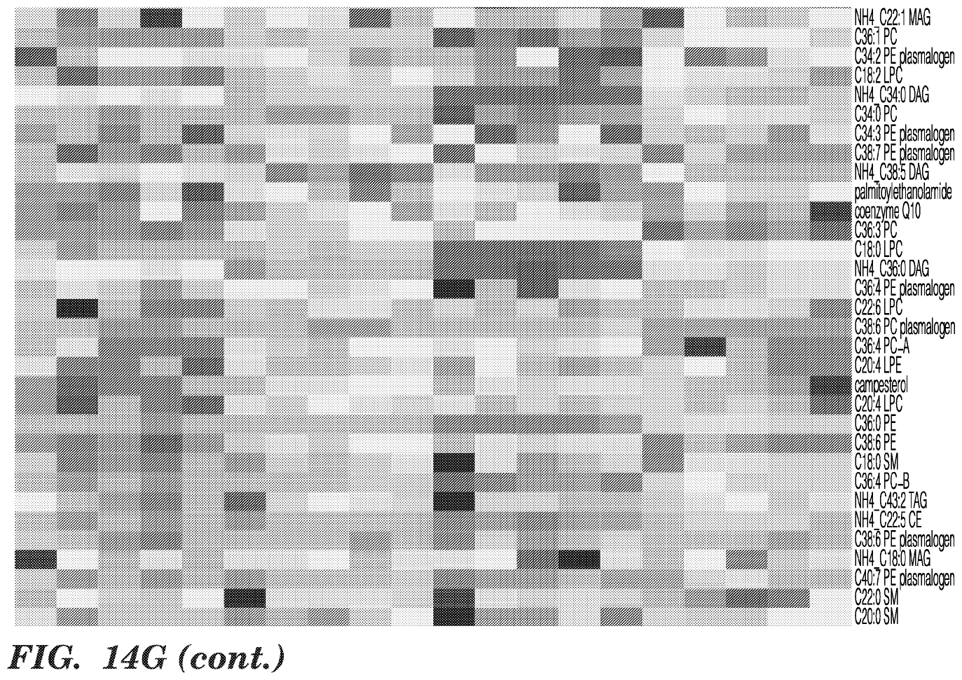

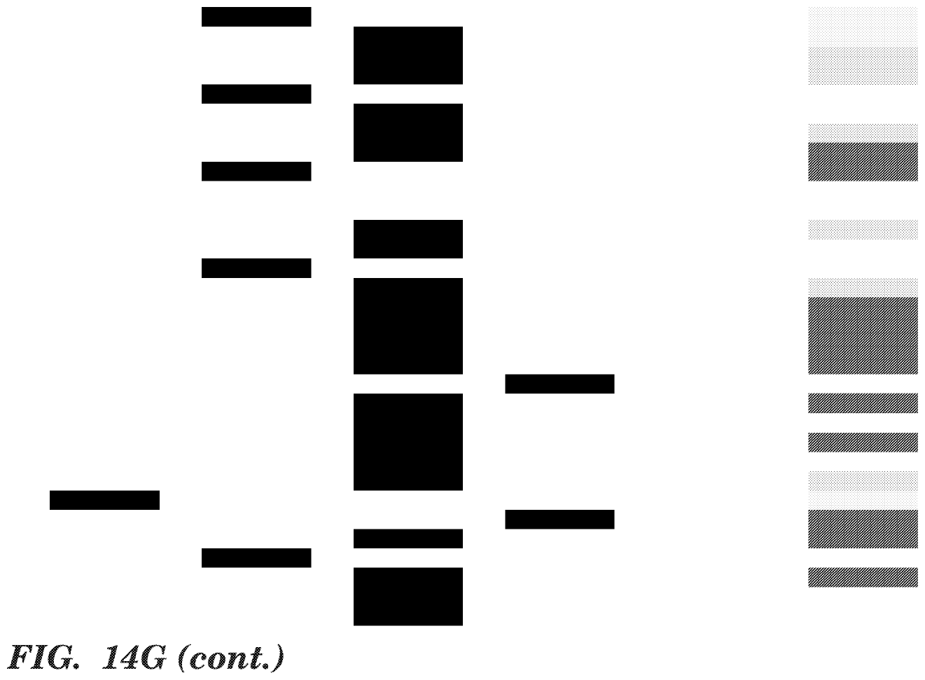

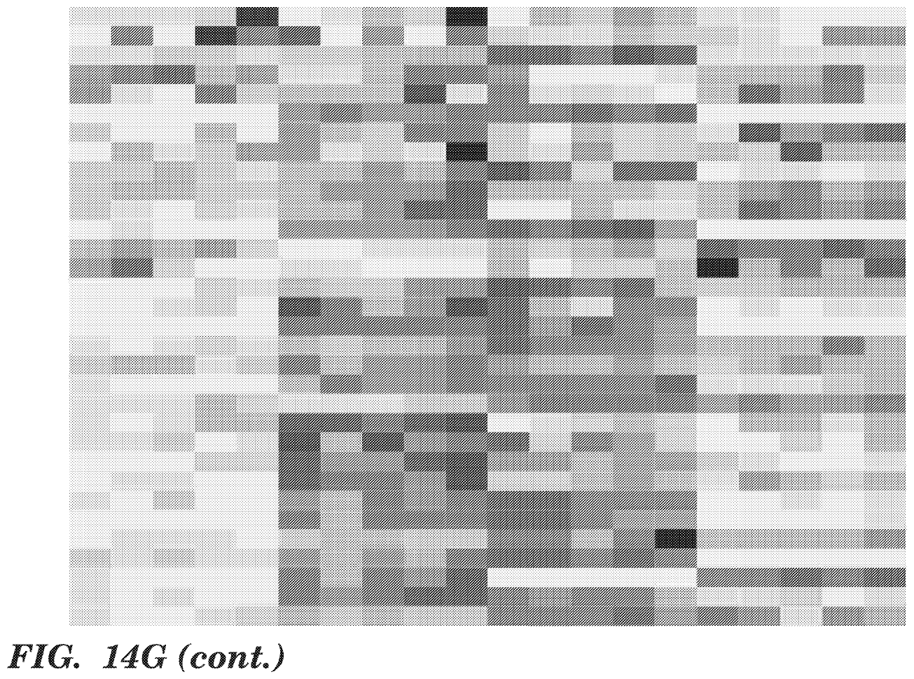

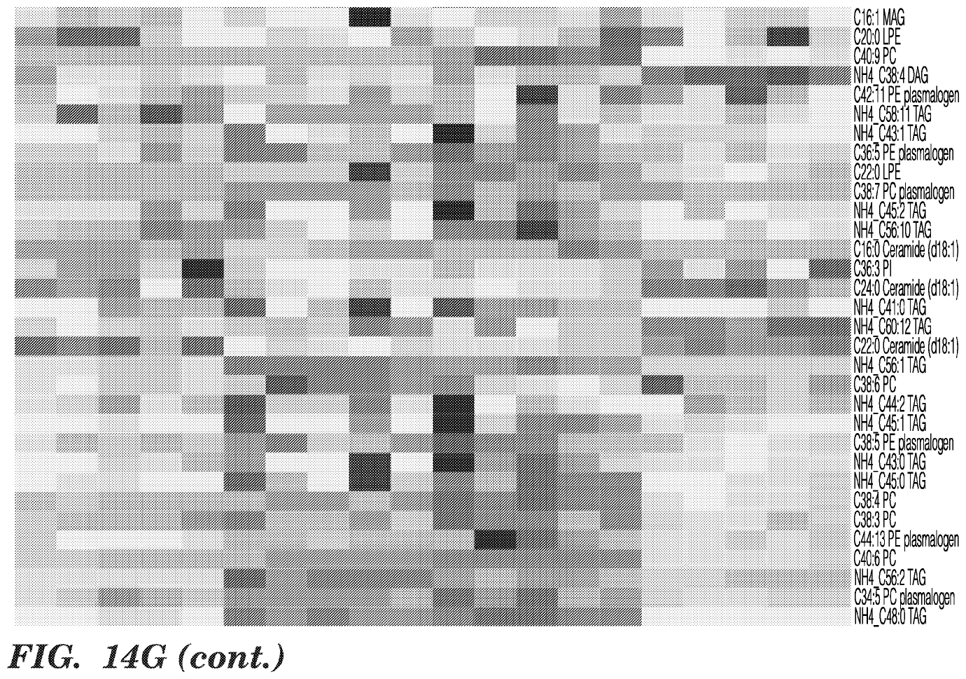

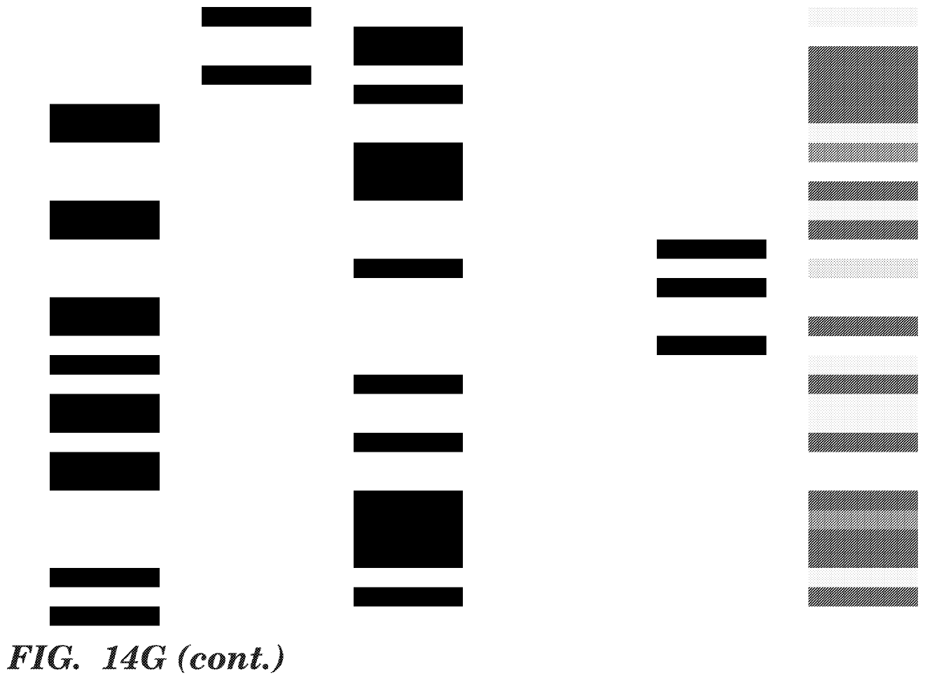

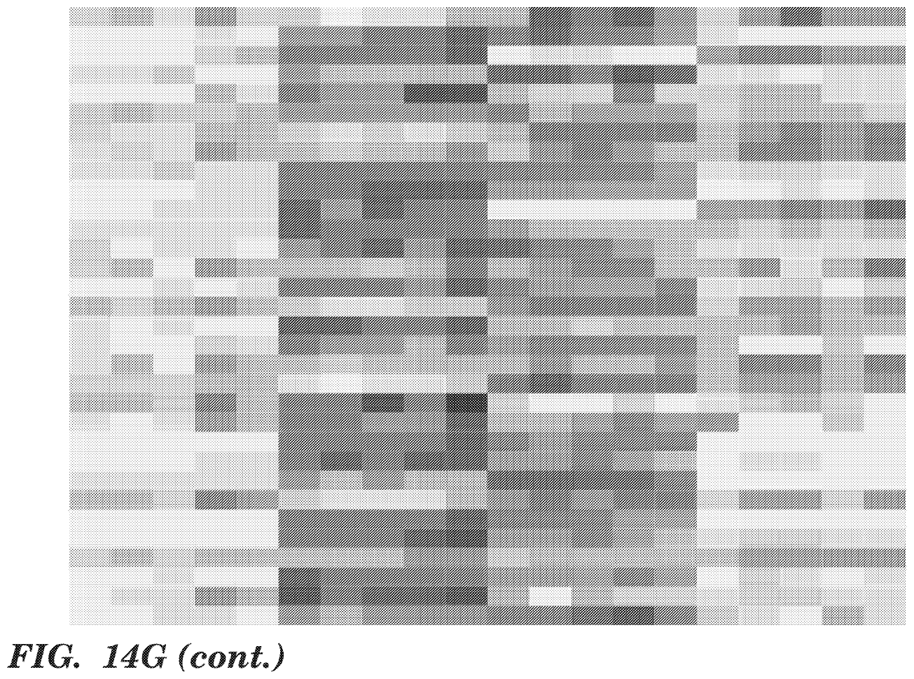

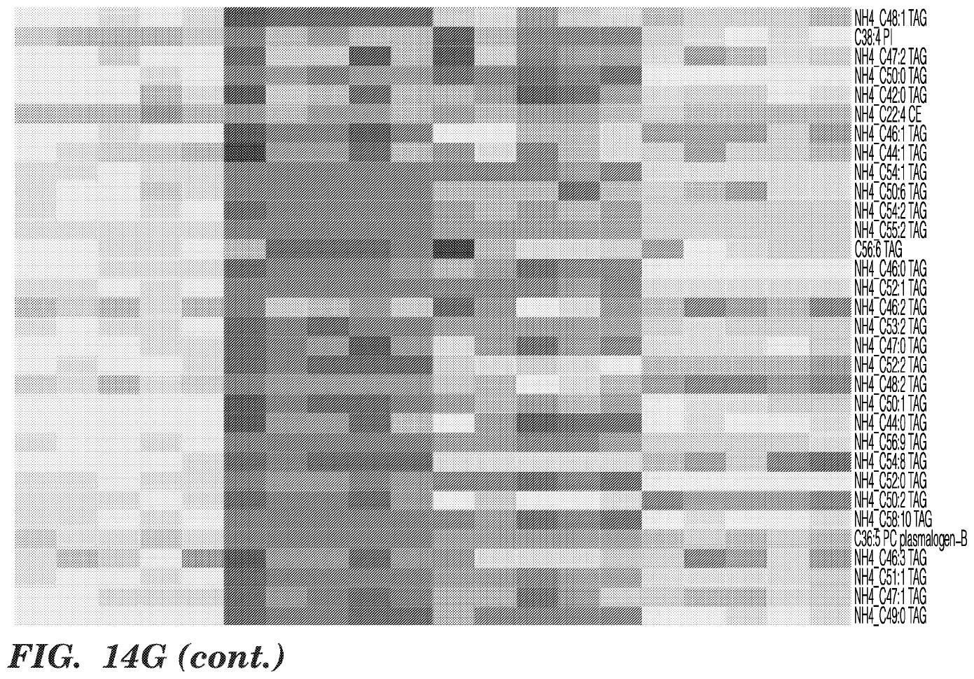

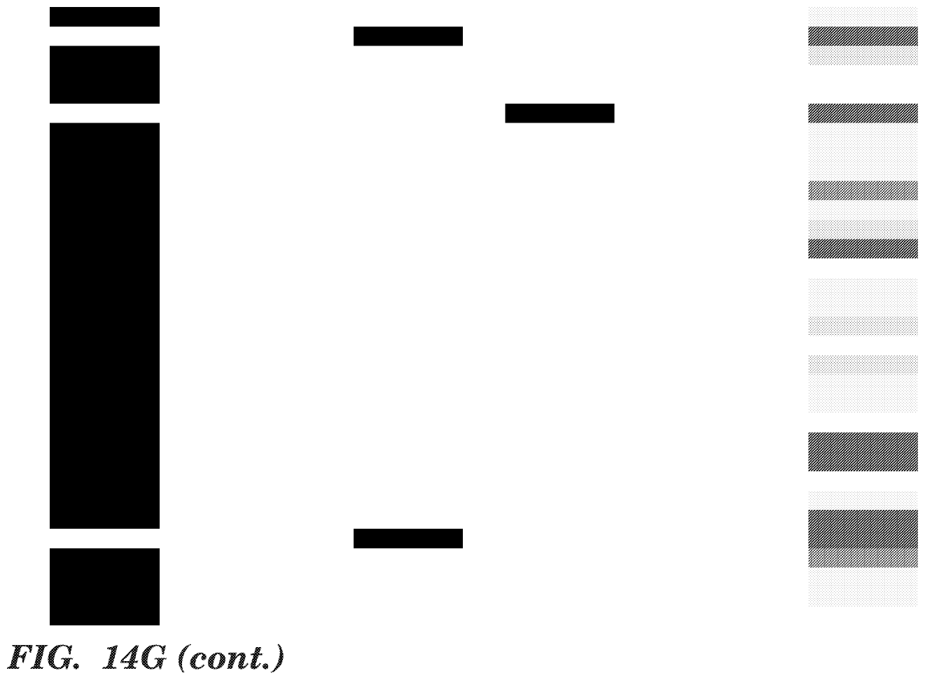

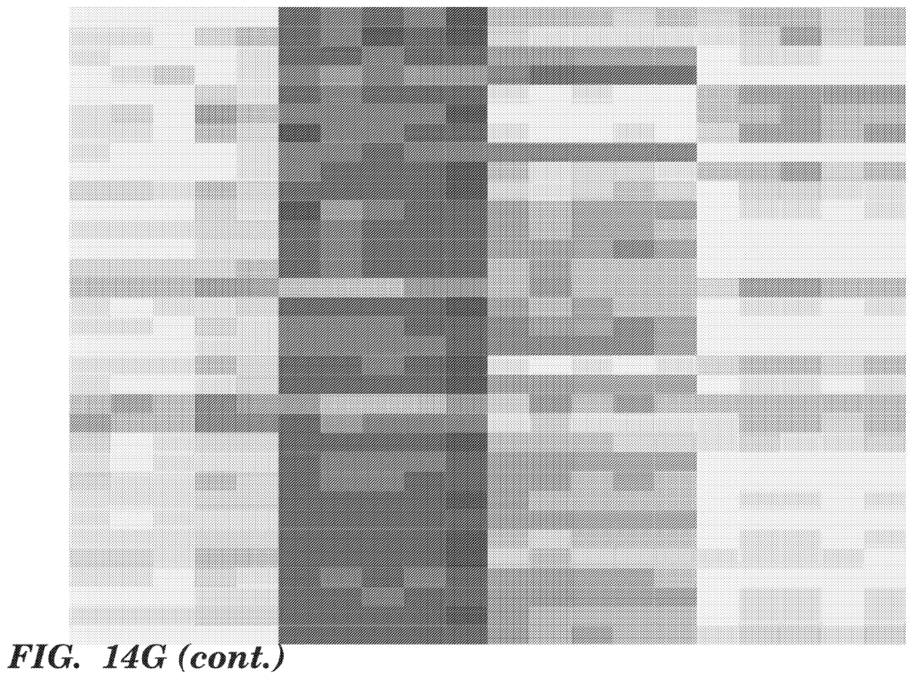

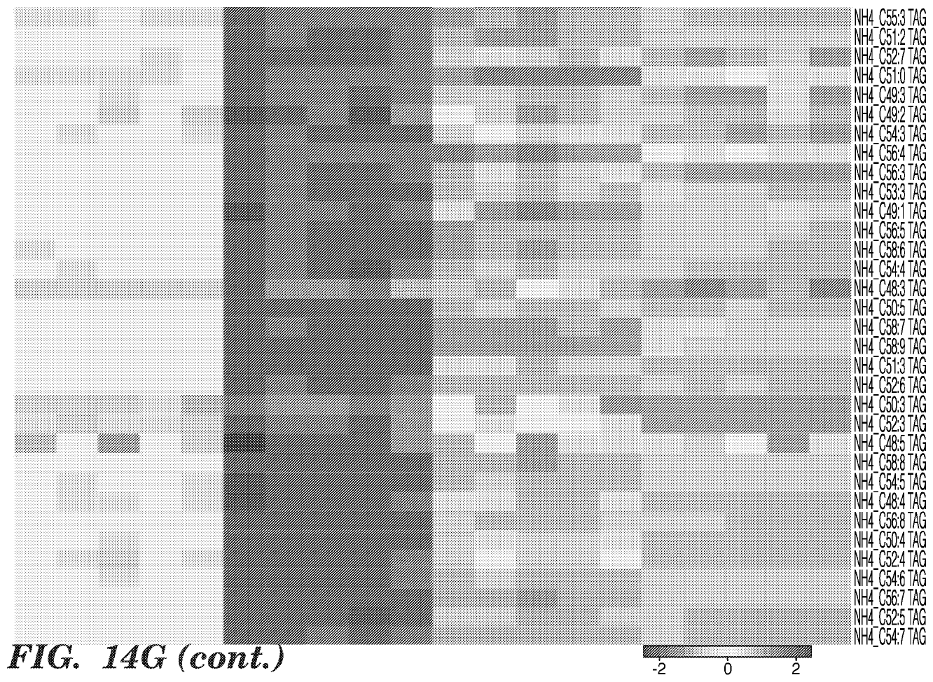

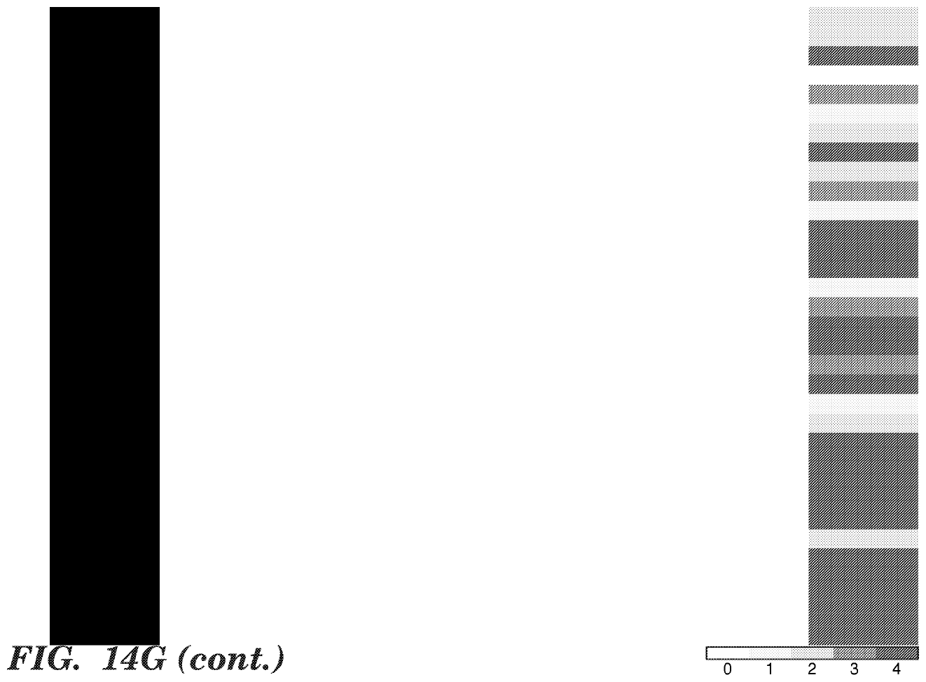

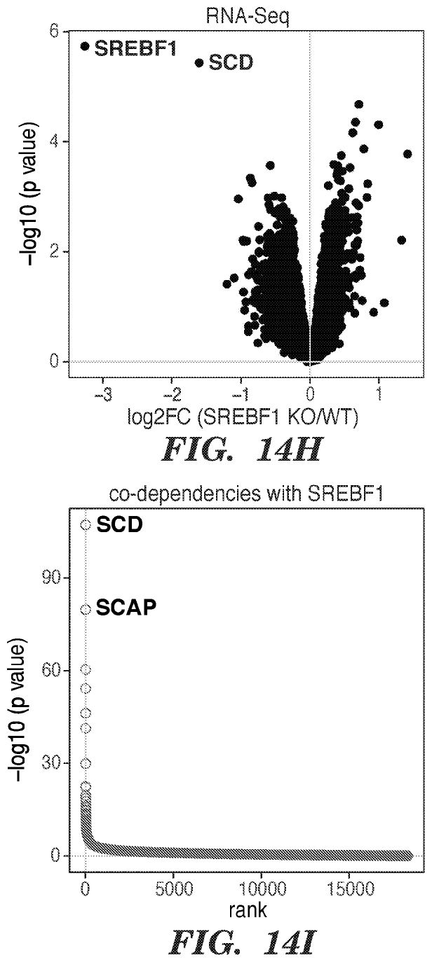

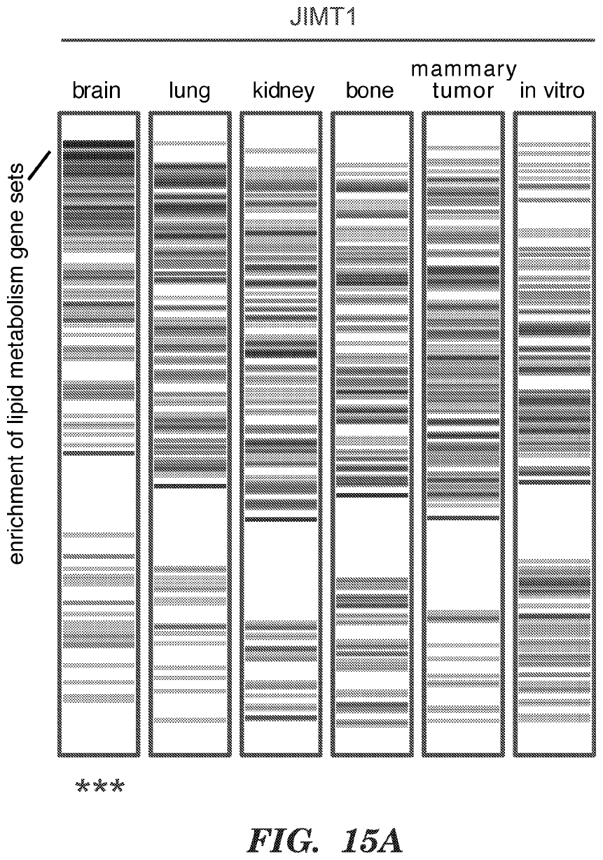

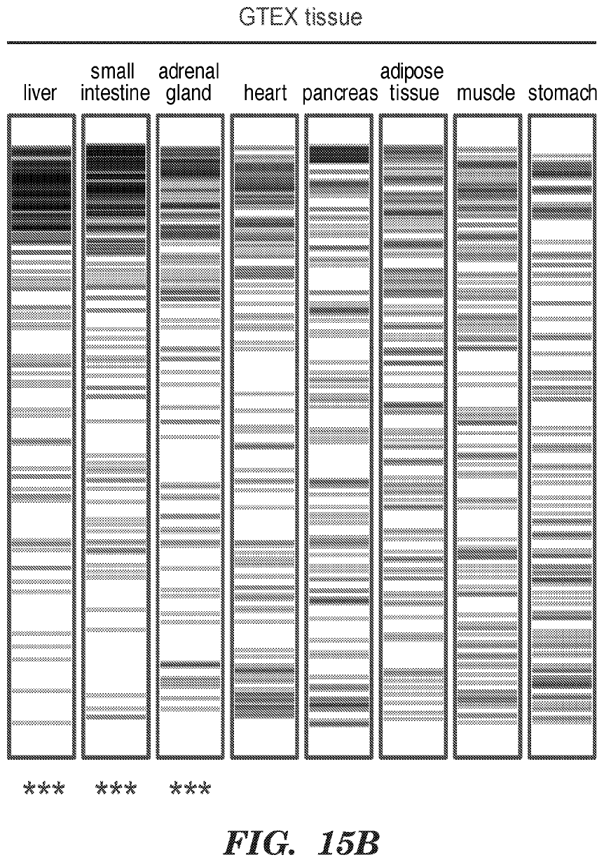

To further investigate the functional significance of a lipid metabolic profile of cells with brain metastatic potential, genome-wide CRISPR/Cas9 viability screening data was analyzed to identify vulnerabilities associated with the brain-metastatic state (Meyers et al., Nat. Genet., 49: 1779-84 (2017), the contents of which are incorporated herein by reference in their entirety). Remarkably, SREBF1 was the top correlated dependency (i.e., cancer cells rely on SREBF1 to switch to a brain-metastatic state in vitro) for brain metastasis (p=5.9e-8, FDR=0.001, FIG. 14A). Interestingly, SREBF1 was selectively required in vitro for growth of brain-metastatic cell lines compared to breast cancers that had low or no brain metastatic potential (FIG. 14B, 14C). No association was seen between SREBF1 and metastasic potential to other organs (FIG. 14D). Such association was re-captured specifically in breast cancer when analyzing MetMap125 and MetMap500 datasets, suggesting the strong reproducibility of this finding (FIGS. 14E). Of note, the SREBF1 paralog SREBF2 was not associated with brain metastatic potential (FIG. 14C).

SREBF1 is a pivotal transcription factor that mediates lipid synthesis downstream of PI3K pathway. To understand if SREBF1 confers the lipid state observed in brain metastatic cells, lipidomics were performed after knocking-out SREBF1 in brain metastatic cell lines JIMT1 (PIK3CA-mut) and HCC1806 (8p-loss). SREBF1 knock-out (KO) resulted in a dramatic shift in intracellular lipid content (FIG. 14F), including down-regulation of cholesterol, membrane lipids (PC, LPC, PE, SM), and DAGs (diacylglycerols, precursors of TAGs). Instead, TAGs switched from a low to a high state, presumably reflecting increased scavenging from the media containing lipid-rich serum. Indeed, culture in media with delipidated-serum resulted in inability of cells to accumulate TAGs (FIG. 14G). These results suggested that SREBF1 explained the altered lipid metabolic state in brain metastatic cell lines. To interrogate the transcriptional targets of SREBF1, RNA-Seq was performed, which showed Stearoyl-CoA Desaturase (SCD) to be the most consistently downregulated gene by SREBF1 KO in brain metastatic lines (FIG. 14H). Consistent with this result, SCD scored as the top co-dependency of SREBF1 across 734 cell lines in the genome-wide CRISPR/Cas9 viability screening data (FIG. 14I). This is followed by SCAP, the upstream activator of SREBF1. Of note, SREBF1 and its transcriptional target SCD were uniquely upregulated in brain metastasis (FIG. 15D). Similar upregulation was also observed in patient brain metastases compared to extracranial metastases, or to their matched primary tumors (FIGS. 15G, 15H, 15I). Taken together, genetic, metabolic, transcriptomic, and functional genomic evidence all point to an association between SREBF1-mediated lipid metabolism and brain metastasis.

### Example 9

### Gene Perturbation Shows Significance of SREBF1 and the Lipid Metabolism Pathway Genes in Mediating Brain Metastasis Outgrowth

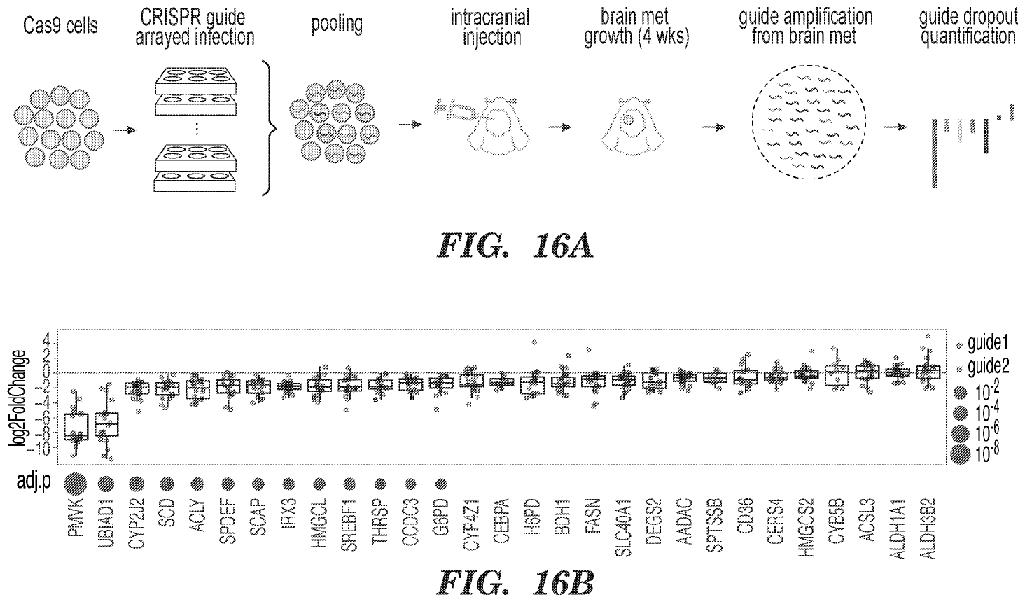

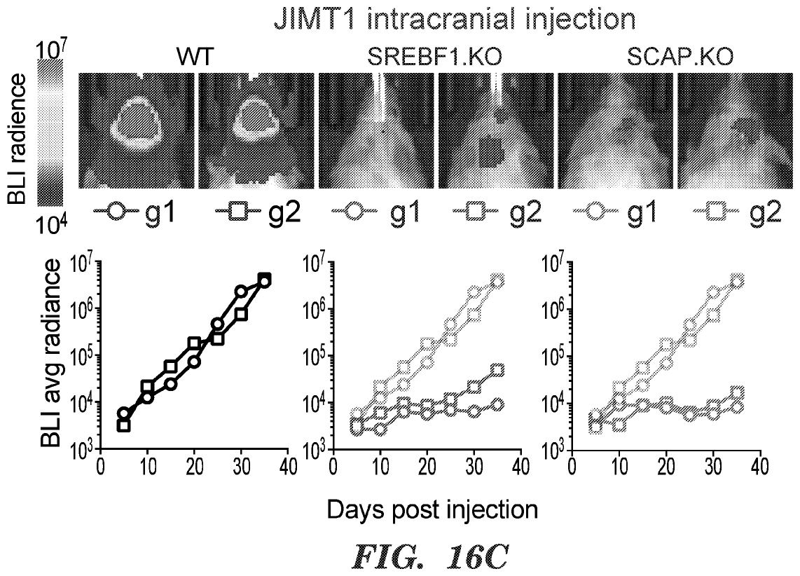

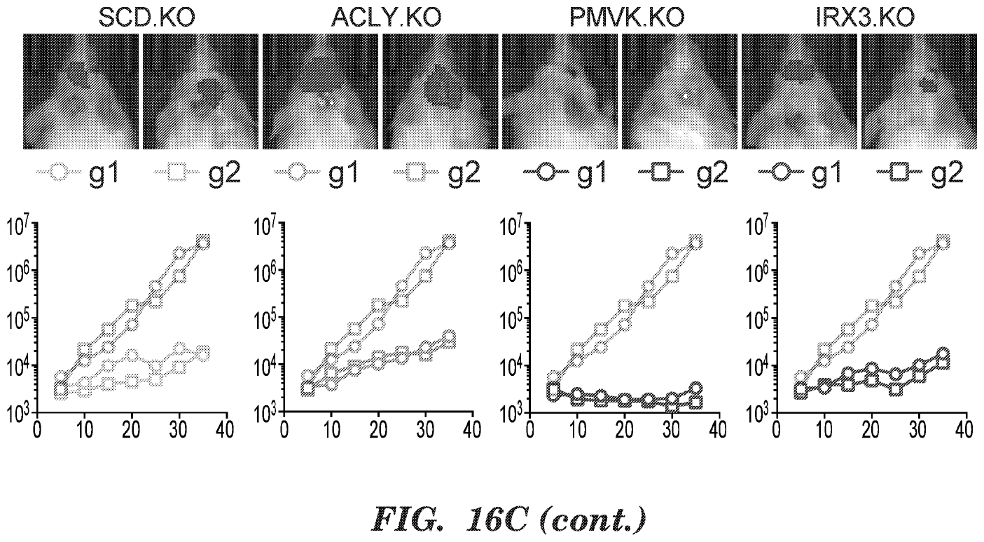

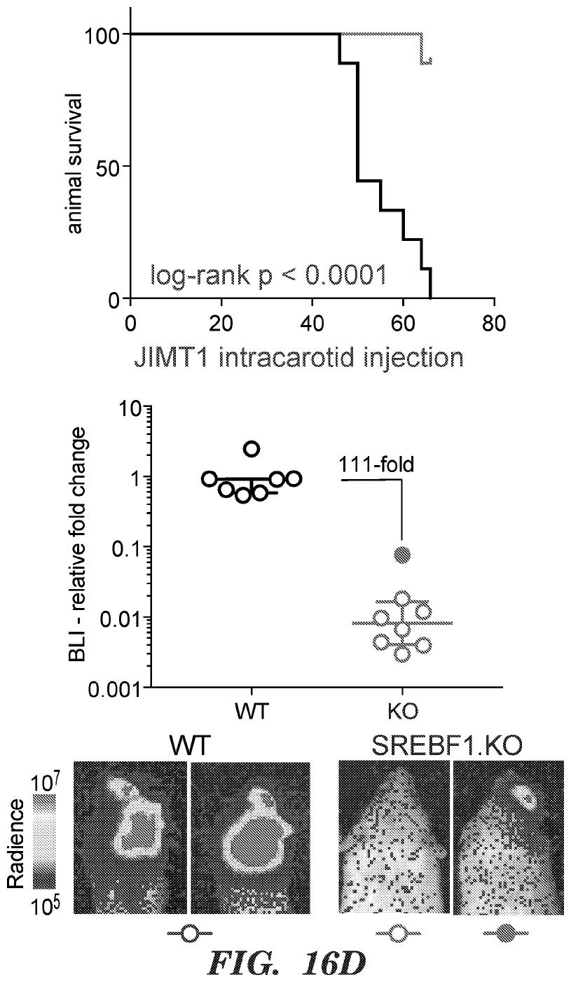

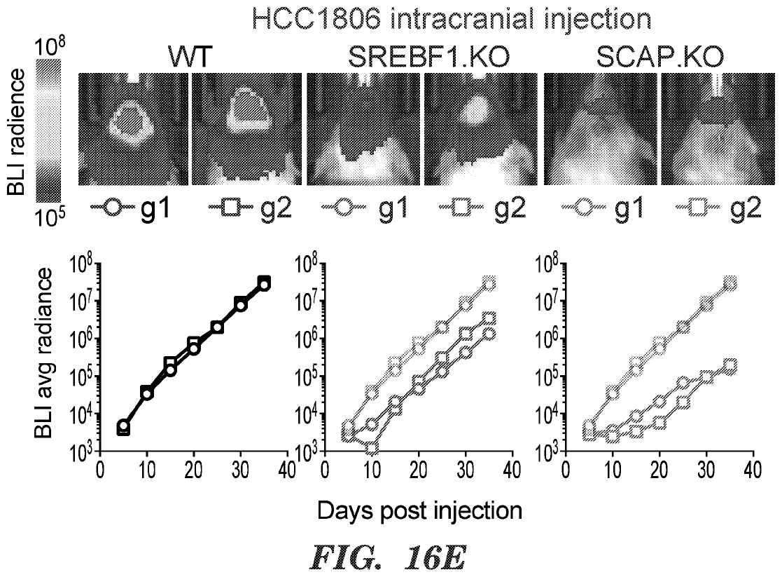

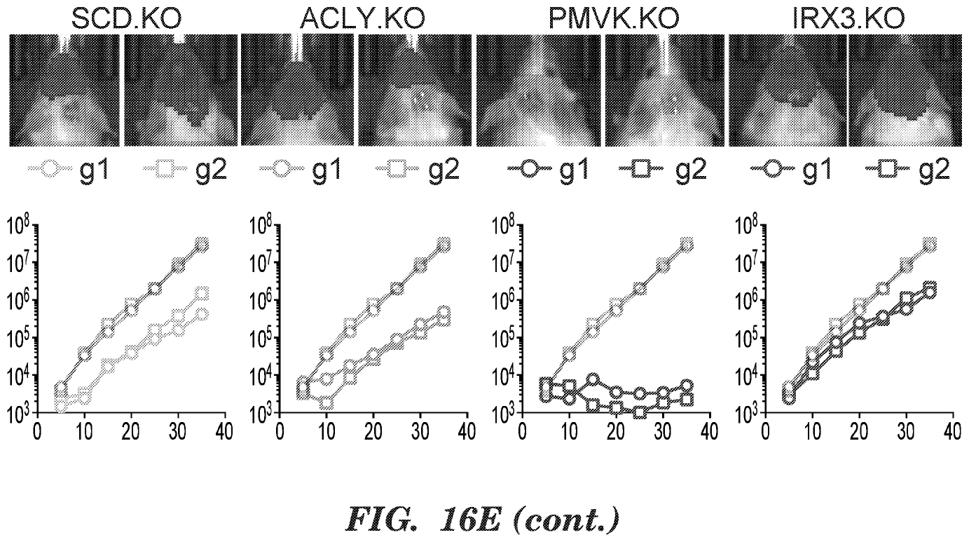

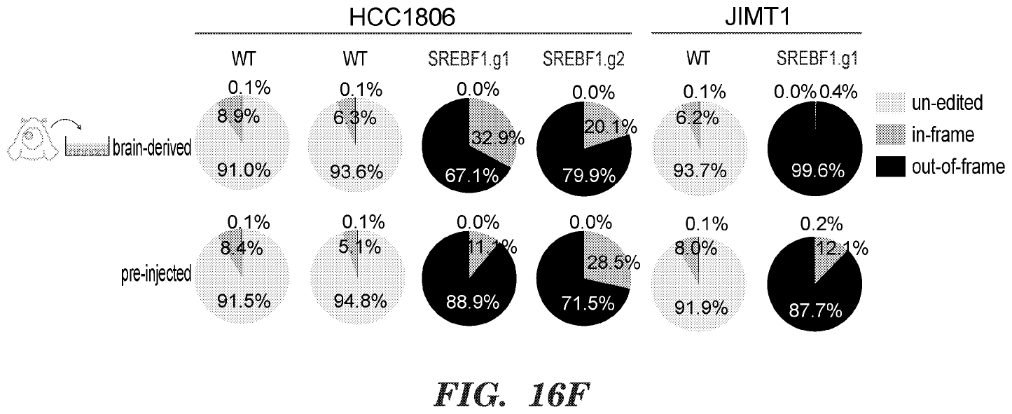

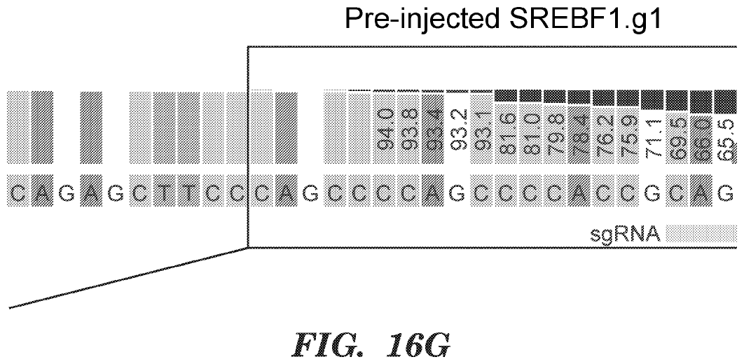

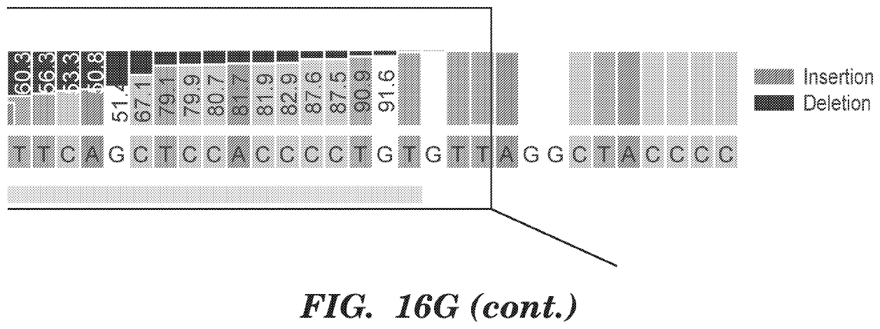

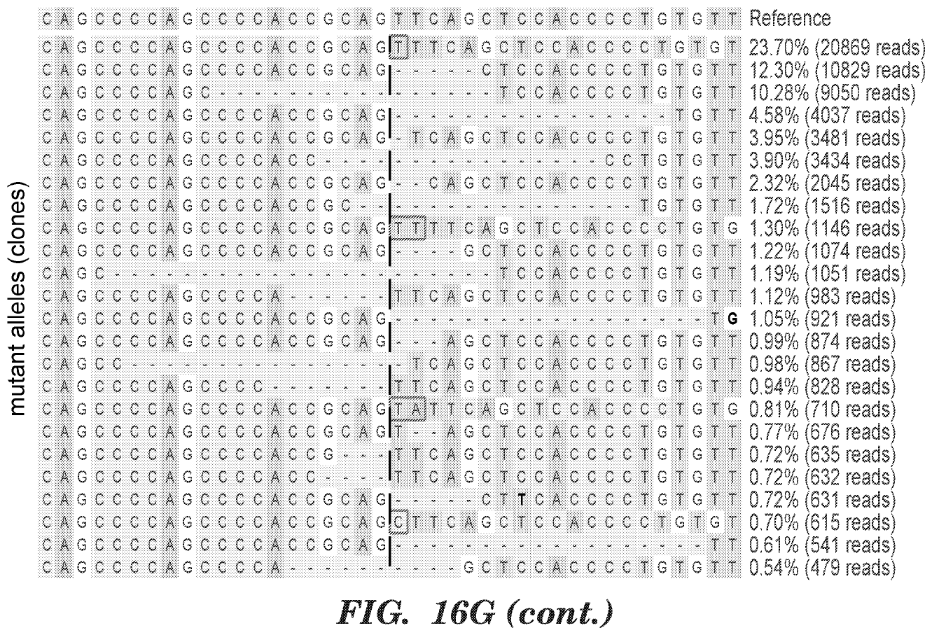

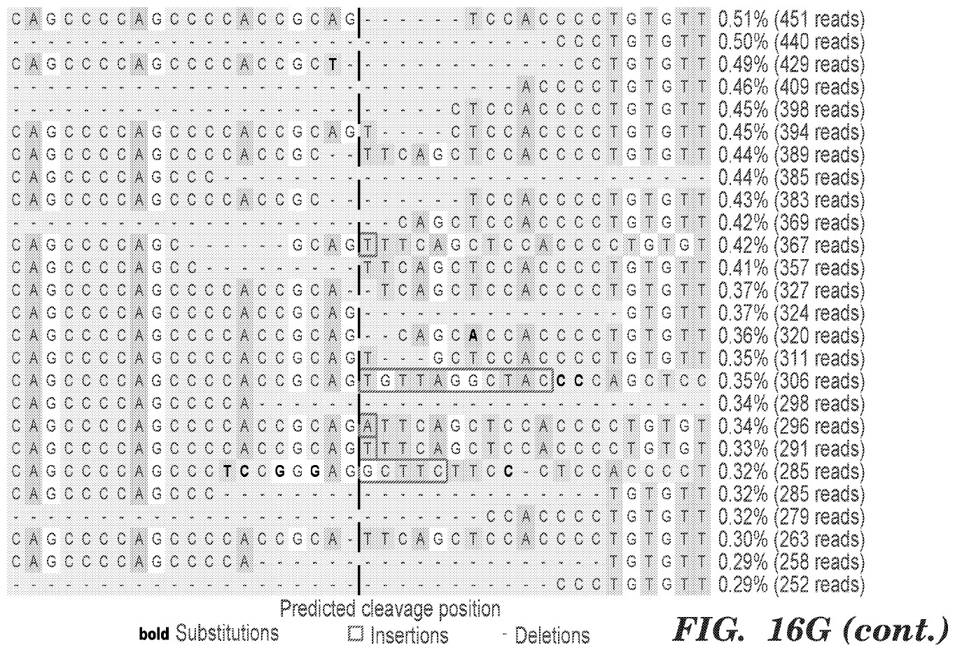

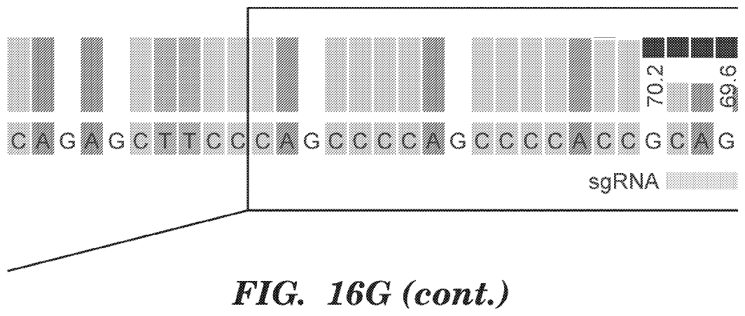

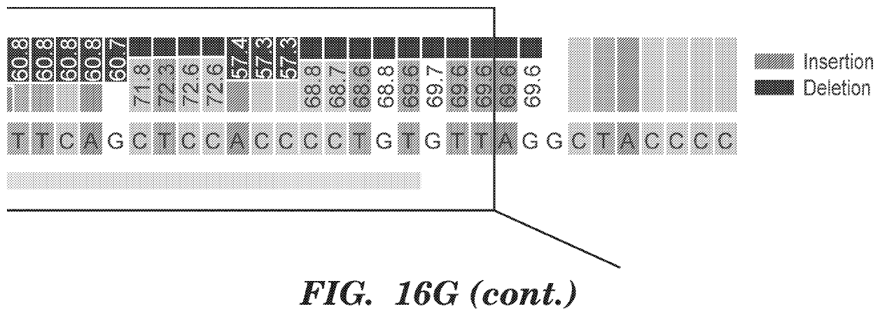

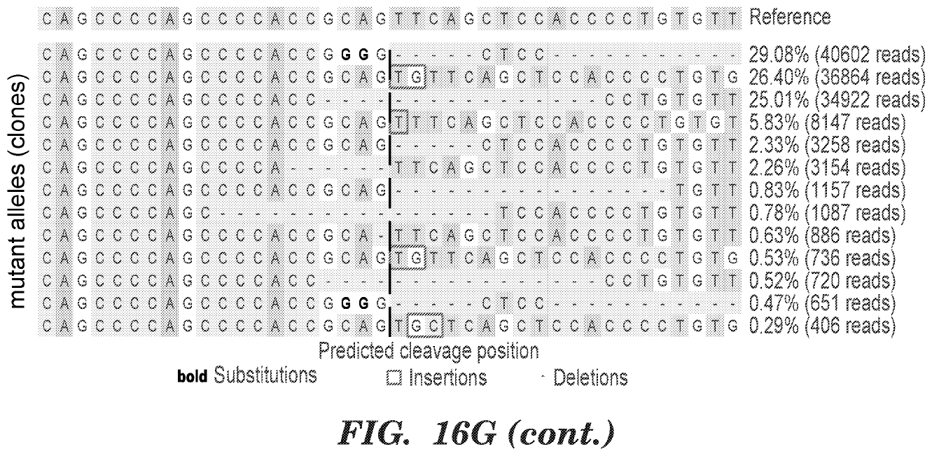

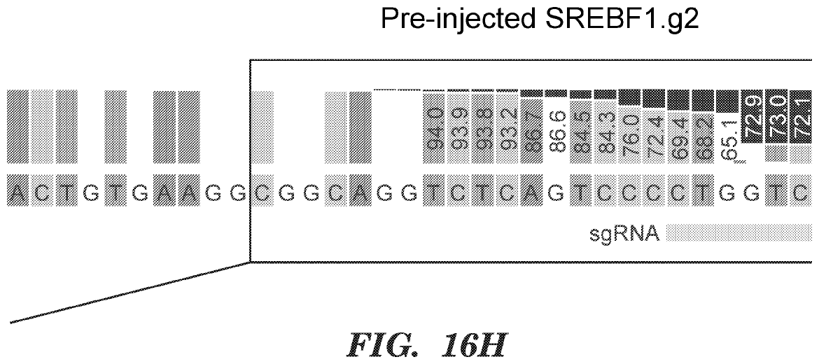

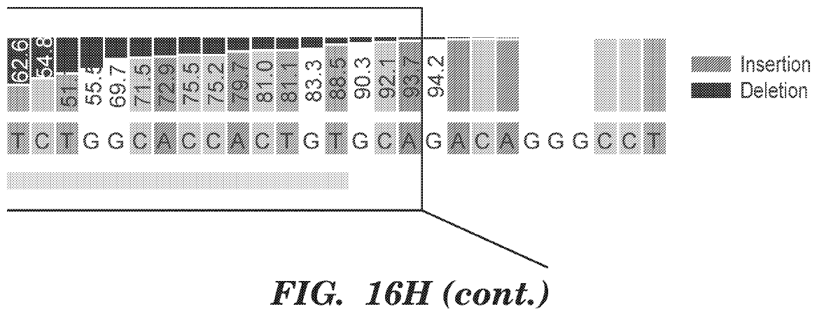

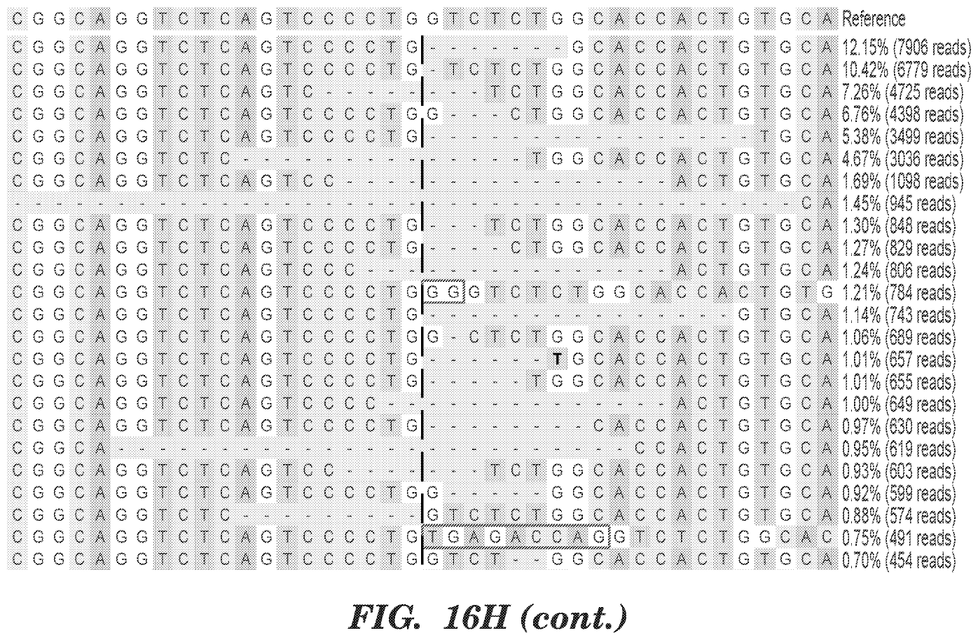

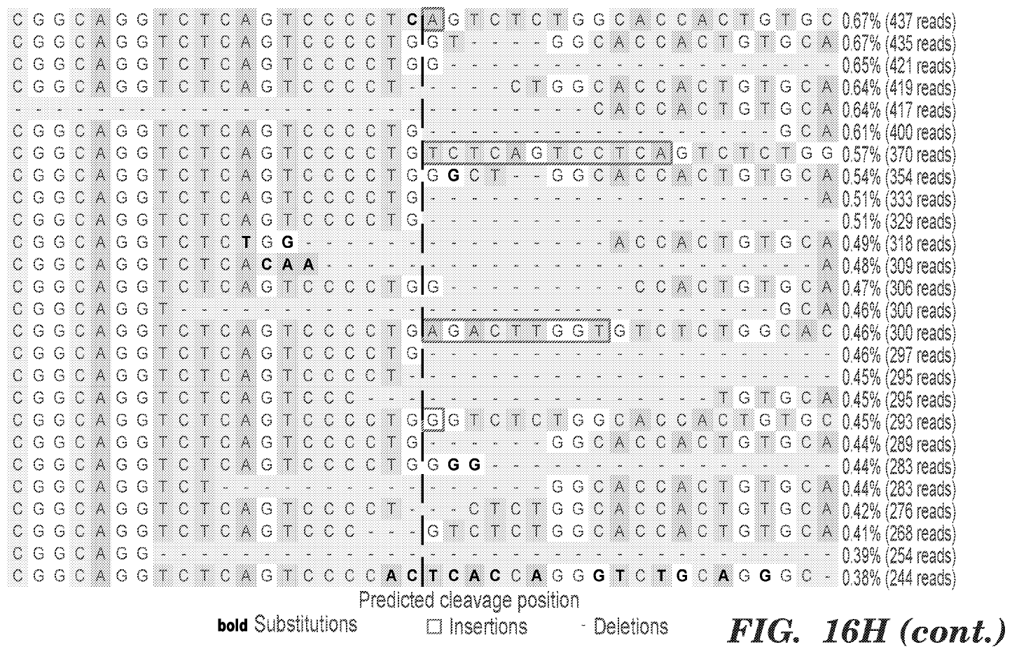

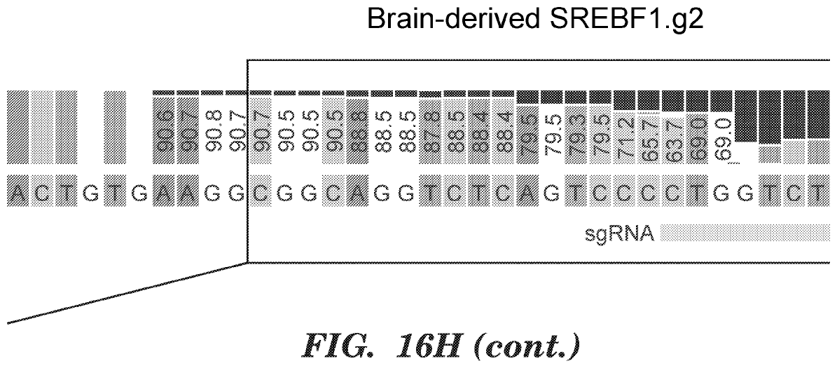

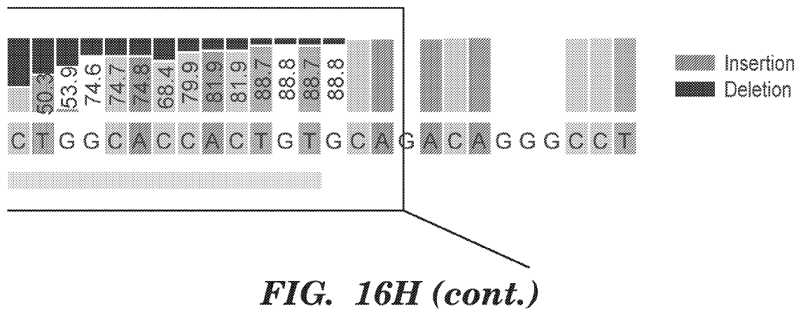

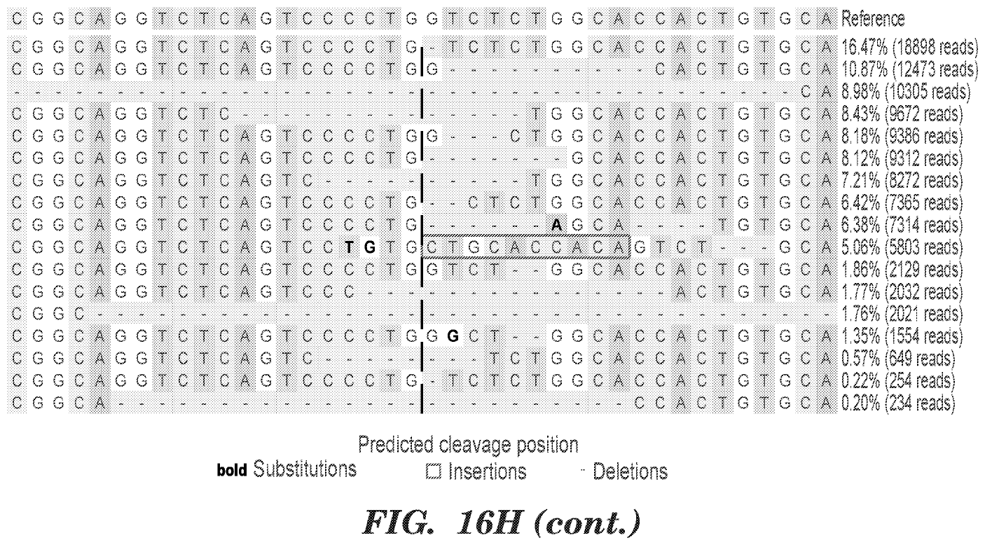

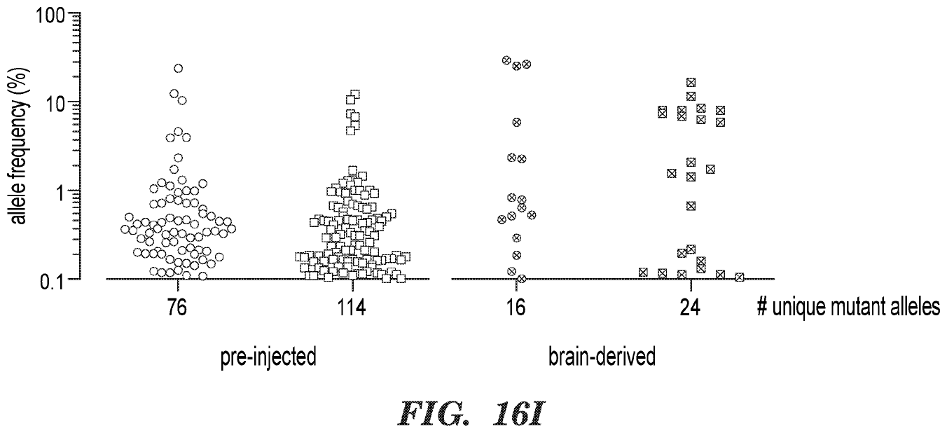

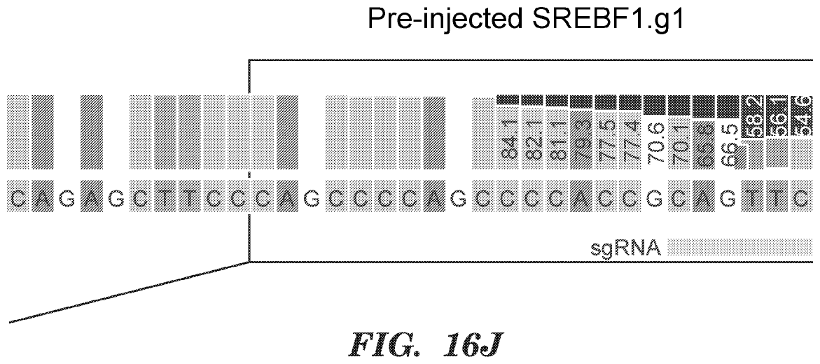

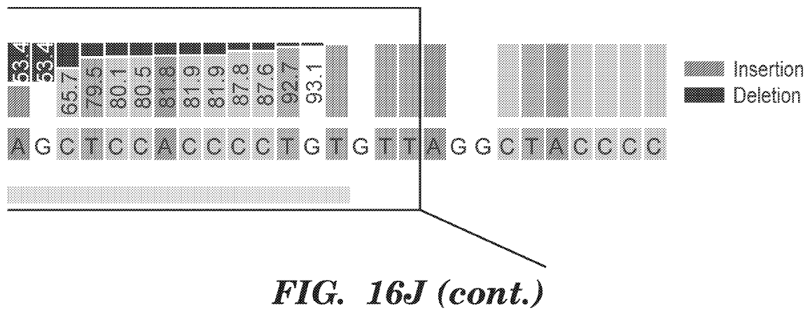

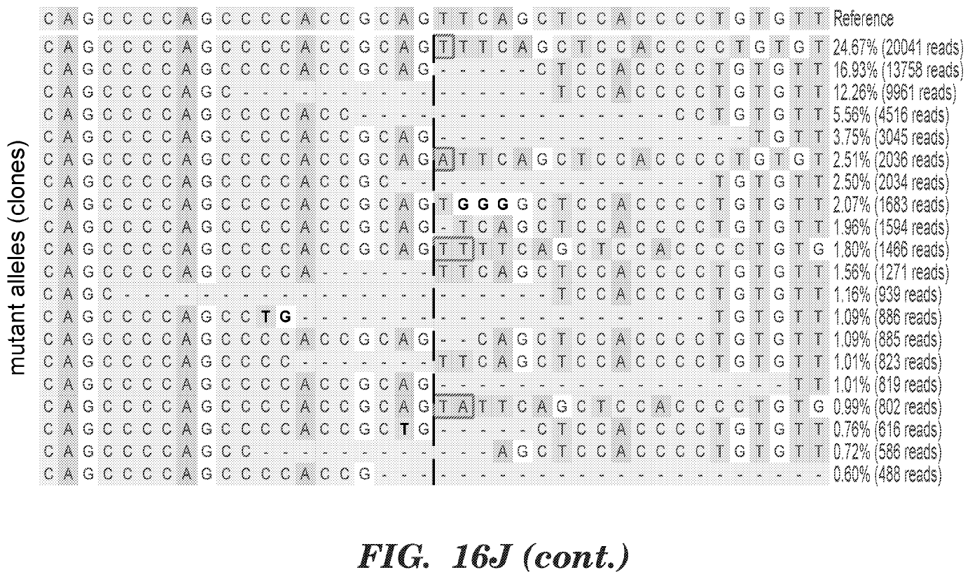

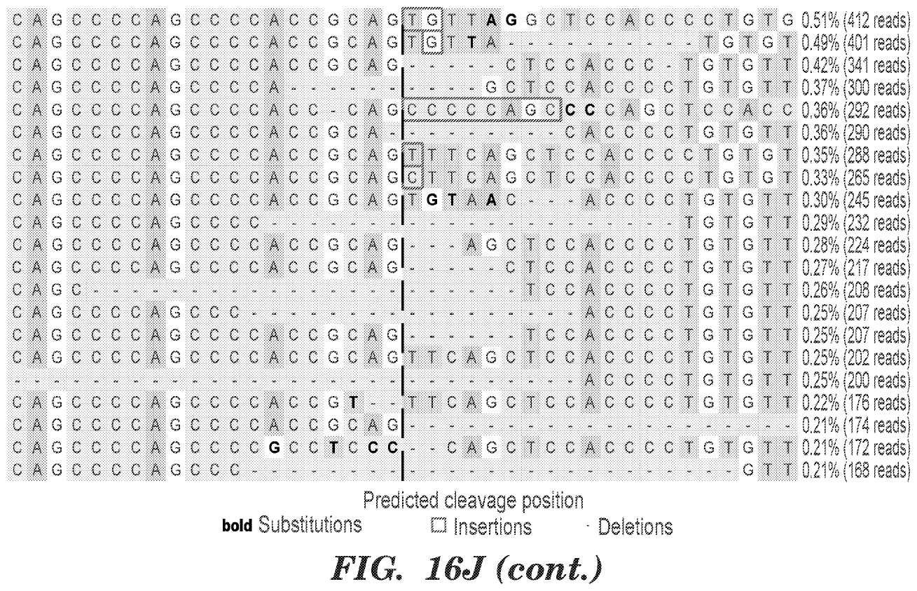

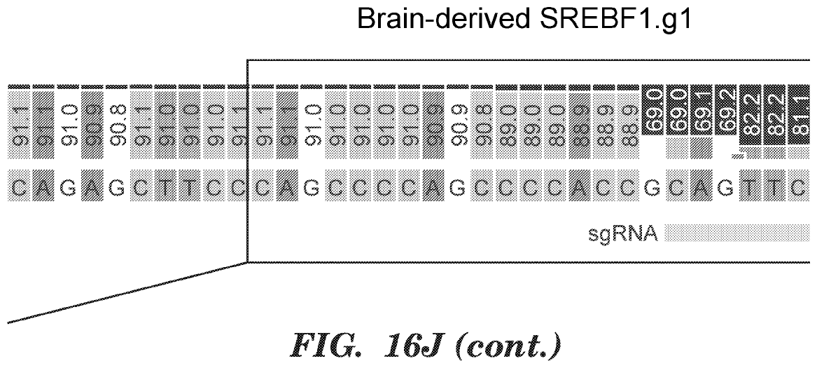

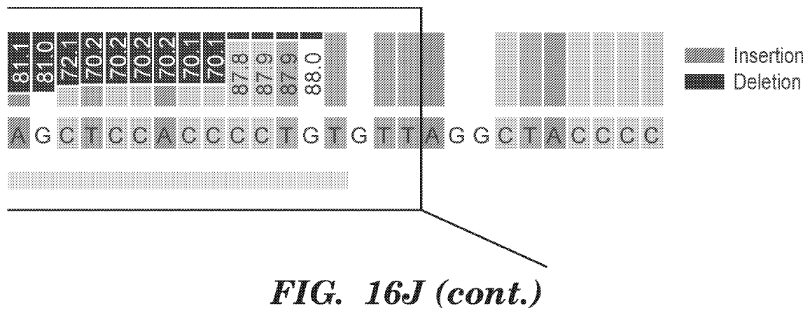

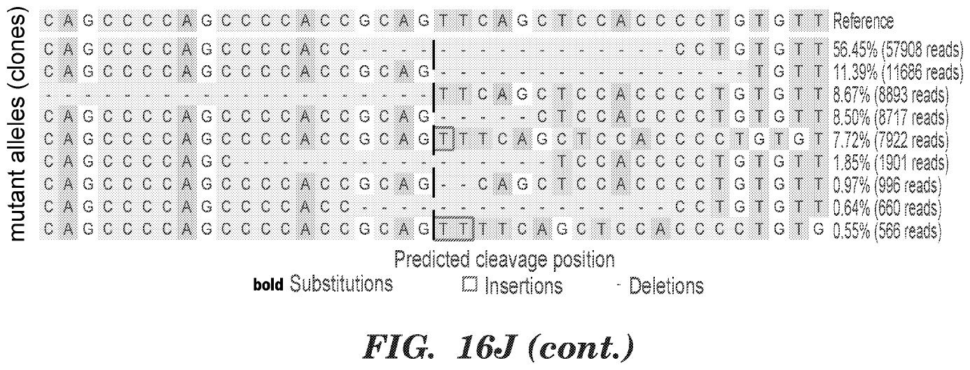

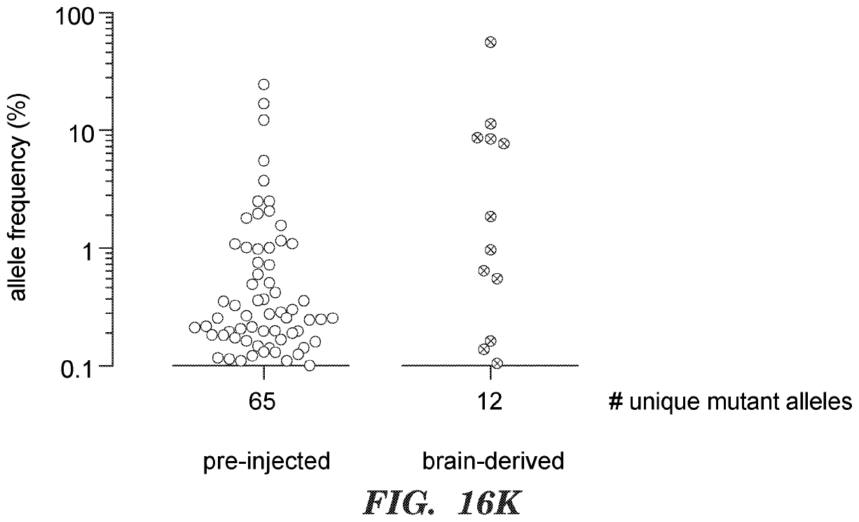

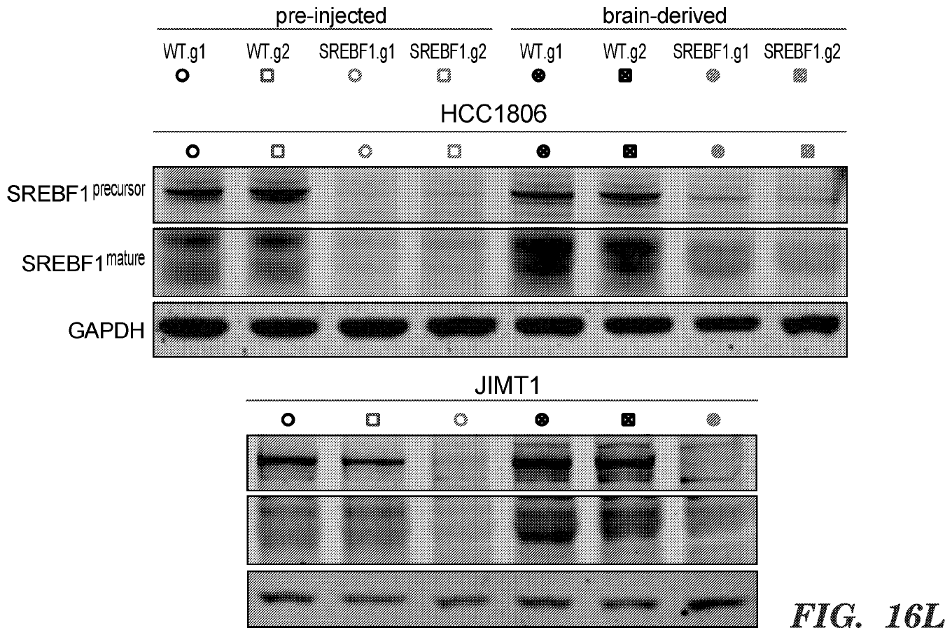

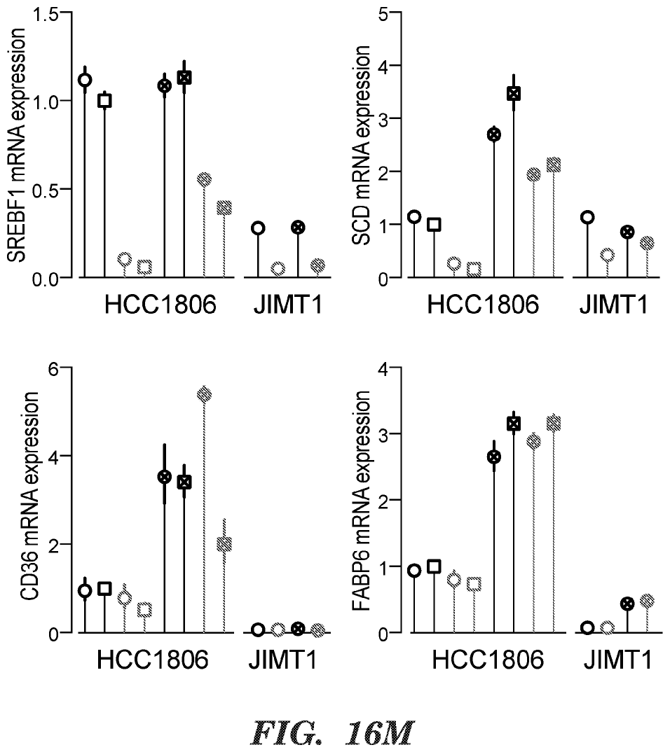

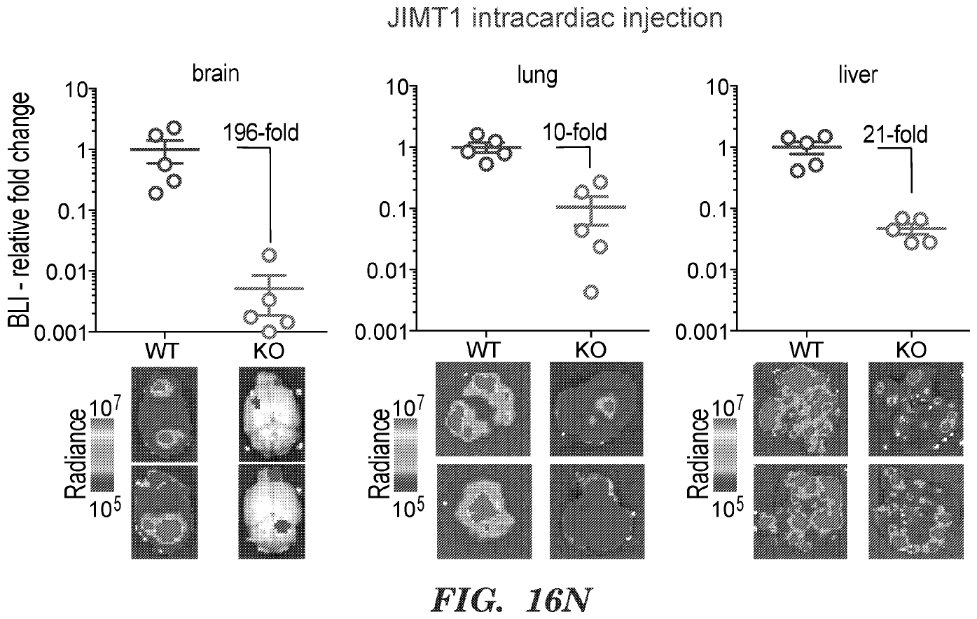

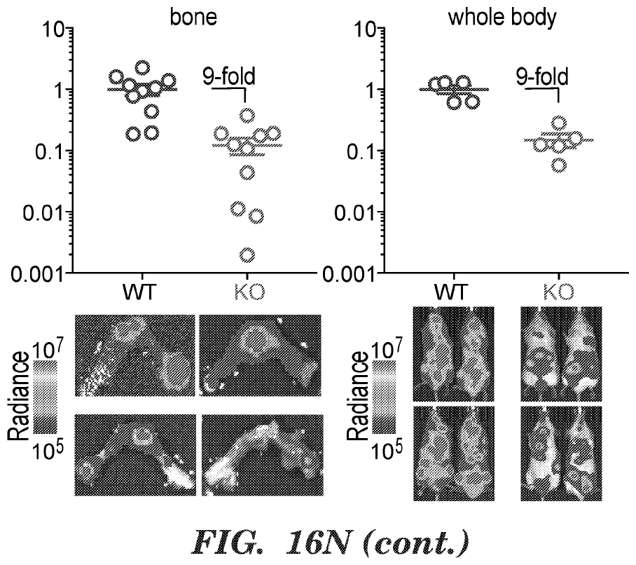

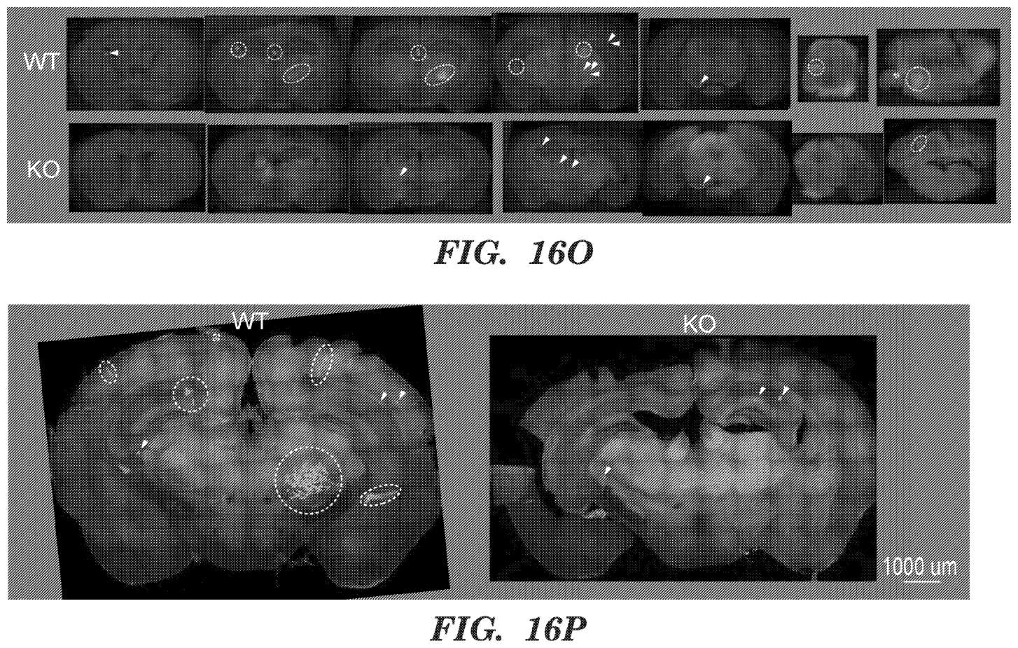

Given the repeated observation of lipid metabolism being associated with brain metastatic potential, the functional impact of perturbing the pathway on brain metastasis formation was assessed. Towards this goal, pooled in vivo CRISPR screen of 29 gene candidates in brain metastatic growth were performed using the JIMT1 model (FIG. 16A). SREBF1, its activator SCAP, and its target SCD scored among the top 13 significant hits, as expected (FIG. 16B). In addition, two genes belonging to the mevalonate-cholesterol metabolism pathway, PMVK and UBIAD1, showed deepest depletion (FIG. 16B). 6 genes were selected for validation one at a time, and all 6 were validated (FIG. 16C). In particular, in contrast to wild-type (WT) cells that displayed exponential growth after injection, SREBF1-KO cells showed minimal growth and displayed a latent phenotype, with low but detectable signal. Perturbing SCAP and SCD phenocopied the effect of SREBF1-KO. Knocking out PMVK regressed the tumor cells after injection, confirming it as the strongest hit from the screen. Collectively, these results pinpointed the significance of the lipid metabolism pathway genes in mediating brain metastasis outgrowth.

To assess how it compared to systemic metastasis, an intracardiac injection assay was performed, focusing on SREBF1. The most dramatic phenotype was that of brain metastasis, where SREBF1-KO cells showed a 196-fold reduction in brain metastasis compared to WT controls (FIG. 16N). Consistent with the in vivo imaging, histologic examination of brains from xenografted animals revealed large metastasis lesions in animals receiving WT cells, but only micrometastases in those receiving SREBF1-KO cells (FIGS. 15E, 15F, 16O, 16P). Note that there were also reduced metastases in other organs, albeit to a substantially reduced extent (9-21 fold reduction compared to 196-fold reduction in brain) (FIG. 16N). To exclude bias in cell seeding by the intracardiac route, cells were introduced selectively to the brain through intracarotid injection. Similar levels were observed of inhibition in brain metastasis load by SREBF1-KO as seen in the intracardiac assay (FIG. 16D). Collectively, through an in vivo screen and three independent validation experiments, a prominent role was confirmed for SREBF1 and that of lipid metabolism in facilitating JIMT1 growth in the brain microenvironment.

### Example 10

### HCC1806 Resort to Lipid Transporter and Binding Protein Upon SREBF1 Deficiency for Growing in Brain Metastasis

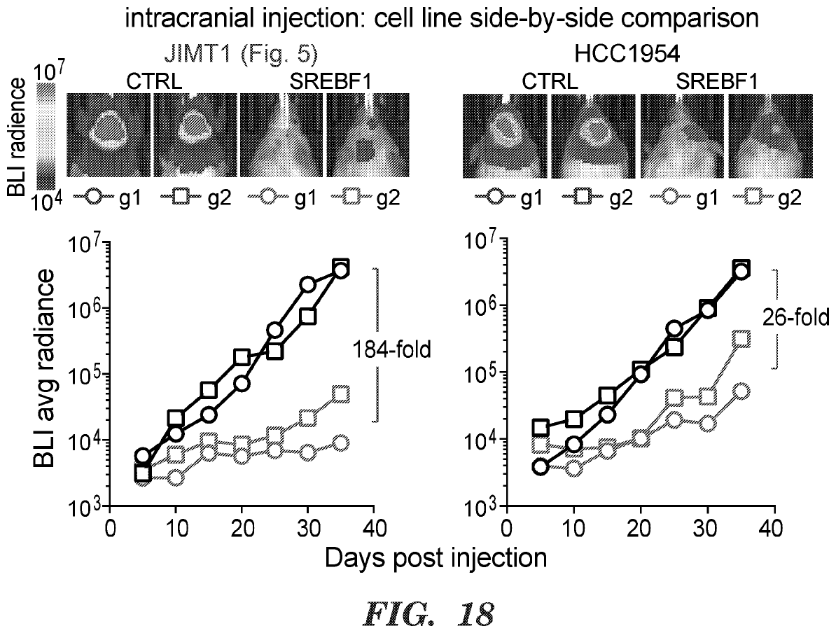

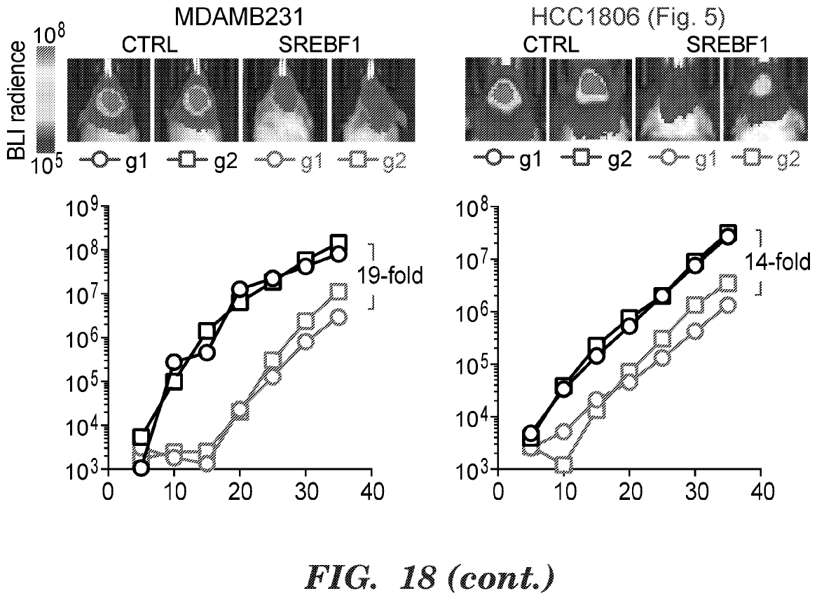

To determine the generality of the SREBF1 requirement for breast cancer growth in the brain, it was knocked out in additional brain metastatic lines including HCC1954, MDAMB231 and HCC1806. As with JIMT1, a significant inhibition in brain metastatic growth was also observed in these lines, although the magnitude and duration of growth inhibition varied (FIG. 18). The least responsive cell line was HCC1806, where SREBF1-knock-out cells displayed a brain growth defect for the first week, but then assumed a growth trajectory that paralleled wild type cells (FIG. 16E). To assess if such discrepancy was peculiar to SREBF1, additional genes that had been validated for JIMT1 were tested in HCC1806 (FIG. 16E). A less prominent effect was seen with KOs of SCAP, SCD, ACLY, and IRX3, with the exception of PMVK-KO which resulted in tumor cell regression.

This restoration of growth was not explained by escape from genome-editing, as brain metastases at the end of the experiment had evidence of editing at the SREBF1 locus (FIGS. 16F-16K) and had minimal SREBF1 protein expression (FIG. 16L). However, this SREBF1-independent growth was associated with up-regulation of the fatty acid transporter CD36 and binding protein FABP6 (FIG. 16M). These results further validate the importance of lipid metabolism in brain metastasis, as cells under the selective pressure of SREBF1 loss upregulate other components of fatty acid metabolism in order to survive in the brain microenvironment. JIMT1 cells failed to upregulate CD36 or FABP6 following SREBF1 knock-out, perhaps explaining their inability to survive in the brain.

The present disclosure describes MetMap as a new large-scale in vivo characterization of human cancer cell lines that adds a missing dimension to in vitro studies. The MetMap resource currently has metastasis profiles of 125 cell lines spanning 22 tumor types—over an order of magnitude more than was previously available. Ideally, all available cancer cell lines would be characterized for their metastatic potential, thus creating an even larger repertoire of models for exploration of metastasis mechanisms. A limitation of the use of human cell lines for such experiments is that they require the use of immunodeficient mice for in vivo characterization, and the extent to which the immune system plays an important role in mediating organ-specific patterns of metastasis remains to be determined (Topalian et al., Cell 161: 185-86 (2015), the contents of which are incorporated herein by reference in their entirety).

Multiple lines of experimental and clinical evidence pointed to the role of lipid metabolism in governing the ability of cells to survive in the brain microenvironment. The importance of lipid metabolism in cancer has been recently highlighted by a number of studies (Pascual et al., Nature 541: 41-45 (2017); Zhang et al., Cancer Discov. 8: 1006-25 (2018); Nieman et al., Nat. Med. 17: 1498-1503 (2011), the contents of each are incorporated herein by reference in their entirety), but its role in brain metastasis has not been previously recognized. Particularly intriguing is the notion that interfering with lipid or cholesterol metabolism might abrogate metastatic growth in the brain. The development of brain-penetrant inhibitors of this pathway would allow for this hypothesis to be tested pharmacologically. More generally, this disclosure highlights the complex interplay between cancer cell survival and metabolic states that can vary widely from organ to organ. Exploiting such tumor microenvironmental differences may prove useful as a therapeutic strategy to combat cancer.

The results reported herein above were obtained using the following methods and materials.

### Breast Cancer Cell Lines and Barcoding

All breast cell lines were obtained from CCLE and cultured under the recommended conditions. Cell line identities were confirmed by SNP fingerprinting as well as RNA-Seq, in comparison to the CCLE results (portals.broadinstitute.org/ccle). The Fluorescence-Luciferase-Barcode (FLB) construct was engineered using the FUW lentiviral vector backbone (a gift from David Baltimore, Addgene plasmid # 14882). Barcodes of 26 nucleotide-long were designed using barcode_generator.py (ver 2.8, comailab.genomecenter.ucdavis.edu/index.php/), and cloned into the landing pad c-terminal to the TGA stop codon of Fluorescence-Luciferase using Gibson assembly (New England Biolabs). Lentivirus preparation and cell infection were performed according to published protocols available at http://www.broadinstitute.org/rnai. Infected cells were subjected to FACS with a fixed gate for GFP or mCherry, using Sony SH4800 sorter.

### Animal Studies

Animal work was performed in accordance with a protocol approved by the Broad Institute Institutional Animal Care and Use Committee (IACUC). NOD scid gamma (NSG) female mice (The Jackson Laboratory) of 5-6 weeks were used. Cancer cells were suspended in PBS+0.4% BSA, and 100 μl of cell suspensions were injected into the left ventricle of anesthetized mice (ketamine 100 mg/kg; xylazine 10 mg/kg). In vivo metastasis progression was monitored via real-time BLI using the IVIS SpectrumCT Imaging System (PerkinElmer), on a weekly basis. Mice were anesthetized with inhaling isoflurane, injected intraperitonially D-Luciferin (150 mg/kg), and imaged with auto exposure setting in prone and supine positions. At the end point, ex vivo BLI was performed by submerging the excised organs in DMEM/F12 media (Thermo Fisher Scientific) containing D-Luciferin for 10 min and imaged with auto exposure setting. BLI analysis was performed using Living Image software (ver 4.5, PerkinElmer). In the case of breast cancer cohort study (pilot, group 1, group 2 in FIGS. 1A and 4A), cell lines were mixed at equal ratio immediately before animal injection, and cell line pools containing 2e04 cells per barcoded line were injected. In the case of single breast cell line validation (FIGS. 5A and 5B), cell lines were injected individually at the density of 2e04 cells, to be comparable with the pooled experiments. In the case of MetMap125 (FIGS. 6A to 6E), the PRISM pool of 25 cell lines were used, and 2.5e5 total cells were injected per animal, corresponding to 1e4 cells per barcoded line. Five PRISM pools were injected separately into cohorts of 5-6 week NSG mice. In the case of MetMap500, 20 PRISM pools of 25 cell lines were combined to form a large pool of 498 cell lines. The large pool was injected into a cohort of 8-10 week NSG mice, with 2.5e05 cells per animal, equivalent to a density of 500 cells per line. Mammary fat pad and subcutaneous injections were performed following published protocols with Matrigel support, at a matching density to their intracardiac assays respectively (FIGS. 7H-7K). For all pooled cell line experiments, animals were sacrificed 5 weeks post injection, in a time-matched manner, unless animals displayed severe paralysis or poor body conditions that they had to be sacrificed slightly earlier. Intracartoid injection of JIMT1 was performed following a published protocol, at a density of 1e5 cells per animal similar to the intracardiac injection (FIGS. 16D, 16N). Intracranial injection was performed as previously described, at a density of 1e3 cells per animal (FIGS. 16C, 16E).

Tissue Processing and Cancer Cell Isolation from Organs

Organs including brain, lung, liver, kidney were dissociated using gentleMACS Octo Dissociator with Heaters (Miltenyi Biotec). Bones (from both hind limbs) were chopped into fine pieces and incubated in the dissociation buffer with vigorous shaking. The dissociated cell suspensions were filtered using 100 μm filters, and washed with DMEM/F12 twice. Cell suspensions were then washed with staining buffer (PBS+2 mM EDTA+0.5% BSA), and incubated with mouse cell depletion beads according to the instructions (Miltenyi Biotec). Cell suspensions were subjected to negative selection using autoMACS Pro Separator (Miltenyi Biotec) to deplete mouse stroma. Brains were subjected to an additional myelin debri depletion step using myelin removal beads II (Miltenyi Biotec). The resultant cell suspensions were then subjected to FACS using Sony SH4800 sorter, with the fixed gate for GFP or mCherry. DAPI staining was used to exclude dead cells. For bulk RNA-Seq, cells were sorted to a single tube in PBS+0.4% BSA+RNasin Plus RNase Inhibitor (Promega), centrifuged at 1500 rpm×10 min, and cell pellets were frozen in −80C for downstream use. For single cell RNA-Seq, single cells were sorted into 96-well plates containing cold TCL buffer (Qiagen) containing 1% b-mercaptoethanol, snap frozen on dry ice, and then stored at -80° C. 90 single cells were sorted per plate, the rest wells were used for negative and positive controls.

### RNA Extraction, Library Preparation and Sequencing

Individual cell lines, cell line pools prior to injection, and cells isolated from metastases were subjected to RNA-Seq. RNA extraction was performed using Quick-RNA MicroPrep according to instructions (Zymo Research). RNA was quantified using RNA 6000 Pico Kit on a 2100 Bioanalyzer (Agilent). RNA samples from cell numbers lower than 500 were not measured but all were used as input for library preparation. cDNA was synthesized using Clontech SmartSeq v4 reagents from up to 2 ng RNA input according to manufacturer's instructions (Clontech). Full length cDNA was fragmented to a mean size of 150 bp with a Covaris M220 ultrasonicator and Illumina libraries were prepared from 2 ng of sheared cDNA using Rubicon Genomics Thruplex DNAseq reagents according to manufacturer's protocol. The finished dsDNA libraries were quantified by Qubit fluorometer, Agilent TapeStation 2200, and RT-qPCR using the Kapa Biosystems library quantification kit. Uniquely indexed libraries were pooled in equimolar ratios and sequenced on Illumina NextSeq500 runs with paired-end 75bp reads at the Dana-Farber Cancer Institute Molecular Biology Core Facilities. RT-qPCR quantification of barcodes was performed using Maxima First Strand cDNA Synthesis Kit, Taqman Fast Advanced Master Mix, custom synthesized Taqman probes, and QuantStudio 6 PCR System (ThermoFisher Scientific). Single cell RNA-Seq was performed as previously described (Ramaswamy, S. et al., Nat. Genet. 33, 49-54 (2003), the contents therein are hereby incorporated by reference in their entirety).

Scalable Metastatic Potential Profiling with Barcoded Cell Line Pools.

To enable profiling of in vivo metastatic potential in a scalable manner, a barcoding vector was designed that contained (1) a fluorescence protein (GFP or mCherry) for cell sorting, (2) a luciferase for real-time in vivo imaging, and (3) a barcode for cell line identity (FIG. 1A). The three elements constituted a single transcription cassette; thus, their expression levels were correlated. This ensured that the labeled cell lines harbored close expression levels (and thus similar copy numbers) of barcodes through gating the fluorescence expression by FACS (FIG. 1B). The designed barcodes could be readout at either DNA or RNA level, by TaqMan assay or by next-generation sequencing, suitable for both low-throughput and high-throughput applications.

The transcribing barcode design allows co-capturing cancer barcodes and cancer transcriptomes of metastases from bulk RNA-Seq, a workflow and analysis method was developed that readout both (FIG. 1C). The resultant transcriptomic profiles represent an ensemble from multiple constituent cell lines, and would yield consensus gene programs and generalizable molecular insights about organ-specific metastases. An example of barcode mapping result from the pilot experiment is presented (FIG. 1D). The barcodes were expressed at high levels, among the top 10% highly expressed genes, allowing robust quantification (FIGS. 1E, 1F).

To validate RNA-Seq-quantitated barcode results from the pilot study, RT-qPCR was performed using Taqman assays against the barcodes. An examination of individual barcoded lines showed that the Taqman probes were highly specific to the engineered barcodes and there was no cross detection (FIG. 2A). Consistent with RNA-Seq (FIG. 1I), RT-qPCR showed even distribution of 4 cell lines in the pre-injected pool, but selective enrichment of specific cell lines in different organs (FIG. 2B). To validate further at single cell resolution, single cell RNA-Seq was performed on the isolated cancer cells from different organs, one organ per 96-well plate (FIG. 3A). Principal component analysis (PCA) stratified cells into 2 clusters. One cluster was characterized by high expression of genes on the HER2 amplicon (ERBB2, ORMDL3, GRB7, PGAP3), consistent with the HCC1954 (HER2+) identity (FIGS. 4C, 4E). The other cluster was characterized by high expression of VIM (vimentin) and low expression of CDKN2A (P16), consistent with MDAMB231 harboring P16-loss and being vimentin-high (FIG. 4C). Mapping cell line identities to their organ origins indicated that HCC1954 was abundant in the brain, whereas MDAMB231 dominated lung, liver and bone (FIG. 4F). In both approaches, BT549 and CAL851 were not detected. Collectively, these results validated the pilot study with independent methods.

Having validated the feasibility of in vivo barcoding approach, efforts were focused on mapping the metastatic behaviors of basal-like breast cancers from Cancer Cell Line Encyclopedia (CCLE), a breast cancer subtype that displays substantial heterogeneity in metastasis patterns from patient to patient. Principal component analysis (PCA) of expression profiles stratified breast cancer cell lines into 3 categories: (1) one group all initiated with HS and displaying fibroblast characteristics, (2) one enriched in luminal subtype, and (3) one enriched in basal subtype (FIG. 4A). Since 8 barcoded lines could be pooled without obvious bottleneck from the pilot study, it was surmised that a pool size of 10 would be suitable, and the collected additional 17 lines were split into 2 pools (group1 and group2, FIG. 4A). The two non-metastatic lines BT549 and CAL851 were included again in these two larger pools for re-assessment. Cell lines were individually barcoded, pooled at equal numbers, and injected into mice (Table 2). BLI imaging indicated comparable tumor progression kinetics as the pilot experiment (FIG. 4B, 4C), thus all mice were sacrificed 5 weeks post injection, in a time-matched manner. The total cell numbers and barcode-quantitated cell line compositions from each organ sample are presented in FIGS. 4D-4G.

TABLE 2

| Cell line | | | Barcode | Barcode seq (5′flank_barcode_ | |
|---|---|---|---|---|---|
| name | Pool | Fluor. | ID | 3′flank) | Site of origin |
| MDAMB231 | pilot (pi) | GFP | BC01 | CGTGTAAAGTTAACCTCGAGGGaaccaa | pleura |
| | | | | aacgctgcagctggcctacgCGATATCA | (metastasis) |
| | | | | AGCTTATCGATAATCAA | |
| HCC1954 | pilot (pi) | GFP | BC02 | CGTGTAAAGTTAACCTCGAGGGaggaat | primary |
| | | | | tacaccqacgcgggactgCGATATCAAG | |
| | | | | CTTATCGATAATCAA | |
| BT549 | pilot (pi) | GFP | BC03 | CGTGTAAAGTTAACCTCGAGGGagtcgct | primary |
| | | | | gcgggttcaatcctggatgCGATATCAAG | |
| | | | | CTTATCGATAATCAA | |
| CAL851 | pilot (pi) | GFP | BC04 | CGTGTAAAGTTAACCTCGAGGGcatcag | primary |
| | | | | ccagacgcctaaggtcatCGATATCAAG | |
| | | | | CTTATCGATAATCAA | |
| MDAMB231 | pilot (pi) | mCherry | BC05 | CGTGTAAAGTTAACCTCGAGGGccgacgc | pleura |
| | | | | gagggttgttctgtqatgaCGATATCAAG | (metastasis) |
| | | | | CTTATCGATAATCAA | |
| HCC1954 | pilot (pi) | mCherry | BC06 | CGTGTAAAGTTAACCTCGAGGGgtccgaa | primary |
| | | | | gacgtctcgcctgcatcaaCGATATCAAG | |
| | | | | CTTATCGATAATCAA | |
| BT549 | pilot (pi) | mCherry | BC07 | CGTGTAAAGTTAACCTCGAGGGgtgtgac | primary |
| | | | | agaaattcctgcaggcggcCGATATCAAG | |
| | | | | CTTATCGATAATCAA | |
| CAL851 | pilot (pi) | mCherry | BC08 | CGTGTAAAGTTAACCTCGAGGGttcggcc | primary |
| | | | | gctcgaaccacgtaagtcaCGATATCAAG | |
| | | | | CTTATCGATAATCAA | |
| BT549 | group 1 | GFP | BC03 | CGTGTAAAGTTAACCTCGAGGGagtcgct | primary |
| | (g1) | | | gcqggttcaatcctggatgCGATATCAAG | |
| | | | | CTTATCGATAATCAA | |
| HDQP1 | group 1 | GFP | BC06 | CGTGTAAAGTTAACCTCGAGGGgtccgaa | primary |
| | (g1) | | | gacgtctcgcctgcatcaaCGATATCAA | |
| | | | | GCTTATCGATAATCAA | |
| JIMT1 | group 1 | GFP | BC08 | CGTGTAAAGTTAACCTCGAGGGttcggc | pleura |
| | (g1) | | | cgctcgaaccacgtaagtcaCGATATCA | (metastasis) |
| | | | | AGCTTATCGATAATCAA | |
| MDAMB157 | group 1 | GFP | BC09 | CGTGTAAAGTTAACCTCGAGGGacagct | pleura |
| | (g1) | | | ttcgacgggtccaagcagccCGATATCA | (metastasis) |
| | | | | AGCTTATCGATAATCAA | |
| MDAMB436 | group 1 | GFP | BC10 | CGTGTAAAGTTAACCTCGAGGGacgtcc | pleura |
| | (g1) | | | ggcccctcacaagcacattcCGATATCA | (metastasis) |
| | | | | AGCTTATCGATAATCAA | |
| HCC1806 | group 1 | GFP | BC11 | CGTGTAAAGTTAACCTCGAGGGataagc | primary |
| | (g1) | | | ggcgctcggtagactgcggtCGATATCA | |
| | | | | AGCTTATCGATAATCAA | |
| HMC18 | group 1 | GFP | BC12 | CGTGTAAAGTTAACCTCGAGGGcctggg | pleura |
| | (g1) | | | cattcgtgtgtccaccccttCGATATCA | (metastasis) |
| | | | | AGCTTATCGATAATCAA | |
| MDAMB468 | group 1 | GFP | BC13 | CGTGTAAAGTTAACCTCGAGGGcgccga | pleura |
| | (g1) | | | ggttgaagcacggttggaacCGATATCA | (metastasis) |
| | | | | AGCTTATCGATAATCAA | |
| DU4475 | group 1 | GFP | BC14 | CGTGTAAAGTTAACCTCGAGGGcatgca | skin |
| | (g1) | | | ggcaatacctgcgagtaacgCGATATCA | (metastasis) |
| | | | | AGCTTATCGATAATCAA | |
| CAL851 | group 2 | GFP | BC04 | CGTGTAAAGTTAACCTCGAGGGcatgca | primary |
| | (g2) | | | gccagacgccctaaggtcatCGATATCA | |
| | | | | AGCTTATCGATAATCAA | |
| HCC1143 | group 2 | GFP | BC05 | CGTGTAAAGTTAACCTCGAGGGccgacg | primary |
| | (g2) | | | cgagggttgttctgtgatgaCGATATCA | |
| | | | | AGCTTATCGATAATCAA | |
| HCC70 | group 2 | GFP | BC07 | CGTGTAAAGTTAACCTCGAGGGgtgtga | primary |
| | (g2) | | | cagaaattcctgcaggcggcCGATATCA | |
| | | | | AGCTTATCGATAATCAA | |
| HCC1395 | group 2 | GFP | BC15 | CGTGTAAAGTTAACCTCGAGGGagact | primary |
| | (g2) | | | tgtccagccgcggcgtagatcCGATAT | |
| | | | | CAAGCTTATCGATAATCAA | |
| HCC1187 | group 2 | GFP | BC16 | CGTGTAAAGTTAACCTCGAGGGcaatc | primary |
| | (g2) | | | aggtagacgggacgcgtgacgCGATAT | |
| | | | | CAAGCTTATCGATAATCAA | |
| HCC38 | group 2 | GFP | BC17 | CGTGTAAAGTTAACCTCGAGGGcaggc | primary |
| | (g2) | | | acctcgtagcagtgctttgccCGATAT | |
| | | | | CAAGCTTATCGATAATCAA | |
| HCC1569 | group 2 | GFP | BC18 | CGTGTAAAGTTAACCTCGAGGGcccca | primary |
| | (g2) | | | ctgtgcccgttcaccagtactCGATAT | |
| | | | | CAAGCTTATCGATAATCAA | |
| HCC1937 | group 2 | GFP | BC19 | CGTGTAAAGTTAACCTCGAGGGccgcc | primary |
| | (g2) | | | tgccagagctaaggtcggttaCGATAT | |
| | | | | CAAGCTTATCGATAATCAA | |
| BT20 | group 2 | GFP | BC20 | CGTGTAAAGTTAACCTCGAGGGcggcc | primary |
| | (g2) | | | ctcggtatcctcagatgtccaCGATAT | |
| | | | | CAAGCTTATCGATAATCAA | |
| HCC1599 | group 2 | GFP | BC21 | CGTGTAAAGTTAACCTCGAGGGcgtag | primary |
| | (g2) | | | cagcaagcgcctagccagtctCGATAT | |
| | | | | CAAGCTTATCGATAATCAA | |

To quantify the cell line metastatic potentials on an absolute scale, the cell number was inferred for each cell line based on the total cancer cell counts and their barcode-quantitated compositions from each organ. This metric was used to compare cell lines across the 3 pool studies. For data visualization, a petal plot was developed that encodes 3 information: (1) metastatic potential as quantified by inferred cell number, (2) its confidence interval that estimates animal variability, (3) and penetrance—percentage of animals in the cohort that the particular cell line was detected (FIG. 4H). This visualization method effectively displayed a diversity of metastatic patterns and differential aggressiveness of cell lines. Four cell lines including MDAMB231, HCC1187, JIMT1, HCC1806 were pan-metastatic. Other cell lines showed more selective patterns. Among the 21 cell lines, DU4475 and HCC1599 were suspension cells and both displayed selective colonization towards bone and lung. Whether the in vivo pattern was associated with cell culture status remained unclear.

Drafting MetMap with PRISM Cell Line Pools

Expansion of metastatic potential mapping beyond breast cancer was attempted as was drafting a comprehensive MetMap for all solid tumor types. Focusing on one cancer type at a time would result in custom pooling and different group sizing, which was neither scalable nor standardizable. For pan-cancer characterization, it also didn't make sense to perform bulk RNA-Seq on mixed cancer types, as lineage would be a strong confounder. In this case, readout at DNA level would be sufficient. PRISM, a barcoded cell line mixture approach developed for high-throughput in vitro drug screen, was used. It was asked whether the PRISM platform could be applied for the in vivo MetMap purpose.

As part of PRISM profiling, cell lines were pooled based on their in vitro doubling time across mixed lineages, with a size of 25 lines per pool. PRISM barcoded cells did not harbor GFP or luciferase, thus in the first study, it was addressed whether it was critical to introduce the labeling markers for cancer cell purification. One PRISM pool (of 25 cell lines) was chosen that contained JIMT1, labeled with GFP-luciferase vector, and then sorted for GFP⁺ cells (FIG. 6A). Consistent with different susceptibilities of cell lines to virus infection, 6/25 cell lines showed strong dropout after GFP labeling, but all lines were still detectable (FIG. 6B). In contrast, cell lines prior to labeling displayed a more even barcode distribution, close to equal ratio pooling. The GFP-labeled and unlabeled cell pools were then subjected to the same animal work flow, tissue dissociation, and mouse cell depletion. The GFP-labeled group was further sorted to purify cancer cells. The isolated cancer cells (GFP-labeled group) or the tissue lysates (unlabeled group) were then subjected to barcode amplification and sequencing (FIG. 6A). A comparison of the two experiments showed highly concordant results. Although the initial barcode distribution of the pre-injected pools had altered, the enrichment (fold change) of barcode abundance showed strong positive correlation after normalizing to the pre-injected input (FIG. 6C, one exception was U2OS).

The positive control JIMT1 was pan-metastatic as expected. Importantly, cell lines such as MELHO, MHHES1 and PC14 substantially dropped in their initial abundance after GFP labeling, yet they gained similar in vivo enrichment as in the non-labeled experiment. These results suggested that we could quantitatively detect barcodes from crude lysates without the need of pure cancer cell isolation from PRISM.

The simplified workflow using PRISM pools for pan-cancer mapping was employed, and a total of 503 cancer cell lines across 21 cancer types were profiled (FIGS. 6E). Profiling was carried out in two different pooling formats (MetMap500 and MetMap125), with 120 cell lines and 4 target organs shared in common that allowed reproducibility assessment (FIGS. 7A, 7F, 7G). Prior to injection, most cell lines displayed even barcode distribution, consistent with equal ratio pooling (FIGS. 7B, 7C). In MetMap500, 10 cell lines had low initial abundance and could not be detected in any in vivo organ thus were excluded from analysis, leaving effective data for 488 cell lines. PRISM sequencing detected relative barcode abundance, which was reflective of relative cell abundance in organs. The metastatic potential was defined as enrichment of barcodes in the in vivo organs relative to the pre-injected input, and used this metric to compare between cell lines. A comparison of normalized with non-normalized barcode counts showed strong linearity (FIGS. 7D, 7E), reflecting that subtle differences in the initial abundance had little impact on barcode quantification from in vivo samples. A similar petal plot view was employed to display metastatic patterns, including relative metastatic potential as readout by PRISM barcode, its confidence interval that depicts animal variability, and penetrance data that provides qualitative measures of cell line xenograftability (FIGS. 7S, 7T).

Analysis of In Vivo Metastasis Transcriptomes with Polyclonal Cell Lines

RNA-Seq co-captured cancer cell composition and averaged in vivo transcriptomes of metastases from cell line pools in the breast cancer cohort study. To understand what metastasis transcriptomes encoded, differential analysis was performed on the in vivo transcriptomes to cells in vitro. To properly account for the different cell line compositions in each metastasis, a composite in vitro transcriptome was modeled using the barcode composition and single cell line in vitro transcriptomes, and then compared to the actual in vivo results (FIG. 12A). In this way, the resultant differentially expressed genes were uniquely attributed to the in vivo context but not due to cell composition changes. These genes were (1) either commonly induced (or selected for) in multiple cell lines, or (2) were uniquely enriched in the dominant line. In either case, the revealed genes or pathways would be interesting for further study. As expected, the transcriptomes of the pre-injected population which was a direct mixture of in vitro cell lines showed a very tight correlation with the in silico profiles and few genes were differentially expressed (FIG. 12B). In contrast, the transcriptomes from in vivo samples showed genes with large fold changes and the correlation was weaker. These results justified the comparison method and showed that the in vivo environment was inducing substantial transcriptional changes.

To assess whether such comparison identified genes relevant to metastasis, the top differentially expressed genes were inspected. Notably, MUCL1 (also termed small breast epithelial mucin, SBEM) and SCGB2A2 (also known as Mammaglobin, MGB1) were strongly induced in brain metastases as well as in other sites (FIG. 12D). These genes are breast lineage markers, whose expression is known to be induced during breast tumorigenesis from clinical specimens. Their expression has been used as a marker, indicative of hematogenous spread, micrometastasis and breast cancer metastasis in the brain differentiating from primary brain tumors. The results suggested that although relevant marker genes were non-expressed or at a low level when cells were cultured in a dish, their expression could be induced in the in vivo metastasis context. These results highlighted the biological relevance of the in vivo transcriptomic results.

Since MDAMB231 is the most investigated cell line in breast cancer metastasis, it was asked whether genes previously identified and validated as metastasis mediators were induced in the in vivo transcriptomic profiles. In the pilot group experiments, MDAMB231 dominated lung, liver, kidney and bone metastases in most samples (FIG. 1I), thus the majority of the gene expression changes were attributed to MDAMB231. Twenty-seven out of 32 lung metastasis genes reported by Minn et al. were upregulated in the lung metastasis profiles, showing a very strong agreement (p value=3.9e-16, FIG. 12E). These genes were also enriched in metastases at other sites but to a lesser extent. Indeed, although these genes were initially identified as lung metastasis mediators, many were shown to function in a pleiotropic fashion, mediating primary tumor growth or metastasis at other sites. For example, VCAM1 has been shown to mediate both lung and bone metastasis through juxtacrine interaction with myeloid lineage cells. TNC, which is a secreted molecule that boosts breast cancer stemness, promotes lung and bone metastasis. Collectively, these results suggested that the in vivo “induced” genes not only included metastasis associated markers but also previously validated mediators.

Having confirmed the validity of these profiles, pathway enrichment analysis was performed to query consensus programs that the differential genes encode in the 5 organ sites (FIG. 15C). The results revealed a response to diverse external stimuli in vivo, consistent with much richer environmental factors in the in vivo context. In contrast, proliferation and cycling related pathways are much attenuated in vivo compared to cells cultured in vitro (FIG. 15C). Consistent with this result, in vitro culture media is optimized to maximize cell proliferation by supplementing excess nutrients and supportive elements. Comparing between organs, it was found that brain metastases shared less commonality and weaker correlation with metastases in extracranial organs (FIGS. 15C, 17), suggestive of a more unique microenvironment in the brain. More specifically, inflammatory responses including TNF, interleukin and interferon signaling were more prominent in lung, liver, kidney, bone than in brain, consistent with less immune response in the brain compared to extracranial organs. Similarly, evidence of TGFβ activation and epithelial-mesenchymal transition (EMT) in extracranial metastatic lesions was observed, but not in brain (FIG. 15C). Confirming such experimental observations, brain metastasis samples from patients showed less TGFβ response and EMT, in comparison to extracranial metastases (FIGS. 12F, 12G, 15G) or matched primary breast tumors (FIGS. 15H-15J). Together, these results revealed distinct transcriptional states between in vitro and in vivo, and between different organ sites.

### Bioinformatic Analysis

Barcode Quantification from RNA-Seq of Metastases

Since the RNA-Seq library preparation sheared the cDNA randomly into small pieces, demultiplexed RNA-Seq reads were mapped to the barcode references using Bowtie 2 (Langmead et al., Nat. Methods 9: 357-59 (2012), the contents of which are incorporated herein by reference in their entirety) local mode for barcode detection and quantification. Mapped reads were filtered with the criteria that reads (either 5′ or 3′) must cover over 50% of the barcodes from either end, and counted using samtools. Barcode percentage corresponding to cell composition was calculated for single cell lines, pre-injected cell mixtures, and in vivo metastasis samples.

### Metastatic Potential Quantification and Feature Associations

For breast cohort study, metastatic potential of cell line j targeting organ i, was calculated as:

$${M}_{i , j} = \frac{1}{n} {\sum }_{k = 0}^{n} {c}_{i} {p}_{j} ,$$

where cᵢ is the total cancer cell number isolated from organ i and pⱼ is the fractional proportion of cell line j estimated by barcode quantification, and n is the number of replicates of mice. To identify features that associate with brain metastatic potential, a 2-class comparison method was used (Ritchie et al., Nucleic Acids Res. 43: e47 (2015), the contents of which are incorporated herein by reference in their entirety). The analysis was performed on mutation, copy number, metabolite (available at https://portals.broadinstitute.org/ccle/), and CRISPR-gene dependency (CERES scores, available at https://depmap.org/portal/) separately. Copy number data were binarized using a cutoff of <=−1 (loss) and >=1 (gain).
Cancer Transcriptomic Analysis from RNA-Seq of Metastases

Potential mouse contaminating reads were removed by competitive mapping to the human/mouse hybrid genome using BBSplit (https://sourceforge.net/projects/bbmap/). Reads that uniquely mapped to the human genome were then used as input for mapping and gene-level counting with the RSEM package (Li et al., BMC Bioinformatics, 12: 323 (2011), the contents of which are incorporated herein by reference in their entirety). Gene count estimates were normalized using the TMM method (Robinson et al., Bioinformatics 26: 139-40 (2010), the contents of which are incorporated herein by reference in their entirety). For differential analysis, to properly account for the cancer cell composition differences in each in vivo sample, an in silico modeled in vitro mixture was generated first. For each in silico metastasis model, the estimated expression g of gene i is computed as a weighted average of the cell lines present in the corresponding in vivo sample:

ĝᵢ=Σⱼ₌₁^{M}g_{i,j}pⱼ, where g_{i,j} is the baseline in vitro expression of gene i in cell line j and pj is the fractional proportion of cell line j in the in vivo sample, as estimated by barcode quantification, and M is the number of cell lines present in the in vivo sample. The in vivo and in silico counterpart were then compared using a paired design for each organ in voom-limma (Ritchie et al.). The three studies, pilot, group 1, and group 2, were analyzed separately. Overlap significance test of two-set or multi-set intersection was performed using cpsets function in the SuperExactTest package (Wang et al., Sci. Rep. 5: 16923 (2015), the contents of which are incorporated herein by reference in their entirety). Gene set enrichment analysis (GSEA) was performed using the GSEA-preranked method in GSEA package (Subramanian et al., Proc. Natl. Acad. Sci. USA 102: 15545-50 (2005), the contents of which are incorporated herein by reference in their entirety. ssGSEA signature projection was performed in GenePattern (genepattern.broadinstitute.org) (Barbie et al., Nature 462: 108-12 (2009), the contents of which are incorporated herein by reference in their entirety). Gene signature data sets were from MSigDB (software.broadinstitute.org/gsea/msigdb/).

SREBF1 ChIP-Seq peak data were from ENCODE (www.encodeproject.org/) (Consortium et al., Nature 489, 57-74 (2012), the contents of which are incorporated herein by reference in their entirety) and analyzed using ChIPseeker (Yu et al., Bioinformatics 31: 2382-83 (2015), the contents of which are incorporated herein by reference in their entirety).

### PRISM In Vivo Assay

All PRISM cell lines were initially obtained from CCLE. Cell lines were adapted to the same culture condition in pheno red-free RPMI1640 media (ThermoFisher Scientific), and barcoded as previously described (Yu et al., Nat. Biotechnol. 34: 419-23 (2016), the contents of which are incorporated herein by reference in their entirety). PRISM cell lines were pooled based on their in vitro doubling speed bins, at equal number, in the format of 25 lines per pool. Cells were thawed and recovered for 48 hours prior to in vivo injection. To form the large pool of 498 cell lines, 20 PRISM pools were mixed at equal total number right before injection.

Post in vivo experiments, organs were subjected to tissue dissociation, mouse stroma depletion, and the dissociated cell pellets were frozen in −80° C. as discussed above. The pellets (<=50 mg dry weight) were lysed in 200 μL freshly prepared lysis buffer (with proteinase K), heat digested at 60° C., and denatured at 95° C. for 10 minutes. 20 μL of lysates were used for barcode amplification per 100 μL PCR volume (multiple technical replicates per sample). PCR was performed using the following conditions: 95° C. for 3 minutes; 98° C. for 20 seconds, 57° C. for 15 seconds, 72° C. for 10 seconds (30 cycles); 72° C. for 5 minutes; 4° C. stop. PCR libraries (technical replicates combined) were quantified using 2100 Bioanalyzer (Agilent), normalized, pooled, and gel-purified using QIAquick Gel Extraction Kit (Qiagen). Purified samples were quantified, and 2 nM of libraries with 25% spike-in PhiX DNA were sequenced on Illumina MiSeq or HiSeq at 800 K/mm² cluster density.

De-multiplexed sequencing reads were mapped to the barcode reference to generate a table of cell line barcode counts for each sample/condition. Library-size normalized read counts for each sample were used for calculation of relative metastatic potential. Relative metastatic potential of cell line j targeting organ i, rM_{i,j} was defined as:

$${rM}_{i , j} = \frac{1}{n} {\sum }_{k = 0}^{n} {c}_{i , j} \text{/} \frac{1}{m} {\sum }_{k = 0}^{m} {p}_{j} , ,$$

where c_{i,j} is the read counts of cell line j from organ i, pⱼis the read counts of cell line j from pre-injected population, n (n=4˜5) is the number of replicate samples of mice, m (m=4˜5) is the number of replicates of pre-injected population. Confidence intervals reflecting animal variance were calculated using bootstrap.

### In Vivo CRISPR Screen and Gene Validation

CRISPR/Cas9 versions of cell lines were generated by infecting luciferized cells with Cas9-Blast lentivirus and selecting in 5 μg/mL Blasticidin for 10 days with continuous passaging until non-infected controls were killed. For pooled in vivo screen, JIMT1-Cas9 cells were infected with a CRISPR guide library (Table 3) in an arrayed-fashion in 6-well plates, and selected in 2 μg/mL Puromycin for 4 days. At this time, non-infected controls were killed, and no growth defect was observed in the perturbed cell lines. Post antibiotic selection, cells were pooled and subjected to intracranial injection at 6e4 cells per animal in 1 of PBS. This was equivalent to 1e3 cells per guide on average per animal. Intracranial growth was allowed for progression for 4 weeks, and brain tissues were processed adopting the workflow of PRISM in vivo assay, except that guides were amplified using primers targeting the guide vector. De-multiplexed sequencing reads were mapped to the guide reference to generate a table of barcode counts for each guide for each sample. Sequencing-depth was normalized using the upper quartile method and relative depletion was quantitated using a linear model in limma. For individual gene validation (FIGS. 16C, 16E, 18), Cas9-cells of different cell lines were infected with corresponding guides, selected in 2 μg/mL Puromycin for 4 days, and subjected to intracranial injection at 1e3 cells per animal in 1 of PBS. Two independent guides per gene were tested, with one animal per guide. Intracranial growth was monitored by BLI following injection.

TABLE 3

| gene | guide sequence | guide # | exon # |
|---|---|---|---|
| AADAC | AGTCTGAAGCACTAAGAAGG | 1 | 2 |
| AADAC | GTTATGACTTGCTGTCAAGA | 2 | 3 |
| ACLY | AGAGCAATTCGAGATTACCA | 1 | 11 |
| ACLY | GCCAGCGGGAGCACATCGGT | 2 | 11 |
| ACSL3 | TATCTAAAGTATCACATCCA | 1 | 4 |
| ACSL3 | GTGGTGAAGAGTAACCAATG | 2 | 9 |
| ALDH1A1 | AGCATCCATAGTACGCCACG | 1 | 3 |
| ALDH1A1 | TTCCAAATGAGCATAACCAA | 2 | 6 |
| ALDH3B2 | GGCGCCCACCAGGAGCACCA | 1 | 4 |
| ALDH3B2 | AAGCCGTCAGAAATCAGCCA | 2 | 5 |
| CD36 | TTCACTATCAGTTGGAACAG | 1 | 8 |
| CD36 | AGGATAAAACAGACCAACTG | 2 | 9 |
| CERS4 | GGTTACCACCCAATGTCACG | 1 | 3 |
| CERS4 | GCTGACCAAGAAGTTCTGTG | 2 | 5 |
| CYP2J2 | GTTCTCGCATAGGGGTCACG | 1 | 2 |
| CYP2J2 | TTGCTGAAGAGAGTTTGGTG | 2 | 5 |
| CYP4Z1 | CCCACAAGGGAACAGCACAT | 1 | 2 |
| CYP4Z1 | AGCCAGGTTTCACAATCTGG | 2 | 4 |
| DEGS2 | CCACGACATCTCGCACAACG | 1 | 2 |
| DEGS2 | CTCCTTCAAGAAGTACCACG | 2 | 2 |
| DGAT2 | CTGGCTCAATAGGTCCAAGG | 1 | 2 |
| DGAT2 | CCAGGCCCATGATACCATGG | 2 | 5 |
| FASN | GATGTATTCAAATGACTCAG | 1 | 7 |
| FASN | GAGCATGCTGAACGACATCG | 2 | 9 |
| SCAP | CTGCTGGACATAAGCCACCG | 1 | 4 |
| SCAP | TGTTCCTGGGAAGTACAGCG | 2 | 6 |
| SCD | CAGGTGTAGAACTTGCAGGT | 1 | 2 |
| SCD | ATGATCAGAAAGAGCCGTAG | 2 | 3 |
| SCD | GATCCTCATAATTCCCGACG | 3 | 4 |
| BDH1 | CCGTCGGACTTATGCCAGTG | 1 | 4 |
| BDH1 | GTGGCAGAAGTGAACCTTTG | 2 | 7 |
| HMGCS2 | GATACTTGGCCAAAGGACGT | 1 | 2 |
| HMGCS2 | GACATTGCCGTCTATCCCAG | 2 | 3 |
| HMGCL | TGCCCTTCAAGACTTCAGTG | 1 | 4 |
| HMGCL | AGTCAGCCAATATTTCTGTG | 2 | 5 |
| G6PD | CTTGCCCCCGACCGTCTACG | 1 | 5 |
| G6PD | CTTGAAGGTGAGGATAACGC | 2 | 7 |
| H6PD | GAAAAAGGTCCCGAGTTCTG | 1 | 2 |
| H6PD | CCTCCAGAACCATCTGACGG | 2 | 4 |
| SLC40A1 | AAGTAGAGAGAGAATGACCA | 1 | 2 |
| SLC40A1 | TCATCAGGATGATTCCACAC | 2 | 4 |
| CEBPA | CAGTTCCAGATCGCGCACTG | 1 | 1 |
| SPTSSB | TTAAACATAGATCGCTCCCA | 1 | 3 |
| IRX3 | GGACGAGAGCACGTTGGACA | 1 | 1 |
| IRX3 | CCGTCCCAAGAACGCCACCA | 2 | 2 |
| THRSP | GAAGTAGGTGTAGAGATCAG | 1 | 1 |
| THRSP | CATGCTCAAGGCCATCTGTG | 2 | 1 |
| SPDEF | CTTTGACATGCTGTACCCTG | 1 | 2 |
| SPDEF | TGTGGACAGAGCACCAATAC | 2 | 3 |
| UBIAD1 | CAAGTGCTCCAGTTTCAGAG | 1 | 1 |
| UBIAD1 | AGGAATTGGATTCAAGTACG | 2 | 2 |
| CCDC3 | TCTTGGAAATTGACTCCGTG | 1 | 2 |
| CCDC3 | AAACAAGGCCTTCTGCACCG | 2 | 3 |
| SREBF1 | ACAGGGGTGGAGCTGAACTG | 1 | 2 |
| SREBF1 | ACAGTGGTGCCAGAGACCAG | 2 | 4 |
| PMVK | TACTGCTGTTCAGCGGCAAG | 1 | 2 |
| PMVK | CGGGAAGGACTTCGTGACCG | 2 | 3 |
| CYB5B | TGTCACCCGCTTCCTCAACG | 1 | 2 |

### Western Blot

Protein lysates were prepared in RIPA Lysis Buffer (ThermoFisher Scientific)+cOmplete Mini EDTA-free Protease Inhibitor Cocktail (Roche). Western blot was performed using NuPAGE gel (ThermoFisher Scientific)+Wet/Tank Blotting (Bio-Rad)+Odyssey detection system (LI-COR). SREBF1 primary antibodies (14088-1-AP, Proteintech), GAPDH (D16H11) XP® Rabbit mAb (Cell Signaling), and IRDye® 800CW Goat anti-Mouse IgG, IRDye® 680RD Goat anti-Rabbit IgG secondary antibodies (LI-COR) were used.

### SREBF1 CRISPR Knockout Generation

JIMT1 luciferized cells were infected with Cas9-Blast lentivirus (Sanjana et al., Nat. Methods 11: 783-84 (2014), the contents of which are incorporated herein by reference in their entirety) and selected in Blasticidin (5 μg/mL) for 10 days with continuous passaging until non-infected controls were all killed. JIMT1-Cas9 cells were then subjected to lentiGuide-Puro virus infection that encode SREBF1-targeting (ACAGGGGTGGAGCTGAACTG) or non-targeting (CTCCGTTATGTGGCATGAGA) guides. Infected cells were selected in Blasticidin (5 μg/mL)+Puromycin (2 μg/mL) for 4 days until non-infected controls were all killed. Verification of knockout was confirmed by western blot 10 days after infection. Protein lysates were prepared in Cell Lysis Buffer (Cell Signaling) plus cOmplete Mini EDTA-free Protease Inhibitor Cocktail (Roche). Western blot was performed using NuPAGE gel (ThermoFisher Scientific) +iBlot 2 transfer (ThermoFisher Scientific) plus Odyssey detection system (LI-COR). SREBF1 primary antibodies (sc-17755, sc-365513, Santa Cruz), GAPDH (D16H11) XP® Rabbit mAb (Cell Signaling), and IRDye® 800CW Goat anti-Mouse IgG, IRDye® 680RD Goat anti-Rabbit IgG secondary antibodies (LI-COR) were used.

### Tumor Sphere Assay

Tumor sphere assay was performed in Aggrewell400 24-well plates, according to manufacturer's instructions (StemCell Technologies). Each well contains approximately 1200 micro-wells. Cells were seeded at a density of 4000 cells/well, corresponding to 1-3 cells per micro-well. At the end point, tumor spheres were imaged and quantified using IncuCyte S3 System (EssenBioscience), using whole-well imaging modality.

### Clinical Data Analysis

METABRIC, TCGA, and MSK targeted sequencing breast cancer datasets were downloaded from cBioPortal. EMC-MSK dataset including 615 primary tumors (GSE2034, GSE2603, GSE5327, GSE12276), and the 65 metastasis sample dataset (GSE14020) were collected and processed as previously described (Zhang, X. H. et al., Cell 154, 1060-1073, (2013), the contents of which are incorporated by reference in their entirety). Paired primary breast tumor and brain metastasis RNA-Seq was available from Vareslija et al. To exclude the confounding effect of brain stroma contamination in this dataset, a contamination indicator generated from GSE52604 was applied, and the contaminating effect was regressed out, generating a corrected gene matrix. PI3K-response signatures were from Gatza et al. and Creighton et al. respectively. Signature analysis was conducted as described (Malladi, S. et al., Cell 165, 45-60, (2016), the contents of which are incorporated by reference in their entirety). Hierarchical clustering and heatmap generation were generated using gplots package. Log-rank tests of survival curve difference were calculated using survival package. A multivariate Cox proportional harzards model was built using coxph function (FIG. 10U). Significance of overlap was calculated using chisq.test or fisher.test function.

### Computer Implemented Systems

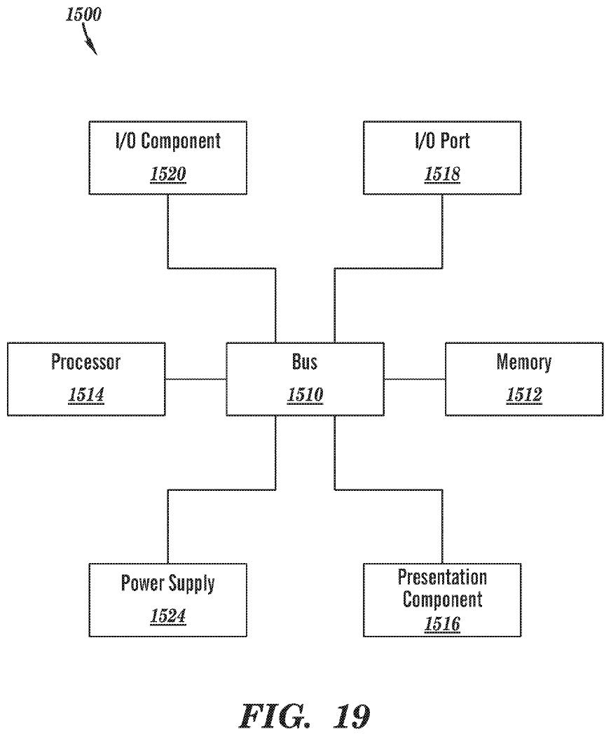

In some embodiments, the steps of the methodologies and analysis provided herein can be implemented and/or supplemented through the use of computing devices. Any suitable computing device can be used to implement the computing devices and methods/functionality described herein and be converted to a specific system for performing the operations and features described herein through modification of hardware, software, and firmware, in a manner significantly more than mere execution of software on a generic computing device, as would be appreciated by those of skill in the art. One illustrative example of such a computing device 1500 is depicted in FIG. 19. The computing device 1500 is merely an illustrative example of a suitable computing environment and in no way limits the scope of the present invention. A “computing device,” as represented by FIG. 19, can include a “workstation,” a “server,” a “laptop,” a “desktop,” a “hand-held device,” a “mobile device,” a “tablet computer,” or other computing devices, as would be understood by those of skill in the art. Given that the computing device 1500 is depicted for illustrative purposes, embodiments of the present invention may utilize any number of computing devices 1500 in any number of different ways to implement a single embodiment of the present invention. Accordingly, embodiments of the present invention are not limited to a single computing device 1500, as would be appreciated by one with skill in the art, nor are they limited to a single type of implementation or configuration of the example computing device 1500.

The computing device 1500 can include a bus 1510 that can be coupled to one or more of the following illustrative components, directly or indirectly: a memory 1512, one or more processors 1514, one or more presentation components 1516, input/output ports 1518, input/output components 1520, and a power supply 1524. One of skill in the art will appreciate that the bus 1510 can include one or more busses, such as an address bus, a data bus, or any combination thereof. One of skill in the art additionally will appreciate that, depending on the intended FIG. 19 applications and uses of a particular embodiment, multiple of these components can be implemented by a single device. Similarly, in some instances, a single component can be implemented by multiple devices. As such, is merely illustrative of an exemplary computing device that can be used to implement one or more embodiments of the present invention, and in no way limits the invention.

The computing device 1500 can include or interact with a variety of computer-readable media. For example, computer-readable media can include Random Access Memory (RAM); Read Only Memory (ROM); Electronically Erasable Programmable Read Only Memory (EEPROM); flash memory or other memory technologies; CD-ROM, digital versatile disks (DVD) or other optical or holographic media; magnetic cassettes, magnetic tape, magnetic disk storage or other magnetic storage devices that can be used to encode information and can be accessed by the computing device 1500.

The memory 1512 can include computer-storage media in the form of volatile and/or nonvolatile memory. The memory 1512 may be removable, non-removable, or any combination thereof. Exemplary hardware devices are devices such as hard drives, solid-state memory, optical-disc drives, and the like. The computing device 1500 can include one or more processors that read data from components such as the memory 1512, the various I/O components 1516, etc. Presentation component(s) 1516 present data indications to a user or other device. Exemplary presentation components include a display device, speaker, printing component, vibrating component, etc.

The I/O ports 1518 can enable the computing device 1500 to be logically coupled to other devices, such as I/O components 1520. Some of the I/O components 1520 can be built into the computing device 1500. Examples of such I/O components 1520 include a microphone, joystick, recording device, game pad, satellite dish, scanner, printer, wireless device, networking device, and the like.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method of characterizing the metastastic potential of a mixture of cancer cells in vivo, the method comprising
(a) systemically delivering to a non-human subject the plurality of cancer cells, each cell comprising a vector encoding as a single transcript a barcode, a detectable marker suitable for in vivo imaging, and a detectable marker suitable for cell selection and/or sorting; and
(b) imaging the cells and their descendants subsequent to delivery to locate where in the body the cell and/or its descendants are present, thereby characterizing the metastatic potential.

2. The method of claim 1, further comprising allowing the plurality of cells to proliferate in the subject for a period of time selected from the group consisting of days, weeks, and months.

3. The method of claim 2, further comprising isolating the cells from the subject and characterizing the identity of the cells and their abundance.

4. The method of claim 3, further comprising sorting the isolated cells.

5. The method of claim 2, wherein the identity and quantity of the cells or the sorted cells is assessed by next-generation sequencing or quantitative PCR.

6. The method of claim 1, wherein single cell RNA sequencing is carried out on each cell, thereby generating a transcriptome for each cell.

7-9. (canceled)

10. The method of claim 1, wherein the marker suitable for imaging is a bioluminescent marker.

11. (canceled)

12. The method of claim 1, wherein the expression levels of the barcode, the detectable marker suitable for in vivo imaging, and the detectable marker suitable for cell selection and/or sorting are correlated.

13. The method of claim 12, wherein the abundance of the barcodes reflects the metastatic potentials of different cells.

14. (canceled)

15. A method of characterizing the metastastic potential of a mixture cancer cells in vivo, the method comprising
(a) systemically delivering to a non-human subject the plurality of cancer cells, each cell comprising a vector encoding a barcode; and
(b) subsequent to delivery detecting the bar code in a cell, tissue, or organ to determine where in the body the cell and/or its descendants are present, thereby characterizing the metastatic potential.

16-21. (canceled)

22. The method of claim 15, wherein single cell RNA sequencing is carried out on each cell, thereby generating a transcriptome for each cell.

23-27. (canceled)

28. A method of generating a metastasis map, the method comprising
(a) systemically delivering to a non-human subject a plurality of cells, each cell comprising a vector encoding as a single transcript a barcode, a detectable marker suitable for in vivo imaging, and a detectable marker suitable for cell selection and/or sorting; and
(b) detecting the cells and their descendants subsequent to delivery to identify where in the body the cell and/or its descendants are present;
(c) compiling the data of step (b) in a database; and
(d) associating the data with the cell's identity, thereby generating a metastasis map.

29-31. (canceled)

32. The method of claim 28, wherein single cell RNA sequencing is carried out on each cell, thereby generating a transcriptome for each cell and the data included in the metastasis map.

33-36. (canceled)

37. The method of claim 28, wherein the data is used to generate a metastasis map that includes a visual representation of the anatomical position of the cells and their proliferation over time.

38. The method of claim 28, wherein drug sensitivity data, CRISPR knockout viability data, shRNA knockdown data, annotated cell line data, a metabolite profile, a genomic profile, a transcriptomic profile, or a proteomic profile is included as an interactive feature within the visual representation.

39. A method of generating a metastasis map, the method comprising
(a) systemically delivering to a non-human subject a plurality of cells, each cell comprising a vector encoding as a barcode;
(b) detecting and quantitating expression of the barcode;
(c) compiling the expression data in a database; and
(d) associating the expression data with the cell's identity, thereby generating a metastasis map.

40-49. (canceled)

50. The method of claim 39, wherein drug sensitivity data, CRISPR knockout viability data, shRNA knockdown data, annotated cell line data, a metabolite profile, a genomic profile, a transcriptomic profile, or a proteomic profile is included in the metastasis map.

51. (canceled)

52. A vector comprising a single transcription cassette comprising a detectable marker suitable for cell selection and/or sorting, a marker suitable for imaging a cell in vivo, and a barcode.

53-57. (canceled)

58. A method for identifying the molecular features characteristic of a metastatic cell, the method comprising using the metastasis map generated using the method of claim 1 to identify organ-specific patterns of metastasis.

59-60. (canceled)

61. A computer implemented method of generating a metastasis map quantifying metastatic potential, the method comprising:
receiving, by a processor, a listing of vectors encoded as barcodes, the vectors being associated with a plurality of cells systemically delivered to a non-human subject;
receiving, from an imaging device, images of the plurality of cells and their descendants within the non-human subject;
storing, by the processor, the images of the plurality of cells and their descendants in a database;
identifying, by the processor, locations of the plurality of cells and their descendants from the images using the barcodes; and
generating, by the processor, the metastasis map based on the locations of the plurality of cells and their descendants.

62-72. (canceled)

73. A system for generating a metastasis map quantifying metastatic potential, the system comprising:
a CPU, a computer readable memory and a computer readable storage medium;
program instructions to receive a listing of vectors encoded as barcodes, the vectors being associated with a plurality of cells systemically delivered to a non-human subject;
program instructions to receive images of the plurality of cells and their descendants within the non-human subject from an imaging device;
program instructions to store the images of the plurality of cells and their descendants in a database;
program instructions to identify locations of the plurality of cells and their descendants from the images using the barcodes; and
program instructions to generate the metastasis map based on the locations of the plurality of cells and their descendants.
